SEQUENCE LISTING (911 sequences)

SEQ ID NO: 1 - DNA, 2175 bp, Adeno-associated virus 5
  source 1..2175: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 2 - protein, 724 aa, Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 3 - DNA, 2211 bp, Adeno-associated virus 9
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 4 - protein, 736 aa, Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 20 aa, synthetic construct
SSQAGIPVRAPRSPRAPEPA

SEQ ID NO: 6 - protein, 20 aa, synthetic construct
TSRMSTSQIPGRVASSGLQS

SEQ ID NO: 7 - protein, 20 aa, synthetic construct
KQLNQQHSQIKEIENQLRRT

SEQ ID NO: 8 - protein, 20 aa, synthetic construct
VLGAPRAGSGAHTAVISPQD

SEQ ID NO: 9 - protein, 20 aa, synthetic construct
QGASVNSSSQTAAQVTKGVK

SEQ ID NO: 10 - protein, 20 aa, synthetic construct
KALAAAGYDVEKNNSRIKLS

SEQ ID NO: 11 - protein, 20 aa, synthetic construct
LEALSAGPGTSGSHLDRSTV

SEQ ID NO: 12 - protein, 20 aa, synthetic construct
TSTMPNSSQSSHSSSGTLSS

SEQ ID NO: 13 - protein, 20 aa, synthetic construct
GIKSLVSKGTLVQTKGTGAS

SEQ ID NO: 14 - protein, 20 aa, synthetic construct
VVYTGHDTKLMQNSTKAPLK

SEQ ID NO: 15 - protein, 20 aa, synthetic construct
STMPNSSQSSHSSSGTLSSL

SEQ ID NO: 16 - protein, 20 aa, synthetic construct
RNNMIQTKPTGTQQSTNTAV

SEQ ID NO: 17 - protein, 20 aa, synthetic construct
HDTKLMQNSTKAPLKRSNVE

SEQ ID NO: 18 - protein, 20 aa, synthetic construct
ANGDKEIGNIISDAMKKVGR

SEQ ID NO: 19 - protein, 20 aa, synthetic construct
AGLTRISKEILTQGQINSTS

SEQ ID NO: 20 - protein, 20 aa, synthetic construct
QTAHVGVGISGNEGMQATNN

SEQ ID NO: 21 - protein, 20 aa, synthetic construct
SKYVNKEIQNAVNGVKQIKT

SEQ ID NO: 22 - protein, 20 aa, synthetic construct
NHFGTASSTGNLVVKDPTRV

SEQ ID NO: 23 - protein, 20 aa, synthetic construct
TAHVGVGISGNEGMQATNNS

SEQ ID NO: 24 - protein, 20 aa, synthetic construct
EQLGGTVTNPGGSGTSSRLE

SEQ ID NO: 25 - protein, 20 aa, synthetic construct
YNSAGTGPSSATVNVTTRKP

SEQ ID NO: 26 - protein, 20 aa, synthetic construct
ALAAGGYDVEKNNSRIKLGL

SEQ ID NO: 27 - protein, 20 aa, synthetic construct
VDFRIHVENQTRARDDVSRK

SEQ ID NO: 28 - protein, 20 aa, synthetic construct
LSLTQSDISHIGSMRVEGIV

SEQ ID NO: 29 - protein, 20 aa, synthetic construct
SDGSLEDDDYHATTETIITM

SEQ ID NO: 30 - protein, 20 aa, synthetic construct
DFRIHVENQTRARDDVSRKQ

SEQ ID NO: 31 - protein, 20 aa, synthetic construct
SSRGTTGNTQVQSPSNATTA

SEQ ID NO: 32 - protein, 20 aa, synthetic construct
HVAMEMKEDINTKKEDMMFH

SEQ ID NO: 33 - protein, 20 aa, synthetic construct
AGVAHAITAACTQGNLSDCG

SEQ ID NO: 34 - protein, 20 aa, synthetic construct
RGTTGNTQVQSPSNATTATG

SEQ ID NO: 35 - protein, 20 aa, synthetic construct
AIAAGATARATTASRLPSSA

SEQ ID NO: 36 - protein, 20 aa, synthetic construct
GSVGTQGRACNKTAPQASGC

SEQ ID NO: 37 - protein, 20 aa, synthetic construct
GTTGNTQVQSPSNATTATGV

SEQ ID NO: 38 - protein, 20 aa, synthetic construct
TSPATPSAGGAASSRTPNRI

SEQ ID NO: 39 - protein, 20 aa, synthetic construct
AEDVRGNLRGNTEGLQKSLA

SEQ ID NO: 40 - protein, 20 aa, synthetic construct
IKVSNTLESRLDLSAKQKMP

SEQ ID NO: 41 - protein, 20 aa, synthetic construct
SPATPSAGGAASSRTPNRIS

SEQ ID NO: 42 - protein, 20 aa, synthetic construct
QPAVVHLQGQGSAIQVKNDL

SEQ ID NO: 43 - protein, 20 aa, synthetic construct
VSSLPTTNSTTNSATKITAN

SEQ ID NO: 44 - protein, 20 aa, synthetic construct
QDAASSSSSSSSSATTTTPE

SEQ ID NO: 45 - protein, 20 aa, synthetic construct
NNGSMRNTSQDREREVDNNR

SEQ ID NO: 46 - protein, 20 aa, synthetic construct
SLPTTNSTTNSATKITANTT

SEQ ID NO: 47 - protein, 20 aa, synthetic construct
SLKASSTMAKSENLVKSHVQ

SEQ ID NO: 48 - protein, 20 aa, synthetic construct
KENGGARLAEYHAKATEHLS

SEQ ID NO: 49 - protein, 20 aa, synthetic construct
AVSRVADTIMSGPSNSQQVP

SEQ ID NO: 50 - protein, 20 aa, synthetic construct
KASSTMAKSENLVKSHVQLQ

SEQ ID NO: 51 - protein, 20 aa, synthetic construct
KQINDYVEKGTQGKIVDLVK

SEQ ID NO: 52 - protein, 20 aa, synthetic construct
MIVAAIQAEKNRAGSSRQSI

SEQ ID NO: 53 - protein, 20 aa, synthetic construct
SSTMAKSENLVKSHVQLQNY

SEQ ID NO: 54 - protein, 20 aa, synthetic construct
SNAIKNLPPPLGGAAGHPGS

SEQ ID NO: 55 - protein, 20 aa, synthetic construct
KSEVSTDLIQANPKQGALAK

SEQ ID NO: 56 - protein, 20 aa, synthetic construct
STMAKSENLVKSHVQLQNYS

SEQ ID NO: 57 - protein, 20 aa, synthetic construct
PEFSNDIKKRKVDDKDSSHY

SEQ ID NO: 58 - protein, 20 aa, synthetic construct
YGGGATNANARAKGGTGQTG

SEQ ID NO: 59 - protein, 20 aa, synthetic construct
TMAKSENLVKSHVQLQNYSK

SEQ ID NO: 60 - protein, 20 aa, synthetic construct
FSNDIKKRKVDDKDSSHYDS

SEQ ID NO: 61 - protein, 20 aa, synthetic construct
GGATNANARAKGGTGQTGGL

SEQ ID NO: 62 - protein, 20 aa, synthetic construct
SALAGQRQGTQAESNLSSKF

SEQ ID NO: 63 - protein, 20 aa, synthetic construct
NVINNAILRANLQKGENPSH

SEQ ID NO: 64 - protein, 20 aa, synthetic construct
ATNANARAKGGTGQTGGLTQ

SEQ ID NO: 65 - protein, 20 aa, synthetic construct
ALAGQRQGTQAESNLSSKFQ

SEQ ID NO: 66 - protein, 20 aa, synthetic construct
VASVSLLQLGQETDKLKTRN

SEQ ID NO: 67 - protein, 20 aa, synthetic construct
ISSDSIGVLGYQKTVDHTKV

SEQ ID NO: 68 - protein, 20 aa, synthetic construct
FQFSSNKEQNGVQDPQHERI

SEQ ID NO: 69 - protein, 20 aa, synthetic construct
EVHNAKTKPREEQYNSTYRV

SEQ ID NO: 70 - protein, 20 aa, synthetic construct
PLTPANNLPDTQSSGPAHSK

SEQ ID NO: 71 - protein, 20 aa, synthetic construct
SSFQGSKRGLNSSFETSPKK

SEQ ID NO: 72 - protein, 20 aa, synthetic construct
AARPYSATAAAATGGMANVK

SEQ ID NO: 73 - protein, 20 aa, synthetic construct
TSVFLKNNKIAGVNNRTEYV

SEQ ID NO: 74 - protein, 20 aa, synthetic construct
VEVHNAKTKPREEQFNSTYR

SEQ ID NO: 75 - protein, 20 aa, synthetic construct
TNSNNHLAADSQAYDQKNIR

SEQ ID NO: 76 - protein, 20 aa, synthetic construct
LKNNKIAGVNNRTEYVETTS

SEQ ID NO: 77 - protein, 20 aa, synthetic construct
EFSVDETGQMSATAKGRVRL

SEQ ID NO: 78 - protein, 20 aa, synthetic construct
KRSSQSTLERSEQQIRAASS

SEQ ID NO: 79 - protein, 20 aa, synthetic construct
APANTHNNNIEGQAIVYQNK

SEQ ID NO: 80 - protein, 20 aa, synthetic construct
AIVKANANAAAINNLASSIQ

SEQ ID NO: 81 - protein, 20 aa, synthetic construct
RFVQNALNGNGDPNNMDKAV

SEQ ID NO: 82 - protein, 20 aa, synthetic construct
KNTALDKNYLLNNKIPSNNV

SEQ ID NO: 83 - protein, 20 aa, synthetic construct
NAVARVADTIGSGPSNSQAV

SEQ ID NO: 84 - protein, 20 aa, synthetic construct
PLTPVNNLPDTRSSGPAHSK

SEQ ID NO: 85 - protein, 20 aa, synthetic construct
LGIEQGVGVPGGSPGSRHRQ

SEQ ID NO: 86 - protein, 20 aa, synthetic construct
YISFNPKENKITVKSKNLPE

SEQ ID NO: 87 - protein, 20 aa, synthetic construct
SATLNSVLNSNAIKNLPPPL

SEQ ID NO: 88 - protein, 20 aa, synthetic construct
GGGGPGGSGGVGGGSGGYTY

SEQ ID NO: 89 - protein, 20 aa, synthetic construct
MAKSENLVKSHVQLQNYSKV

SEQ ID NO: 90 - protein, 20 aa, synthetic construct
VTSGKSIDDNSTKSEKQTPR

SEQ ID NO: 91 - protein, 20 aa, synthetic construct
SAGPGTSGSHLDRSTVSSFS

SEQ ID NO: 92 - protein, 20 aa, synthetic construct
MIETYNQTSPRSAATGLPIS

SEQ ID NO: 93 - protein, 20 aa, synthetic construct
VATKGLGVHAKSSDWGLQAG

SEQ ID NO: 94 - protein, 20 aa, synthetic construct
AARDSLSLSSGSGGGSASAS

SEQ ID NO: 95 - protein, 20 aa, synthetic construct
LPEAVLALYNSTRDRVAGES

SEQ ID NO: 96 - protein, 20 aa, synthetic construct
LENNKTMNRAENGGRPPHHP

SEQ ID NO: 97 - protein, 20 aa, synthetic construct
ILTQGQINSTSGLNKESFRY

SEQ ID NO: 98 - protein, 20 aa, synthetic construct
HCRQEATNATRVIHDNYDQL

SEQ ID NO: 99 - protein, 20 aa, synthetic construct
MWVDDQEVINKASNSNKIRL

SEQ ID NO: 100 - protein, 20 aa, synthetic construct
RALGTSPELPTGVTGSSGTR

SEQ ID NO: 101 - protein, 20 aa, synthetic construct
VTNNVKDVTKLVANLPKDYM

SEQ ID NO: 102 - protein, 20 aa, synthetic construct
KVQGLVPAGGSSSNSTREPP

SEQ ID NO: 103 - protein, 20 aa, synthetic construct
LKENGGARLAEYHAKATEHL

SEQ ID NO: 104 - protein, 20 aa, synthetic construct
NSVLNSNAIKNLPPPLGGAA

SEQ ID NO: 105 - protein, 20 aa, synthetic construct
GIVVYTGHDTKLMQNSTKAP

SEQ ID NO: 106 - protein, 20 aa, synthetic construct
GLRGGAGPPGPEGGKGAAGP

SEQ ID NO: 107 - protein, 20 aa, synthetic construct
EPPPELENNKTMNRAENGGR

SEQ ID NO: 108 - protein, 20 aa, synthetic construct
ETGWTPGLRSLENDGTKLLD

SEQ ID NO: 109 - protein, 20 aa, synthetic construct
RTLVSSKPQAHGAPAPPSGS

SEQ ID NO: 110 - protein, 20 aa, synthetic construct
ATLPQCQAANKTCPTNYMWN

SEQ ID NO: 111 - protein, 20 aa, synthetic construct
WETGAVPGLRSLENDGTRLL

SEQ ID NO: 112 - protein, 20 aa, synthetic construct
VDNEDDHGRGDLGRLKKQKD

SEQ ID NO: 113 - protein, 20 aa, synthetic construct
KVVPEGIRMNKTVAVRTLDP

SEQ ID NO: 114 - protein, 20 aa, synthetic construct
WRSRYPHKPEINSTTHPGAD

SEQ ID NO: 115 - protein, 20 aa, synthetic construct
MGDMANSSIEFHPKPQQQRD

SEQ ID NO: 116 - protein, 20 aa, synthetic construct
GSHGTQDRQCNKTSNGSDSC

SEQ ID NO: 117 - protein, 20 aa, synthetic construct
NFSAAFSVNYDTKSGPKNMT

SEQ ID NO: 118 - protein, 20 aa, synthetic construct
EGTICKRARGDDMDDYCNGK

SEQ ID NO: 119 - protein, 20 aa, synthetic construct
LLREGNTVRSFRAAAAETLE

SEQ ID NO: 120 - protein, 20 aa, synthetic construct
TSGLLETNFTASARTTGSGL

SEQ ID NO: 121 - protein, 20 aa, synthetic construct
QDNTCEEYHTVGPHSCHIPK

SEQ ID NO: 122 - protein, 20 aa, synthetic construct
LPGDPTSNATSRGAEGPLPP

SEQ ID NO: 123 - protein, 20 aa, synthetic construct
QRSGASASGVYTIQVSNATK

SEQ ID NO: 124 - protein, 20 aa, synthetic construct
GSSGTGGTATWKPGSSGPGS

SEQ ID NO: 125 - protein, 20 aa, synthetic construct
TLGADLVDSTDNTKINRGIF

SEQ ID NO: 126 - protein, 20 aa, synthetic construct
RTSGLLETNFTASARTTGSG

SEQ ID NO: 127 - protein, 20 aa, synthetic construct
LQGAGNSAAGRGAIADTFRS

SEQ ID NO: 128 - protein, 20 aa, synthetic construct
WNRQSSTSVIETNKTSVELS

SEQ ID NO: 129 - protein, 20 aa, synthetic construct
VPEGIRMNKTVAVRTLDPER

SEQ ID NO: 130 - protein, 20 aa, synthetic construct
MKGASRSPEDSSPDAARIRV

SEQ ID NO: 131 - protein, 20 aa, synthetic construct
GSFGTRDRTCNVTSHGIDGC

SEQ ID NO: 132 - protein, 20 aa, synthetic construct
EHGAVSQEEVIRAYDTTKKK

SEQ ID NO: 133 - protein, 20 aa, synthetic construct
GAPRAGSGAHTAVISPQDPT

SEQ ID NO: 134 - protein, 20 aa, synthetic construct
SIGVLGYQKTVDHTKVNSKL

SEQ ID NO: 135 - protein, 20 aa, synthetic construct
SNRVRVSIQTGTAFQPDAAM

SEQ ID NO: 136 - protein, 20 aa, synthetic construct
TFFRGLESQTKLVNGQSHIS

SEQ ID NO: 137 - protein, 20 aa, synthetic construct
AGVVCTNETRSTHTLDLSRE

SEQ ID NO: 138 - protein, 20 aa, synthetic construct
FNDMANAAKGLEWISNKISK

SEQ ID NO: 139 - protein, 20 aa, synthetic construct
GSAGPPGATGFPGAAGRVGP

SEQ ID NO: 140 - protein, 20 aa, synthetic construct
LFGTTISNNIKYGRDDVTDE

SEQ ID NO: 141 - protein, 20 aa, synthetic construct
VAEIPFNSTNKYQLSIHETE

SEQ ID NO: 142 - protein, 20 aa, synthetic construct
MSTSQIPGRVASSGLQSVVH

SEQ ID NO: 143 - protein, 20 aa, synthetic construct
EILELAGNASRDNKKTRIIP

SEQ ID NO: 144 - protein, 20 aa, synthetic construct
RTILPMDQEFTYRVKNREQA

SEQ ID NO: 145 - protein, 20 aa, synthetic construct
TPGPQGIAGQRGVVGLPGQR

SEQ ID NO: 146 - protein, 20 aa, synthetic construct
GTAGRVCNLTSRGMDSCEVM

SEQ ID NO: 147 - protein, 20 aa, synthetic construct
LTATLGADLVDSTDNTKINR

SEQ ID NO: 148 - protein, 20 aa, synthetic construct
VEGTARGVVVATGDRTVMGR

SEQ ID NO: 149 - protein, 20 aa, synthetic construct
YWGSVTGSQSNAVSPTPAPR

SEQ ID NO: 150 - protein, 20 aa, synthetic construct
VEVGGSAKYLNNTKKYQNQS

SEQ ID NO: 151 - protein, 20 aa, synthetic construct
PFPGLGTAAAPAQGGAHLKQ

SEQ ID NO: 152 - protein, 20 aa, synthetic construct
FNSHINASNNSEPSRMNMTS

SEQ ID NO: 153 - protein, 20 aa, synthetic construct
QSAKDNNIKFRAIGHSDNSV

SEQ ID NO: 154 - protein, 20 aa, synthetic construct
ARARELSSGQKDQGQAANML

SEQ ID NO: 155 - protein, 20 aa, synthetic construct
ENNKTMNRAENGGRPPHHPF

SEQ ID NO: 156 - protein, 20 aa, synthetic construct
PELENNKTMNRAENGGRPPH

SEQ ID NO: 157 - protein, 20 aa, synthetic construct
KPQKQLTAQSTPSTSGVNSQ

SEQ ID NO: 158 - protein, 20 aa, synthetic construct
KHSTIFENLANKADRDQYEL

SEQ ID NO: 159 - protein, 20 aa, synthetic construct
DNRTRQHIPYREDKNLTGTA

SEQ ID NO: 160 - protein, 20 aa, synthetic construct
RLEALKENGGARLAEYHAKA

SEQ ID NO: 161 - protein, 20 aa, synthetic construct
PPPELENNKTMNRAENGGRP

SEQ ID NO: 162 - protein, 20 aa, synthetic construct
PRGAAPPGQKGQANATRIPA

SEQ ID NO: 163 - protein, 20 aa, synthetic construct
GVDNEDDHGRGDLGRLKKQK

SEQ ID NO: 164 - protein, 20 aa, synthetic construct
GGSSSNSTREPPASGRVALV

SEQ ID NO: 165 - protein, 20 aa, synthetic construct
MLTCPEANATKVCGSDGVTY

SEQ ID NO: 166 - protein, 20 aa, synthetic construct
TAGSAMGAASLTLSAQSRTL

SEQ ID NO: 167 - protein, 20 aa, synthetic construct
LNSVLNSNAIKNLPPPLGGA

SEQ ID NO: 168 - protein, 20 aa, synthetic construct
TWLVRHTENVTKTKTPEDYA

SEQ ID NO: 169 - protein, 20 aa, synthetic construct
GRPGLPGAAGARGNDGARGS

SEQ ID NO: 170 - protein, 20 aa, synthetic construct
YRWNRQSSTSVIETNKTSVE

SEQ ID NO: 171 - protein, 20 aa, synthetic construct
TGHDTKLMQNSTKAPLKRSN

SEQ ID NO: 172 - protein, 20 aa, synthetic construct
VRHIPDRGAPTRASEPASAA

SEQ ID NO: 173 - protein, 20 aa, synthetic construct
CVVADGNSTRSPETNGLLCG

SEQ ID NO: 174 - protein, 20 aa, synthetic construct
SQTIPQISNRTLNTLVQDNS

SEQ ID NO: 175 - protein, 20 aa, synthetic construct
PSSAGSWNSGSSGPGSTGNR

SEQ ID NO: 176 - protein, 20 aa, synthetic construct
MIQTLTNKSKIQNVGGTYEL

SEQ ID NO: 177 - protein, 20 aa, synthetic construct
GSSSNSTREPPASGRVALVK

SEQ ID NO: 178 - protein, 20 aa, synthetic construct
RGSSSRQSSSSAMSSSSASS

SEQ ID NO: 179 - protein, 20 aa, synthetic construct
CRHERDAGVVCTNETRSTHT

SEQ ID NO: 180 - protein, 20 aa, synthetic construct
SDSIGVLGYQKTVDHTKVNS

SEQ ID NO: 181 - protein, 20 aa, synthetic construct
DLSGITGARNLAVSQVVHKA

SEQ ID NO: 182 - protein, 20 aa, synthetic construct
LLGTCDQAPKSGSHEVRCNL

SEQ ID NO: 183 - protein, 20 aa, synthetic construct
LVRHTENVTKTKTPEDYADS

SEQ ID NO: 184 - protein, 20 aa, synthetic construct
VYADSFVIRGDEVRQIAPGQ

SEQ ID NO: 185 - protein, 20 aa, synthetic construct
SHGTQDRQCNKTSNGSDSCD

SEQ ID NO: 186 - protein, 20 aa, synthetic construct
PELKTPLTANITKSGNTFRP

SEQ ID NO: 187 - protein, 20 aa, synthetic construct
VRLVLTNPEGRKEGKEVTFQ

SEQ ID NO: 188 - protein, 20 aa, synthetic construct
TQGRACNKTAPQASGCDLMC

SEQ ID NO: 189 - protein, 20 aa, synthetic construct
GTQGRECNRTSEGADGCNLL

SEQ ID NO: 190 - protein, 20 aa, synthetic construct
DKMFSQNDTRCAGPEAVKGQ

SEQ ID NO: 191 - protein, 20 aa, synthetic construct
GLANGVRRYASQTELYDAPG

SEQ ID NO: 192 - protein, 20 aa, synthetic construct
LPKPQSNLPDTKASGPAHSK

SEQ ID NO: 193 - protein, 20 aa, synthetic construct
SSDSNNLKLNNVRLPRENMS

SEQ ID NO: 194 - protein, 20 aa, synthetic construct
LERAASRANTVRSFHHEESL

SEQ ID NO: 195 - protein, 20 aa, synthetic construct
TVQAARTPGSLEGGSQPWRP

SEQ ID NO: 196 - protein, 20 aa, synthetic construct
TDANQLWTLKRDNTIRSNGK

SEQ ID NO: 197 - protein, 20 aa, synthetic construct
LLTVTSGKSIDDNSTKSEKQ

SEQ ID NO: 198 - protein, 20 aa, synthetic construct
GAGVAGRITGSMSLASGKSL

SEQ ID NO: 199 - protein, 20 aa, synthetic construct
EKYIDQEELNKTKPIWTRNP

SEQ ID NO: 200 - protein, 20 aa, synthetic construct
ENVDFRIHVENQTRARDDVS

SEQ ID NO: 201 - protein, 20 aa, synthetic construct
TAGAAMGAASLTLSAQSRTL

SEQ ID NO: 202 - protein, 20 aa, synthetic construct
ASSTMAKSENLVKSHVQLQN

SEQ ID NO: 203 - protein, 20 aa, synthetic construct
PEAVLALYNSTRDRVAGESA

SEQ ID NO: 204 - protein, 20 aa, synthetic construct
ENGGARLAEYHAKATEHLST

SEQ ID NO: 205 - protein, 20 aa, synthetic construct
LGTAGRVCNLTSRGMDSCEV

SEQ ID NO: 206 - protein, 20 aa, synthetic construct
PGDPTSNATSRGAEGPLPPP

SEQ ID NO: 207 - protein, 20 aa, synthetic construct
AARLEALKENGGARLAEYHA

SEQ ID NO: 208 - protein, 20 aa, synthetic construct
GTQDRQCNKTSNGSDSCDLM

SEQ ID NO: 209 - protein, 20 aa, synthetic construct
EFSNDIKKRKVDDKDSSHYD

SEQ ID NO: 210 - protein, 20 aa, synthetic construct
SLAGRAPAGAAAVSASGHGR

SEQ ID NO: 211 - protein, 20 aa, synthetic construct
IAPVVSSRVSPGTRPTAMGS

SEQ ID NO: 212 - protein, 20 aa, synthetic construct
LREGNTVRSFRAAAAETLER

SEQ ID NO: 213 - protein, 20 aa, synthetic construct
VEGKVQGLVPAGGSSSNSTR

SEQ ID NO: 214 - protein, 20 aa, synthetic construct
PAGGSSSNSTREPPASGRVA

SEQ ID NO: 215 - protein, 20 aa, synthetic construct
AKSENLVKSHVQLQNYSKVE

SEQ ID NO: 216 - protein, 20 aa, synthetic construct
STSTMPNSSQSSHSSSGTLS

SEQ ID NO: 217 - protein, 20 aa, synthetic construct
GPEFSNDIKKRKVDDKDSSH

SEQ ID NO: 218 - protein, 20 aa, synthetic construct
RMDIDVVEKTNKIKDRYWDS

SEQ ID NO: 219 - protein, 20 aa, synthetic construct
PKPTSNVHVSVAGSHSQAAN

SEQ ID NO: 220 - protein, 20 aa, synthetic construct
QQPYITNRTFMLAKEASLAD

SEQ ID NO: 221 - protein, 20 aa, synthetic construct
RALTQALPAPTGQNTQVSSH

SEQ ID NO: 222 - protein, 20 aa, synthetic construct
RDSPHSERYVSALTTPARLS

SEQ ID NO: 223 - protein, 20 aa, synthetic construct
EAVLALYNSTRDRVAGESAE

SEQ ID NO: 224 - protein, 20 aa, synthetic construct
PSTDAVLGNSTAVNRGDSSI

SEQ ID NO: 225 - protein, 20 aa, synthetic construct
LAGQRQGTQAESNLSSKFQF

SEQ ID NO: 226 - protein, 20 aa, synthetic construct
KLTDMEAQLLNQTSRMDAQM

SEQ ID NO: 227 - protein, 20 aa, synthetic construct
TITTNEIRNKLSYSFDGAGG

SEQ ID NO: 228 - protein, 20 aa, synthetic construct
RGGSQSVYGVDQTHSGLPLH

SEQ ID NO: 229 - protein, 20 aa, synthetic construct
GQQYPGQGPPSGQPPYGGHQ

SEQ ID NO: 230 - protein, 20 aa, synthetic construct
LALAPNSTARRQLASLLEGT

SEQ ID NO: 231 - protein, 20 aa, synthetic construct
IVGDAGVRGTAQQPRLFVGM

SEQ ID NO: 232 - protein, 20 aa, synthetic construct
AEILELAGNASRDNKKTRII

SEQ ID NO: 233 - protein, 20 aa, synthetic construct
GDADNDMVKIENLTKVYKSR

SEQ ID NO: 234 - protein, 20 aa, synthetic construct
KTPLTANITKSGNTFRPEVH

SEQ ID NO: 235 - protein, 20 aa, synthetic construct
VTWLVRHTENVTKTKTPEDY

SEQ ID NO: 236 - protein, 20 aa, synthetic construct
PGTQGRECNRTSEGADGCNL

SEQ ID NO: 237 - protein, 20 aa, synthetic construct
RVTNNVKDVTKLVANLPKDY

SEQ ID NO: 238 - protein, 20 aa, synthetic construct
SMGLQLNLTYERKDNTTVTR

SEQ ID NO: 239 - protein, 20 aa, synthetic construct
SFVIRGDEVRQIAPGQTGKI

SEQ ID NO: 240 - protein, 20 aa, synthetic construct
VGTQGRLCNRTSPGADGCDT

SEQ ID NO: 241 - protein, 20 aa, synthetic construct
YISNRVRVSIQTGTAFQPDA

SEQ ID NO: 242 - protein, 20 aa, synthetic construct
GRNVQEERSAGATANLPLRS

SEQ ID NO: 243 - protein, 20 aa, synthetic construct
YLTAEILELAGNASRDNKKT

SEQ ID NO: 244 - protein, 20 aa, synthetic construct
SSSNSTREPPASGRVALVKV

SEQ ID NO: 245 - protein, 20 aa, synthetic construct
IGNVGAPGAKGARGSAGPPG

SEQ ID NO: 246 - protein, 20 aa, synthetic construct
IRKLTDMEAQLLNQTSRMDA

SEQ ID NO: 247 - protein, 20 aa, synthetic construct
DMIQTLTNKSKIQNVGGTYE

SEQ ID NO: 248 - protein, 20 aa, synthetic construct
LLQGAGNSAAGRGAIADTFR

SEQ ID NO: 249 - protein, 20 aa, synthetic construct
SLGTAGRVCNLTSRGMDSCE

SEQ ID NO: 250 - protein, 20 aa, synthetic construct
VLALYNSTRDRVAGESAEPE

SEQ ID NO: 251 - protein, 20 aa, synthetic construct
EPYADQLRTQVNTQAEQLRR

SEQ ID NO: 252 - protein, 20 aa, synthetic construct
HPELKTPLTANITKSGNTFR

SEQ ID NO: 253 - protein, 20 aa, synthetic construct
YLGTSLGNITRDQKILNPSA

SEQ ID NO: 254 - protein, 20 aa, synthetic construct
LSVGRGGSPRLQPDSRALHY

SEQ ID NO: 255 - protein, 20 aa, synthetic construct
NSTWDPQSNSIAKYNYPNGK

SEQ ID NO: 256 - protein, 20 aa, synthetic construct
DVLYAGHNSTWDPQSNSIAK

SEQ ID NO: 257 - protein, 20 aa, synthetic construct
AAGSTMGAASMTLTVQARQL

SEQ ID NO: 258 - protein, 20 aa, synthetic construct
VKIQRVNNSSTNDNLVRKND

SEQ ID NO: 259 - protein, 20 aa, synthetic construct
LTVTSGKSIDDNSTKSEKQT

SEQ ID NO: 260 - protein, 20 aa, synthetic construct
AAMGAASLTLSAQSRTLLAG

SEQ ID NO: 261 - protein, 20 aa, synthetic construct
QAAELQNDVRQAVEGAIGRV

SEQ ID NO: 262 - protein, 20 aa, synthetic construct
NNRGKANSTGTLVITDPTRI

SEQ ID NO: 263 - protein, 20 aa, synthetic construct
GATNANARAKGGTGQTGGLT

SEQ ID NO: 264 - protein, 20 aa, synthetic construct
VQGLVPAGGSSSNSTREPPA

SEQ ID NO: 265 - protein, 20 aa, synthetic construct
RWNRQSSTSVIETNKTSVEL

SEQ ID NO: 266 - protein, 20 aa, synthetic construct
VLVEAAASQRSSSLASNRLK

SEQ ID NO: 267 - protein, 20 aa, synthetic construct
LDLNMTRSHSGGELESLARG

SEQ ID NO: 268 - protein, 20 aa, synthetic construct
KSALVLSAESSSMGGTLTVQ

SEQ ID NO: 269 - protein, 20 aa, synthetic construct
RRAAPEASGTPSSDAVSRLE

SEQ ID NO: 270 - protein, 20 aa, synthetic construct
WKAAIPSALDTNSSKSTSSF

SEQ ID NO: 271 - protein, 20 aa, synthetic construct
RITMTNSVDDALINSTKIYS

SEQ ID NO: 272 - protein, 20 aa, synthetic construct
VVSSRVSPGTRPTAMGSFSS

SEQ ID NO: 273 - protein, 20 aa, synthetic construct
EENKIRQEDLTKIISSAQKV

SEQ ID NO: 274 - protein, 20 aa, synthetic construct
IPSALDTNSSKSTSSFPCPA

SEQ ID NO: 275 - protein, 20 aa, synthetic construct
TVSGKITHSPQAHVNPQEKR

SEQ ID NO: 276 - protein, 20 aa, synthetic construct
LLLAGDRASSNPNATSSSSQ

SEQ ID NO: 277 - protein, 20 aa, synthetic construct
EISSDSIGVLGYQKTVDHTK

SEQ ID NO: 278 - protein, 20 aa, synthetic construct
FKGVDNEDDHGRGDLGRLKK

SEQ ID NO: 279 - protein, 20 aa, synthetic construct
VLNSNAIKNLPPPLGGAAGH

SEQ ID NO: 280 - protein, 20 aa, synthetic construct
QDNKIVVKNATKSNVNNAVN

SEQ ID NO: 281 - protein, 20 aa, synthetic construct
LTGRQDTSRMSTSQIPGRVA

SEQ ID NO: 282 - protein, 20 aa, synthetic construct
PFNHIKSSLLGSTSDSNLNK

SEQ ID NO: 283 - protein, 20 aa, synthetic construct
LPATLPQCQAANKTCPTNYM

SEQ ID NO: 284 - protein, 20 aa, synthetic construct
SRMSTSQIPGRVASSGLQSV

SEQ ID NO: 285 - protein, 20 aa, synthetic construct
SAISNLIRKHVSEARLVEDI

SEQ ID NO: 286 - protein, 20 aa, synthetic construct
NRTSGLLETNFTASARTTGS

SEQ ID NO: 287 - protein, 20 aa, synthetic construct
GRQDTSRMSTSQIPGRVASS

SEQ ID NO: 288 - protein, 20 aa, synthetic construct
QRVNNSSTNDNLVRKNDQVY

SEQ ID NO: 289 - protein, 20 aa, synthetic construct
VLFGTTISNNIKYGRDDVTD

SEQ ID NO: 290 - protein, 20 aa, synthetic construct
LGGTVTNPGGSGTSSRLEPR

SEQ ID NO: 291 - protein, 20 aa, synthetic construct
TAAHPELKTPLTANITKSGN

SEQ ID NO: 292 - protein, 20 aa, synthetic construct
GKSIDDNSTKSEKQTPREHS

SEQ ID NO: 293 - protein, 20 aa, synthetic construct
ARLEALKENGGARLAEYHAK

SEQ ID NO: 294 - protein, 20 aa, synthetic construct
LKASSTMAKSENLVKSHVQL

SEQ ID NO: 295 - protein, 20 aa, synthetic construct
VVLANASRIHYDGGAGTNTV

SEQ ID NO: 296 - protein, 20 aa, synthetic construct
ATAGSAMGAASLTLSAQSRT

SEQ ID NO: 297 - protein, 20 aa, synthetic construct
ELKTPLTANITKSGNTFRPE

SEQ ID NO: 298 - protein, 20 aa, synthetic construct
FATDRTSMPSANIIRSFKNE

SEQ ID NO: 299 - protein, 20 aa, synthetic construct
PLPSVTKLTEDRWNEHQKMQ

SEQ ID NO: 300 - protein, 20 aa, synthetic construct
YSLKASSTMAKSENLVKSHV

SEQ ID NO: 301 - protein, 20 aa, synthetic construct
TSGKSIDDNSTKSEKQTPRE

SEQ ID NO: 302 - protein, 20 aa, synthetic construct
STYMAASGLNASTYDQVGRS

SEQ ID NO: 303 - protein, 20 aa, synthetic construct
IGVLGYQKTVDHTKVNSKLS

SEQ ID NO: 304 - protein, 20 aa, synthetic construct
KEGGPDHNYTKEKIKIVEGI

SEQ ID NO: 305 - protein, 20 aa, synthetic construct
SYSQMQSQAGGNNTGSTPLR

SEQ ID NO: 306 - protein, 20 aa, synthetic construct
SSMSNHTKERVTMTKVTLEN

SEQ ID NO: 307 - protein, 20 aa, synthetic construct
KPCAPGTFSNTTSSTDICRP

SEQ ID NO: 308 - protein, 20 aa, synthetic construct
AATLEQPASSSHAQGTHLRL

SEQ ID NO: 309 - protein, 20 aa, synthetic construct
NDWKHNITQREARALELASQ

SEQ ID NO: 310 - protein, 20 aa, synthetic construct
SMSNHTKERVTMTKVTLENF

SEQ ID NO: 311 - protein, 20 aa, synthetic construct
RQAVEGAIGRVADTIRSGPS

SEQ ID NO: 312 - protein, 20 aa, synthetic construct
LLAPLNDTRVVHAAKAALAA

SEQ ID NO: 313 - protein, 20 aa, synthetic construct
RSGASASGVYTIQVSNATKP

SEQ ID NO: 314 - protein, 20 aa, synthetic construct
AMAGSAMGATSLTLSAQSRT

SEQ ID NO: 315 - protein, 20 aa, synthetic construct
EILELAGNAARDNKKTRIIP

SEQ ID NO: 316 - protein, 20 aa, synthetic construct
ISNRVRVSIQTGTAFQPDAA

SEQ ID NO: 317 - protein, 20 aa, synthetic construct
LKEGTICKRARGDDMDDYCN

SEQ ID NO: 318 - protein, 20 aa, synthetic construct
LVEVGGSAKYLNNTKKYQNQ

SEQ ID NO: 319 - protein, 20 aa, synthetic construct
AAHPELKTPLTANITKSGNT

SEQ ID NO: 320 - protein, 20 aa, synthetic construct
DLTGRQDTSRMSTSQIPGRV

SEQ ID NO: 321 - protein, 20 aa, synthetic construct
QSAVEILLTIDDTKRAGMKE

SEQ ID NO: 322 - protein, 20 aa, synthetic construct
GLVEVGGSAKYLNNTKKYQN

SEQ ID NO: 323 - protein, 20 aa, synthetic construct
GSTYMAASGLNASTYDQVGR

SEQ ID NO: 324 - protein, 20 aa, synthetic construct
VGYLDRNLEKNSTKQEILAA

SEQ ID NO: 325 - protein, 20 aa, synthetic construct
TPGLRSLENDGTKLLDRIRD

SEQ ID NO: 326 - protein, 20 aa, synthetic construct
VTVSGKITHSPQAHVNPQEK

SEQ ID NO: 327 - protein, 20 aa, synthetic construct
ATLNSVLNSNAIKNLPPPLG

SEQ ID NO: 328 - protein, 20 aa, synthetic construct
QAQKDKMFSQNDTRCAGPEA

SEQ ID NO: 329 - protein, 20 aa, synthetic construct
PGVGLTPSAAQTARQHPKMH

SEQ ID NO: 330 - protein, 20 aa, synthetic construct
FDNSTQMNMIRDTLGNPTVK

SEQ ID NO: 331 - protein, 20 aa, synthetic construct
EYPERPASRANTVRSFHHEE

SEQ ID NO: 332 - protein, 20 aa, synthetic construct
SVGTQGRACNKTAPQASGCD

SEQ ID NO: 333 - protein, 20 aa, synthetic construct
DQEVINKASNSNKIRLEKGR

SEQ ID NO: 334 - protein, 20 aa, synthetic construct
PLLAPLNDTRVVHAAKAALA

SEQ ID NO: 335 - protein, 20 aa, synthetic construct
QNGKSYSLKASSTMAKSENL

SEQ ID NO: 336 - protein, 20 aa, synthetic construct
LVQIRSNFSKPDLMASLLPQ

SEQ ID NO: 337 - protein, 20 aa, synthetic construct
LQSAKDNNIKFRAIGHSDNS

SEQ ID NO: 338 - protein, 20 aa, synthetic construct
TGGSGGFHLAQQASASGSVS

SEQ ID NO: 339 - protein, 20 aa, synthetic construct
LNNAILRANLPKSKGNPAAY

SEQ ID NO: 340 - protein, 20 aa, synthetic construct
TPLTANITKSGNTFRPEVHL

SEQ ID NO: 341 - protein, 20 aa, synthetic construct
LTQGDGVAKLSINTHPSQKP

SEQ ID NO: 342 - protein, 20 aa, synthetic construct
ATLGADLVDSTDNTKINRGI

SEQ ID NO: 343 - protein, 20 aa, synthetic construct
TMWVDDQEVINKASNSNKIR

SEQ ID NO: 344 - protein, 20 aa, synthetic construct
RSLAGRAPAGAAAVSASGHG

SEQ ID NO: 345 - protein, 20 aa, synthetic construct
KDMIQTLTNKSKIQNVGGTY

SEQ ID NO: 346 - protein, 20 aa, synthetic construct
VVPEGIRMNKTVAVRTLDPE

SEQ ID NO: 347 - protein, 20 aa, synthetic construct
ATAGSAMGAASLTVSAQSRT

SEQ ID NO: 348 - protein, 20 aa, synthetic construct
SGLLETNFTASARTTGSGLL

SEQ ID NO: 349 - protein, 20 aa, synthetic construct
RVNNSSTNDNLVRKNDQVYI

SEQ ID NO: 350 - protein, 20 aa, synthetic construct
NGGARLAEYHAKATEHLSTL

SEQ ID NO: 351 - protein, 20 aa, synthetic construct
YTGHDTKLMQNSTKAPLKRS

SEQ ID NO: 352 - protein, 20 aa, synthetic construct
NQDNKIVVKNATKSNVNNAV

SEQ ID NO: 353 - protein, 20 aa, synthetic construct
HQASLSKQPTSQPRGDPTGP

SEQ ID NO: 354 - protein, 20 aa, synthetic construct
FRIHVENQTRARDDVSRKQL

SEQ ID NO: 355 - protein, 20 aa, synthetic construct
DDVSNQTSCRLAGLKPGTVY

SEQ ID NO: 356 - protein, 20 aa, synthetic construct
LVEAAASQRSSSLASNRLKI

SEQ ID NO: 357 - protein, 20 aa, synthetic construct
SLPATLPQCQAANKTCPTNY

SEQ ID NO: 358 - protein, 20 aa, synthetic construct
LVKSHVQLQNYSKVERHPVT

SEQ ID NO: 359 - protein, 20 aa, synthetic construct
AAPMKEANIRGQGGLAYPGV

SEQ ID NO: 360 - protein, 20 aa, synthetic construct
ELENNKTMNRAENGGRPPHH

SEQ ID NO: 361 - protein, 20 aa, synthetic construct
GKSVSGDVELTNIIKNSSFK

SEQ ID NO: 362 - protein, 20 aa, synthetic construct
FGPVLDKATRESAFVHAIAS

SEQ ID NO: 363 - protein, 20 aa, synthetic construct
PPGQKGQANATRIPAKTPPA

SEQ ID NO: 364 - protein, 20 aa, synthetic construct
VDDQEVINKASNSNKIRLEK

SEQ ID NO: 365 - protein, 20 aa, synthetic construct
SATYDDEHHSTLVSTRHYRA

SEQ ID NO: 366 - protein, 20 aa, synthetic construct
GLSTINQTRLDFHFSSDRTA

SEQ ID NO: 367 - protein, 20 aa, synthetic construct
CSSMSNHTKERVTMTKVTLE

SEQ ID NO: 368 - protein, 20 aa, synthetic construct
GAPGTPGPQGIAGQRGVVGL

SEQ ID NO: 369 - protein, 20 aa, synthetic construct
RNGPEFSNDIKKRKVDDKDS

SEQ ID NO: 370 - protein, 20 aa, synthetic construct
MFSQNDTRCAGPEAVKGQTL

SEQ ID NO: 371 - protein, 20 aa, synthetic construct
SGPSVLGTGTGGSGGFHLAQ

SEQ ID NO: 372 - protein, 20 aa, synthetic construct
VRHTENVTKTKTPEDYADSS

SEQ ID NO: 373 - protein, 20 aa, synthetic construct
SGKSIDDNSTKSEKQTPREH

SEQ ID NO: 374 - protein, 20 aa, synthetic construct
GVAGNIVRHIPDRGAPTRAS

SEQ ID NO: 375 - protein, 20 aa, synthetic construct
VKDRTHNVTEKVTQVLSLGA

SEQ ID NO: 376 - protein, 20 aa, synthetic construct
LISNLSNTIEKINTLGLDSK

SEQ ID NO: 377 - protein, 20 aa, synthetic construct
KPTSNVHVSVAGSHSQAANF

SEQ ID NO: 378 - protein, 20 aa, synthetic construct
NRQSSTSVIETNKTSVELSL

SEQ ID NO: 379 - protein, 20 aa, synthetic construct
SIPTPSNRVKTQQKSNLELL

SEQ ID NO: 380 - protein, 20 aa, synthetic construct
TSNVHVSVAGSHSQAANFAA

SEQ ID NO: 381 - protein, 20 aa, synthetic construct
SVLNSNAIKNLPPPLGGAAG

SEQ ID NO: 382 - protein, 20 aa, synthetic construct
LLTFSDVTSNQSSAVLGKSI

SEQ ID NO: 383 - protein, 20 aa, synthetic construct
GPMGPRGAPGERGRPGLPGA

SEQ ID NO: 384 - protein, 20 aa, synthetic construct
DKRRNGPEFSNDIKKRKVDD

SEQ ID NO: 385 - protein, 20 aa, synthetic construct
YGERSLLQTNQISNFIDRKN

SEQ ID NO: 386 - protein, 20 aa, synthetic construct
KTIVKAGPEYGQGMNPISRL

SEQ ID NO: 387 - protein, 20 aa, synthetic construct
GTQGRLCNRTSPGADGCDTM

SEQ ID NO: 388 - protein, 20 aa, synthetic construct
AVLALYNSTRDRVAGESAEP

SEQ ID NO: 389 - protein, 20 aa, synthetic construct
GFENEESDYHTLPRARIMQR

SEQ ID NO: 390 - protein, 20 aa, synthetic construct
RQEATNATRVIHDNYDQLEV

SEQ ID NO: 391 - protein, 20 aa, synthetic construct
VPAGGSSSNSTREPPASGRV

SEQ ID NO: 392 - protein, 20 aa, synthetic construct
RQDTSRMSTSQIPGRVASSG

SEQ ID NO: 393 - protein, 20 aa, synthetic construct
IQTLTNKSKIQNVGGTYELH

SEQ ID NO: 394 - protein, 20 aa, synthetic construct
DTKLMQNSTKAPLKRSNVEK

SEQ ID NO: 395 - protein, 20 aa, synthetic construct
TGRQDTSRMSTSQIPGRVAS

SEQ ID NO: 396 - protein, 20 aa, synthetic construct
KIQRVNNSSTNDNLVRKNDQ

SEQ ID NO: 397 - protein, 20 aa, synthetic construct
QRITMTNSVDDALINSTKIY

SEQ ID NO: 398 - protein, 20 aa, synthetic construct
DTQRRTSMGGTQQQFVEGVR

SEQ ID NO: 399 - protein, 20 aa, synthetic construct
WVDDQEVINKASNSNKIRLE

SEQ ID NO: 400 - protein, 20 aa, synthetic construct
SSDSIGVLGYQKTVDHTKVN

SEQ ID NO: 401 - protein, 20 aa, synthetic construct
REALMASGSGGHNTMPSGGN

SEQ ID NO: 402 - protein, 20 aa, synthetic construct
NAIKNLPPPLGGAAGHPGSA

SEQ ID NO: 403 - protein, 20 aa, synthetic construct
PLNDTRVVHAAKAALAAFNA

SEQ ID NO: 404 - protein, 20 aa, synthetic construct
LSRVNAAVANRDSANGFINK

SEQ ID NO: 405 - protein, 20 aa, synthetic construct
SHVQLQNYSKVERHPVTALE

SEQ ID NO: 406 - protein, 20 aa, synthetic construct
QGLVPAGGSSSNSTREPPAS

SEQ ID NO: 407 - protein, 20 aa, synthetic construct
GPIGNVGAPGAKGARGSAGP

SEQ ID NO: 408 - protein, 20 aa, synthetic construct
LLASLQKSPEDEEKNFDQTR

SEQ ID NO: 409 - protein, 20 aa, synthetic construct
SVGTQGRLCNRTSPGADGCD

SEQ ID NO: 410 - protein, 20 aa, synthetic construct
NPRGSSSRQSSSSAMSSSSA

SEQ ID NO: 411 - protein, 20 aa, synthetic construct
NGPEFSNDIKKRKVDDKDSS

SEQ ID NO: 412 - protein, 20 aa, synthetic construct
KNNKIAGVNNRTEYVETTST

SEQ ID NO: 413 - protein, 20 aa, synthetic construct
QAKFVSNQTELDREVLVGKV

SEQ ID NO: 414 - protein, 20 aa, synthetic construct
LVGYLDRNLEKNSTKQEILA

SEQ ID NO: 415 - protein, 20 aa, synthetic construct
WLVRHTENVTKTKTPEDYAD

SEQ ID NO: 416 - protein, 20 aa, synthetic construct
PGAKGANGAPGIAGAPGFPG

SEQ ID NO: 417 - protein, 20 aa, synthetic construct
RGDADNDMVKIENLTKVYKS

SEQ ID NO: 418 - protein, 20 aa, synthetic construct
TIFDTVNGKLVKKVNLDTTH

SEQ ID NO: 419 - protein, 20 aa, synthetic construct
GSSSRQSSSSAMSSSSASSS

SEQ ID NO: 420 - protein, 20 aa, synthetic construct
SALDTNSSKSTSSFPCPAGH

SEQ ID NO: 421 - protein, 20 aa, synthetic construct
SFNSHINASNNSEPSRMNMT

SEQ ID NO: 422 - protein, 20 aa, synthetic construct
FVIRGDEVRQIAPGQTGKIA

SEQ ID NO: 423 - protein, 20 aa, synthetic construct
CAPGTFSNTTSSTDICRPHQ

SEQ ID NO: 424 - protein, 20 aa, synthetic construct
ISNRVGVSIRTGTAFQPDPA

SEQ ID NO: 425 - protein, 20 aa, synthetic construct
GARGSAGPPGATGFPGAAGR

SEQ ID NO: 426 - protein, 20 aa, synthetic construct
QTLTNKSKIQNVGGTYELHV

SEQ ID NO: 427 - protein, 20 aa, synthetic construct
ANEKHMLALAPNSTARRQLA

SEQ ID NO: 428 - protein, 20 aa, synthetic construct
GGGATNANARAKGGTGQTGG

SEQ ID NO: 429 - protein, 20 aa, synthetic construct
VVDDVSNQTSCRLAGLKPGT

SEQ ID NO: 430 - protein, 20 aa, synthetic construct
ISSDSNNLKLNNVRLPRENM

SEQ ID NO: 431 - protein, 20 aa, synthetic construct
TVLVEAAASQRSSSLASNRL

SEQ ID NO: 432 - protein, 20 aa, synthetic construct
HGTQDRQCNKTSNGSDSCDL

SEQ ID NO: 433 - protein, 20 aa, synthetic construct
DSIGVLGYQKTVDHTKVNSK

SEQ ID NO: 434 - protein, 20 aa, synthetic construct
GRGSAGGNAHSPLGVPGGGL

SEQ ID NO: 435 - protein, 20 aa, synthetic construct
FGTRDRTCNVTSHGIDGCDL

SEQ ID NO: 436 - protein, 20 aa, synthetic construct
IQRVNNSSTNDNLVRKNDQV

SEQ ID NO: 437 - protein, 20 aa, synthetic construct
RLVTTGVLKQTKGVGASGSF

SEQ ID NO: 438 - protein, 20 aa, synthetic construct
SESIPTPSNRVKTQQKSNLE

SEQ ID NO: 439 - protein, 20 aa, synthetic construct
VHAKSSDWGLQAGYIPVNPN

SEQ ID NO: 440 - protein, 20 aa, synthetic construct
DINGFTTGRRGDLATIHGMN

SEQ ID NO: 441 - protein, 20 aa, synthetic construct
GSVGTQGRLCNRTSPGADGC

SEQ ID NO: 442 - protein, 20 aa, synthetic construct
LRGDADNDMVKIENLTKVYK

SEQ ID NO: 443 - protein, 20 aa, synthetic construct
QGLSNPSTLSAQPQLSRSHS

SEQ ID NO: 444 - protein, 20 aa, synthetic construct
SAGTGPSSATVNVTTRKPPP

SEQ ID NO: 445 - protein, 20 aa, synthetic construct
GHDTKLMQNSTKAPLKRSNV

SEQ ID NO: 446 - protein, 20 aa, synthetic construct
GARGPPGPAGANGAPGLRGG

SEQ ID NO: 447 - protein, 20 aa, synthetic construct
QTIPQISNRTLNTLVQDNSY

SEQ ID NO: 448 - protein, 20 aa, synthetic construct
LINDYVKNGTRGKITDLIKD

SEQ ID NO: 449 - protein, 20 aa, synthetic construct
QAVPTGSFMMVNSSGRASGQ

SEQ ID NO: 450 - protein, 20 aa, synthetic construct
HCVVADGNSTRSPETNGLLC

SEQ ID NO: 451 - protein, 20 aa, synthetic construct
STQNKRANRTEENVSDGSPN

SEQ ID NO: 452 - protein, 20 aa, synthetic construct
GPAGKEGGKGPRGETGPAGR

SEQ ID NO: 453 - protein, 20 aa, synthetic construct
DDQEVINKASNSNKIRLEKG

SEQ ID NO: 454 - protein, 20 aa, synthetic construct
VLVSRGGSQSVYGVDQTHSG

SEQ ID NO: 455 - protein, 20 aa, synthetic construct
QMGPRGLPGERGRPGAPGPA

SEQ ID NO: 456 - protein, 20 aa, synthetic construct
IINPRSSLVLAATSGNSGTT

SEQ ID NO: 457 - protein, 20 aa, synthetic construct
ERAASRANTVRSFHHEESLE

SEQ ID NO: 458 - protein, 20 aa, synthetic construct
HVPEGQTIKGRVGTVGYMAP

SEQ ID NO: 459 - protein, 20 aa, synthetic construct
AAIPSALDTNSSKSTSSFPC

SEQ ID NO: 460 - protein, 20 aa, synthetic construct
SLNLSGVPSQDKTRVEPPQY

SEQ ID NO: 461 - protein, 20 aa, synthetic construct
KFKGVDNEDDHGRGDLGRLK

SEQ ID NO: 462 - protein, 20 aa, synthetic construct
RAASRANTVRSFHHEESLEE

SEQ ID NO: 463 - protein, 20 aa, synthetic construct
LDRNLEKNSTKQEILAALEK

SEQ ID NO: 464 - protein, 20 aa, synthetic construct
YKAVGSLDPSADLSSQRGKV

SEQ ID NO: 465 - protein, 20 aa, synthetic construct
HSDAVFTDNYTALRKQMAVK

SEQ ID NO: 466 - protein, 20 aa, synthetic construct
TATLGADLVDSTDNTKINRG

SEQ ID NO: 467 - protein, 20 aa, synthetic construct
RSADSHGHPRENRTIGATMV

SEQ ID NO: 468 - protein, 20 aa, synthetic construct
CQDCPLLAPLNDTRVVHAAK

SEQ ID NO: 469 - protein, 20 aa, synthetic construct
KAAIPSALDTNSSKSTSSFP

SEQ ID NO: 470 - protein, 20 aa, synthetic construct
TLRPGEVTALVGPNGSGKST

SEQ ID NO: 471 - protein, 20 aa, synthetic construct
PPELENNKTMNRAENGGRPP

SEQ ID NO: 472 - protein, 20 aa, synthetic construct
NVDFRIHVENQTRARDDVSR

SEQ ID NO: 473 - protein, 20 aa, synthetic construct
RAHQNSQTHQASLSKQPTSQ

SEQ ID NO: 474 - protein, 20 aa, synthetic construct
VQLQNYSKVERHPVTALEKM

SEQ ID NO: 475 - protein, 20 aa, synthetic construct
VKSHVQLQNYSKVERHPVTA

SEQ ID NO: 476 - protein, 20 aa, synthetic construct
AMGAGVAGRITGSMSLASGK

SEQ ID NO: 477 - protein, 20 aa, synthetic construct
TLNSVLNSNAIKNLPPPLGG

SEQ ID NO: 478 - protein, 20 aa, synthetic construct
GLVPAGGSSSNSTREPPASG

SEQ ID NO: 479 - protein, 20 aa, synthetic construct
RPRGGGAGGSSVGTGGGGTG

SEQ ID NO: 480 - protein, 20 aa, synthetic construct
GITAFNHPLNLTKQQLSEVA

SEQ ID NO: 481 - protein, 20 aa, synthetic construct
DFDVIRNVIKDNATFSRKNP

SEQ ID NO: 482 - protein, 20 aa, synthetic construct
TPKGQLPGGKAPPKAGSVPS

SEQ ID NO: 483 - protein, 20 aa, synthetic construct
KSHVQLQNYSKVERHPVTAL

SEQ ID NO: 484 - protein, 20 aa, synthetic construct
YPERPASRANTVRSFHHEEH

SEQ ID NO: 485 - protein, 20 aa, synthetic construct
AGPAQTLIRPQDMKGASRSP

SEQ ID NO: 486 - protein, 20 aa, synthetic construct
SGASASGVYTIQVSNATKPR

SEQ ID NO: 487 - protein, 20 aa, synthetic construct
IVVYTGHDTKLMQNSTKAPL

SEQ ID NO: 488 - protein, 20 aa, synthetic construct
AVHVPEGQTIKGRVGTVGYM

SEQ ID NO: 489 - protein, 20 aa, synthetic construct
DVWGKGTTVTVSSASPTSPK

SEQ ID NO: 490 - protein, 20 aa, synthetic construct
RKNGPIDISNTDNFVRKNDL

SEQ ID NO: 491 - protein, 20 aa, synthetic construct
SKKQLSLKLSSVNAADTAVY

SEQ ID NO: 492 - protein, 20 aa, synthetic construct
NRVTNNVKDVTKLVANLPKD

SEQ ID NO: 493 - protein, 20 aa, synthetic construct
RRNGPEFSNDIKKRKVDDKD

SEQ ID NO: 494 - protein, 20 aa, synthetic construct
GFPGPKGNDGAPGKNGERGG

SEQ ID NO: 495 - protein, 20 aa, synthetic construct
LRGDFSSANNRDNTYNRVSE

SEQ ID NO: 496 - protein, 20 aa, synthetic construct
LPGMTNQDNKIVVKNATKSN

SEQ ID NO: 497 - protein, 20 aa, synthetic construct
KPGSSGPGSTGSWNSGSSGT

SEQ ID NO: 498 - protein, 20 aa, synthetic construct
LNSNAIKNLPPPLGGAAGHP

SEQ ID NO: 499 - protein, 20 aa, synthetic construct
NSQTIPQISNRTLNTLVQDN

SEQ ID NO: 500 - protein, 20 aa, synthetic construct
GSGPSVLGTGTGGSGGFHLA

SEQ ID NO: 501 - protein, 20 aa, synthetic construct
LLQSAKDNNIKFRAIGHSDN

SEQ ID NO: 502 - protein, 20 aa, synthetic construct
LNLSGVPSQDKTRVEPPQYM

SEQ ID NO: 503 - protein, 20 aa, synthetic construct
ALKENGGARLAEYHAKATEH

SEQ ID NO: 504 - protein, 20 aa, synthetic construct
ERPASRANTVRSFHHEEHLE

SEQ ID NO: 505 - protein, 20 aa, synthetic construct
PGMTNQDNKIVVKNATKSNV

SEQ ID NO: 506 - protein, 20 aa, synthetic construct
GVQLGKRYNGSDPASGPSVQ

SEQ ID NO: 507 - protein, 20 aa, synthetic construct
GSLGTAGRVCNLTSRGMDSC

SEQ ID NO: 508 - protein, 20 aa, synthetic construct
GYLDRNLEKNSTKQEILAAL

SEQ ID NO: 509 - protein, 20 aa, synthetic construct
GIEQGVGVPGGSPGSRHRQK

SEQ ID NO: 510 - protein, 20 aa, synthetic construct
LVPAGGSSSNSTREPPASGR

SEQ ID NO: 511 - protein, 20 aa, synthetic construct
ESIPTPSNRVKTQQKSNLEL

SEQ ID NO: 512 - protein, 20 aa, synthetic construct
LKTPLTANITKSGNTFRPEV

SEQ ID NO: 513 - protein, 20 aa, synthetic construct
RFDVSAMEKNASNLVKAEFR

SEQ ID NO: 514 - protein, 20 aa, synthetic construct
VYLGTSLGNITRDQKILNPS

SEQ ID NO: 515 - protein, 20 aa, synthetic construct
KYIDQEELNKTKPIWTRNPD

SEQ ID NO: 516 - protein, 20 aa, synthetic construct
QRIAVETAATARKSSPPASS

SEQ ID NO: 517 - protein, 20 aa, synthetic construct
YISNRVGVSIRTGTAFQPDP

SEQ ID NO: 518 - protein, 20 aa, synthetic construct
HVQLQNYSKVERHPVTALEK

SEQ ID NO: 519 - protein, 20 aa, synthetic construct
AAGVAHAITAACTQGNLSDC

SEQ ID NO: 520 - protein, 20 aa, synthetic construct
SFGTRDRTCNVTSHGIDGCD

SEQ ID NO: 521 - protein, 20 aa, synthetic construct
TAFNHPLNLTKQQLSEVALM

SEQ ID NO: 522 - protein, 20 aa, synthetic construct
LEYPERPASRANTVRSFHHE

SEQ ID NO: 523 - protein, 20 aa, synthetic construct
QSSTSVIETNKTSVELSLPF

SEQ ID NO: 524 - protein, 20 aa, synthetic construct
MLALAPNSTARRQLASLLEG

SEQ ID NO: 525 - protein, 20 aa, synthetic construct
ETGLSTINQTRLDFHFSSDR

SEQ ID NO: 526 - protein, 20 aa, synthetic construct
AGGSSSNSTREPPASGRVAL

SEQ ID NO: 527 - protein, 20 aa, synthetic construct
KDNNIKFRAIGHSDNSVPPF

SEQ ID NO: 528 - protein, 20 aa, synthetic construct
GTFSNTTSSTDICRPHQICN

SEQ ID NO: 529 - protein, 20 aa, synthetic construct
RDLFFNEIKARSGQDDKTVY

SEQ ID NO: 530 - protein, 20 aa, synthetic construct
YSIGVLGYQKTVDHTKVNSK

SEQ ID NO: 531 - protein, 20 aa, synthetic construct
GFENEESDYHTLPRAGIMQK

SEQ ID NO: 532 - protein, 20 aa, synthetic construct
YSIGVLGYQKTEDHTKVNSK

SEQ ID NO: 533 - protein, 20 aa, synthetic construct
YSIGVLGNQKTVDHTKVNSK

SEQ ID NO: 534 - protein, 20 aa, synthetic construct
DAPLGTGANKDLELPWLEQQ

SEQ ID NO: 535 - protein, 20 aa, synthetic construct
YSIGVLSYQKTVDHTKVNSK

SEQ ID NO: 536 - protein, 20 aa, synthetic construct
YSIGVLGYQKTVDHTKVNGK

SEQ ID NO: 537 - protein, 20 aa, synthetic construct
YSIGVLVYQKTVDHTKVNSK

SEQ ID NO: 538 - protein, 20 aa, synthetic construct
FTGEPSLLPVSPVGHLHASL

SEQ ID NO: 539 - protein, 20 aa, synthetic construct
YSIGVQGYQKTVDHTKVNSK

SEQ ID NO: 540 - protein, 20 aa, synthetic construct
LGKGTGGSGGFHRAQQASGS

SEQ ID NO: 541 - protein, 20 aa, synthetic construct
KKDRVTEALNATRAAVEEGF

SEQ ID NO: 542 - protein, 20 aa, synthetic construct
HDTNLMQNSTKAHLKRSNVE

SEQ ID NO: 543 - protein, 20 aa, synthetic construct
QDNTCEEYHTMGPHSSHIPK

SEQ ID NO: 544 - protein, 20 aa, synthetic construct
LNSNAIKNLPTPLGGAAGHQ

SEQ ID NO: 545 - protein, 20 aa, synthetic construct
FSIGVLGYQKTVDHTKVNSK

SEQ ID NO: 546 - protein, 20 aa, synthetic construct
DKRRNGPEFSNYIKKRKVDD

SEQ ID NO: 547 - protein, 20 aa, synthetic construct
YSIGVLGYQKSVDHTKVNSK

SEQ ID NO: 548 - protein, 20 aa, synthetic construct
YSIGVLGYQKTVDHTKVNST

SEQ ID NO: 549 - protein, 20 aa, synthetic construct
WGQGSTVAASSGSASAPTLF

SEQ ID NO: 550 - protein, 20 aa, synthetic construct
LSATQSVVSHIGSKRVEGIV

SEQ ID NO: 551 - protein, 20 aa, synthetic construct
FGPSAALDSQSGLLKGQEPW

SEQ ID NO: 552 - protein, 20 aa, synthetic construct
SDSICVLGYQKTVDHTKVNS

SEQ ID NO: 553 - protein, 20 aa, synthetic construct
AGGSSSNSTREPPASGLVAF

SEQ ID NO: 554 - protein, 20 aa, synthetic construct
YKRRNGPEFSNDIKKRKVDD

SEQ ID NO: 555 - protein, 20 aa, synthetic construct
YSIGVLGNQKTVDHTKENSK

SEQ ID NO: 556 - protein, 20 aa, synthetic construct
DINGLTTGRRGDMAAIHGMN

SEQ ID NO: 557 - protein, 20 aa, synthetic construct
NVDFSINVENQTRARDDVSR

SEQ ID NO: 558 - protein, 20 aa, synthetic construct
DSIGVLGYKKTVDNTKVNSK

SEQ ID NO: 559 - protein, 20 aa, synthetic construct
VLNSNAIKNLHQPLGGAAGH

SEQ ID NO: 560 - protein, 20 aa, synthetic construct
ETGLSAINQTRLDFNNSSDR

SEQ ID NO: 561 - protein, 20 aa, synthetic construct
YQEVINKASNSNKIRLEKGR

SEQ ID NO: 562 - protein, 20 aa, synthetic construct
ISSDSICALGYQKTVDHTKV

SEQ ID NO: 563 - protein, 20 aa, synthetic construct
VQAGGSSSNSTREQPASGRV

SEQ ID NO: 564 - protein, 20 aa, synthetic construct
SSDSIGVHGYQKTVDHTKVN

SEQ ID NO: 565 - protein, 20 aa, synthetic construct
QVHPDSGISSEAMGIVNSFV

SEQ ID NO: 566 - protein, 20 aa, synthetic construct
SSTSTMPNSSRSSHSSSWTL

SEQ ID NO: 567 - protein, 20 aa, synthetic construct
VTAGGSSSNSTREQPASGRV

SEQ ID NO: 568 - protein, 20 aa, synthetic construct
TAAHPELKTQLTANITKSGN

SEQ ID NO: 569 - protein, 20 aa, synthetic construct
LVPACCSSSNSTREPPASGR

SEQ ID NO: 570 - protein, 20 aa, synthetic construct
VPAGGSSSNSTREQHASGRV

SEQ ID NO: 571 - protein, 20 aa, synthetic construct
SSTSTMPNSSRSSNSSSGTL

SEQ ID NO: 572 - protein, 20 aa, synthetic construct
LQALVREMSGSPASGIPAEV

SEQ ID NO: 573 - protein, 20 aa, synthetic construct
IARVADTISSGPVNSESFPT

SEQ ID NO: 574 - protein, 20 aa, synthetic construct
APPTELHRTRKEGVATYAAA

SEQ ID NO: 575 - protein, 20 aa, synthetic construct
MIQTLTNKSKIQNVGWTYEF

SEQ ID NO: 576 - protein, 20 aa, synthetic construct
VLNSNAIKNLPQQLGGAAGH

SEQ ID NO: 577 - protein, 20 aa, synthetic construct
DKEVINKASNSNKIRLEKGR

SEQ ID NO: 578 - protein, 20 aa, synthetic construct
TATMGADLVDSTDNTKINRG

SEQ ID NO: 579 - protein, 20 aa, synthetic construct
RTSMGGTQQQFVKGARMEEI

SEQ ID NO: 580 - protein, 20 aa, synthetic construct
TATLGADLVDSTDNTKINRC

SEQ ID NO: 581 - protein, 20 aa, synthetic construct
LGGTVANPGGSGTSSRLEPK

SEQ ID NO: 582 - protein, 20 aa, synthetic construct
SSDSICVLGYQKTVDHTKVN

SEQ ID NO: 583 - protein, 20 aa, synthetic construct
ACGSSSNSTREPPASGLVAL

SEQ ID NO: 584 - protein, 20 aa, synthetic construct
TLGADLVDSTDNTKINLGIF

SEQ ID NO: 585 - protein, 20 aa, synthetic construct
ISSDSIGVLGYKKTVDHTKV

SEQ ID NO: 586 - protein, 20 aa, synthetic construct
SVLNSNAIKNLPQQLGGAAG

SEQ ID NO: 587 - protein, 20 aa, synthetic construct
LVPAGGSSSNSTREQHASGR

SEQ ID NO: 588 - protein, 20 aa, synthetic construct
ENVDFRINVENQTRARDDVS

SEQ ID NO: 589 - protein, 20 aa, synthetic construct
AKSAVEILLTIDDTKRAGMK

SEQ ID NO: 590 - protein, 20 aa, synthetic construct
DFRIHVENQTLALDDVSRKQ

SEQ ID NO: 591 - protein, 20 aa, synthetic construct
SDSIGVLCYQKTVDHTKVNS

SEQ ID NO: 592 - protein, 20 aa, synthetic construct
PACGSSSNSTREPPASGLVA

SEQ ID NO: 593 - protein, 20 aa, synthetic construct
ISSDSIGALGYKKTVDHTKV

SEQ ID NO: 594 - protein, 20 aa, synthetic construct
RRVGDSSGAAAAHKVLPRGL

SEQ ID NO: 595 - protein, 20 aa, synthetic construct
DSIGVLGYQKTVDHTNVNSN

SEQ ID NO: 596 - protein, 20 aa, synthetic construct
PAGCSSSNSTREPPASGLVA

SEQ ID NO: 597 - protein, 20 aa, synthetic construct
LASIQKSPEDEEKNYDQTRF

SEQ ID NO: 598 - protein, 20 aa, synthetic construct
VPAGGTSSNSTREPPASGLV

SEQ ID NO: 599 - protein, 20 aa, synthetic construct
LGGTVTHPGGSGTSSRLEPK

SEQ ID NO: 600 - protein, 20 aa, synthetic construct
GSATIYWHDIRTEMDETRKN

SEQ ID NO: 601 - protein, 20 aa, synthetic construct
RRNGPEFSNYIKKRKVDDKD

SEQ ID NO: 602 - protein, 20 aa, synthetic construct
SDSIGVLGYQKTVDHTNVNS

SEQ ID NO: 603 - protein, 20 aa, synthetic construct
KAAIPSALDTNSSKSGSSFP

SEQ ID NO: 604 - protein, 20 aa, synthetic construct
SVLNTNAIKNLPTPLGGAAG

SEQ ID NO: 605 - protein, 20 aa, synthetic construct
SRNGPEFSNDIKKRKVDDKD

SEQ ID NO: 606 - protein, 20 aa, synthetic construct
LPEAVLALHNSTRDLVAGES

SEQ ID NO: 607 - protein, 20 aa, synthetic construct
GSEGTQGRACNKTAPQASCC

SEQ ID NO: 608 - protein, 20 aa, synthetic construct
VLALNNSTSDRVAGESAEPE

SEQ ID NO: 609 - protein, 20 aa, synthetic construct
CKFLKEGTICKRARCDDMDD

SEQ ID NO: 610 - protein, 20 aa, synthetic construct
VQAGGSSSNSTRELPASGRV

SEQ ID NO: 611 - protein, 20 aa, synthetic construct
LNTVLNSNAIKNLPQPLGGA

SEQ ID NO: 612 - protein, 20 aa, synthetic construct
VKGAGGESRVLPSGQGINNL

SEQ ID NO: 613 - protein, 20 aa, synthetic construct
QGLVQAGGSSSNSTRELPAS

SEQ ID NO: 614 - protein, 20 aa, synthetic construct
NRAVFKGGKDNIPVLKRDVA

SEQ ID NO: 615 - protein, 20 aa, synthetic construct
VLNSNAIKNLPPPLCCAAGH

SEQ ID NO: 616 - protein, 20 aa, synthetic construct
NRAVFKGCQDNIPVLKRDVA

SEQ ID NO: 617 - protein, 20 aa, synthetic construct
GDKANSSIEFHSKPQHQRDV

SEQ ID NO: 618 - protein, 20 aa, synthetic construct
LKTQLTANITKSGNTFRHEV

SEQ ID NO: 619 - protein, 20 aa, synthetic construct
VMLVLTNPEGRKEGKEVTFQ

SEQ ID NO: 620 - protein, 20 aa, synthetic construct
VPACGSSSNSTREPPASGLV

SEQ ID NO: 621 - protein, 20 aa, synthetic construct
ARLVLTNHEGRKEGKEVTFQ

SEQ ID NO: 622 - protein, 20 aa, synthetic construct
RRNGPEFSNDIKKRKVDYKD

SEQ ID NO: 623 - protein, 20 aa, synthetic construct
NCKFLKEGTICKRARCDDMD

SEQ ID NO: 624 - protein, 20 aa, synthetic construct
VPAGGSSSNSTLEPPASGLV

SEQ ID NO: 625 - protein, 20 aa, synthetic construct
HVQLQNYSKVERHHVTALEN

SEQ ID NO: 626 - protein, 20 aa, synthetic construct
DSIGVLGYQKTGDHTKVNSK

SEQ ID NO: 627 - protein, 20 aa, synthetic construct
GGLAYPGVRTHGTLESVNWP

SEQ ID NO: 628 - protein, 20 aa, synthetic construct
SVLNSNAIKNLPPPLGCAAW

SEQ ID NO: 629 - protein, 20 aa, synthetic construct
VRLVLTNHEGRKEGKEVSFQ

SEQ ID NO: 630 - protein, 20 aa, synthetic construct
LKTQLTANTTKSGNTFRPEV

SEQ ID NO: 631 - protein, 20 aa, synthetic construct
VRLVLTNPEGRKECKEVTFQ

SEQ ID NO: 632 - protein, 20 aa, synthetic construct
NCKFLKEGTICKRARGYDMD

SEQ ID NO: 633 - protein, 20 aa, synthetic construct
SMGTQGRACNKTAPQASGCD

SEQ ID NO: 634 - protein, 20 aa, synthetic construct
SESIPTLSNRVKTQQKSNLE

SEQ ID NO: 635 - protein, 20 aa, synthetic construct
NCKFLKECTICKRARGDDMD

SEQ ID NO: 636 - protein, 20 aa, synthetic construct
KLARTIRTARTQAEEREMIQ

SEQ ID NO: 637 - protein, 20 aa, synthetic construct
ARLVLTNPEGRKDGKEVTFQ

SEQ ID NO: 638 - protein, 20 aa, synthetic construct
MVNSSGRASGQMAHQLLPTL

SEQ ID NO: 639 - protein, 20 aa, synthetic construct
SNCRHEIDAGVVCTNETRST

SEQ ID NO: 640 - protein, 20 aa, synthetic construct
CKFLKEGTICKRARGDDVDV

SEQ ID NO: 641 - protein, 20 aa, synthetic construct
GLESEESEYHTLPRARIMQR

SEQ ID NO: 642 - protein, 20 aa, synthetic construct
CRHERDAGVVCTNETRSTNT

SEQ ID NO: 643 - protein, 20 aa, synthetic construct
QDNTCEEYHTMGPHSNHIPK

SEQ ID NO: 644 - protein, 20 aa, synthetic construct
KLSSTSRLKSVGLNLEGNGE

SEQ ID NO: 645 - protein, 20 aa, synthetic construct
SSQAGVPVRVPRSPRAPESA

SEQ ID NO: 646 - protein, 20 aa, synthetic construct
VRLVLTNPEGRKEGKEVSFK

SEQ ID NO: 647 - protein, 20 aa, synthetic construct
VRRVLTNPEGRKEGMEVTFQ

SEQ ID NO: 648 - protein, 20 aa, synthetic construct
LQSAKDNNIKFRAIGHSDSG

SEQ ID NO: 649 - protein, 20 aa, synthetic construct
NCKFLKEGTICKRARGDYMD

SEQ ID NO: 650 - protein, 20 aa, synthetic construct
QGISNSSTSSMPNSSQSSHS

SEQ ID NO: 651 - protein, 20 aa, synthetic construct
FLEVSEQILAKIPSGKNKQT

SEQ ID NO: 652 - protein, 20 aa, synthetic construct
ARLVLTNTEGRKEGKEVTFQ

SEQ ID NO: 653 - protein, 20 aa, synthetic construct
FRGVAGGTALSRSAVHESTG

SEQ ID NO: 654 - protein, 20 aa, synthetic construct
NRAVFKGGQDNIHVLKRDVA

SEQ ID NO: 655 - protein, 20 aa, synthetic construct
SNCRHERYAGVVCTNETRST

SEQ ID NO: 656 - protein, 20 aa, synthetic construct
GHDTKLMQNSTKAPLKSSTV

SEQ ID NO: 657 - protein, 20 aa, synthetic construct
LREIRSYQRTTEPLIRKLPF

SEQ ID NO: 658 - protein, 20 aa, synthetic construct
QDDTCEEYHSAQPNSCHISK

SEQ ID NO: 659 - protein, 20 aa, synthetic construct
LLPSSTVTPRAPHLSHSSQP

SEQ ID NO: 660 - protein, 20 aa, synthetic construct
GHDTKLMQNSTKAPLNRSYV

SEQ ID NO: 661 - protein, 20 aa, synthetic construct
ELIRTIRSARTQAEERGVIQ

SEQ ID NO: 662 - protein, 20 aa, synthetic construct
WNGQDNTCEEYHSVGPHSCH

SEQ ID NO: 663 - protein, 20 aa, synthetic construct
DFSIHVENQTRARDDVSSKQ

SEQ ID NO: 664 - protein, 20 aa, synthetic construct
SSDNIGVLGYQKTVDHTKVK

SEQ ID NO: 665 - protein, 20 aa, synthetic construct
PGVLPLLTVDPGSAKQRPPR

SEQ ID NO: 666 - protein, 20 aa, synthetic construct
NGGARLAEYHAKTTEHQSTL

SEQ ID NO: 667 - protein, 20 aa, synthetic construct
GLENEESDYHTLPRARIMQK

SEQ ID NO: 668 - protein, 20 aa, synthetic construct
EYPERQASRANTVRSFNHEE

SEQ ID NO: 669 - protein, 20 aa, synthetic construct
HQANLSKQPTSQPRGDPTGP

SEQ ID NO: 670 - protein, 20 aa, synthetic construct
DARHIVTQSDDPHFHEKKQI

SEQ ID NO: 671 - protein, 20 aa, synthetic construct
VARHVGSQSDDPHFHEKKQI

SEQ ID NO: 672 - protein, 20 aa, synthetic construct
IRLGLSHKADMQTREVLMKL

SEQ ID NO: 673 - protein, 20 aa, synthetic construct
KWVDDQEVINKASNSNKIRP

SEQ ID NO: 674 - protein, 20 aa, synthetic construct
GFENEESEYHTLPRARIMQK

SEQ ID NO: 675 - protein, 20 aa, synthetic construct
GVDNEDYHGRGDLGRLKTQK

SEQ ID NO: 676 - protein, 20 aa, synthetic construct
SSDSIGVLGYQKTEDHTKVK

SEQ ID NO: 677 - protein, 20 aa, synthetic construct
GMRLPRGAPVDIPSSDLTGR

SEQ ID NO: 678 - protein, 20 aa, synthetic construct
PAFRNEEYHKSVLENQATFF

SEQ ID NO: 679 - protein, 20 aa, synthetic construct
YLESGSINEKDYHSRLNIDE

SEQ ID NO: 680 - protein, 20 aa, synthetic construct
VDFRIHVENQTLARDDVSRN

SEQ ID NO: 681 - protein, 20 aa, synthetic construct
NSVLNSNAIKNLPQPLGGTA

SEQ ID NO: 682 - protein, 20 aa, synthetic construct
LLRECNTVRSFLAAAAETLE

SEQ ID NO: 683 - protein, 20 aa, synthetic construct
GFENEESDYHTLPRARIVQK

SEQ ID NO: 684 - protein, 20 aa, synthetic construct
MIQTLTNKSKIQNVGVTYEF

SEQ ID NO: 685 - protein, 20 aa, synthetic construct
GHDTKLMQNSTKAPLKRPDV

SEQ ID NO: 686 - protein, 20 aa, synthetic construct
NRQSSTSVLETNKTSVVLSS

SEQ ID NO: 687 - protein, 20 aa, synthetic construct
GDMANSSIEFHPKPRQQREV

SEQ ID NO: 688 - protein, 20 aa, synthetic construct
VVYTGRDTKIMQNSTKAPLK

SEQ ID NO: 689 - protein, 20 aa, synthetic construct
HVHEGQTVKGRVGTVGYLAP

SEQ ID NO: 690 - protein, 20 aa, synthetic construct
HKENGGARLVEYHAKASEHL

SEQ ID NO: 691 - protein, 20 aa, synthetic construct
GFENEESDYHALPRARIMQK

SEQ ID NO: 692 - protein, 20 aa, synthetic construct
LVPKAAGNSNGGFVKPNPND

SEQ ID NO: 693 - protein, 20 aa, synthetic construct
RSADSYGHPSENRTIGTTMV

SEQ ID NO: 694 - protein, 20 aa, synthetic construct
LRLADREHSSGESSFEYTVT

SEQ ID NO: 695 - protein, 20 aa, synthetic construct
GIESEESEYHTLPRARIMQR

SEQ ID NO: 696 - protein, 20 aa, synthetic construct
KFKGVDNVDYHGRGDLGRPE

SEQ ID NO: 697 - protein, 20 aa, synthetic construct
GDSIGVLGYQKTVDHTKVNR

SEQ ID NO: 698 - protein, 20 aa, synthetic construct
HKENGGAGLAEYHAKATEHL

SEQ ID NO: 699 - protein, 20 aa, synthetic construct
DFRINVENKTRARDDVSRKQ

SEQ ID NO: 700 - protein, 20 aa, synthetic construct
GFENVESDYHTLPRARIMQK

SEQ ID NO: 701 - protein, 20 aa, synthetic construct
HDTKLMQNSTKAHLKRSNVE

SEQ ID NO: 702 - protein, 20 aa, synthetic construct
ELENNKTMNRAENGGRPPLN

SEQ ID NO: 703 - protein, 20 aa, synthetic construct
RIKLTYIHKGSAMQDLAEVS

SEQ ID NO: 704 - protein, 20 aa, synthetic construct
NVDIRINVENQTRARDDVSR

SEQ ID NO: 705 - protein, 20 aa, synthetic construct
ENNKTMNRAENGGRPPNHHF

SEQ ID NO: 706 - protein, 20 aa, synthetic construct
GGGGPGGSGGVGGDSGGYTY

SEQ ID NO: 707 - protein, 20 aa, synthetic construct
PELVNNKTMNRAENGGRPPN

SEQ ID NO: 708 - protein, 20 aa, synthetic construct
QDNTCEEYHSVGPHSCRIPK

SEQ ID NO: 709 - protein, 20 aa, synthetic construct
SLLESTSDSNLNKYSTIHKI

SEQ ID NO: 710 - protein, 20 aa, synthetic construct
GGSSSNSTRVPPASGLVALV

SEQ ID NO: 711 - protein, 20 aa, synthetic construct
ESLVGRKESPHSRSGSIVSS

SEQ ID NO: 712 - protein, 20 aa, synthetic construct
EPQPVLENNKTMNRAENGGR

SEQ ID NO: 713 - protein, 20 aa, synthetic construct
PIGNQEKNVPLPVIAELPPK

SEQ ID NO: 714 - protein, 20 aa, synthetic construct
AESLELAGNATRDNKKTRII

SEQ ID NO: 715 - protein, 20 aa, synthetic construct
GFENEESDYHTLPRAGIMQM

SEQ ID NO: 716 - protein, 20 aa, synthetic construct
ENNKTMNRAENGGRPHNHPF

SEQ ID NO: 717 - protein, 20 aa, synthetic construct
SSDSIGVLGYQKTVDHTKEK

SEQ ID NO: 718 - protein, 20 aa, synthetic construct
VDFRIHVENQTRARDDESSK

SEQ ID NO: 719 - protein, 20 aa, synthetic construct
MIQTITNKSKIQNVGGTYES

SEQ ID NO: 720 - protein, 20 aa, synthetic construct
YGSTTDDDDHHSTLVSSRHY

SEQ ID NO: 721 - protein, 20 aa, synthetic construct
EDGSARLAEYHAKATERLGT

SEQ ID NO: 722 - protein, 20 aa, synthetic construct
LKTTLTANITKSGNTFRHEV

SEQ ID NO: 723 - protein, 20 aa, synthetic construct
RNGPEVSNDIKKRKVDDKDS

SEQ ID NO: 724 - protein, 20 aa, synthetic construct
SALDTNSSKSTSSFPCPAVH

SEQ ID NO: 725 - protein, 20 aa, synthetic construct
GFENEESDYHTLPRARTMQK

SEQ ID NO: 726 - protein, 20 aa, synthetic construct
IDAQGEPHGYRQGDPSYRSF

SEQ ID NO: 727 - protein, 20 aa, synthetic construct
RHLESQSDDPHFHEKKQIPF

SEQ ID NO: 728 - protein, 20 aa, synthetic construct
QDNSCEEYHTAGPHSGHIPK

SEQ ID NO: 729 - protein, 20 aa, synthetic construct
KENGGSRLAEYHAKATEHLS

SEQ ID NO: 730 - protein, 20 aa, synthetic construct
QTFDDPHVSSFHGSKRGLNS

SEQ ID NO: 731 - protein, 20 aa, synthetic construct
QRGKGLSHWVPEHPALPTAS

SEQ ID NO: 732 - protein, 20 aa, synthetic construct
QRSGASASGVYTIKASNATK

SEQ ID NO: 733 - protein, 20 aa, synthetic construct
YGQDNTCEEYHTVGPHSSPI

SEQ ID NO: 734 - protein, 20 aa, synthetic construct
QRSWASASGVYTIQASNATK

SEQ ID NO: 735 - protein, 20 aa, synthetic construct
AKSFDWGLQAGYITVNSNLS

SEQ ID NO: 736 - protein, 20 aa, synthetic construct
QKGIQKPKSGTKGNYDDDWK

SEQ ID NO: 737 - protein, 20 aa, synthetic construct
GFENEESDYHTLPRARITQK

SEQ ID NO: 738 - protein, 20 aa, synthetic construct
ICVLGYQKTVDHTKVNSKLS

SEQ ID NO: 739 - protein, 20 aa, synthetic construct
VKENGGARLAEYHTKATEHL

SEQ ID NO: 740 - protein, 20 aa, synthetic construct
LKENGGAILAEYHTKATEHL

SEQ ID NO: 741 - protein, 20 aa, synthetic construct
GFEDEESDYHTLPRARITQR

SEQ ID NO: 742 - protein, 20 aa, synthetic construct
RWLQAQKDKMLSKNDTRSAG

SEQ ID NO: 743 - protein, 20 aa, synthetic construct
NMTKATVNYRKEKTHMMSAV

SEQ ID NO: 744 - protein, 20 aa, synthetic construct
FGSATLDDEHHSTLVSTSHY

SEQ ID NO: 745 - protein, 20 aa, synthetic construct
QRSWASASGVNTIQVSNATK

SEQ ID NO: 746 - protein, 20 aa, synthetic construct
FGSATIDVEHHSTLASTRHY

SEQ ID NO: 747 - protein, 20 aa, synthetic construct
LALYNSTRDRKAGVSAEPEP

SEQ ID NO: 748 - protein, 20 aa, synthetic construct
LGAPALVQRPQGNRPGLAKA

SEQ ID NO: 749 - protein, 20 aa, synthetic construct
GGARLVEYHAKATENLSTLS

SEQ ID NO: 750 - protein, 20 aa, synthetic construct
SIGVLGYKKTVDHTKVNSKP

SEQ ID NO: 751 - protein, 20 aa, synthetic construct
GSATYEDEHHSTLVSTRQYG

SEQ ID NO: 752 - protein, 20 aa, synthetic construct
QRSWASASGAYTIQVSNATK

SEQ ID NO: 753 - protein, 20 aa, synthetic construct
YWGSVTGSQSNAVSHTQAPR

SEQ ID NO: 754 - protein, 20 aa, synthetic construct
QAYKAPVVVEGKVQGLVQAG

SEQ ID NO: 755 - protein, 20 aa, synthetic construct
IMELAGNAARDNKTTRILPR

SEQ ID NO: 756 - protein, 20 aa, synthetic construct
LYRSLAGRAPAWAAAVSASG

SEQ ID NO: 757 - protein, 20 aa, synthetic construct
SICVLGYQKTVDHTKANSKL

SEQ ID NO: 758 - protein, 20 aa, synthetic construct
SVSRALTQALPAPTCQNTQV

SEQ ID NO: 759 - protein, 20 aa, synthetic construct
QKGIKKPKSGTQGNYDDDWK

SEQ ID NO: 760 - protein, 20 aa, synthetic construct
VPTGSFMMVNSSGRASGKKA

SEQ ID NO: 761 - protein, 20 aa, synthetic construct
RSDLTTLTESYANRQGLNKN

SEQ ID NO: 762 - protein, 20 aa, synthetic construct
SSQAGVPVRAPRSPRAPESA

SEQ ID NO: 763 - protein, 20 aa, synthetic construct
TAGSTMGAASITQTVLARNL

SEQ ID NO: 764 - protein, 20 aa, synthetic construct
KENGGARLAEYHAKSTEHLS

SEQ ID NO: 765 - protein, 20 aa, synthetic construct
WVDDQKVINKASNSNKIRLE

SEQ ID NO: 766 - protein, 20 aa, synthetic construct
LLSASSVGSAHGDEGAGYTD

SEQ ID NO: 767 - protein, 20 aa, synthetic construct
GMIQTANVGVGISGNEGMQA

SEQ ID NO: 768 - protein, 20 aa, synthetic construct
EQLITQEPKEANSTTSKKQS

SEQ ID NO: 769 - protein, 20 aa, synthetic construct
PGAVLALYNSTSDRVAGESA

SEQ ID NO: 770 - protein, 20 aa, synthetic construct
ISEERFRQLEKIKTVGSTYM

SEQ ID NO: 771 - protein, 20 aa, synthetic construct
IQGQLQPSLKTGSEEIRGQY

SEQ ID NO: 772 - protein, 20 aa, synthetic construct
ERVPVHATVVGDVVSPEMGE

SEQ ID NO: 773 - protein, 20 aa, synthetic construct
QNAVNVVKQIKTLIEKTNEE

SEQ ID NO: 774 - protein, 20 aa, synthetic construct
SVAGSLSLAANSAAQKTHQD

SEQ ID NO: 775 - protein, 20 aa, synthetic construct
ARLLGSLDPSADLSSQRGKV

SEQ ID NO: 776 - protein, 20 aa, synthetic construct
HYLGLARRAPAGAAAVSASG

SEQ ID NO: 777 - protein, 20 aa, synthetic construct
KQQDSLPDTKAGGPAHSKVV

SEQ ID NO: 778 - protein, 20 aa, synthetic construct
FGSASYDEEHHSTLVSTRHY

SEQ ID NO: 779 - protein, 20 aa, synthetic construct
KWKTTFDLKKTRMEPFHFKT

SEQ ID NO: 780 - protein, 20 aa, synthetic construct
QNAVNWVKQIKTLIEKTNEE

SEQ ID NO: 781 - protein, 20 aa, synthetic construct
SFMIRGDEVRQTASGQTGKI

SEQ ID NO: 782 - protein, 20 aa, synthetic construct
LVTAGGSSSNSTREQPASGR

SEQ ID NO: 783 - protein, 20 aa, synthetic construct
ENVDFRIHVENKTRARDDVS

SEQ ID NO: 784 - protein, 20 aa, synthetic construct
RGLQVTVLGENKTGNSPVER

SEQ ID NO: 785 - protein, 20 aa, synthetic construct
PELKTQLTANITKSGDTFRP

SEQ ID NO: 786 - protein, 20 aa, synthetic construct
LAMAGSAVGTTSLTLSAQSR

SEQ ID NO: 787 - protein, 20 aa, synthetic construct
LVKIRSNSSKPDLMASLLPQ

SEQ ID NO: 788 - protein, 20 aa, synthetic construct
TGLLPAAGEKTCGPLGNLAE

SEQ ID NO: 789 - protein, 20 aa, synthetic construct
TGGSSSNSTREPHASGRVAL

SEQ ID NO: 790 - protein, 20 aa, synthetic construct
LKTPLTTNITKSGNTFLPEV

SEQ ID NO: 791 - protein, 20 aa, synthetic construct
YSFGVLGYQKTVDHTKVNSK

SEQ ID NO: 792 - protein, 20 aa, synthetic construct
AELKPANLPVTKPFTVGDNK

SEQ ID NO: 793 - protein, 20 aa, synthetic construct
QTIPQISNRTRNTMVQDNSY

SEQ ID NO: 794 - protein, 20 aa, synthetic construct
PSFCTQKPRKFYCELPDISL

SEQ ID NO: 795 - protein, 20 aa, synthetic construct
LVQIRSNSSKPDLMASMLPQ

SEQ ID NO: 796 - protein, 20 aa, synthetic construct
VNNSSTNDNLVRKNDKVYIN

SEQ ID NO: 797 - protein, 20 aa, synthetic construct
EKMLVLDAEQRVTAGEALAN

SEQ ID NO: 798 - protein, 20 aa, synthetic construct
GSQASGIPAKVSATVSSPGS

SEQ ID NO: 799 - protein, 20 aa, synthetic construct
PELENNKTMSRAENGGRPPN

SEQ ID NO: 800 - protein, 20 aa, synthetic construct
WLVAKRVQDHTTVVGDVVSQ

SEQ ID NO: 801 - protein, 20 aa, synthetic construct
GNVDFRIHVENQTRALDDVS

SEQ ID NO: 802 - protein, 20 aa, synthetic construct
YKVLKQVHPDSGVSSQAMGI

SEQ ID NO: 803 - protein, 20 aa, synthetic construct
QTIPKISNRTLNTQVQDNSY

SEQ ID NO: 804 - protein, 20 aa, synthetic construct
SPANGSLGPTLNLSSWESRL

SEQ ID NO: 805 - protein, 20 aa, synthetic construct
GLVQAGGSSSNSTREPHASG

SEQ ID NO: 806 - protein, 20 aa, synthetic construct
QAGIPVRAPRSPRAQEHALE

SEQ ID NO: 807 - protein, 20 aa, synthetic construct
IGILSQGVASGMAGSAMSSK

SEQ ID NO: 808 - protein, 20 aa, synthetic construct
TAAHPELKTTLTANITKSGN

SEQ ID NO: 809 - protein, 20 aa, synthetic construct
LKQVHPDTCISSKAMGIMNS

SEQ ID NO: 810 - protein, 20 aa, synthetic construct
MSQVPLQNYSKVERHPVTAK

SEQ ID NO: 811 - protein, 20 aa, synthetic construct
HSWTTLYNPSLESRVTMSVD

SEQ ID NO: 812 - protein, 20 aa, synthetic construct
VPACCSSSNSTREPPASGRV

SEQ ID NO: 813 - protein, 20 aa, synthetic construct
QNTAIILLKEDSLDATRAAI

SEQ ID NO: 814 - protein, 20 aa, synthetic construct
AKSSDWGLQAGYIHVNPNLS

SEQ ID NO: 815 - protein, 20 aa, synthetic construct
YSIGVLGYQKTVDLTKVNSK

SEQ ID NO: 816 - protein, 20 aa, synthetic construct
DNVDFRIHVENQTRARDDVS

SEQ ID NO: 817 - protein, 20 aa, synthetic construct
LLAGGLHAWKVIKGEEISVV

SEQ ID NO: 818 - protein, 20 aa, synthetic construct
EGKVQGLVQAGGSSSNSTRE

SEQ ID NO: 819 - protein, 20 aa, synthetic construct
QSAVEILLTIDDTKMAGMKE

SEQ ID NO: 820 - protein, 20 aa, synthetic construct
ETGWTPGLRSLENYGTKLLD

SEQ ID NO: 821 - DNA, 2211 bp, Adeno-associated virus
  source 1..2211: mol_type = genomic DNA
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttggaggaaaccttgggaaggctgttttccaggctaaaaagaggcttctcgaacctcttggcctggttgagacgccggataaaacggcgcctgcggcaaaaaagaggcctctagagcagagtcctcaagagccagactcctcgagcggatttggcaaaaagggcaaacagcctgccagaaagagactcaattttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggcacgagcaacgccagcgacaccttcaacggattctccaccccctggggatactttgacttcaaccgcttccactgccacttctcgccaagagactggcaaaggctcatcaacaaccactgggggctgcgccccaaaagcatgcaagtccgcatcttcaacatccaagttaaggaggtcacgacgtctaacggagagacgaccgtatcaaacaacctcaccagcacggtccagatctttgcggacagcacgtacgagctcccgtacgtgatggatgcgggtcaggagggcagcttgccccctttccccaacgacgtgttcatggtgccccagtacgggtactgcggactggtaaccggaggcagctctcaaaaccagacagacagaaacgccttctactgtctggagtactttcccagccagatgctgagaacaggaaacaactttgagatggtgtacaagtttgagaacgtgcccttccactccatgtacgctcacagccagagcctggataggctgatgaacccgctgctggaccagtacctgtgggaactccagtctaccacatctggaggaaatctcaaccagggcacgtcagccacaaactttgccaagctgaccaaaacaaacttttctggctaccgcaaaaactggcttccgggacccatgatgaagcagcagagattctccaagactgccaatcaaaactacaagattccccaagggggaaataacagtctgctccattatgagaccagaactaccctcgacggaagatggagcaattttgccccgggaacggccatggcaaccgcggccaacgatgccaccgacttctcccaggcccagctcatctttgcggggcccaacatcaccggcaataccacgacagacgccaacaacctgatgttcacttcagaagatgaactcagggccaccaacccccgggacactgacctgtttggccacctggcaaccaaccagcaaaacgccaccaccgttcctaccgcagacgacgtggacggagtcggcgtgtacccgggaatggtgtggcaggacagagacatttattaccaagggcccatctgggccaagattccccatacggatggacactttcacccatctcccctcattggcggatttggactgaaaagcccgcctccacaaatattcatcaaaaacacccctgtacccgccaatcccgcgacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 822 - protein, 736 aa, Adeno-associated virus
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRLLEPLGLVETPDKTAPAAKKRPLEQSPQEPDSSSGFGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGTSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGNLNQGTSATNFAKLTKTNFSGYRKNWLPGPMMKQQRFSKTANQNYKIPQGGNNSLLHYETRTTLDGRWSNFAPGTAMATAANDATDFSQAQLIFAGPNITGNTTTDANNLMFTSEDELRATNPRDTDLFGHLATNQQNATTVPTADDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 823 - DNA, 2211 bp, Adeno-associated virus
  source 1..2211: mol_type = genomic DNA
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttgggggaaaccttgggaaggctgttttccaggctaaaaagaggattctcgaacctcttggcctggttgagacgccggataaaacggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 824 - protein, 736 aa, Adeno-associated virus
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 825 - DNA, 2151 bp, Adeno-associated virus
  source 1..2151: mol_type = genomic DNA
atgtcgttctttgattatctaggtaaaaaatacgctcaaggagccgcggattggtgggacgcgaaagcaggaccacctcctccgaaaaaggccaaagggaaaggcttcgtctttcccggacacaactacctaggtcccggcaacgacctagacaacggtaaacccgttgacgcggacgacgctgccgccaaaaagcacgacgaagcttacgacgagcagctcaaacagggcgacaacccgtacgtgaaatataatcacgcggacgcccagctgcaagaagacctcaaagacgtcaatacctttgaggccaatctcgccaaaggagtcttccaggccaagaaacgggtcctcgaaccgtttggtctggtagaagaactggccgcaaagaagcgacctcactctgagatcgaatcgcccgattccagctcgggaatcggcaagaagggagaccaacccgcacgacgtcggttggactttgaccaggaagacagacaacaagccgctcaagacggagccaccggtaccggaagtctcgagccgtcctcaaatttgggatctgatacgatgtctacaggcggtggcgctccaatggcaggagataaccaaggcgccgatggagtgggtaatgcctcaggaaattggcattgcgataccacatggatgggagaccgagtggtcacccgctccactcgcacctggttgctgcccagctacaacaaccacgtgtacagaagaatcaacgccggatcaaacagcaacgccttcttcggcttcagcaccccgtgggggtactttgactacaacagattccactgccacttttctccacgagactggcaacgactcgtcaacaacaactggggcattcgacccaagcacctcaagttcaaactcttcaacatacaagttaaagaatacacggtccaggagggcggaggcgagaccgtcgccaataacctcacaagcaccgtgcaggtctttacggactcggactatcagctaccgtacgtgctaggttcggcgcaagagggtgccatgccaccgttccccgccgacgtgtttatgttgcctcaatacgcgtactgcactcggcagaacggaaccaacccgacgcccagaagcgccttttactgtctggaatactttcccagtcagatgctgagaaccggcaacaactttgacttctcgtacgagttcgagaacgtgcccttccacagtatgtgggctcacaaccagactcttgatcgtctgatgaatcctctggtggatcaatacttgtggagactacaaaatgtagctaacgtaagcggaacaaagaccaatcagttcagccagaacgtggccggcgacctacaactgtttactcggaactggctacccgggcccgcttacagaacgcagtcttggtcgaccaacacggctcagaacgcgactcagaccaacatcccgaatcaggaaaacgtctactttctggaaggaagaactaacaccatcgcacccggcatggccatggggcccacaaccgctacacactttcaatccaacggagtgatattcgccaagaacgaatcgggtactccggcaactggtgccaacattaaccagttgaacatcacacgggaatcagaagtcaactcgaccaatcctctgctgtcaagaccacccctggctattaccaacaacgagcagaccgcagctgccacgccagacagagccaactggaatctggctcccgtcatgcccggacaagtctggatggacaaagacatctacctgcaaggccagatctgggccaagatcccacacacggacggacactttcatccgtctcctctcatgggaggattcggactgaaacaccctccacctcaaatcttcatcaagaacactcccgtacccggatcacccgctcccaactttaccgtggacaaggtcaacaagttcatcaaccaatactctaccggtcaagtaaccgtggagattgaatgggaactgcagaaggaatcgtccaagagatggaatccagagatccaatacacggacgacgcatccgccaccgacggatctatcctgcactttgctcccaacgacgaaggattgtacacccagttccgatctatcgggactcggtacctcacccgccctctgtaa

SEQ ID NO: 826 - protein, 716 aa, Adeno-associated virus
MSFFDYLGKKYAQGAADWWDAKAGPPPPKKAKGKGFVFPGHNYLGPGNDLDNGKPVDADDAAAKKHDEAYDEQLKQGDNPYVKYNHADAQLQEDLKDVNTFEANLAKGVFQAKKRVLEPFGLVEELAAKKRPHSEIESPDSSSGIGKKGDQPARRRLDFDQEDRQQAAQDGATGTGSLEPSSNLGSDTMSTGGGAPMAGDNQGADGVGNASGNWHCDTTWMGDRVVTRSTRTWLLPSYNNHVYRRINAGSNSNAFFGFSTPWGYFDYNRFHCHFSPRDWQRLVNNNWGIRPKHLKFKLFNIQVKEYTVQEGGGETVANNLTSTVQVFTDSDYQLPYVLGSAQEGAMPPFPADVFMLPQYAYCTRQNGTNPTPRSAFYCLEYFPSQMLRTGNNFDFSYEFENVPFHSMWAHNQTLDRLMNPLVDQYLWRLQNVANVSGTKTNQFSQNVAGDLQLFTRNWLPGPAYRTQSWSTNTAQNATQTNIPNQENVYFLEGRTNTIAPGMAMGPTTATHFQSNGVIFAKNESGTPATGANINQLNITRESEVNSTNPLLSRPPLAITNNEQTAAATPDRANWNLAPVMPGQVWMDKDIYLQGQIWAKIPHTDGHFHPSPLMGGFGLKHPPPQIFIKNTPVPGSPAPNFTVDKVNKFINQYSTGQVTVEIEWELQKESSKRWNPEIQYTDDASATDGSILHFAPNDEGLYTQFRSIGTRYLTRPL

SEQ ID NO: 827 - DNA, 2157 bp, Adeno-associated virus
  source 1..2157: mol_type = genomic DNA
atgtcttctttgtttaaagagtacctgcagagtaccggactggtgggtatccagtcgggagcgccgaagccaaaggccggccagcagaagcaggatacagggtcctttgagtggaaaaagaaggaggacacagccagagggctggtccttcccggttacaagtacctcgggcccttcaacggtctagagcggggcgagcctgtgaatgccgcggacgccgcggcccagcgacacgaccgacagtacgatcgtattctacaacaagggggtaatccatacctcacgtacaaccacgccgaccgagagttccaggaggagctccagtctgacgagtcttttgggggaaatcttggcaaggctgtgtttcaggccaagaagcgaatcctagagcccctcggtctggttgaagaggatccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 828 - protein, 718 aa, Adeno-associated virus
MSSLFKEYLQSTGLVGIQSGAPKPKAGQQKQDTGSFEWKKKEDTARGLVLPGYKYLGPFNGLERGEPVNAADAAAQRHDRQYDRILQQGGNPYLTYNHADREFQEELQSDESFGGNLGKAVFQAKKRILEPLGLVEEDPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 829 - DNA, 2208 bp, Adeno-associated virus
  source 1..2208: mol_type = genomic DNA
atgtcttttttagaagcgtttgaggactggtacgaaaaagcagcggctacttggcgacatctcgaacctggcgccccccgtcctaaagccaaccagcagcatcaggaacagcatcatccggttcctgctgaagagcctaaagatcaagctcgaggccttgtgctacctggctacaagtacctcggcccctttaacggccttgataaaggtgatccggttaatcaagcggacgctgcggcgcttgaacacgataaagcgtacaaccaccttctcgaagaaggacaaaacccgtacctcgtgtataacgaggctgataagcatttccaggaaactctacagggggatacgagcttcggcggtaacctcggcaaagcagtcttccaaggcaagaaacgagtcctagagccctttggactcgttgacgaggagcctcacgaactggctccacctccaaaaaagaagacaaaacaggaatacaaagacactctagaggctattcctcctcctagcagagaggaacagactcctcagaagggatcttcagcatccaaaaacggagcctcatcttcagcagcagcagcagcccccagtaatttgggatctggtatcatggcagaaggcggtggcgcaccaatgggcgacaatcaacagggtgccgatggagtgggtaatgcctcgggaaattggcattgcgattcccactggatgggcgacaaagtcgtcacccgcaccaccagaacctgggtcctgcccacctacaacaaccacctctaccaagccatcgacgagaatactactctcggatccgccaacaacttctacggattcagcaccccatgggggtactttgactttaaccgcttccactgccacttctccccacgtgactggcaaagactcatcaacaacaactggggcatccgacccagaagcctcagattcaagctgttcaacatccaagtcaaggaggtcacgacgacgtcgggcgaaacgaccatcgccaataaccttaccagcacggttcaggtttttgcggatgaagaaatgcacctcccgtacgtcttagggagtgcccacgacgggtgctttcctccatttccaccagacatttacatgctgccccaatacgggtactgcactctggactatcagggactcaccacagacaggagcgccttctactgtctggagtacttcccttctcagatgctgagaacgggcaacaactttgagcttacttacaactttgagaagctgcctttccacagcatgtggatgcacaaccagtccctggacaggatcatgaatcctctcatcgatcagtacctgtatcgcttcgcctccaagactggcaacacatggacataccaaaaaggcagcaaagacgacaagaccgctcaagccagaaactggcttcctggaccctccatcagaaatcagcctctgtactcgagtggtaatcaaaccaacctctcgggatacgatatcgctccaaaaacagaagtcaacgagaaacagatgactgtgtttcctggactttctatggccaccgaaaacgagaggagcggaaccatcaaagagcagccaacgtccaatctgctcatctttgccaacaaagcggatgcggctacctctcagaccacttctagcgtgggcagtacagcgacactcctggtcaccaacgaaaacgaagtcaagactgtcaatccctccgccaccgaagcgtggggttcggctgcggccaacaaacagacatctgctaccgcctctcaaagcgtggctattcacgctcaaggcgtggtgccaggcatggtctggcaagacagagacatctactttcagggccccatctgggccaagatcccgcatacagatggacactttcacccatctcctctcatgggtggattcggactcaaacatcctcctccacaaattttcattaaaaacactcctgtcccctccaatcctgctaccacgtacaccaccgccaagtacaactccttcatcacccagtactccaccggtcaagtgactgtggagattgactgggaactccagaaggagacctccaaaaagtggaaccccgagatccagttcacctccaattacaacgggacagacacgctcggattcgctccagatgccgctggagactacaccgaaccacgtgccatcggaacccgataccttacccgacccctttaa

SEQ ID NO: 830 - protein, 735 aa, Adeno-associated virus
MSFLEAFEDWYEKAAATWRHLEPGAPRPKANQQHQEQHHPVPAEEPKDQARGLVLPGYKYLGPFNGLDKGDPVNQADAAALEHDKAYNHLLEEGQNPYLVYNEADKHFQETLQGDTSFGGNLGKAVFQGKKRVLEPFGLVDEEPHELAPPPKKKTKQEYKDTLEAIPPPSREEQTPQKGSSASKNGASSSAAAAAPSNLGSGIMAEGGGAPMGDNQQGADGVGNASGNWHCDSHWMGDKVVTRTTRTWVLPTYNNHLYQAIDENTTLGSANNFYGFSTPWGYFDFNRFHCHFSPRDWQRLINNNWGIRPRSLRFKLFNIQVKEVTTTSGETTIANNLTSTVQVFADEEMHLPYVLGSAHDGCFPPFPPDIYMLPQYGYCTLDYQGLTTDRSAFYCLEYFPSQMLRTGNNFELTYNFEKLPFHSMWMHNQSLDRIMNPLIDQYLYRFASKTGNTWTYQKGSKDDKTAQARNWLPGPSIRNQPLYSSGNQTNLSGYDIAPKTEVNEKQMTVFPGLSMATENERSGTIKEQPTSNLLIFANKADAATSQTTSSVGSTATLLVTNENEVKTVNPSATEAWGSAAANKQTSATASQSVAIHAQGVVPGMVWQDRDIYFQGPIWAKIPHTDGHFHPSPLMGGFGLKHPPPQIFIKNTPVPSNPATTYTTAKYNSFITQYSTGQVTVEIDWELQKETSKKWNPEIQFTSNYNGTDTLGFAPDAAGDYTEPRAIGTRYLTRPL

SEQ ID NO: 831 - DNA, 2181 bp, Adeno-associated virus
  source 1..2181: mol_type = genomic DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcacaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgcacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactattggggcttcagaccccggtctctcagagtcaaaatcttcaacatccaagtcaaagaggtcacggtgcaggactccaccaccaccatcgccaacaacctcacctccaccgtccaagtgtttacggacgacgactaccaactcccgtacgtcgtcggcaacgggaccgagggatgcctgccggccttccccccgcaggtctttacgctgccgcagtacggctacgcgacgctgaaccgagacaacggagacaacccgacagagcggagcagcttcttttgcctagagtactttcccagcaagatgctgaggacgggcaacaactttgagtttacctacagctttgaagaggtgcccttccactgcagcttcgccccgagccagaacctctttaagctggccaacccgctggtggaccagtacctgtaccgcttcgtgagcacctcggccacgggcgccatccagttccaaaagaacctggcgggcagatacgccaacacctacaaaaactggttcccggggcccatgggccgaacccagggctggaacacgagctctggcagcagcaccaacagagtcagcgtcaacaacttttccgtctcaaaccggatgaacctggagggggccagctaccaagtgaacccccagcccaacgggatgacaaacacgctccaaggcagcaaccgctacgcgctggaaaacaccatgatcttcaacgctcaaaacgccacgccgggaactacctcggtgtacccagaggacaatctactgctgaccagcgagagcgagactcagcccgtcaaccgggtggcttacaacacgggcggtcagatggccaccaacgcccagaacgccaccacggctcccacggtcgggacctacaacctccaggaagtgcttcctggcagcgtatggatggagagggacgtgtacctccaaggacccatctgggccaagatcccagagacgggggcgcactttcacccctctccggccatgggcggattcggactcaaacacccgccgcccatgatgctcatcaaaaacacgccggtgcccggcaacatcaccagcttctcggacgtgcccgtcagcagcttcatcacccagtacagcaccgggcaggtcaccgtggagatggaatgggagctcaaaaaggaaaactccaagaggtggaacccagagatccagtacaccaacaactacaacgacccccagtttgtggactttgctccagacggctccggcgaatacagaaccaccagagccatcggaacccgatacctcacccgacccctttaa

SEQ ID NO: 832 - protein, 726 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNGDNPTERSSFFCLEYFPSKMLRTGNNFEFTYSFEEVPFHCSFAPSQNLFKLANPLVDQYLYRFVSTSATGAIQFQKNLAGRYANTYKNWFPGPMGRTQGWNTSSGSSTNRVSVNNFSVSNRMNLEGASYQVNPQPNGMTNTLQGSNRYALENTMIFNAQNATPGTTSVYPEDNLLLTSESETQPVNRVAYNTGGQMATNAQNATTAPTVGTYNLQEVLPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDGSGEYRTTRAIGTRYLTRPL

SEQ ID NO: 833 - DNA, 2151 bp, Adeno-associated virus
  source 1..2151: mol_type = genomic DNA
atgtcgtttgttgatcaccctccagattggcttgaggagattggtgagggtctaaaggagtttttgggactcgaacctggcccacccaaaccgaagcccaaccagcagaagcaagacgacgcccgtggtcttgtactgcctggatataattacctgggacccggaaacggtctcgaccgcggagaacctgtcaaccgggctgacgaggtcgcgcgagagcacgacatctcgtacaacgagcagctccaggcgggagacaacccctacctcaagtacaaccacgcggacgccgagtttcaggagaagctcgcggacgacacctccttcgggggcaacctcggcaaggcagtctttcaggccaaaaaaagggttctcgaaccttttggcctggttgaggagcctgttaagacggctgctaaaggcgagcggatagacgaccactatcccaaaaagaagaaggctcggatcgaagagaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacgtggatgggggaccgagtcatcaccaagtccacccgaacctgggtgctgcccagctacaacaaccatcagtaccttgagatccacagcggttccgtcgacggaagcaacgctaacgcttattttggatacagcaccccctgggggtactttgacttcaaccgcttccacagccactggagcccccgagactggcagcgactcgtcaacaactactggggattcagaccccggtccctcaaggtcaagatctttaacatccaagtcaaggaagtcacgacgcaggacggcaccaccaccatcgccaacaacctcacctccaccgtccaagtgtttacggacaacgactaccagctaccgtacgtcatcggcaacggaacggaggggtgcctgccggccttccctccgcaggtctttacgctgccgcagtacggctacgcgacactgaaccgtaacaacaccgacgatcccaccgagcggagcagtttcttctgcctggaatactttcccagcaagatgctgcggacgggcaacaactttgaattcacctacagcttcgaggaggtgcccttccactgcagcttcgctcccagccagaacctcttcaagctggccaatccgctggtggaccagtacctgtaccgctttgtgagcaccgacacttccggtaacctacagttccaaaagaacttgaaggccagatatgccaacacttacaagaattggtttccggggcccatgtgccggacccagggctggtacacaagcgcgggcacatataacaacaaaggcgttgccaactttgatacttcaaacaagatggaactggagggggctagttaccaagtaaaccctcaaccaaatggaatgacaaacacgcttcaggatagtaacaaatacgcgcttgaaaacaccatgatcttcaacgcacagaacgcccctccgggaacgacctctctgtaccaggagaacaatcttttgataaccagcgagagcgagacgcagcctgtgaaccgattggcctacaacaccggtggtcaggtatcaaacaacaaccagaattcaaatacacatcctacggtcggagtatacaatcaccaggaagtgttgcctggtagcgtgtggatggacagagacgtataccttcagggccccatctgggccaaaatcccggagacaggggcacactttcatccttctccggctatgggcggattcggactcaaacacccaccgcccatgatgctcatcaagaacacaccggtacctagcaacgtcgctgccttctctgacgtgcccgttaaaagcttcatcacccagtacagcaccggacaggtcacggtggagattgaatgggagctcaagaaagaaaactccaagaggtggaatcccgagatacagtacaccaacaactacaacaaccctacattcgtggactttgctccagacacctccggcgagtacaggactacgagggctattggaacccgttaccttacccgacccctgtaa

SEQ ID NO: 834 - protein, 716 aa, Adeno-associated virus
MSFVDHPPDWLEEIGEGLKEFLGLEPGPPKPKPNQQKQDDARGLVLPGYNYLGPGNGLDRGEPVNRADEVAREHDISYNEQLQAGDNPYLKYNHADAEFQEKLADDTSFGGNLGKAVFQAKKRVLEPFGLVEEPVKTAAKGERIDDHYPKKKKARIEETEAGTSGAQQLQIPAQPASSLGADTMSAGGGSPLGDNNQGADGVGNASGDWHCDSTWMGDRVITKSTRTWVLPSYNNHQYLEIHSGSVDGSNANAYFGYSTPWGYFDFNRFHSHWSPRDWQRLVNNYWGFRPRSLKVKIFNIQVKEVTTQDGTTTIANNLTSTVQVFTDNDYQLPYVIGNGTEGCLPAFPPQVFTLPQYGYATLNRNNTDDPTERSSFFCLEYFPSKMLRTGNNFEFTYSFEEVPFHCSFAPSQNLFKLANPLVDQYLYRFVSTDTSGNLQFQKNLKARYANTYKNWFPGPMCRTQGWYTSAGTYNNKGVANFDTSNKMELEGASYQVNPQPNGMTNTLQDSNKYALENTMIFNAQNAPPGTTSLYQENNLLITSESETQPVNRLAYNTGGQVSNNNQNSNTHPTVGVYNHQEVLPGSVWMDRDVYLQGPIWAKIPETGAHFHPSPAMGGFGLKHPPPMMLIKNTPVPSNVAAFSDVPVKSFITQYSTGQVTVEIEWELKKENSKRWNPEIQYTNNYNNPTFVDFAPDTSGEYRTTRAIGTRYLTRPL

SEQ ID NO: 835 - DNA, 2151 bp, Adeno-associated virus
  source 1..2151: mol_type = genomic DNA
atgtcgtttgttgatcaccctccagattggcttgaggagattggtgagggtctaaaggagtttttgggactcgaacctggcccacccaaaccgaagcccaaccagcagaagcaagacaacgcccgtggtcttgtactgcctggatataattacctgggacccggcaacggtctcgaccgcggagaacctgtcaaccgggccgacgaggtcgcgcgagagcacgacatctcgtacaacgagcagctccaggcgggagacaacccctacctcaagtacaaccacgcggacgccgagtttcaagagaagctcgcggacgacacctccttcgggggcaacctcggcaaggcagtctttcaggccaagaaaagggttctcgaaccttttggcctggttgaggatcctgttaagacggctgctaaaggcgagcggatagacgaccactatcccaagaagaagaaggctcgggttgaagaaaccgaagctggaaccagcggagcccagcagctgcagatcccagcccaaccagcctcaagtttgggagctgatacaatgtctgcgggaggtggcagcccactgggcgacaataaccaaggcgccgatggagtgggcaatgcctcgggagattggcattgcgattccacatggatgggggacagagtcatcaccaagtccacccgaacctgggtgctgcccagctacaataaccacctgtacaaagagatccacaacaacggggtcgacggcagcaccgccaacgcttactttggatacagtaccccctgggggtactttgacttcaaccgcttccacagccactggagccctcgagactggcagcgactcatcaacaactactggggcttcagaccccggtccctcaaggtcaagattttcaacatccaagtcaaagaggtcacggtgcaggacaacaccaccaccatcgccaacaacctcacctccaccgtccaagtgtttacggacaacgactaccagctgccgtacgtcatcggcaacgggaccgaggggtgtctaccggccttccctccgcaggtctttacgctgccgcagtatggctacgcgacgctgaatcgcaacaatagcgaaaatcccaccgagcgaagcagcttcttctgtctggagtactttcccagcaagatgctgcggacgggcaacaactttgagttcacatacagcttcgaggaggtacccttccactgcagcttcgcgcccagccaaaacctcttcaaactggctaacccgttggtggatcagtacctttaccgcttcgtgagcacggacacctccggtgccgtccagttcaaaaagaacctggcgggcagatacgccaacacctacaagaactggttcccaggacccatgtgccgaacccagggctggtacacaggaacgggtatatataacaacaaaggcgctaccagctttaacacctcaaacagaatggacctagagggagccagttatcaagtgcctccccagcccaacgggatgacaaacacggttcaggacagcaacctttacgcgctggaaaacaccatgatttttaacgcacaaaacgccaccccgggaacgaatacaacgtatccggaggagaaccttttgataaccagtgagagcgagactcaacccgtgaacagagtggcttacagctccggaggacaaatagccaacaacaatcagaataccaacacggctcctactgcaggaacctacaaccaccaggaaatactacctggcagcgtgtggatggacagggacgtgtacctccagggtcccatctgggccaagatcccagagacaggggcacactttcatccttctccagccatgggcgggttcggactcaaatacccgcctcccatgatgctaatcaagaacacgccagtgcccggaaacatcaccaccttctcggacgtgcccgtccaaagttttattacccagtacagcaccggacaagtcaccgtggagatggagtgggagctcaagaaggaaaactctaagaggtggaaccccgagatacagtacaccaacaactacaacaaccctacgttcgtggactttgctccagacacagaaggagaatacaggaccactagggctattggaacccgctaccttacccgacctctgtaa

SEQ ID NO: 836 - protein, 716 aa, Adeno-associated virus
MSFVDHPPDWLEEIGEGLKEFLGLEPGPPKPKPNQQKQDNARGLVLPGYNYLGPGNGLDRGEPVNRADEVAREHDISYNEQLQAGDNPYLKYNHADAEFQEKLADDTSFGGNLGKAVFQAKKRVLEPFGLVEDPVKTAAKGERIDDHYPKKKKARVEETEAGTSGAQQLQIPAQPASSLGADTMSAGGGSPLGDNNQGADGVGNASGDWHCDSTWMGDRVITKSTRTWVLPSYNNHLYKEIHNNGVDGSTANAYFGYSTPWGYFDFNRFHSHWSPRDWQRLINNYWGFRPRSLKVKIFNIQVKEVTVQDNTTTIANNLTSTVQVFTDNDYQLPYVIGNGTEGCLPAFPPQVFTLPQYGYATLNRNNSENPTERSSFFCLEYFPSKMLRTGNNFEFTYSFEEVPFHCSFAPSQNLFKLANPLVDQYLYRFVSTDTSGAVQFKKNLAGRYANTYKNWFPGPMCRTQGWYTGTGIYNNKGATSFNTSNRMDLEGASYQVPPQPNGMTNTVQDSNLYALENTMIFNAQNATPGTNTTYPEENLLITSESETQPVNRVAYSSGGQIANNNQNTNTAPTAGTYNHQEILPGSVWMDRDVYLQGPIWAKIPETGAHFHPSPAMGGFGLKYPPPMMLIKNTPVPGNITTFSDVPVQSFITQYSTGQVTVEMEWELKKENSKRWNPEIQYTNNYNNPTFVDFAPDTEGEYRTTRAIGTRYLTRPL

SEQ ID NO: 837 - DNA, 2130 bp, Adeno-associated virus
  source 1..2130: mol_type = genomic DNA
atgtcgttttttgattacataggtaaaaaatacgctcaaggagcggccgattggtggaacgcagaagcgggaccacctcctccaaaaaaagccaaagggaaaggattcgtctttcccggtcacaactatctaggtccgggaaacgacctagataacgggaatcccgtcgacgaagacgacgctgccgccaaaaagcacgacgaggcttacgacgagcagctcaagcagggcgacaatccgtacgtgaagtataatcacgcggacgctcagcttcaggaagacctcaaggacgtcaataccttcgaggccaatctcgctaaaggaatctttcaggccaaaaaacgggtcctagaaccgtttggcctggtggaagaactggccgggaaaaagaggcctcatactgaacaagagtcccccgattccagcgcaggagtcggaaagaaaggcgaacagccagctaagcgtcggttggatttctccgacgcagaagcgcctcaagacggagcagcagcagcgccctctggtgtggaagatattacaatgtctggcggaggaggcgctccgatggacggaaatcaacaaggtgccgatggagtgggcaatgcctcgggaaattggcattgcgattcccaatggatggggaacagagtcgttacccattccaccagaacctgggtcctgcccagctacaacgaccacatctaccgacaaatcagcagcggagcccagaccgaccactccaagcaatactttggatacagcaccccctgggggtactttgactttaaccgattccactgccacttttcaccgcgagactggcagcgattggtcaacaaccactggggattcagacccaagaagctgcacgtcaagctctttaacatccaagtcaaggagtgggtcactcaagatgggactaacgtcgtatccaacaacctcacgagcaccgtacaagtgttcacggacgacgagtaccagctcccctacgtgctcggcaacgccacagaaggatgtctccccgccttccctccgacggtctttacgctgccgcagtacggctacgcgacgctgaacgacccgggggacgacgctccgaccaacaggagctcgttcttttgcctggagtactttcccagtcagatgctgagaaccggcaacaactttgagttcagctactcgttcgaagacgtgccctttcacacgggatgggctcccaaccagcatctgtttaagacggcgaatcctctggtggaccagtacctgtatcaactggtcggaaccaacaacaccggagacgttcagtttgagaaagtgctggccggtaactacggagacgcgttcaagaactggatcacgggagccaacaggcgtaccaactcttggctgctgggcggaggaaacagagccaacgtcaccgctctgaatccaaactacgtccagatcgaaggagacttttacaagtcgcagccggaacccgccggcatggccaacaacctcagcggatcggacgcggttgccctgggcaacagtctgatattcaaccctcagcagacggcgccaggaactaccaccagactgccggcgcagaacgtactggtgaccagagaagacgagacgcagcccgtcaacacgtacgcgtccaactctaccggatacgtggcgaccaatcagcagagtactacgtcggctcctcagaacggacagtaccactttcaaggcatcatgccaggggccgtgtggatggacagagacgtgtacctgcacggacccatctgggccaagattccgaatacgggagcgcactttcatccttcacccatgatgggcggattcggattgaaggctcctcctcccatgatcctgatgaagaatacgcccgttcccggagtgacgcaggcgttcagcgaggtcgtgtccaacaacttcatcaaccagtattctacgggtcaggtgacagtgtccatcgaatgggagctgcagaaggaaaacagcaagcggtggaatccggagattcagtacaccaacaactacaacgacccccagtttgtggactttgccccggacagcaccggggaatacagaaccaccagacctatcggaacccgataccttacccgacccctttaa

SEQ ID NO: 838 - protein, 709 aa, Adeno-associated virus
MSFFDYIGKKYAQGAADWWNAEAGPPPPKKAKGKGFVFPGHNYLGPGNDLDNGNPVDEDDAAAKKHDEAYDEQLKQGDNPYVKYNHADAQLQEDLKDVNTFEANLAKGIFQAKKRVLEPFGLVEELAGKKRPHTEQESPDSSAGVGKKGEQPAKRRLDFSDAEAPQDGAAAAPSGVEDITMSGGGGAPMDGNQQGADGVGNASGNWHCDSQWMGNRVVTHSTRTWVLPSYNDHIYRQISSGAQTDHSKQYFGYSTPWGYFDFNRFHCHFSPRDWQRLVNNHWGFRPKKLHVKLFNIQVKEWVTQDGTNVVSNNLTSTVQVFTDDEYQLPYVLGNATEGCLPAFPPTVFTLPQYGYATLNDPGDDAPTNRSSFFCLEYFPSQMLRTGNNFEFSYSFEDVPFHTGWAPNQHLFKTANPLVDQYLYQLVGTNNTGDVQFEKVLAGNYGDAFKNWITGANRRTNSWLLGGGNRANVTALNPNYVQIEGDFYKSQPEPAGMANNLSGSDAVALGNSLIFNPQQTAPGTTTRLPAQNVLVTREDETQPVNTYASNSTGYVATNQQSTTSAPQNGQYHFQGIMPGAVWMDRDVYLHGPIWAKIPNTGAHFHPSPMMGGFGLKAPPPMILMKNTPVPGVTQAFSEVVSNNFINQYSTGQVTVSIEWELQKENSKRWNPEIQYTNNYNDPQFVDFAPDSTGEYRTTRPIGTRYLTRPL

SEQ ID NO: 839 - DNA, 2205 bp, Adeno-associated virus
  source 1..2205: mol_type = genomic DNA
atgtcttttttcgattggataggtcgtaagtacgccaacggcgccgctgagttctgggatctcgagccgggtcctccgaagcccaaaaaggccagagtcgacgactcggccggcttcaacttccccgggcacaagtacttgggccccggcaacggtctcgaccgaggggagcccgtggatgccgacgacgcggcggcccagaaacacgaccaggaatatcaggctctgcttgagagcggtgagaatccttacctcacttttaatcacgctgatcggcagctgcagaaggacctcgcggaggaccaatctttcgaggggaacctcgcccggggtctgttcgaagccaagaagctcgcggcacaggtcgtaggcgtcgaggagccggagctggctcctccagtcaagcgtccacactcgcccgagaaaaccccagagaaccagaagggtcagccgcgaccggatccccggacgccggccaagaagaggctcgagttctccgatcagcctggatcttcagcagacttacctgcatcctcacaacagtcccagcctcccgcaggggttcccggtgtggttcctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctcagacgcctccggcgtgcacagcctccccgggagccgatactttggctacagcaccccctgggggtacttcgacttcaatcgcttccactgccacttctcgcccagagactggcagcgcctcgtcaataaccactggggcttccgacccaagagactgcgagtcaaactcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccccccggacgtcttcacgctgccgcagtacggctacgcgacgctgaacgacagcaacaatggcaaccccaccaacagatcgagcttcttctgtctggaatactttcccagcaagatgctgagaaccgggaacaactttgaatttaccttcgagtttgaagacgtgcccttccacacgggcttcgccccctgtcagaacctgttcaagctgagcaatcccctggtggaccagtacctgtaccgcttccagggtactgacaactcctccagcagcacgcccggggtggtcaagttcgagaaatgcgtggcccacaactacgcccagcagtacaagaactggctgaccggtgctttccagaggaaccaggactataactacgtgtctggtactagcaactacaagggcgtggttggatctaatcagaataacctgcagcgcatagagaatgtgcagttcgccattgctccggacgtgcccagcatgtgtaaccatctggagggcaccaatatgatcgctctggacaacagcctgatcttccgggatgtctctaccgcccctggtgacacgactcagtataacatcaaccaggtgatcgtgaccagcgaagccgagactcaatctgtcaatgcgtactcgggcgacacctgtggccgcattgtgaataacagccagaatagcggaaccaacgctgggaccaccggcatcaactacaaaggtaccatgccgtcctcggtctggatggaccgtgacgtctacctgcagggcccgatctgggccaagattccccacacgggggcgcactttcacccgtctcccatgatggggggcttcggcctgaggaaccctcctccgatgatgctgatcaagaacacgcccgttcctggcaacgtgaccaccttcaccgaggtcaaggttaaccagttcatcacccagtactcgaccgggcagatcaccgtggacgtggaatgggagctgcagaaggagaactccaaacgatggaaccccgagatccagtacacaaataactacagtaataatacttttgtggactttgcccccaatgctaatggagattaccagatgacccgccccattggaacccgctacccgacccgccccccgtaa

SEQ ID NO: 840 - protein, 734 aa, Adeno-associated virus
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPENQKGQPRPDPRTPAKKRLEFSDQPGSSADLPASSQQSQPPAGVPGVVPGTMSAGGGAPVDDAQQGADGVGNASGDWHCDSKWLGNRVLTRSTRTWVLPSYNNHLYKQISDASGVHSLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTFEFEDVPFHTGFAPCQNLFKLSNPLVDQYLYRFQGTDNSSSSTPGVVKFEKCVAHNYAQQYKNWLTGAFQRNQDYNYVSGTSNYKGVVGSNQNNLQRIENVQFAIAPDVPSMCNHLEGTNMIALDNSLIFRDVSTAPGDTTQYNINQVIVTSEAETQSVNAYSGDTCGRIVNNSQNSGTNAGTTGINYKGTMPSSVWMDRDVYLQGPIWAKIPHTGAHFHPSPMMGGFGLRNPPPMMLIKNTPVPGNVTTFTEVKVNQFITQYSTGQITVDVEWELQKENSKRWNPEIQYTNNYSNNTFVDFAPNANGDYQMTRPIGTRYPTRPP

SEQ ID NO: 841 - DNA, 2205 bp, Adeno-associated virus
  source 1..2205: mol_type = genomic DNA
atgtcttttttcgattggataggtcgtaagtacgccaacggcgccgctgagttctgggacctcgagccgggtcctccgaagcccaaaaaggccagagtcgacgactcggccggcttcaacttccccgggcacaagtacctgggccccggcaacggtctcgaccgaggcgagcccgtggatgccgacgacgcggcggcccagaagcacgaccaggaatatcaggctctgcttgagagcggcgagaatccttacctcacttttaatcacgctgatcggcagctgcagaaggacctcgcggaggaccaatctttcgaggggaacctcgcccggggcctgttcgaagccaagaagctcgcggcgcaggtcgtaggcgtcgaggagcctgagctggctcctccagtcaagcgtccacactctcccgagaagaccccggaagaccagaagggtcagccgcgaccggatccccggacgccggccaagaagcggctcgagttctccgatcagcctggatctgcagcagacttacgtacatcctcacaacagtcccagcctcccgcaggggttcccggtgtggtttctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctccgacgcctccggcgtgcacggcctccccgggagtcgatactttggctacagcaccccctgggggtacttcgacttcaatcgcttccactgccacttctcgcccagagactggcagcgcctcgtcaacaaccactggggcttccgacccaagcgactgcgagtcaagctcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccctccggacgtcttcacgctgccgcagtacggctacgcgacgctgaacgacagcaacaacggcaaccccaccaacagatcgagctttttctgtctggaatactttcccagcaagatgctgaggaccgggaacaactttgaattcaccttcgagttcgaggacgtgcctttccacacgggcttcgccccctgtcagaacctgttcaagctgagtaaccccctggtggaccagtacctgtatcgcttccaaggaacagatgcctccactagcagtactcccggaatcgtcaagttcgagaaatgcgtagctcataattacgcccagcagtacaagaactggctgactggagctttccagaggaatcaggactacaactatgtgtctggtaccactaattataaaggaatcgtgggatccaaccagaataacttgcagcgcatagagaacgttcagtttgccatcgctccggacgtgcccagcatgtgtaaccatctcgaaggcaccaatatgatcgctctggataatagccttatcttcagagatgtgtctaccgcccccggcgacacgactcagtataacatcaaccaggtgatcgtgaccagcgaggccgagactcaacctgtcaattcgtactcgggcgacaactgtggccgcattgtgaataacagccagaataacgggaccaacgccgggactaccggcatcaactacaaaggtaccatgccttcctcggtctggatggaccgtgacgtctacctgcagggcccgatctgggccaagattccccacacgggggcgcactttcacccgtctcccatgatggggggcttcggcctgaagaaccctcccccgatgatgctgatcaagaacacgcccgttcctggcaacgtgaccaccttcaccgaggtcaaggttaaccagttcatcacccagtactcgaccgggcagatcaccgtggacgtggaatgggagctgcagaaggagaactccaagagatggaaccccgagatccagtacacaaacaactacagcaataacacttttgtggactttgcccccaatgccaatggagattaccagatgacccgccctatcggaacccgataccttacccgacccctttaa

SEQ ID NO: 842 - protein, 734 aa, Adeno-associated virus
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPEDQKGQPRPDPRTPAKKRLEFSDQPGSAADLRTSSQQSQPPAGVPGVVSGTMSAGGGAPVDDAQQGADGVGNASGDWHCDSKWLGNRVLTRSTRTWVLPSYNNHLYKQISDASGVHGLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTFEFEDVPFHTGFAPCQNLFKLSNPLVDQYLYRFQGTDASTSSTPGIVKFEKCVAHNYAQQYKNWLTGAFQRNQDYNYVSGTTNYKGIVGSNQNNLQRIENVQFAIAPDVPSMCNHLEGTNMIALDNSLIFRDVSTAPGDTTQYNINQVIVTSEAETQPVNSYSGDNCGRIVNNSQNNGTNAGTTGINYKGTMPSSVWMDRDVYLQGPIWAKIPHTGAHFHPSPMMGGFGLKNPPPMMLIKNTPVPGNVTTFTEVKVNQFITQYSTGQITVDVEWELQKENSKRWNPEIQYTNNYSNNTFVDFAPNANGDYQMTRPIGTRYLTRPL

SEQ ID NO: 843 - DNA, 2205 bp, Adeno-associated virus
  source 1..2205: mol_type = genomic DNA
atgtctttcttcgattggataggtcgtaagtacgccaacggcgccgctgagttctgggacctcgagccgggtcctccgaagcccaaaaaggcccgagtcgacgactcggccggcttcaacttccccgggcacaagtacctgggccccggcaacggtctcgaccgcggggagcccgtggatgccgacgacgcggcggcccagaagcacgaccaggaatatcaggctctgcttgagagcggcgagaatccttacctcacttttaatcacgctgatcggcagctgcagaaggacctcgcggaggaccaatctttcgaggggaacctcgcccggggcctgttcgaagccaagaagctcgcggcgcaggtcgtaggcgtcgaggagccggagctggctcctccagtcaagcgtccacactcgcccgagaagaccccggaggaccagaagggtcagccgcgaccggatccccggacgccggcaaagaagcggctcgagttctccgatcagcctggatcttcagcagacttacctgcatcctcacaacagtcccagcctcccgcaggggttcccggtgtggttcctggtacgatgtctgcaggaggaggcgctccagtggacgatgctcaacaaggtgccgacggagtgggcaatgcctcgggagattggcattgcgattccaaatggctgggcaaccgagttctcacccgatccacccggacctgggtgctgcccagctacaacaaccacctgtacaagcagatctccgacgcctccggcgtgcacggcctccccgggagccgatactttggctacagcaccccctgggggtacttcgacttcaatcgcttccactgccacttctcgcccagagactggcagcgactcgtcaacaaccactggggcttccgacccaagcgtctccgagtcaagctcttcaacatccaggtcaaggaggtcacgactactgattcgacgaccacggtctccaacaacctcacgagcacggtccaggtcttcacagacgacgagtaccagctgccgtacgtctgcggcaacgccaccgagggatgcctgccgccgttccctccggacgtcttcacgctgccgcagtacggctacgcgacgctgaacgacagcaacaacggcaaccccaccaacagatcgagcttcttctgtctggaatactttcccagcaagatgctgaggaccgggaacaactttgaattcacctacgagttcgaggaagtgcccttccacacgggcttcgccccctgtcagaacctgttcaagctgagtaaccccctggtggaccagtacctgtatcgcttccaaggaacagatgcctcaactagcagcactcccggaatcgtcaagttcgagaaatgcgtagcccataattacgcccagcagtacaagaactggctgactggagctttccagaggaaccaggactacaactatgtgtctggcaccactaattataaaggcatcgtgggatctaatcagaataacctgcagcgcatagagaatgtgcagttcgccattgctccggacgtgcccagcatgtgtaaccatctggagggcaccaacatgattgccctggacaacagcctgatcttccgagatgtctctaccgcccccggtgacacgactcagtataacatcaaccaggtgatcgtgaccagcgaggccgagactcaacccgtcaatgcgtactcgggcgataactgtggccgcattgtgaataatagccagaataacgggaccaacgccgggaccaccggcatcaactacaaaggtaccatgccttcctcggtctggatggaccgtgacgtctacctgcagggcccgatctgggccaagattccccacacgggggcgcactttcacccgtctcccatgatggggggcttcggcctgaagaaccctcctccgatgatgctgatcaagaacacgcccgttcctggcaacgtgatcaccttcaccgaggtcaaggtcaaccagttcatcacccagtactcgaccgggcagatcaccgtggacgtggaatgggagctgcagaaggagaactccaagagatggaaccccgagatccagtacacaaacaactacagcaacaacacttttgtggactttgcccccaatgccaatggagattaccagatgacccgccctatcggaacccgataccttacccgacccctttaa

SEQ ID NO: 844 - protein, 734 aa, Adeno-associated virus
MSFFDWIGRKYANGAAEFWDLEPGPPKPKKARVDDSAGFNFPGHKYLGPGNGLDRGEPVDADDAAAQKHDQEYQALLESGENPYLTFNHADRQLQKDLAEDQSFEGNLARGLFEAKKLAAQVVGVEEPELAPPVKRPHSPEKTPEDQKGQPRPDPRTPAKKRLEFSDQPGSSADLPASSQQSQPPAGVPGVVPGTMSAGGGAPVDDAQQGADGVGNASGDWHCDSKWLGNRVLTRSTRTWVLPSYNNHLYKQISDASGVHGLPGSRYFGYSTPWGYFDFNRFHCHFSPRDWQRLVNNHWGFRPKRLRVKLFNIQVKEVTTTDSTTTVSNNLTSTVQVFTDDEYQLPYVCGNATEGCLPPFPPDVFTLPQYGYATLNDSNNGNPTNRSSFFCLEYFPSKMLRTGNNFEFTYEFEEVPFHTGFAPCQNLFKLSNPLVDQYLYRFQGTDASTSSTPGIVKFEKCVAHNYAQQYKNWLTGAFQRNQDYNYVSGTTNYKGIVGSNQNNLQRIENVQFAIAPDVPSMCNHLEGTNMIALDNSLIFRDVSTAPGDTTQYNINQVIVTSEAETQPVNAYSGDNCGRIVNNSQNNGTNAGTTGINYKGTMPSSVWMDRDVYLQGPIWAKIPHTGAHFHPSPMMGGFGLKNPPPMMLIKNTPVPGNVITFTEVKVNQFITQYSTGQITVDVEWELQKENSKRWNPEIQYTNNYSNNTFVDFAPNANGDYQMTRPIGTRYLTRPL

SEQ ID NO: 845 - DNA, 2172 bp, Adeno-associated virus
  source 1..2172: mol_type = genomic DNA
atgtctttctttgattgggtaggtaaaaagtacgctcaaggagctgctgaattctgggatctgaagtccggtcctcctgcaccaaaaaaggcgcgaaaggacggatctgccggattcaatttccccgggcataaatatctcggacccggtaactctctcgatcgaggagatccggttgacgccgacgacgccgctgctcaaaagcacgatcaagcttatcaagagcagcttgaagcgggagataatccctatctgaaatacaatcacgccgatcgcgagttccaagaggcgttgaaagacgatacctcgtttgaaggaaatctcgcgagaggactctttgaggccaagaagctcgtggccgagcctctaggtctcgttgaaccggaactggctccggggaagaaacgcccagcggcttcccccgttcaatcgccagactcgtcttccggtatcggcaagaagggcgcgcaaccttcccgccgaagattggactttgaccaggccgatcgagaaaccgctgccgaggctggagccacaggcaccggaagtctaccacccgcctctaatttgggacctgttacaatgtctgctgggggaggcgcaccgatggacgacaatcaacaaggtgccgatggagtgggcaatgcctcgggaaattggcattgcgattcccaatggctgggcgacagagtcattacaaagtccacccgaacctggagcctgcccacctacaacaaccacttgtacaagaagatctccagcaacgaagcttcaggagacaagacttggtttggattcagtaccccttggggatactttgacttcaaccgcttccactgtcacttctccccccgagactggcagcgtctcatcaacaaccactggggcatccgcccgaagaagatgcacgtcaagctcttcaatattcaagttaaggaggtcacgacgcaagactcgacgacgaccatcgctaataaccttaccagtacggttcaggtctttgcggactcggaatatcagctgccgtacgtagccgggaatgcgcacgaaggatgcttgccaccttttccagctgatgtctttatgctgccgcagtacggctactgcactctccagaacagcgcttccaacgatccagtggcccagagtgccttctactgtctggaatatttccccagtcagatgctgagaacgggaaataactttgtcttcagctacgagttcgaagacgtcccgttccacagtatgtacatgcacaaccaatcgttggaccgcttgatgaacccgctgattgatcagtacttgtactatctctccgccaccaatgctcccgccaatacaaataaatttaccaagtcaaataaaggtgatctaccgtctcagctcaggaactggattcctggaccttcatttcaacaagcagcttggactctagacggacagagtcaaacccaaacgaacaacacgtgggattatgccaataaataccaattggatgcacgaaagaacaagttggctcccggtccagttaatgcacctcgagaaggacagtttcaagcaaatacgctgatattcggcaaagatccaaccgtctcttctaccttggctgaacaagacaaagttaacattacacgagacaatgaaactcggacagtaaacccaattctgggaacggctcagatatttgacatcatcaacaacgacactccgcgtgcgaacggctcaataagcaaaatcaacatgaacatctgtcctgtcatgcctggaagcgtctggcaagatcgagatatctacctacaaggaccaatctgggccaaggttcctcgcacagatggaaagttccatccctctcctctcatgggtggatttggacttagatgtccacctccacagattttcattaagaatactccggttcctggcaaccctccaacaacatttgaaccaaccaaagtggccagcttcatcacgcagtactcgaccggacaagtcaccgtcgaaatggaatgggagcttgaaaaggaaaactccaaacgctggaacccggaaatacagttcaccaacaacttctcctcttcggacggaaacatggtccactttgctgtggactctaccggactctactcagaaccccgtcccgtgggcacccgttaccttacccgctctttgtaa

SEQ ID NO: 846 - protein, 723 aa, Adeno-associated virus
MSFFDWVGKKYAQGAAEFWDLKSGPPAPKKARKDGSAGFNFPGHKYLGPGNSLDRGDPVDADDAAAQKHDQAYQEQLEAGDNPYLKYNHADREFQEALKDDTSFEGNLARGLFEAKKLVAEPLGLVEPELAPGKKRPAASPVQSPDSSSGIGKKGAQPSRRRLDFDQADRETAAEAGATGTGSLPPASNLGPVTMSAGGGAPMDDNQQGADGVGNASGNWHCDSQWLGDRVITKSTRTWSLPTYNNHLYKKISSNEASGDKTWFGFSTPWGYFDFNRFHCHFSPRDWQRLINNHWGIRPKKMHVKLFNIQVKEVTTQDSTTTIANNLTSTVQVFADSEYQLPYVAGNAHEGCLPPFPADVFMLPQYGYCTLQNSASNDPVAQSAFYCLEYFPSQMLRTGNNFVFSYEFEDVPFHSMYMHNQSLDRLMNPLIDQYLYYLSATNAPANTNKFTKSNKGDLPSQLRNWIPGPSFQQAAWTLDGQSQTQTNNTWDYANKYQLDARKNKLAPGPVNAPREGQFQANTLIFGKDPTVSSTLAEQDKVNITRDNETRTVNPILGTAQIFDIINNDTPRANGSISKINMNICPVMPGSVWQDRDIYLQGPIWAKVPRTDGKFHPSPLMGGFGLRCPPPQIFIKNTPVPGNPPTTFEPTKVASFITQYSTGQVTVEMEWELEKENSKRWNPEIQFTNNFSSSDGNMVHFAVDSTGLYSEPRPVGTRYLTRSL

SEQ ID NO: 847 - DNA, 2190 bp, Adeno-associated virus
  source 1..2190: mol_type = genomic DNA
atgtcttttgtcgatcaccctccagattggttggaagacgttctcgacggcttcaacgaatttctgggtctcaaatcgggaccacctcagccaaaggccgggcagtacgataagcccaaagacaacaccagaggcctagtccttcctggctacaaatacttgggtccgggcaacggactcgataaaggagagcccgtcaacgaggctgacgccgctgctctcgagcacgacaaggcctacgacgagcagcttaaagcgggagacaacccgtacgtcaaatacaatcacgcagacgccgaattccaagagcgtctgcagggagatacgtcttttggcgggaaccttgctcgtggtgtattccaggccaagaagcgggtcctagagccgctcggtctggtcgaaggagacgagacgggtcccatcaagagaccccatacgcaagagagtcccgagtccagttccgggatcggaaagaagggccatcagcctgcccgcaagaggctcaactttgatcaagctggagaatcgagctctggtgccgcaggacagtctaacggaagcatgtctgcagacggtactgagcgtgcagcagctggcggagctgcggtcgatgcagctcaaggtgccgatggagtgggtaattcctccggtaattggcattgcgattccacgtggggagataatcacgtcaccaccaccagcaccagaacctgggtgctgcccaactacaacaaccacctgtaccgaagactcggcgtcagcaacacctccaacacctacaacggattcagcaccccctgggggtactttgactttaaccgattccactgccacttctctccacgagactggcagcgactcatcaataaccattggggaatcagacccaaacgcatcagcgtcaaaatcttcaacattcaagtcaaggaggtcacggtgcaggacggcaccacgaccgtcgccaacaatctcaccagcaccatacaggtctttgcggacaaggcgtacgacctgccgtacgtgatggacgccggacaggagggcagtctgcctccgttccccaacgacgtctttatgataccgcagtacggctacatgggactggtgacaggcggcagcagccagaaccagacggacagaaacgccttctactgtctagagtactttccctctcagatgctgagaacgggaaacaactttgaaatgagcttcgactttgaagacgtgcccttccactccatgttcgctcacagccagagtttggacagactgatgaatcctctgctggatcagtacctgtggagtctgcactcgaccacagggggagcctatggcaccggcaccaagctcaatacgttcatcaagaaccagaccacagacttttcgtcttatagacgcaactggctgcctggtcctttcatcaaacagcagagatggaacaagggcaataccaactactcggtgcccgtctctggaaacgactcgatgctccaccacgagtctcacacgactctgaacggtcgatggagcaaactggctcccggaccggccatggcaactccagaaaacacggctaccagctttacacaaggccagctcatgtttgccgggcccgggcagaccactaccagtaccaccaactccaacaacatcatcttcacctcggaggacgagctgattgccactaatcccaccgactacgattactggggagccgtcacagacggacagcagacctctaccgtggcacccagcgaacgagacgtggacgcgatgccgactatgcccggcatggtctggcaagacagagacatttactaccagggccccatctgggccaagattcctcataccgacggacgcttccatccgtctcctttgattggaggcttcggtctgcacaagcctcctcctcaaatcttcatcaaaaacacgccggtacctgccaatccagcaaccaccttctcggctactcgcatcaacaccttcctgaaccagtacagcaccggacaagtcgccgtccagatcgaatgggagctgcgcaaagaaaagtccaagcgatggaacccggaaatccagttcacttccaactacggtacccagaattccatgctctgggcgccagatgccgctggaaactacgaagagcccagagccatcggtacccgctacctcacccattgcctgtaa

SEQ ID NO: 848 - protein, 729 aa, Adeno-associated virus
MSFVDHPPDWLEDVLDGFNEFLGLKSGPPQPKAGQYDKPKDNTRGLVLPGYKYLGPGNGLDKGEPVNEADAAALEHDKAYDEQLKAGDNPYVKYNHADAEFQERLQGDTSFGGNLARGVFQAKKRVLEPLGLVEGDETGPIKRPHTQESPESSSGIGKKGHQPARKRLNFDQAGESSSGAAGQSNGSMSADGTERAAAGGAAVDAAQGADGVGNSSGNWHCDSTWGDNHVTTTSTRTWVLPNYNNHLYRRLGVSNTSNTYNGFSTPWGYFDFNRFHCHFSPRDWQRLINNHWGIRPKRISVKIFNIQVKEVTVQDGTTTVANNLTSTIQVFADKAYDLPYVMDAGQEGSLPPFPNDVFMIPQYGYMGLVTGGSSQNQTDRNAFYCLEYFPSQMLRTGNNFEMSFDFEDVPFHSMFAHSQSLDRLMNPLLDQYLWSLHSTTGGAYGTGTKLNTFIKNQTTDFSSYRRNWLPGPFIKQQRWNKGNTNYSVPVSGNDSMLHHESHTTLNGRWSKLAPGPAMATPENTATSFTQGQLMFAGPGQTTTSTTNSNNIIFTSEDELIATNPTDYDYWGAVTDGQQTSTVAPSERDVDAMPTMPGMVWQDRDIYYQGPIWAKIPHTDGRFHPSPLIGGFGLHKPPPQIFIKNTPVPANPATTFSATRINTFLNQYSTGQVAVQIEWELRKEKSKRWNPEIQFTSNYGTQNSMLWAPDAAGNYEEPRAIGTRYLTHCL

SEQ ID NO: 849 - DNA, 2142 bp, Adeno-associated virus
  source 1..2142: mol_type = genomic DNA
atgtctttctttgattggttaggtaaacagtacgctcaaggagctgctgaattctgggatttgaagtccggtcctcctgcaccaaaaaaggcgcgaaaggacggatcagccggattcaatttccctgggcacaaatacctgggtcctggcaattcattggatcgcggagatcccgtggacgctgacgacgccgctgctcaaaagcacgaccagtcgtaccaagagcagcttgaggcgggagacaatccctacctcaagtacaaccacgccgaccgcgagttccaggaggcgttgaaggacgacacctcctttgaaggaaatctcgcgagaggactctttgaggccaagaagctcgtggcagagcctcttggtctcgtggaaccagaactggcgccacccagtggccgtaaacgaccggtgcaatcgagtcaagagtctggttactcgagtagccaagacaagcggcccaacctcgacgtagacgaggaggaccgtgagttcgctgccgctgcagcggagaccgaaactggaagcgctccccccaccggcaatttgggacctggtacgatggctggaggcggtagcgcgccaatcgacgacggctcgtatggtgccgatggagtgggcaatgcctcgggagattggcattgcgattccacatggctggacaactgtgtcatcacccgaaccactcggacctggaatctgccaacctacaacaaccacatctacaaacgactcaacggaacgacctccggagaccaaagctacttcggattcagcaccccctggggatactttgacttcaaccgcttccactgtcatttctcccctcgagactggcaaagactcatcaacaataactggggactccgaccaaagagcctacggttcaaaatctttaacattcaagttaaagaagtcacgacgcaagactcaacgaagatcatctccaataaccttaccagcacggttcaggtatttgcggacacggagtaccagctcccgtacgtgattggatcggctcacgaaggatgtctgcctcctttcccggctgacgtgttcatgctgccgcagtatggatactgtactcgacaagacggaaacagcaacaatccgaccccgagaagcgccttctattgtttggagtactttcccagcaagatgctaagaactgggaacagttttgaatttacatataactttgagaaggtgccgttccacagcatgtgggctcacaaccagagtctggatcgattgatgaatccattgattgatcagtacctgtactaccttgatgtcacttcgagtaccgggtttacctatcaaaaaggagttcacacgaacttgcccgaacaagagcgcaactggctgccgggaccaggaattcgaaatcaagcttggtttaattctgcaactggaaacaacccactaactggtacctggcaatattccaacaaatacgtacttgaaaaccgtgcttcgaagattgctcctggacctgccatgggaattgaatcaacaaaattcgacgggaacggaatcatcttttctaaagaatacatcaccaatgtaaatacagccaatccgaatcaagtaaacatcacacgcgagaccgaaataaactcaacgaaccccttggctggaggatctctcggagcccacgctaataattcacaaaacacaacaacggcaccgacgctagaccacaccaacgtcatgggtgtgtttccgggtagtgtctggcaagacagagacatttaccttcaaggacaaatctgggccaagattccccacacagacggacatttccacccttctcctctcatgggaggatttggactgaagaacccgcctcctcaaattctgatcaaaaacacacctgttccggctgacccaccaactgaattcaatgcgaacaaaatctcttctttcatcactcaatactcaaccggacaagttacagtggaaatggaatgggaacttcagaaagaaacctccaaaagatggaatccagaaatccagtacagcgacgactcgtcttcgacgtctggctccattctgcactttgctccggatgatgttggaaactacaaagagttccgctctatcggaactcgttaccttacccgtcctctgtaa

SEQ ID NO: 850 - protein, 713 aa, Adeno-associated virus
MSFFDWLGKQYAQGAAEFWDLKSGPPAPKKARKDGSAGFNFPGHKYLGPGNSLDRGDPVDADDAAAQKHDQSYQEQLEAGDNPYLKYNHADREFQEALKDDTSFEGNLARGLFEAKKLVAEPLGLVEPELAPPSGRKRPVQSSQESGYSSSQDKRPNLDVDEEDREFAAAAAETETGSAPPTGNLGPGTMAGGGSAPIDDGSYGADGVGNASGDWHCDSTWLDNCVITRTTRTWNLPTYNNHIYKRLNGTTSGDQSYFGFSTPWGYFDFNRFHCHFSPRDWQRLINNNWGLRPKSLRFKIFNIQVKEVTTQDSTKIISNNLTSTVQVFADTEYQLPYVIGSAHEGCLPPFPADVFMLPQYGYCTRQDGNSNNPTPRSAFYCLEYFPSKMLRTGNSFEFTYNFEKVPFHSMWAHNQSLDRLMNPLIDQYLYYLDVTSSTGFTYQKGVHTNLPEQERNWLPGPGIRNQAWFNSATGNNPLTGTWQYSNKYVLENRASKIAPGPAMGIESTKFDGNGIIFSKEYITNVNTANPNQVNITRETEINSTNPLAGGSLGAHANNSQNTTTAPTLDHTNVMGVFPGSVWQDRDIYLQGQIWAKIPHTDGHFHPSPLMGGFGLKNPPPQILIKNTPVPADPPTEFNANKISSFITQYSTGQVTVEMEWELQKETSKRWNPEIQYSDDSSSTSGSILHFAPDDVGNYKEFRSIGTRYLTRPL

SEQ ID NO: 851 - DNA, 2232 bp, Adeno-associated virus
  source 1..2232: mol_type = genomic DNA
atgtctttctttgattggataggtaaacagtacgctcaaggagctgctgaattctgggatttgaagtccggtcctcctgcaccaaaaaaggcgcgaaaggacggatccgccggattcaatttccctgggcacaagtacctgggtcctggcaattcattggatcgcggagatcccgtggacgccgacgacgctgctgctcaaaagcacgaccagtcgtatcaagaacagcttgaggcgggagacaatccctacctcaagtacaatcacgcggatcgcgagttccaggaggcgttgaaaggagacacctccttcgaaggaaatctcgcgagaggactctttgaggccaagaagctcgttgcagagcctctcggccttgttgaaccggaactggcgccacctagtggccgcaagcgaccggtgcaatcgagtcaagaatctggttactcgagtagccaagacaagcggcccaacctcgacttagacgaggaggatcgtgagctcgctgccgctgcagcggagaccgaaactggaagcgctccccccaccggcaatttgggacctggtacgatggctggaggcggtagcgcgccaatcgacgacggctcgtatggtgccgatggagtgggcaatgcctcgggagattggcattgcgattccatatggatggacgaccgtgtcgtcacccgcaccactcggacctggaacctgcccacctacaataaccacgtctacaaacgactcaacggaaacacggaacagaccaccggagatggaagctacttcggattcagcaccccgtggggatacttcgacttcaaccgcttccactgtcatttctcccccagagactggcaaagactcatcaataacaattggggactcagaccaaaaagtctacggttcaaaatctttaacattcaggttaaagaggtcacggtgcaagaccaaacgacgatcatctccaataaccttaccagcacggttcaagtgtttgcggacacggactatcaattgccgtacgtgattggttctgctcacgaaggttgtctgcctcctttcccagccgatgtgtttatgctgccgcagtatgcatactgcactcgacaagacacaaacagcagtaatcccactcccagaagcgctttctattgtttggaatactttcccagcaagatgttgagaactggtaacaactttgaatttacttacaattttgagaaggtgcccttccacagcatgtgggctcataaccagtctttagatcggctgatgaaccctctcatcgatcagtatttgtactacctcaatcaaactacaaatagtgctctatccagccgatctaagactggttacgagcctagccacagaagcgtgttcgtgctgccccagagcaagtacaatagtgctctatcctacaacaaatcaacaaaatcaaatctacctgagcaagaacgcaactggctacctggacctggcatcagaaatcaaacgtggttcagcagtggaactggcaacatggctgtagacgccatgtggaactactcgaataagtatgtgctggagaacagagcttccaagattgctcctggtccagctatggggattgagtcaacaaaagtagatggcaatggactaatcttctctaaagagtatataactaattcaaccaccgcgaatgctgatcaagtgaacgtcactagagaaactgaaatcaactcaaccaatcccttggcgggcaaatcacttggagctcatgcgaacaatatacagaaccagacaacactgccaacacttgctaatactgacatcatgccggtactacctggcagcgtttggcagaatagagatatctaccttcaaggccaaatctgggcgaagattcctcacacagatggacactttcatccttcaccgctaatgggaggattcggtctaaagaatcctcctcctcaaatactgatcaagaacacaccagtgccagcaaatcctccggtagaatttacagctggcaagattaactctttcatcactcagtattcaactggtcaagtcacagtcgaaatggaatgggaacttcagaaagaaacatccaaaagatggaatccagagatccaatacagcgacgactcatcagccaccaaccaatcggtgctgcactttgctccaaacgaaacaggagagtacaaggaattccgttcaattggatcccgttatctcactcgtcctctgtaa

SEQ ID NO: 852 - protein, 743 aa, Adeno-associated virus
MSFFDWIGKQYAQGAAEFWDLKSGPPAPKKARKDGSAGFNFPGHKYLGPGNSLDRGDPVDADDAAAQKHDQSYQEQLEAGDNPYLKYNHADREFQEALKGDTSFEGNLARGLFEAKKLVAEPLGLVEPELAPPSGRKRPVQSSQESGYSSSQDKRPNLDLDEEDRELAAAAAETETGSAPPTGNLGPGTMAGGGSAPIDDGSYGADGVGNASGDWHCDSIWMDDRVVTRTTRTWNLPTYNNHVYKRLNGNTEQTTGDGSYFGFSTPWGYFDFNRFHCHFSPRDWQRLINNNWGLRPKSLRFKIFNIQVKEVTVQDQTTIISNNLTSTVQVFADTDYQLPYVIGSAHEGCLPPFPADVFMLPQYAYCTRQDTNSSNPTPRSAFYCLEYFPSKMLRTGNNFEFTYNFEKVPFHSMWAHNQSLDRLMNPLIDQYLYYLNQTTNSALSSRSKTGYEPSHRSVFVLPQSKYNSALSYNKSTKSNLPEQERNWLPGPGIRNQTWFSSGTGNMAVDAMWNYSNKYVLENRASKIAPGPAMGIESTKVDGNGLIFSKEYITNSTTANADQVNVTRETEINSTNPLAGKSLGAHANNIQNQTTLPTLANTDIMPVLPGSVWQNRDIYLQGQIWAKIPHTDGHFHPSPLMGGFGLKNPPPQILIKNTPVPANPPVEFTAGKINSFITQYSTGQVTVEMEWELQKETSKRWNPEIQYSDDSSATNQSVLHFAPNETGEYKEFRSIGSRYLTRPL

SEQ ID NO: 853 - DNA, 2187 bp, Adeno-associated virus
  source 1..2187: mol_type = genomic DNA
atgtcgtttgttgatcaccctccagattggttagaagaggttggtgaaggccttcacgagtttttggagctcgaagctggcccacccaaaccgaagcccaatcagcaaaaacaagacaacgcccgtggtcttgtactgcctggatataattatctgggacccttcaacggactcgacaagggagagcccgtcaaccgagcggacgctgttgcgcgagagcacgatatctcgtacaacgagcagctccaggcgggagacaacccctacctcaagtacaaccacgcggacgccgagtttcaggagaagctcgccgacgacacctcctttgggggcaacctcggaaaggcaatctttcaggccaagaagagggttctcgaaccttttggcctggttgaagagcctgttaagacggctccggcaaagaagaggcctgtagagcagtctcctgcggaaccgggcccatcctctggcatcggaaaggcgggcgatcagcctgcgagaaagcgactaaactttggtcagactggagacgccgactccgaacccgacccccagcctctcggagaagcaccagcagccaatcccaatttgggttctgctacaatgtctggaggcggaggcgcgccgatggcagatgatcaacagggtgccgatggagtgggtacttcctcaggaaatcggcattgcgattccacatggctggacaagcgaatcattaccaagtccacccgaacctggagcctgcccacctacaacaaccatctctacaacaagatcatgtccaccaacgccaatggagacagcgcttggtttggcttcagcactccctggggatactttgacttcaaccgcttccactgccacttctcgccccgagactggcaacgactcatcaacaacaactggggcatccggcccaagaaaatgcacgtcaacctcttcaatatccaagtcaaggaagtcacgacgcaagactcgacgacgaccaccaccaataaccttaccagcacgattcaggtgtttgcggacacggagtaccagctgccgtacgtagccagcaacgcccacgagggctgtctcccgccctttccggcggacgtcttcatgcttccgcagtacggctactgcacgctgcaacgcgagaactccaacgatcctgtggcccagagctcgttctactgcctggagtacttccccagccagatgctgaggaccggaaacaactactccatcagctacacgttcgaagacgtgcccttccacagcatgtacatgcacaaccagagcctggaccggctgatgaacccgctcatcgaccagtacttgtggtacctgaataccaccgtatcgaccaacaacacaaacaccttctccaagtcgaccaaaggcgacctggcacgccagaaccgaaactggctgcctggacccgccttccagacggtggccttcaacctgaacggacaaggcaactttggatcgaccaatcactggctttacttgaacaagtacacaatgaatggaaaaagcgtggctatcggccccggccccgtcgtggctcccaagagtacaaccttccaggccgacggcatgatcttcgccaagagcgcagatacggcgacctctgctgcccctcaaaatgaggtcaacatcaccagcgaggcggagacgtccaccgtcaatcccatgatgggcaccaatcctctcattatcaattcgagcaatactccatctacctccgctcccacgatgtcaaatgtatccgttaatcccgtcatgcctggctccgtttggcaagacagagacatctaccttcaaggcccgatctgggccaagattccgcgtaccgacggaaccttccacccctcgcctctgatgggaggattcgggctgcgtcgtcctcctcctcaaatcttcatcaagaacacgcccgttccgtccaatcctccaaccacgttcaatcccgccaagatcaactccttcatcacccaatactcgaccggacaggtcaccgttgagatggaatgggagctcgagaaggaaaacagcaagaggtggaacccggaaatacagttcaccaacaatttccaggtttcggacgggaacatggttcccttctgcgtggactcacgtggtgtctactctgaaccccgccccatcggaacccgctaccttacccgaaccctctaa

SEQ ID NO: 854 - protein, 728 aa, Adeno-associated virus
MSFVDHPPDWLEEVGEGLHEFLELEAGPPKPKPNQQKQDNARGLVLPGYNYLGPFNGLDKGEPVNRADAVAREHDISYNEQLQAGDNPYLKYNHADAEFQEKLADDTSFGGNLGKAIFQAKKRVLEPFGLVEEPVKTAPAKKRPVEQSPAEPGPSSGIGKAGDQPARKRLNFGQTGDADSEPDPQPLGEAPAANPNLGSATMSGGGGAPMADDQQGADGVGTSSGNRHCDSTWLDKRIITKSTRTWSLPTYNNHLYNKIMSTNANGDSAWFGFSTPWGYFDFNRFHCHFSPRDWQRLINNNWGIRPKKMHVNLFNIQVKEVTTQDSTTTTTNNLTSTIQVFADTEYQLPYVASNAHEGCLPPFPADVFMLPQYGYCTLQRENSNDPVAQSSFYCLEYFPSQMLRTGNNYSISYTFEDVPFHSMYMHNQSLDRLMNPLIDQYLWYLNTTVSTNNTNTFSKSTKGDLARQNRNWLPGPAFQTVAFNLNGQGNFGSTNHWLYLNKYTMNGKSVAIGPGPVVAPKSTTFQADGMIFAKSADTATSAAPQNEVNITSEAETSTVNPMMGTNPLIINSSNTPSTSAPTMSNVSVNPVMPGSVWQDRDIYLQGPIWAKIPRTDGTFHPSPLMGGFGLRRPPPQIFIKNTPVPSNPPTTFNPAKINSFITQYSTGQVTVEMEWELEKENSKRWNPEIQFTNNFQVSDGNMVPFCVDSRGVYSEPRPIGTRYLTRTL

SEQ ID NO: 855 - DNA, 2175 bp, Adeno-associated virus
  source 1..2175: mol_type = genomic DNA
atgtcgtttgtcgatcaccccccagactggctcgaggagatcggagaggggctctccgagttcatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggcgggggacaacccgtacctcgcctacaaccacgcggacgccgagttccagaaggacctcagcggcgactccagcctagccggcaacgccgcgaacgcgctcttccaggccaagaagagggtcctcgagcccttcggactggtcgagggagagccggagcccaagaagacgccttccgtcaagaggccgcacgcatcgccggactcgtcgagcggcgtcgggaagaagggcgaccagcccgcgcggaagaggctcgactttgggacggagcccgcgggtcaagacggagcgggacgagcggcccaggcagccggagatatggcatctgctgaggtggctgcgggtggtggcggaccagtgggcgacgatgcacaaggtgccgatggagtgggtaatgcctcaggaaattggcattacgattccgtttggatggacggcgctgtcatcaccaagtccacccgaacctggagcctgcccgcctacaacaaccacctctaccgccagatccagtccagcggcaccggagacggcacgtactttggttacagcacgccttggggatacttcgatttcaatcgattccactgccacttttctcccagggactggcagcggctcatcaacaaccactggggcatccgccccaagcggctccactttaagctcttcaacatccaggtcaaggaggtcacgacgaccgacggcaccacgaccatcgccaacaacctcaccagcaccatacaggtctttgcggacacggagtaccagctcccgtacgtgctcggcaacgcccacgagggctgcctgccgccgttcccggcggacgtcttcatgctgccgcagtacgcgtacctgacgctcaacgccaaccccacgaaccagggcgcggccgcgctcagcctgccgcagagcgccttctactgcctggagtactttccgagccagatgctgaggaccgggaacaacttctcgttcagctacgagttcgagaagctgcccttccactcgatgttcatgcacagccagagcctggaccggctcatgaacccgctcatcgaccagtacctctggtacctcaacgccacgaccggcaacaacctctccttcaacaaggccggcgccaagaacttccccgagtacttccgcaactggctgcccgggcccggccagcgcgtgcagcagtggagcacgatcggaacccagaacaacgcgcagaccggcacgtgggccagcgccaacaagtggatcctcatgggcaggtccagcaagatggcccccgggctggcccagccggtcaggaacgcccagaccgtcaccaacggctcgcagctcatcttcaacaacgagaccatcaagggctccaccgcgacggcctccaccgtacactcggggctgctcgtcaccaacgagtcggagaccgccccgaccaaccccaactcggcgacaaagtggggcgtcatgacggacaaccagcagaccacctccactacgccgaccgtcagcgacgacctcgaggcgcacgtcttcccgggcatggtctggcaggaccgcgacatctacctgcagggcccgatatgggccaagatcccggagacggacggccacttccacccgtccccgctcatgggcggcttcgggctcaagaacccgccgccgcagatcctcgtcaagaacacgcccgtgcccgccgtgccgcccacgaccttcacaccgcagaaggtcaactccttcatcacgcagtactccaccggccaggtcaccgtcgagatcgagtgggagctgcgcaaggagaagagcaagcgctggaaccccgagatccagtacacctccaacttcgagaactcggccaacgtacagttctccgtcaacggcgacggcgcgtacatcgagccgcgaccgatcggaacccggtacctcacccacaacctgtaa

SEQ ID NO: 856 - protein, 724 aa, Adeno-associated virus
MSFVDHPPDWLEEIGEGLSEFIGLEAGPPKPKPGYQDRARGLVVPGYKYLGPFNGLDRGEPVNAADAAAKKHDEEYDRLLKAGDNPYLAYNHADAEFQKDLSGDSSLAGNAANALFQAKKRVLEPFGLVEGEPEPKKTPSVKRPHASPDSSSGVGKKGDQPARKRLDFGTEPAGQDGAGRAAQAAGDMASAEVAAGGGGPVGDDAQGADGVGNASGNWHYDSVWMDGAVITKSTRTWSLPAYNNHLYRQIQSSGTGDGTYFGYSTPWGYFDFNRFHCHFSPRDWQRLINNHWGIRPKRLHFKLFNIQVKEVTTTDGTTTIANNLTSTIQVFADTEYQLPYVLGNAHEGCLPPFPADVFMLPQYAYLTLNANPTNQGAAALSLPQSAFYCLEYFPSQMLRTGNNFSFSYEFEKLPFHSMFMHSQSLDRLMNPLIDQYLWYLNATTGNNLSFNKAGAKNFPEYFRNWLPGPGQRVQQWSTIGTQNNAQTGTWASANKWILMGRSSKMAPGLAQPVRNAQTVTNGSQLIFNNETIKGSTATASTVHSGLLVTNESETAPTNPNSATKWGVMTDNQQTTSTTPTVSDDLEAHVFPGMVWQDRDIYLQGPIWAKIPETDGHFHPSPLMGGFGLKNPPPQILVKNTPVPAVPPTTFTPQKVNSFITQYSTGQVTVEIEWELRKEKSKRWNPEIQYTSNFENSANVQFSVNGDGAYIEPRPIGTRYLTHNL

SEQ ID NO: 857 - DNA, 2175 bp, Adeno-associated virus
  source 1..2175: mol_type = genomic DNA
atgtcgtttgtcgatcaccccccagactggctcgaggagatcggagaggggctctccgagtttatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggagggggacaacccgtacctcacctacaaccacgccgacgcccagttccagcaggacctccggggcgacgccagcctagccggcaacgccgcgaacgcgctcttccaggccaagaagagggtcctcgagcccttcggactggtcgagggagagccggagccgaagaagacgccttccgtcaagaggccgcacgcatcgccggactcgtcgagcggcgtcgggaagaagggcgaccagcccgcgaggaagaggctcgactttggaacggagcccgcgagtcaagacggagccggacgagcggcgcaggcaaccggagatatggcatctgctgaagtggctgcgggtggtggcggaccagtgggcgacgatgcacaaggtgccgatggagtgggtagcgcctcgggaaattggcattgcgattccgtttggatggacggcgctgtcatcaccaagtccacccgaacctggagcctgcccgcctacaacaaccacctctaccgccagatccagtccagcggcaccggagacggcacgtactttggttacagcacgccttggggatacttcgatttcaatcgattccactgccacttttctcccagggactggcagcggctcatcaacaaccactggggcatccgccccaagcggctccacttcaagctcttcaacatccaggtcaaggaggtcacgacgaccgacggcaccacgaccatcgccaacaacctcaccagcaccatacaggtctttgcggacacggagtaccagctcccgtacgtgctcggcaacgcccacgagggctgcctgccgccgttcccggcggacgtcttcatgctgccgcagtacgcgtacctgacgctcaacgccaacccgacgtcgcagagcgcggccgcgctcagcctgccgcagagcgccttctactgcctcgagtacttcccgagccagatgctgaggaccgggaacaacttctcgttcagctacgagttcgagaagctgcccttccactcgatgttcatgcacagccagagcctggaccggctcatgaacccgctcatcgaccagtacctctggtacctcaacgccacgaccggcaacaacctctccttcaacaaggccggcgccaagaacttccccgagtacttccgcaactggctgcccgggcccggccagcgcgtgcagcagtggagcacgatcgggacccagaacaacgcgcagaccggcacgtgggccagcgccaacaagtggatcctcatgggcaggtccagcaagatggcccccgggctggcccagccggtccggaacgcccagaccgtcaccaacggctcgcagctcatcttcaacaacgagaccatcaagggctccaccgcgacggcctccaccgtgcactcggggctgctcgtcaccaacgagtcggagaccgccccgaccaaccccaactcggcgacaaagtggggcgtcatgacggacaatcagcagaccacctccactacgccgaccgtcagcgacgacctcgaggcgcacgtcttcccgggcatggtctggcaggaccgcgacatctacctgcagggcccgatctgggccaagatcccggagacggacggccacttccacccgtccccgctcatgggcggcttcgggctcaagaacccgccgccgcagatcctcgtcaagaacacgcccgtgcccgccgtgccgcccacgaccttcacgccgcagaaggtcaactccttcatcacgcagtactccaccggccaggtcaccgtcgagatcgagtgggagctgcgcaaggagaagagcaagcgctggaaccccgagatccagtacacgtccaacttcgagaactcggccaacgttcagttctccgtcaacggcgacggcgcgtacatcgagccccgcccgatcggaacccgatacctcacccacaccctgtaa

SEQ ID NO: 858 - protein, 724 aa, Adeno-associated virus
MSFVDHPPDWLEEIGEGLSEFIGLEAGPPKPKPGYQDRARGLVVPGYKYLGPFNGLDRGEPVNAADAAAKKHDEEYDRLLKEGDNPYLTYNHADAQFQQDLRGDASLAGNAANALFQAKKRVLEPFGLVEGEPEPKKTPSVKRPHASPDSSSGVGKKGDQPARKRLDFGTEPASQDGAGRAAQATGDMASAEVAAGGGGPVGDDAQGADGVGSASGNWHCDSVWMDGAVITKSTRTWSLPAYNNHLYRQIQSSGTGDGTYFGYSTPWGYFDFNRFHCHFSPRDWQRLINNHWGIRPKRLHFKLFNIQVKEVTTTDGTTTIANNLTSTIQVFADTEYQLPYVLGNAHEGCLPPFPADVFMLPQYAYLTLNANPTSQSAAALSLPQSAFYCLEYFPSQMLRTGNNFSFSYEFEKLPFHSMFMHSQSLDRLMNPLIDQYLWYLNATTGNNLSFNKAGAKNFPEYFRNWLPGPGQRVQQWSTIGTQNNAQTGTWASANKWILMGRSSKMAPGLAQPVRNAQTVTNGSQLIFNNETIKGSTATASTVHSGLLVTNESETAPTNPNSATKWGVMTDNQQTTSTTPTVSDDLEAHVFPGMVWQDRDIYLQGPIWAKIPETDGHFHPSPLMGGFGLKNPPPQILVKNTPVPAVPPTTFTPQKVNSFITQYSTGQVTVEIEWELRKEKSKRWNPEIQYTSNFENSANVQFSVNGDGAYIEPRPIGTRYLTHTL

SEQ ID NO: 859 - DNA, 2166 bp, Adeno-associated virus
  source 1..2166: mol_type = genomic DNA
atgtcgtttgtcgatcaccccccagactggctcgaggagatcggagaggggctctccgagtttatcggactagaggcgggaccgccgaagcctaagccgggctaccaggaccgcgcgcgcggcctcgtcgtccccggctacaagtacctcgggcccttcaacggactcgacaggggcgagcccgtcaacgcggccgacgcggcggccaagaagcacgacgaggagtacgaccggctcctcaaggcgggggacaacccgtacctcacctacaaccacgcggacgccgagttccagaaggacctccggggcgacgccagcctagccggcaacgccgcgaacgcgctcttccaggccaagaagagggtcctcgagcccttcggactggtcgagggagagccggagccgaagaagacgccttccgtcaagaggccgcacgcatcgccggactcgtcgagcggaatcgggaagaggggcgaccagcccgcgaggaagaggctcgactttggaacggagcccgcgagtcaagacggagcgggaagagcggccaacgcagccggagatatggcatctgctgaggtggctgcgggtggtggcggaccagtgggcgacgatgcacaaggtgccgatggagtgggtcagtcctcgggaaattggcattgcgattccgtttggatgggcgaccgagtgctcaccaagtccacccggacctggagcctgcccacctacaacaaccacctgtacaagcagatcaacggcagcggaaccggggacgcggtgtactttggttacagcacgccttggggatacttcgatttcaatcgattccactgccacttttctccccgagactggcagcgcctcgtcaacaaccactggggcatccgcccccgaaggctcaacttcaagctcttcaacatccaggtcaaggaggtcacgacgacggacggcaccaagaccatcgccaataacctcacgtccaccgtccaggtgtttgcggacacggagcaccagctcccgtacatcctaggctccgcgcacgagggctgcatgccgcccttccccgcggacgtcttcatgctgccgcagtacggctacctcacgctcaacgggcccggcagcaacaacaacaacctcagcacgccctcgagcgccttctactgcctcgagtacttcccgagccagatgctgcgcacgggcaacaacttcgtcttcacctacgagttcgagaaggtgcccttccactcgatgttcatgcacaaccaggcgctggaccgcctcatgaacccgctcgtcgaccagtacctctggtacctcgacgccacctccggcaacaacctcaccttccgcaaggccggcgccaagaacttccccgagtacttccgcaactggatcccaggcccgggctgccgcaaccagcagtggaacaaggtcggcaccaagaacaacccgcagaccggcacgtgggcgtccgccaacaagtggcggctgcagggccgcctcaacaagtacgcgcccggacagcccaacgcccccgcggagggcttcctcaccaacgccggcgacctggccttcgccaacgccaaggccaccggcgccaccacggccgccggaaccgtgcccgccgacatcctgctcaccagcgagagcgagaccaccaccaccaacatgatgtccaacaacggctggggcgcgatcgcctccaacaaccagaacgcgtccgtcgcgcccaccgtgcagtacgaggactcggcgcacgtgctgcccggcatggtctggcaggaccgcgacatctacctgcagggcccgatctgggccaagatcccggagacggacggccacttccacccctcgccgctcatgggcggcttcggactcaagaacccgcccccgcagatcctcatcaagaacacgcccgtgcccgccgaccccccgacgcagttctcctcgcagaagatcaactccttcatcacgcagtactccaccggccagatgaccgtcgagatcgagtgggagctgcgcaaggagaactccaagcgctggaaccccgagatccagtacacggccaacttcaacaacagcgccaacgcgcagttctccgtcaacaacaacggcctctacatcgaggaccgcaccatcggaacccgatacctcacccacaccctgtaa

SEQ ID NO: 860 - protein, 721 aa, Adeno-associated virus
MSFVDHPPDWLEEIGEGLSEFIGLEAGPPKPKPGYQDRARGLVVPGYKYLGPFNGLDRGEPVNAADAAAKKHDEEYDRLLKAGDNPYLTYNHADAEFQKDLRGDASLAGNAANALFQAKKRVLEPFGLVEGEPEPKKTPSVKRPHASPDSSSGIGKRGDQPARKRLDFGTEPASQDGAGRAANAAGDMASAEVAAGGGGPVGDDAQGADGVGQSSGNWHCDSVWMGDRVLTKSTRTWSLPTYNNHLYKQINGSGTGDAVYFGYSTPWGYFDFNRFHCHFSPRDWQRLVNNHWGIRPRRLNFKLFNIQVKEVTTTDGTKTIANNLTSTVQVFADTEHQLPYILGSAHEGCMPPFPADVFMLPQYGYLTLNGPGSNNNNLSTPSSAFYCLEYFPSQMLRTGNNFVFTYEFEKVPFHSMFMHNQALDRLMNPLVDQYLWYLDATSGNNLTFRKAGAKNFPEYFRNWIPGPGCRNQQWNKVGTKNNPQTGTWASANKWRLQGRLNKYAPGQPNAPAEGFLTNAGDLAFANAKATGATTAAGTVPADILLTSESETTTTNMMSNNGWGAIASNNQNASVAPTVQYEDSAHVLPGMVWQDRDIYLQGPIWAKIPETDGHFHPSPLMGGFGLKNPPPQILIKNTPVPADPPTQFSSQKINSFITQYSTGQMTVEIEWELRKENSKRWNPEIQYTANFNNSANAQFSVNNNGLYIEDRTIGTRYLTHTL

SEQ ID NO: 861 - DNA, 2142 bp, Adeno-associated virus
  source 1..2142: mol_type = genomic DNA
atgattcgagtagtcatgtcgtttgttgactatcctccagattggctgaaagaggttgcgggtggactcaaagagttcattgacctcaaaccgggtgcgccgaagcccaaagcgaatcaacaaaaacaagatcaacgcggtcttgtcctcccgggttacaaatacctgggacccttcaacggccttgataaaggccaaccggtgaacaaagccgacgaagtcgcgcgggagcacgacatcgaatacgacaaactcctgaaagaaggagacaacccgtacgtgaagtacaaccacgccgatcaagagtttcaagagaaactcgagggcgacgaatcttttggcggtaacctcggacgagcggtcttccaagccaagaagcggttactggaaccgtttggtttatcggaagagacacctccgagaaagagacccgcttccgagaacaaccagtcagacgtcccggctaagagacgtctgatcctcgatcccgaagacaacggagccgccgaaggagaaccaatcgaacaacaacaaccagccaacgatatggcatctgctgaaatggctgcaggtggtggcgggccagtgggcgacgatgcacagggtgccgatggagtgggtagcgcctcaggaaattggcattgcgattccatttggatgggtgaccgagtcatcaccaaatccactcgcacctggagcctgcccacctacaacaaccacctctacaagcaaatcaactctaacaccaccgatgccaaagaaacctactttggctacagcactccttgggggtattttgacttcaaccggttccactgccacttctcgccgagggactggcaacgactcatcaacaaccattggggcatgcgaccaaagagactcaactttaaactctttaacatccaagtcaaagaggtcacgacgaacgacggtacgaaaacgatcgcgaacaatcttacctcgacggtgcaagtcttcgcggactcagagtaccaactcccctacgtgctaggcaacgcgcacgagggatgctttccaccgtttccggccgacgtcttcatgctaccgcagtatggatacctgaccttgaactcaaacgacaacaaagccatgccacagagctcgttctactgtctggaatattttccgagccagatgctgagaaccggcaacaacttcaccttcagctatgagttcgagagcgttccattccacagcatgtttatgcataaccaatcgctggacagattgatgaatccattgattgaccagtacttatggtacctacactctaccaacagtctatctctaaattttaaaaaagtgaagagagatcatctgtacaaatactacagaaattgggtacccggacccggtcaaagaagacaacaatggagcgaaagtggtggagcaaacaaacaattgacagacttgtggccatacaacaacaaatacaatctgatgggaagatattcttctttggcaccgggaatgccacaagcggcggcagatggattcatcacagccgatggacaattaatattttctaacgtgctacaagaatcagaagaagccagcggtggaaaaccatctccaaatctattaattacaaacgagtcagaagtcagaccaacaaatccagctatcaacgcaccttggggagatgtcgcattaaatcaacaaacgtcaacagtaacaccatcagtagctgaagacaacgaagcccacatttttcctggcatggtctggcaagaccgagacatctacctgcaaggacccatctgggccaagataccagacaccgacggcagcttccacccgtctccgctcatgggtggatttggactcaaacaccctccgccgcagatcctgatcaagaacacgccggtaccgtcggatcctccgctggaattcaacggcaagaagatccagtccttcatcacgcagtactcgaccggtcaggtgacggtggagatcgagtgggagctgaagaaggagcacagcaagcggtggaatccggagatccagtacacctccaacttcgacaacaccgaccgggtgcagttctcggtcaacggggcgggcaactatgtcgaagatcgaccaatcgggacccgttatctcacccacaatctgtaa

SEQ ID NO: 862 - protein, 713 aa, Adeno-associated virus
MIRVVMSFVDYPPDWLKEVAGGLKEFIDLKPGAPKPKANQQKQDQRGLVLPGYKYLGPFNGLDKGQPVNKADEVAREHDIEYDKLLKEGDNPYVKYNHADQEFQEKLEGDESFGGNLGRAVFQAKKRLLEPFGLSEETPPRKRPASENNQSDVPAKRRLILDPEDNGAAEGEPIEQQQPANDMASAEMAAGGGGPVGDDAQGADGVGSASGNWHCDSIWMGDRVITKSTRTWSLPTYNNHLYKQINSNTTDAKETYFGYSTPWGYFDFNRFHCHFSPRDWQRLINNHWGMRPKRLNFKLFNIQVKEVTTNDGTKTIANNLTSTVQVFADSEYQLPYVLGNAHEGCFPPFPADVFMLPQYGYLTLNSNDNKAMPQSSFYCLEYFPSQMLRTGNNFTFSYEFESVPFHSMFMHNQSLDRLMNPLIDQYLWYLHSTNSLSLNFKKVKRDHLYKYYRNWVPGPGQRRQQWSESGGANKQLTDLWPYNNKYNLMGRYSSLAPGMPQAAADGFITADGQLIFSNVLQESEEASGGKPSPNLLITNESEVRPTNPAINAPWGDVALNQQTSTVTPSVAEDNEAHIFPGMVWQDRDIYLQGPIWAKIPDTDGSFHPSPLMGGFGLKHPPPQILIKNTPVPSDPPLEFNGKKIQSFITQYSTGQVTVEIEWELKKEHSKRWNPEIQYTSNFDNTDRVQFSVNGAGNYVEDRPIGTRYLTHNL

SEQ ID NO: 863 - DNA, 2175 bp, Adeno-associated virus
  source 1..2175: mol_type = genomic DNA
atgagtcgagtagtcatgtcgtttgttgatcaccctccagattggctgaagcgtgtggcgagtggcttgactgagttcatcgacctcgaacccggacccccaaaacccaaacccggctatcaggaccagcagcgcggtctcgtggtacccggatacaaatacctcggtccctttaacggtctcgataaaggagaacccgttaacaaggctgacgaggttgctcgagagcacgacctcgagtacaacaagctacttgagcaaggagataatccttatctcaagtacaaccacgccgataaggagtttcaagagaaactctctgacgacacttcctggggcggcaacctcgggaaggcggtttttcaagccaagaagcgtatcctcgagccctttggcctagccgaagagctcggcggtgaaaagacggctcctggaaaaaagcgtcccaggcctccggaacttccaccttcggctccgcattcggacgacgaagaactgattgctcgaccggcagtcagagctcggctcgaaggtcaaaacggagcagcaaacgcaggaccagcagcagcaccagccgcagatatggcatctactgaagtggcttcaggcggtggcggcccaatgggcgacaataaccaaggttccgatggagtgggtagctcctccggaaattggcattgcgattccatttggatggacggcagagtcatcaccaagtctaccagaacctggagcctgcccacctacaacaaccacctctacaagcaaatcaactcaaatgccgaagtcaaagacgaaaagtactttggatatagtactccctggggatactttgatttcaatcgattccactgccacttttctcccagagactggcagcgactcatcaacaaccactggggaatgcgacccaaaagactcaactttaagctgttcaacatccaggtcaaggaggtcacgacgaatgacggaacgacaacgatcgccaataacctcacgtcgaccgtccaggtgtttgcagacacagagtaccagttgccgtacgtgctcgggaatgcgcacgagggctgcctcccgccgttcccggcagacgtgtttatgctgccgcagtacggatacttgactctaaacgacggcaaccaggtcattcctcagagctctttttactgcttggagtactttcccagtcagatgctgagaaccggaaacaactttacgttcagctacgagtttgagaacgtgcctttccacagcatgtttatgcataatcagtctctggaccgactgatgaatcctttagtcgatcagtacctgtggtacctctcggctaccaacggattaaatttacattttagaaaatccaagaagggagcgtttgccagtcaatacagaaactggatgcccggtcctaaccagcgacgtcagcagtggagtaaaggagctggaaacaacaacgccttgaccaatctctgggaatacaaaaacaagttccacatcatgaacagagactcggtactggctccaggactcgcacaggctgcggcggatggatatttcacagccgacggacaattaatttttgctaacgctacacaacaatcggacgaggcttccggtggaaatccaccgagcagcatgttaattaccggtgaatccgaaactcgacccactaatcccggagcaaacaccaagtggggtgaaatggccgacaaccaacagtctacaacggcaaagccgaccatggttatcgacaacgaggctcacatctttcccggcatggtctggcaggatcgggacatttacctgcaagggcccatctgggccaagattccagagacggacggacactttcatccttctccgctgatgggcggattcggtctcaagaatcctcctcctcagatactcatcaagaatacacccgttccggctaatcctccggaagcgttcaccgccaacaagataaactccttcatcacgcagtactcgaccggacaagtaacggtggaaattgaatgggagctcaggaaggagcacagcaagcgctggaacccagaggtgcagtacacgtccaactttgactctacagacagtctgcagtttaccgttaacaacgacggactctacatcgagactcgaccaatcggaacccgtttcctttcccacaatatttaa

SEQ ID NO: 864 - protein, 724 aa, Adeno-associated virus
MSRVVMSFVDHPPDWLKRVASGLTEFIDLEPGPPKPKPGYQDQQRGLVVPGYKYLGPFNGLDKGEPVNKADEVAREHDLEYNKLLEQGDNPYLKYNHADKEFQEKLSDDTSWGGNLGKAVFQAKKRILEPFGLAEELGGEKTAPGKKRPRPPELPPSAPHSDDEELIARPAVRARLEGQNGAANAGPAAAPAADMASTEVASGGGGPMGDNNQGSDGVGSSSGNWHCDSIWMDGRVITKSTRTWSLPTYNNHLYKQINSNAEVKDEKYFGYSTPWGYFDFNRFHCHFSPRDWQRLINNHWGMRPKRLNFKLFNIQVKEVTTNDGTTTIANNLTSTVQVFADTEYQLPYVLGNAHEGCLPPFPADVFMLPQYGYLTLNDGNQVIPQSSFYCLEYFPSQMLRTGNNFTFSYEFENVPFHSMFMHNQSLDRLMNPLVDQYLWYLSATNGLNLHFRKSKKGAFASQYRNWMPGPNQRRQQWSKGAGNNNALTNLWEYKNKFHIMNRDSVLAPGLAQAAADGYFTADGQLIFANATQQSDEASGGNPPSSMLITGESETRPTNPGANTKWGEMADNQQSTTAKPTMVIDNEAHIFPGMVWQDRDIYLQGPIWAKIPETDGHFHPSPLMGGFGLKNPPPQILIKNTPVPANPPEAFTANKINSFITQYSTGQVTVEIEWELRKEHSKRWNPEVQYTSNFDSTDSLQFTVNNDGLYIETRPIGTRFLSHNI

SEQ ID NO: 865 - DNA, 2211 bp, Adeno-associated virus
  source 1..2211: mol_type = genomic DNA
atgagtcgagtagtcatgtcgtttgtcgatcaccctccagattggctgaaagaggccgcgggcggactacgcgagtttctcgatctcgagccgggagcgcctaaacccaaacctaatcaacaaaaacaggaccagcgaggtctggttctgccgggatacaaatatctcggacctttcaacggccttgataaaggccagcccgtcaataaggcggacgaggtcgcgcgagagcacgatctcgagtataacaagctacttgaggcgggcgacaatccctacctcaagtacaaccacgcagaccaagagtttcaggagaaactcgagggggacacttccttcggaggtaacctcggtcgcgcggtattccaggccaagaagagggtgctagagccctttggattggccgaagaagctccggcgaagacggctcctgggaagaaacgtcccaggcccatcgaaccccctccttcctcggagaactcggttgacgaggacttggtcgagcgccaagctcagcgtcaacgtctcggatttcaagacggacccggaaacagcgctaaccaagtccagcaacaaccagccgcaaatatggcatctgctgaagtggctgcgggcagtggcggcccaatgggcgacaatgcacaaggtgccgatggagtgggtagctcctccggaaattggcattgcgattccctttggatgggcgatagggtgatgcgattcccttggatgggagaccgagtcatcaccaagtccacccgaacctggagcctgcccacctacaacaaccatctctacaagcaaatcaattccaactcggatgtcaccgacgaaaagtactttggatacagtaccccgtggggatactttgactttaaccgcttccactgccacttttcaccccgagactggcagcgcctcatcaacaaccattggggtatgcgcccgaagaggctcaactttaagctgttcaacatccaggttaaggaggtcacgacgaacgacggcaccacgaccatcgccaacaacctcacgtctaccgtccagattttcgccgacagcgagtaccagttgccgtacgtgctcgggaatgcgcacgagggctgcctcccgccgttcccggcagacgtgttcatgctgccgcagtacgggtacctcacgttgaacgacggaaatcaagtgattccacaaagttccttctactgtctggagtattttcccagccagatgctgagaaccggcaacaacttttccttcagctacgaatttgaaaacgtgcctttccacagcatgtttatgcacaaccagtctttggatcggttaatgaaccccctcatggaccagtacctgtggtacctgacggccaccaacggcgttcagttgcactttaaaaagtccaagaagggggcgtttgccagccagtttaggaactggctgccgggtcccaaccagcggcgtcagcagtggagcaaaggggccggtaacaacaacgctcagaccaacctgtggagttacaaaaacaagtaccggatcatgaaccgggacagcgtggcggctcctggactggctcaagcggcggccgatggcttcatcacggccgacggacagctcatcttctccaacgccggacaacagtcggatgaagcggctggagctaaaccaccgaccacgttgctgattacagacgagagtgaaactcgtcctaccaacccgggtgccaacaccaaatggggagaagtggcagacaaccaacagacatcttccacggcccccaccatggccatcgacaacgaggcccacatctttcccggaatggtgtggcaggatcgagacatttacctgcagggtcccatctgggccaagattccagagacggacggtcactttcacccgtctcctctgatgggaggattcggactgcgaaatcctcctcctcagattctcatcaaaaacacaccggtaccggccaatccaccagagaccttcaacgccaacaagatcaactcattcatcacccagtactcaaccggacaggttacagtcgaaatagagtgggagctgaagaaggagcacagcaaacgctggaacccggaggtgcagtacacgtccaattttgcctcggcggacagcctccaattcaccgtcaacggagaaggcgtgtacatcgaagacagaccaatcggaacccgctacctctcccacaacatttaa

SEQ ID NO: 866 - protein, 736 aa, Adeno-associated virus
MSRVVMSFVDHPPDWLKEAAGGLREFLDLEPGAPKPKPNQQKQDQRGLVLPGYKYLGPFNGLDKGQPVNKADEVAREHDLEYNKLLEAGDNPYLKYNHADQEFQEKLEGDTSFGGNLGRAVFQAKKRVLEPFGLAEEAPAKTAPGKKRPRPIEPPPSSENSVDEDLVERQAQRQRLGFQDGPGNSANQVQQQPAANMASAEVAAGSGGPMGDNAQGADGVGSSSGNWHCDSLWMGDRVMRFPWMGDRVITKSTRTWSLPTYNNHLYKQINSNSDVTDEKYFGYSTPWGYFDFNRFHCHFSPRDWQRLINNHWGMRPKRLNFKLFNIQVKEVTTNDGTTTIANNLTSTVQIFADSEYQLPYVLGNAHEGCLPPFPADVFMLPQYGYLTLNDGNQVIPQSSFYCLEYFPSQMLRTGNNFSFSYEFENVPFHSMFMHNQSLDRLMNPLMDQYLWYLTATNGVQLHFKKSKKGAFASQFRNWLPGPNQRRQQWSKGAGNNNAQTNLWSYKNKYRIMNRDSVAAPGLAQAAADGFITADGQLIFSNAGQQSDEAAGAKPPTTLLITDESETRPTNPGANTKWGEVADNQQTSSTAPTMAIDNEAHIFPGMVWQDRDIYLQGPIWAKIPETDGHFHPSPLMGGFGLRNPPPQILIKNTPVPANPPETFNANKINSFITQYSTGQVTVEIEWELKKEHSKRWNPEVQYTSNFASADSLQFTVNGEGVYIEDRPIGTRYLSHNI

SEQ ID NO: 867 - protein, 20 aa, synthetic construct
DYGNFLANHTNPLTVSKIDT

SEQ ID NO: 868 - protein, 20 aa, synthetic construct
LTSVVFILICCFIILENIFV

SEQ ID NO: 869 - protein, 20 aa, synthetic construct
RYLVLAVDRPAGAWRGSGLA

SEQ ID NO: 870 - protein, 20 aa, synthetic construct
QQVSSPHTKAPLHKDSANGF

SEQ ID NO: 871 - protein, 20 aa, synthetic construct
VAGHFESQSDDPHFRGKKQL

SEQ ID NO: 872 - protein, 20 aa, synthetic construct
GVHAKSSDWGLQAGYIPVNP

SEQ ID NO: 873 - protein, 20 aa, synthetic construct
RWYGQDNTCEEYHTVGPHSC

SEQ ID NO: 874 - protein, 20 aa, synthetic construct
FGSATYDDEHHSTLVSTRHY

SEQ ID NO: 875 - protein, 20 aa, synthetic construct
YLESRSINEKDYHSRRYIDE

SEQ ID NO: 876 - protein, 20 aa, synthetic construct
GDMANSSIEFHPKPQQQRDV

SEQ ID NO: 877 - protein, 20 aa, synthetic construct
PQSWGRGSAGGNTHSPLGVP

SEQ ID NO: 878 - protein, 20 aa, synthetic construct
FKGVDNEDDHGRGHLGRLKK

SEQ ID NO: 879 - protein, 20 aa, synthetic construct
GKGNYAERAGAGAPAYLEAV

SEQ ID NO: 880 - protein, 20 aa, synthetic construct
LVSKGPLVQTKGTGASGSFR

SEQ ID NO: 881 - protein, 20 aa, synthetic construct
GFAIGIVGAAGERGTAQQPR

SEQ ID NO: 882 - protein, 20 aa, synthetic construct
ATLERPASSSHAQGTHLQLR

SEQ ID NO: 883 - protein, 20 aa, synthetic construct
LATAGSAMGAASLTVSAQSR

SEQ ID NO: 884 - protein, 20 aa, synthetic construct
LGAVGSTMGAGSLTLTEQAR

SEQ ID NO: 885 - protein, 20 aa, synthetic construct
TAGAAMGAAGPTLSAQSRSL

SEQ ID NO: 886 - protein, 20 aa, synthetic construct
IGAGVAGGITGSISLANGKS

SEQ ID NO: 887 - protein, 20 aa, synthetic construct
AVGGAIGRVANTIRSGPSNS

SEQ ID NO: 888 - protein, 20 aa, unidentified
SQGISSSSTSTMPNSSQSSH

SEQ ID NO: 889 - protein, 20 aa, synthetic construct
VKDRSHNVTENVTQVLSLGA

SEQ ID NO: 890 - protein, 20 aa, synthetic construct
EILELAGNATRDSNKTRIIP

SEQ ID NO: 891 - protein, 20 aa, synthetic construct
TQNKRANRTEENVSDGSPTA

SEQ ID NO: 892 - protein, 20 aa, synthetic construct
FGPVLDNATRKSASVHAIAS

SEQ ID NO: 893 - protein, 20 aa, synthetic construct
DNRTRQHIQYREDKNLTGTA

SEQ ID NO: 894 - protein, 20 aa, synthetic construct
KPQVTRGDVFTMSEDEHTVY

SEQ ID NO: 895 - protein, 20 aa, synthetic construct
AGMDASDRQVIISSNLTRPK

SEQ ID NO: 896 - protein, 20 aa, synthetic construct
TDRTSLPSANITRSFKNEDL

SEQ ID NO: 897 - protein, 20 aa, synthetic construct
NRDGTVLESNRTNSLRKINS

SEQ ID NO: 898 - protein, 20 aa, synthetic construct
NLPGDPTSNATSRGAEGPLP

SEQ ID NO: 899 - protein, 20 aa, synthetic construct
PMPDQAVPTGSFMMVNSSRR

SEQ ID NO: 900 - protein, 20 aa, synthetic construct
QQQSYLDSGIHSGATTTAPS

SEQ ID NO: 901 - protein, 20 aa, synthetic construct
GTQGRLCNKTSECMDGCELM

SEQ ID NO: 902 - protein, 20 aa, synthetic construct
DHTGRHDTPRMSTSQTPGRA

SEQ ID NO: 903 - protein, 20 aa, synthetic construct
VAEIPFNSTNKYQLYIHETE

SEQ ID NO: 904 - protein, 20 aa, synthetic construct
LVQIRSNSSKPDLMASLLPK

SEQ ID NO: 905 - protein, 20 aa, synthetic construct
VLKQGHPDTGISSKAMGIIY

SEQ ID NO: 906 - protein, 20 aa, synthetic construct
ISNRVLVSNQTGTAFQPDAA

SEQ ID NO: 907 - protein, 20 aa, synthetic construct
AMGAGVVGRTTGSMSLASGK

SEQ ID NO: 908 - protein, 20 aa, synthetic construct
RLTAGTGGREVGARGEPSGI

SEQ ID NO: 909 - protein, 20 aa, synthetic construct
ILQRGSGTAAVDFTKKDNTA

SEQ ID NO: 910 - protein, 20 aa, synthetic construct
WGQGKLVSVSPDSTQGPSVF

SEQ ID NO: 911 - protein, 20 aa, synthetic construct
SNLHDTKASGPAHSKEVPTL

(19) United States
(12) Patent Application Publication
(10) Pub. No.: US 2026/0355350 A1
(43) Pub. Date: Nov. 12, 2026

(54) REPROGRAMMING TROPISM VIA DISPLAYED PEPTIDES TILING RECEPTOR-LIGANDS

(71) Applicant: The Regents of the University of California, Oakland, CA (US)

(72) Inventors: Prashant Mali, La Jolla, CA (US); Andrew Portell, La Jolla, CA (US); Kyle Ford, La Jolla, CA (US); Amanda Suhardjo, La Jolla, CA (US)

(21) Appl. No.: 19/106,382

(22) PCT Filed: Sep. 8, 2023
(86) PCT No.: PCT/US2023/073808

§ 371 Date: Feb. 25, 2025

Related U.S. Application Data
(60) Provisional application No. 63/405,360, filed on Sep. 9, 2022.

(51) Int. Cl. A61K 48/00; C07K 14/005; C12N 15/113; C12N 15/86

(52) U.S. Cl. CPC A61K 48/0041; A61K 48/0075; C07K 14/005; C12N 15/113; C12N 15/86; C12N 2310/141; C12N 2750/14143; C12N 2750/14145; C12N 2750/14151

(57) ABSTRACT

Described herein are methods for engineering proteins and viruses to improve tropism, and proteins and viruses made by using said methods.

### CROSS REFERENCE TO RELATED APPLICATIONS

This application claims priority under 35 U.S.C. § 119 from Provisional Application Ser. No. 63/405,360, filed Sep. 9, 2022, the disclosures of which are incorporated herein by reference in its entirety.

### STATEMENT REGARDING FEDERALLY SPONSORED RESEARCH

This invention was made with Government support under OD032742, CA222826, and GM123313 awarded by the National Institutes of Health and W81XWH-22-1-0401 awarded by the Department of Defense. The government has certain rights in the invention.

### TECHNICAL FIELD

Described herein are methods for engineering proteins and viruses to improve tropism, and proteins and viruses made by using said methods.

### INCORPORATION BY REFERENCE OF SEQUENCE LISTING

Accompanying this filing is a Sequence Listing entitled “00015-417WO1_SL.xml”, created on Sep. 8, 2023 and having 872,924 bytes of data, machine formatted on IBM-PC, MS-Windows operating system. The sequence listing is hereby incorporated herein by reference in its entirety for all purposes.

### BACKGROUND

Nucleic acid and protein based therapeutics to modulate healthy and diseased states are poised to enable the next frontier of human medicine. However their successful deployment is contingent on the ability to deliver them efficiently and in a safe and targeted manner. In this regard, a host of viral and non-viral delivery formulations have been developed, but the ability to directly modulate their tropism remains challenging.

### SUMMARY

The disclosure provides a method of improving tropism of a virus or other delivery agent, the method comprising identifying ligand protein sequences derived from all known receptor-interacting ligands; systematically tile the ligand peptides into 5-50 or 10-20 or 20 amino acid peptides which are inserted into surface-exposed loops of AAV capsids; assessing the engineered capsids for their packaging capacity, in vivo tropism, and enhanced protein interactions. In one embodiment, the virus is an adeno-associated virus (AAV). In a further embodiment, the AAV is selected from the group consisting of AAV1, AAV2, AAV5, AAV6, AAV7, AAV8, and AAV9. In still a further embodiment, the AAV is AAV5 or AAV9. In another embodiment, peptide sequences were generated via pooled oligonucleotide synthesis and inserted into 4 distinct loop regions: AAV5-Loop1 (N443), AAV5-Loop2 (S576), AAV9-Loop1 (Q456), and AAV9-Loop2 (A587) to generate over 1 million AAV variants. In another embodiment, a 20'mer ligand peptide is inserted into one or both of 2 surface-exposed loops of AAV5 (SEQ ID NO: 2) or AAV9 (SEQ ID NO: 4).

The disclosure provides a recombinant vector comprising capsid proteins containing one or two peptides inserted into one or both of two surface-exposed loops in the capsid protein, wherein the vector has a desired tropism or immune-orthogonality and wherein the peptides are independently selected from the group consisting of SEQ ID NOs:5 to 820 and 870-911. In one embodiment, the vector is an adeno-associated virus (AAV). In a further embodiment, the AAV vector is an AAV5 serotype. In still a further embodiment, the AAV5 comprises a capsid protein having a sequence as set forth in SEQ ID NO:2. In another embodiment, the AAV vector is an AAV9 serotype. In a further embodiment, the AAV9 comprises a capsid protein having a sequence as set forth in SEQ ID NO: 4. In another embodiment, the vector has tropism to pancreas, heart, brain, lung, liver, kidney, muscle, spleen or intestine. In another or further embodiment, a peptide of SEQ ID NOs:530-820, 870-910 or 911 is inserted into loop 1 and/or loop 2 of an AAV5 capsid or an AAV9 capsid. In another embodiment, the vector is immune orthogonal. In a further embodiment, the vector is an adeno-associated virus (AAV). In still a further embodiment, the AAV comprises a capsid protein of any one of SEQ ID NOs:822, 824, 826, 828, 830, 832, 834, 836, 838, 840, 842, 844, 846, 848, 850, 852, 854, 856, 858, 860, 862, 864, or 866, or a sequence that it at least 85% to 99% identical to any of the foregoing sequences.

The disclosure also provides a method of making a delivery vehicle or vector with a desired tropism, the method comprising selecting a peptide sequence from any one of SEQ ID NOs: 5-820 or 870-911 and (i) cloning a nucleic acid sequence encoding the peptide into a coding sequence for a capsid protein at an exposed loop site to obtain a recombinant capsid coding sequence and producing the vector using the recombinant capsid coding sequence or (ii) inserting the peptide into an exposed surface of the delivery vehicle. In one embodiment, the vector is an adeno-associated virus (AAV). In a further embodiment, the AAV is selected from the group consisting of AAV1, AAV2, AAV5, AAV6, AAV7, AAV8, and AAV9. In still a further embodiment, the AAV is AAV5 or AAV9. In a further embodiment, the AAV5 capsid coding sequence comprises SEQ ID NO:1. In another embodiment, the AAV9 capsid coding sequence comprises SEQ ID NO:2. The disclosure also provide an AAV vector comprising a capsid protein modified by any of the foregoing embodiments.

The disclosure also provides an AAV vector comprising a capsid protein wherein the capsid protein expresses a peptide of any one of SEQ ID NO: 5-820 or 870-911 in surface exposed loop 1 and/or loop 2 of the capsid protein. In one embodiment, the wild-type capsid protein sequence comprises SEQ ID NO:2 or 4. In another embodiment, the AAV vector has a AAV5 or AAV9 serotype. In still another embodiment, the vector has a desired tropism. In a further embodiment, the AAV vector has a tropism to pancreas, heart, brain, lung, liver, kidney, muscle, spleen or intestine.

The disclosure also provides a viral vector having a capsid protein comprising a heterologous targeting peptide of 10-30 amino acids in length inserted into a surface exposed portion of the capsid protein and wherein the targeting peptide is set forth in any one of SEQ ID NOs: 5-820 or 870-911. In one embodiment, the heterologous targeting peptide is about 15-25 amino acids in length. In a further embodiment, the heterologous targeting peptide is about 20 amino acids in length. In another embodiment, the viral vector is an adeno-associated virus (AAV). In still another embodiment, the viral vector is a lentiviral vector. In another embodiment, the capsid protein is a VP1 capsid protein. In still another embodiment, the capsid protein is a VP2 capsid protein. In yet another embodiment, the capsid protein is a VP3 capsid protein. In another embodiment, the heterologous targeting peptide is inserted into an AAV capsid protein at loop 1 and/or loop 2. In yet another embodiment, the viral vector is an AAV5. In another embodiment, the viral vector is an AAV9. In still another embodiment, the heterologous targeting peptide is flanked by a linker peptide at the N-terminal and C-terminal ends of the heterologous targeting peptide. In another embodiment, the heterologous targeting peptide targets the viral vector to hepatocytes or liver tissue. In yet still another embodiment, the heterologous targeting peptide targets the viral vector to neuronal cells or brain tissue. In still another embodiment, the heterologous targeting peptide targets the viral vector to pancreatic cells or pancreas tissue. In yet another embodiment, the heterologous targeting peptide targets the viral vector to cardiac cells or heart tissue. In another embodiment, the heterologous targeting peptide targets the viral vector to lung tissue. In still yet another embodiment, the heterologous targeting peptide targets the viral vector to intestinal tissue. In yet another embodiment, the heterologous targeting peptide targets the viral vector to spleen tissue. In still another embodiment, the heterologous targeting peptide targets the viral vector to renal cells or kidney tissue. In another embodiment, the heterologous targeting peptide targets the viral vector to muscle cells or tissue.

The disclosure also provides an adeno-associated virus (AAV) capsid protein comprising a heterologous targeting peptide cloned into loop 1 and/or loop 2 of the capsid protein, wherein the heterologous targeting peptide is about 10-30 amino acids in length and is contained within or comprises any one of the peptides of SEQ ID NO:5-820 or 870-911. In one embodiment, the capsid protein is a VP1 capsid protein. In another embodiment, the capsid protein is a VP2 capsid protein. In still another embodiment, the capsid protein is a VP3 capsid protein. In another embodiment, the heterologous targeting peptide is about 15-25 amino acids in length. In yet another embodiment, the heterologous targeting peptide is about 20 amino acids in length. In a still another embodiment, the heterologous targeting peptide is flanked by a linker peptide at the N-terminal and C-terminal ends of the heterologous targeting peptide. In another embodiment, the heterologous targeting peptide targets hepatocytes or liver tissue. In still another embodiment, the heterologous targeting peptide targets neuronal cells or brain tissue. In another embodiment, the heterologous targeting peptide targets pancreatic cells or pancreas tissue. In yet another embodiment, the heterologous targeting peptide targets cardiac cells or heart tissue. In another embodiment, the heterologous targeting peptide targets lung tissue. In yet another embodiment, the heterologous targeting peptide targets intestinal tissue. In another embodiment, the heterologous targeting peptide targets spleen tissue. In another embodiment, the heterologous targeting peptide targets renal cells or kidney tissue. In yet another embodiment, the heterologous targeting peptide targets muscle cells or tissue. The disclosure also provides a recombinant AAV (rAAV) comprising a capsid protein of any of the foregoing embodiments.

The disclosure provides a recombinant AAV (rAAV) comprising a capsid protein having a targeting peptide in loop 1 and/or loop 2 wherein the targeting peptide is independently selected from SEQ ID Nos: 5-820 or 870-911. In one embodiment, a targeting peptide is present in both Loop 1 and Loop 2. In a further embodiment, the targeting peptide has the same tropism. In another embodiment, the recombinant AAV further comprises a heterologous polynucleotide for gene delivery. In a further embodiment, the heterologous polynucleotide is a therapeutic gene. In still another or further embodiment, of any of the foregoing embodiments, the rAAV is present in a pharmaceutical composition.

The disclosure also provides a method for delivering a transgene to a subject comprising: administering a recombinant AAV (rAAV) to a subject, wherein the rAAV comprises: (i) a capsid protein of the disclosure, and (ii) at least one transgene, and wherein the rAAV infects cells of a target tissue of the subject. In one embodiment, the at least one transgene encodes a protein. In a further embodiment, the protein is an immunoglobulin heavy chain or light chain or fragment thereof. In another embodiment, the at least one transgene encodes a small interfering nucleic acid. In a further embodiment, the small interfering nucleic acid is a miRNA. In another embodiment, the small interfering nucleic acid is a miRNA sponge or TuD RNA that inhibits the activity of at least one miRNA in the subject or animal. In yet another embodiment, the miRNA is expressed in a cell of the target tissue. In still another embodiment, the target tissue is skeletal muscle, heart, liver, pancreas, brain or lung. In another embodiment, the transgene expresses a transcript that comprises at least one binding site for a miRNA, wherein the miRNA inhibits activity of the transgene, in a tissue other than the target tissue, by hybridizing to the binding site. In yet another embodiment, the at least one transgene encodes a gene product that mediates genome editing. In another embodiment, the transgene comprises a tissue specific promoter or inducible promoter. In a further embodiment, the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. In another embodiment, the rAAV is administered intravenously, intravascularly, transdermally, intraocularly, intrathecally, orally, intramuscularly, subcutaneously, intranasally, or by inhalation. In still another embodiment, the subject is selected from a mouse, a rat, a rabbit, a dog, a cat, a sheep, a pig, and a non-human primate. In yet another embodiment, the subject is a human.

The disclosure also provides an isolated nucleic acid encoding an AAV capsid protein containing an amino acid sequence selected from the group consisting of SEQ ID No: 5-820 and 870-911.

The disclosure also provides a delivery vehicle for delivery of a small molecule drug or biological agent having a desired tropism, wherein the delivery vehicle comprises a peptide or peptide fragment of at least 10-20 amino acids of any one of SEQ ID NOs:5-820 or 870-911. In one embodiment, the delivery vehicle is selected from the group consisting of a liposome, a nanoparticle, a bacteria, a bacteriophage, a virus-like particle (VLP), a erythrocyte ghost, and an exosome. In another embodiment, the biological agent comprises an siRNA, an antisense molecule, a protein or polypeptide, insulin, a vaccine, or an antibody. In still another embodiment, the small molecule drug comprises a chemotherapeutic agent, an anti-inflammatory, a steroid, and an antibiotic.

The disclosure also provides a biological agent having a desired tropism, the biological agent linked to a peptide or peptide fragment of at least 10-20 amino acids of any one of SEQ ID NOs:5-820 or 870-911. In one embodiment, the biological agent is a nucleic acid, a protein, a polypeptide, a peptide, an antibody, an antibody fragment, a non-immunoglobulin binding agent, or an enzyme.

### DESCRIPTION OF DRAWINGS

FIG. 1A-B Design of AAV libraries displaying peptides tiling receptor-ligands. (a) Schematic of approach for rationally engineering and characterizing AAV variants. Ligand protein sequences derived from all known receptor-interacting ligands are systematically tiled into 20 amino acid peptides which are inserted into surface-exposed loops of AAV capsids. These engineered variants were then assessed for their packaging capacity, in vivo tropism, and enhanced protein interactions. (b) Protein class distribution of the ligands which were tiled to compose the screening library. These include known receptor-interacting ligands (orange), cell membrane permeable proteins (green) and protein domains (blue), and stop codon containing negative controls (purple). Peptide sequences were generated via pooled oligonucleotide synthesis and inserted into 4 distinct loop regions: AAV5-Loop1 (N443), AAV5-Loop2 (S576), AAV9-Loop1 (Q456), and AAV9-Loop2 (A587) to generate over 1 million AAV variants. Capsid surface residues are colored according to their distance from the core of the capsid with the insertion site flanking residues shown in red.

FIG. 2A-F. AAV library packaging analyses reveal biophysical features contributing to capsid fitness. (a) Schematic illustrating recombinant production of pooled AAV libraries in HEK293T cells. (b) Normalized abundance for each inserted peptide in the plasmid libraries versus DNA isolated from recombinantly produced AAV variant capsid libraries. Dotted-red line shows where the plasmid abundance is equal to the capsid abundance. (c) Normalized abundance for negative control peptides containing stop codons, in both the plasmid libraries and recombinantly produced AAV capsid libraries (d) Distribution of peptide biophysical parameters relevant to packaging. Peptide charge, alpha-helical content, flexibility, and hydrophobicity distributions are shown for peptides which are enriched in the capsid pool (“Packaging”) and those which are depleted (“Non Packaging”). Statistical significance between groups was calculated via a T-test (****p<0.0001). (e) Biophysical parameters of inserted peptides were used as features to train a support vector machine classifier predicting which AAV variants successfully package into capsids. The receiver operating characteristic curve is shown for the resulting model, with an area under the curve of 0.89. (f) UMAP embedding for each AAV variant, colored by packaging status. Inserted peptide charge, alpha-helical content, flexibility, and hydrophobicity were used as input features for the embedding.

FIG. 3A-D. In vivo screen analyses enable predictive computational models of tropism. (a) Overview of in vivo screening methodology. The four AAV variant libraries were injected retro-orbitally into C57/BL6 mice in duplicate. Two weeks post-injection, nine organs were harvested from each mouse and the inserted peptide-containing region of the AAV capsid was amplified and subjected to next generation sequencing. (b) Replicate Pearson correlation for the log₂ fold change (log₂FC) values (organ vs. capsid) for each library and organ. (c) Overview of screen results. Significantly enriched AAV variants in a particular organ are defined as those with a log₂FC>1 and an FDR-adjusted p-value <0.05. Bar plots show the number of significantly enriched variants detected per organ, as well as a comparison of peptide hits for each loop insertion site, for both AAV5 and AAV9. (d) Overview of classification model predicting AAV tissue tropism from peptide sequence alone. Inserted peptide sequences were converted to a binary one-hot encoding (for each peptide, 20 rows corresponding to position, and 20 columns corresponding to presence of a particular amino acid). This one-hot encoding scheme was then used as input to a convolutional neural network (CNN) to predict organ targeting. Model performance was separately evaluated on each organ, via accuracy, area under the receiver operator characteristic curve (AUROC), F1 score, and Matthews Correlation Coefficient (MCC). Models were trained on ⅔ of the data, and the remaining 4 was held out as a validation dataset to evaluate performance. Figure discloses SEQ ID NOs:867-869, respectively, in order of appearance.

FIG. 4A-D. In vivo screen identified AAV variants demonstrate reprogrammed tropism. (a) Heatmap showing log₂FC values for each AAV variant which was significantly enriched in at least one organ. Rows are individual variants, and columns are organs (n=2 per organ). (b) UMAP embedding of significantly enriched AAV variants. Each dot represents a variant, colored by the organ with max log₂FC. (c) AAVs were chosen for validation from the pool of significant hits on the basis of their tissue specificity, broad tropism, and/or internal consistency. Internal consistency was quantified by counting the number of similar (>50% homology) inserted peptides also detected as hits for a given organ. AAV variants were characterized structurally via transmission electron microscopy, and functionally via delivery of the mCherry transgene in vivo. Heatmaps depict all variants chosen for validation (n=21), with the left heatmap showing the Z-normalized log 2FC values from the pooled in vivo screen (n=2), and the right heatmap showing the Z-normalized mCherry expression quantified by RT-qPCR, relative to AAV9 (n=2). (d) Comparison between liver RT-qPCR quantification of mCherry delivery, versus protein level quantification via fluorescent microscopy.

FIG. 5A-D. Characterization and mechanistic exploration of AAV variants with displayed ligand peptides. (a) Full characterization experiments for the variant AAV9.DKK1. In the top left, lung screen count values for all AAV9Loop2 variants with inserted DKK1 derived peptides are shown. The x-axis indicates the position in the DKK1 structure a given peptide starts on. Shown in blue are the lung counts, and shown in orange are the capsid counts. Red arrow indicates the location of the peptide inserted in AAV9.DKK1. Bar plot shows the RT-qPCR quantification (n=2) of mCherry transgene expression via individual validation, normalized to that of AAV9. Also shown are electron microscope images for AAV variant capsids, as well as fluorescent microscopy of mCherry protein expression levels in the lung and liver. (b) Knockout experiments to validate AAV9.DKK1 receptor dependency. Cas9-containing lentivirus was produced with either non-targeting controls (NTC) and LRP6 targeting sgRNA (n=2 each). HEK293T cells were then transduced with lentivirus and positively selected with puromycin. Lentivirally transduced cells were then plated in a 24-well plate and transduced with either wild-type AAV9 (4×10⁹ viral genomes) or AAV9.DKK1 virus (1×10⁹ viral genomes). Flow cytometry was then used to quantify the mean fluorescence intensity (MFI) of the infected cells. Shown to the right is the crystal structure of a 7-mer peptide DKK1 peptide (contained within AAV9.DKK1) in complex with LRP6 (58). (c) Full characterization experiments for the variant AAV9.PDGFC. In the top left, muscle screen count values for all AAV9Loop1 variants with inserted PDGFC derived peptides are shown. The x-axis indicates the position in the PDGFC structure a given peptide starts on. Shown in blue are the muscle counts, and shown in orange are the capsid counts. Red line indicates the location of the peptide inserted in AAV9.PDGFC. Bar plot shows the RT-qPCR quantification (n=2) of mCherry transgene expression via individual validation, normalized to that of AAV9. Also shown are electron micrographs for variant capsids, as well as fluorescent microscopy of mCherry protein expression levels in the heart and muscle. (d) Receptor overexpression studies for the variant AAV9.PDGFC. Known receptors for the PDGFC ligand were cloned into an overexpression plasmid and transfected into HEK293T cells in a 24-well plate. After 24 hours, either AAV9 (4×10⁹ viral genomes) or AAV9.PDGFC (4×10⁹ viral genomes) were used to transduce the cells. Following 24 hours of transduction, the cells were collected and mCherry expression was quantified via flow cytometry. Bar plots show the MFI normalized to the average MFI of the AAV9 transduced cells with an empty vector overexpressed. Statistical significance between groups was calculated via a T-test (*p<0.05, **p<0.01, ***p<0.001, ****p<0.0001).

FIG. 6A-D. Inserted peptide facilitates efficient liver de-targeting across multiple AAV scaffolds in a mouse strain-independent manner. (a) Significantly enriched AAV variants from all capsids are projected into 2 dimensions via UMAP. The distance between points is calculated explicitly via the Levenshtein distance of the amino acid sequences of the inserted peptides, and subsequently embedded via UMAP. AAV variants are colored by their log 2 fold change in the brain. Select variant clusters which are highly enriched in the brain are highlighted. AAV variants are also colored by capsid insertion site in the embedding on the right. (b) Brain and capsid counts for all AAV5Loop2 and AAV9Loop1 variants with inserted APOA1 derived peptides. Shown in blue are the brain counts, and shown in orange are the capsid counts. Red arrow indicates the location of the peptide inserted in AAV5.APOA1 and AAV9.APOA1 respectively. Also shown are electron micrographs for AAV5.APOA1 and AAV9.APOA1. (c) RT-qPCR values (transduction relative to AAV9) for individual in vivo validations of AAV5.APOA1 and AAV9.APOA1 in C57/BL6 mice, confirming liver detargeting. (d) Performance of AAV5.APOA1 in BALB/c mice. Bar graph shows RT-qPCR values (transduction relative to AAV9 in BALB/c), for AAV5.APOA1 across 8 organs, confirming liver detargeting is strain independent.

FIG. 7A-E. Novel immune-orthogonal AAV serotype mining, characterization and tropism reprogramming via displayed ligand peptides. (a) Schematic of the computational pipeline used to identify novel AAV serotypes for testing. Basic local alignment search tool (BLAST) was used to identify 687 initial capsids with sequence homology to the AAV2 cap gene. This initial list was then filtered to exclude truncated genomes, redundant samples, human and non-mammalian serotypes, as well as close orthologs. The final resulting list contained 23 AAV capsid sequences for subsequent investigation. (b) Hierarchical clustering dendrogram of AAV capsid sequences. Shown in red are previously identified AAV serotypes currently in use. Novel AAVs identified and used for downstream testing are shown in black. (c) Two stage filtering of novel AAVs. AAVs were first assessed by measuring their ability to package, and then by their ability to transduce the liver (using an mCherry transgene) in vivo. All values shown relative to the orthologous wild-type AAV5. (d) The four novel AAVs which could infect the liver (AAV MM2, AAV MG2, AAV MG1 and AAV CH1), were tested for immune cross-reactivity with AAV8. Mice were immunized with the indicated AAV, and then 3 weeks post-injection tested for antibody cross reactivity via an ELISA. (e) The PDGFC peptide from AAV9.PDGFC was inserted onto loop1 of AAV MG2 to yield AAV MG2.PDGFC. AAV MG2 and AAV MG2.PDGFC were injected into C57BL/6 mice, quantifying muscle transduction via RT-qPCR after three weeks. Bar plots show muscle transduction relative to wild-type AAV MG2. Statistical significance between groups was calculated via a T-test (*p<0.05, **p<0.01, ***p<0.001, ****p<0.0001).

### DETAILED DESCRIPTION

As used herein and in the appended claims, the singular forms “a,” “an,” and “the” include plural referents unless the context clearly dictates otherwise. Thus, for example, reference to “a cell” includes a plurality of such cells and reference to “the fragment” includes reference to one or more fragments and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this disclosure is not limited to the particular methodology, protocols, and reagents, etc., described herein and as such may vary. The terminology used herein is for the purpose of describing particular embodiments or aspects only and is not intended to limit the scope of the present disclosure.

Other than in the operating examples, or where otherwise indicated, all numbers expressing quantities of ingredients or reaction conditions used herein should be understood as modified in all instances by the term “about”. The term “about” when used to describe the present invention, in connection with percentages means±1%. The term “about”, as used herein can mean within an acceptable error range for the particular value as determined by one of ordinary skill in the art, which can depend in part on how the value is measured or determined, e.g., the limitations of the measurement system. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value can be assumed. Also, where ranges and/or subranges of values are provided, the ranges and/or subranges can include the endpoints of the ranges and/or subranges. In some cases, variations can include an amount or concentration of 20%, 10%, 5%, 1%, 0.5%, or even 0.1% of the specified amount.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. Non-limiting exemplary serotypes useful in the methods disclosed herein include any of the 11 or 12 serotypes, e.g., AAV2, AAV5, and AAV8, or variant serotypes such as AAV-DJ. The AAV structural particle is composed of 60 protein molecules made up of VP1, VP2 and VP3. Each particle contains approximately 5 VP1 proteins, 5 VP2 proteins and 50 VP3 proteins ordered into an icosahedral structure. Non-limiting exemplary VP1 sequences useful in the methods disclosed herein are provided below.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and 0-phosphoserine. In some embodiments, an amino acid analog refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. In some embodiments, an amino acid mimetic refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature. In certain instances, one or more D-amino acids can be used in various peptide compositions of the disclosure. The disclosure provides various peptides that are useful for treating various diseases and infections. These peptides can comprise naturally occurring amino acid. In other embodiments, the peptides can comprise non-natural amino acids. The use of non-natural amino acids can improve the peptides stability, decrease degradation and/or improve biological activity. For example, in some embodiments, one or more D-amino acids. In other embodiments, retroinverso peptides are contemplated using various amino acid configurations.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “Cas9” refers to a CRISPR-associated, RNA-guided endonuclease such as Streptococcus pyogenes Cas9 (spCas9; see Accession Number Q99ZW2.1, the sequence of which is incorporated herein by reference) and orthologs and biological equivalents thereof. Biological equivalents of Cas9 include, but are not limited to, C2c1 from Alicyclobacillus acidoterrestris and Cpf1 (which performs cutting/cleaving functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 (“dCas9”), which lack endonuclease activity. Cas9 may also refer to “split-Cas9” in which Cas9 is split into two halves—C-terminal Cas9 (C-Cas9) and an N-terminal Cas-9 (N-Cas9)—which can be fused with two intein moieties. See, e.g., U.S. Pat. No. 9,074,199 B1; Zetsche et al. (2015) Nat Biotechnol. 33(2):139-42; Wright et al. (2015) PNAS 112(10) 2984-89. Non-limiting examples of commercially available sources of SpCas9 comprising plasmids can be found under the following AddGene reference numbers:
42230: PX330; SpCas9 and single guide RNA;
48138: PX458; SpCas9-2A-EGFP and single guide RNA;
62988: PX459; SpCas9-2A-Puro and single guide RNA;
48873: PX460; SpCas9n (D10A nickase) and single guide RNA;
48140: PX461; SpCas9n-2A-EGFP (D10A nickase) and single guide RNA;
62987: PX462; SpCas9n-2A-Puro (D10A nickase) and single guide RNA; and
48137: PX165; SpCas9;
all of which are incorporated herein by reference.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guideRNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278.

As used herein, the term “delivery vehicle” refers to a composition useful for delivering a payload to a cell, tissue or subject. The delivery vehicle can deliver various payloads including biological agents and small molecule drugs. Exemplary, but non-limiting, delivery vehicles include a liposome, a nanoparticle, a bacteria, a bacteriophage, a virus-like particle (VLP), a erythrocyte ghost, and an exosome.

As used herein, the term “domain” can refer to a particular region of a larger molecule (e.g., a particular region of a protein or polypeptide), which can be associated with a particular function. For example, “a domain which binds to a cognate” can refer to the domain of a protein that binds one or more receptors or other protein moieties. Similarly, a corresponding coding sequence for a particular polypeptide domain can be referred to as a polynucleotide domain.

The term “encode” as it is applied to polynucleotides can refer to a polynucleotide which is said to “encode” a polypeptide if, in its native state or when manipulated by methods well known to those skilled in the art, it can be transcribed and/or translated to produce the mRNA for the polypeptide and/or a fragment thereof. In some cases, the antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality.

As used herein, “expression” can refer to the process by which polynucleotides are transcribed into mRNA and/or the process by which the transcribed mRNA is subsequently being translated into peptides, polypeptides, or proteins. If the polynucleotide is derived from genomic DNA, expression can include splicing of the mRNA in a eukaryotic cell.

As used herein, the term “functional” may be used to modify any molecule, biological, or cellular material to intend that it accomplishes a particular, specified effect.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12): 1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. J of Biotechnology 233 (2016) 74-83).

“Homology” or “identity” or “similarity” can refer to sequence similarity between two peptides or between two nucleic acid molecules. Homology can be determined by comparing a position in each sequence which can be aligned for purposes of comparison. For example, when a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the disclosure.

Homology refers to a percent (%) identity of a sequence to a reference sequence. As a practical matter, any particular sequence can be at least 50%, 60%, 70%, 80%, 85%, 90%, 92%, 95%, 96%, 97%, 98% or 99% identical to any sequence described herein. Whether such particular peptide, polypeptide or nucleic acid sequence has a particular identity/homology can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity is calculated over the full length of the reference sequence and that gaps in homology of up to 5% of the total reference sequence are allowed. A number of sequences are provided herein, it is contemplated that sequences having at least 50, 60%, 65%, 70%, 75%, 80%, 85%, 90%, 95%, 98%, 99% and 100% to any one of the sequences herein find use in any of the compositions and methods described herein.

For example, in a specific embodiment the identity between a reference sequence (query sequence, i.e., a sequence of the disclosure) and a subject sequence, also referred to as a global sequence alignment, can be determined using the FASTDB computer program based on the algorithm of Brutlag et al. (Comp. App. Biosci. 6:237-245 (1990)). In some cases, parameters for a particular embodiment in which identity is narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever is shorter. According to this embodiment, if the subject sequence is shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that are lateral to the N- and C-terminal of the subject sequence, which are not matched/aligned with a corresponding subject residue, as a percent of the total bases of the query sequence. A determination of whether a residue is matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence are considered for this manual correction. For example, a 90 residue subject sequence can be aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity can be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequence are manually corrected for.

“Hybridization” can refer to a reaction in which one or more polynucleotides react to form a complex that is stabilized via hydrogen bonding between the bases of the nucleotide residues. The hydrogen bonding can occur by Watson-Crick base pairing, Hoogstein binding, or in any other sequence-specific manner. The complex can comprise two strands forming a duplex structure, three or more strands forming a multi-stranded complex, a single self-hybridizing strand, or any combination of these. A hybridization reaction can constitute a step in a more extensive process, such as the initiation of a PC reaction, or the enzymatic cleavage of a polynucleotide by a ribozyme.

Examples of stringent hybridization conditions include: incubation temperatures of about 25 °C to about 37 °C; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40 °C to about 50 °C; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 °C to about 68 °C; buffer concentrations of about 1×SSC to about 0.lx SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “immune orthogonal” refers to a lack of immune cross-reactivity between two or more antigens. In some embodiments, the antigens are proteins (e.g., Cas9). In some embodiments, the antigens are viral antigens associated with a particular viral vector (e.g., AAV). As is recognized in the art, antigens typically include antigenic determinants having a particular sequence of 3 dimensional structure. Moreover, an antigenic determinant can comprise a domain or subsequence of a larger polypeptide or molecular sequence. In some embodiments, antigens that are immune orthogonal do not share an amino acid sequence of greater than 5, greater than 6, greater than 7, greater than 8, greater than 9, greater than 10, greater than 11, greater than 12, greater than 13, greater than 14, greater than 15, or greater than 16 consecutive amino acids. In some embodiments, antigens that are immune orthogonal do not share any highly immunogenic peptides. In some embodiments, antigens that are immune orthogonal do not share affinity for a major histocompatibility complex (e.g., MHC class I or class II). Antigens that are immune orthogonal are amenable for sequential dosing to evade a host immune system.

The term “immunosilent” refers to an epitope or foreign peptide, polypeptide or protein that does not elicit an immune response from a host upon administration. In some embodiments, the peptide, polypeptide or protein does not elicit an adaptive immune response. In some embodiments, the peptide, polypeptide or protein does not elicit an innate immune response. In some embodiments, the peptide, polypeptide or protein does not elicit either an adaptive or an innate immune response. In some embodiments, an immunosilent peptide, polypeptide or protein has reduced immunogenicity.

The term “isolated” as used herein can refer to molecules or biologicals or cellular materials being substantially free from other materials. In one aspect, the term “isolated” can refer to nucleic acid, such as DNA or RNA, or protein or polypeptide (e.g., an antibody or derivative thereof), or cell or cellular organelle, or tissue or organ, separated from other DNAs or RNAs, or proteins or polypeptides, or cells or cellular organelles, or tissues or organs, respectively, that are present in the natural source. The term “isolated” also can refer to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and may not be found in the natural state. In some cases, the term “isolated” is also used herein to refer to polypeptides which are isolated from other cellular proteins and is meant to encompass both purified and recombinant polypeptides. In some cases, the term “isolated” is also used herein to refer to cells or tissues that are isolated from other cells or tissues and is meant to encompass both cultured and engineered cells or tissues.

“Messenger RNA” or “mRNA” is a nucleic acid molecule that is transcribed from DNA and then processed to remove non-coding sections known as introns. In some cases, the resulting mRNA is exported from the nucleus (or another locus where the DNA is present) and translated into a protein. The term “pre-mRNA” can refer to the strand prior to processing to remove non-coding sections. mRNA has “U” in place of “T” in cDNA coding sequences.

The term “ortholog” is used in reference of another gene or protein and intends a homolog of said gene or protein that evolved from the same ancestral source or which are evolved artificially using molecular biology and genetic engineering. Orthologs may or may not retain the same function as the gene or protein to which they are orthologous. Non-limiting examples of Cas9 orthologs include S. aureus Cas9 (“spCas9”), S. thermophiles Cas9, L. pneumophilia Cas9, N. lactamica Cas9, N. meningitides Cas9, B. longum Cas9, A. muciniphila Cas9, and O. laneus Cas9.

The term “payload” refers to a therapeutic and diagnostic agents that can be loaded into or onto a delivery vehicle. Such payload include biological and small molecule entities. Exemplary payload agents include small molecule drugs, biological molecules, viruses, therapeutic agents, prodrugs, gene silencing agents, chemotherapeutics, diagnostic agents, and/or components of gene editing systems. Examples of biological molecules include, but are not limited to, nucleic acids (e.g., DNA, RNA, mRNA, modified mRNA, small RNAs, siRNA, miRNA, genes, and transgenes), peptides/proteins (including antibodies, enzymes, transcription factors, etc.), viruses, hormones, carbohydrates, lipids, and vitamins. Examples of gene silencing agents, include siRNA, chRNAs, miRs, ribozymes, morpholinos, and esiRNAs. Examples of gene editing systems include, but are not limited to, CRISPR-Cas systems, zinc finger nucleases, and TALENs. Examples of diagnostic agents, include but are not limited to, dyes and stains, radioactive tracers, and contrast agents. Examples, of anticancer agents and chemotherapeutics that can be used with or loaded into a delivery vehicle include, but are not limited to, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® (docetaxel) (Rhone-Poulenc Rorer, Antony, France); chloranbucil; GEMZAR® (gemcitabine); 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included anticancer agents are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; antibodies such as trastuzumab and pharmaceutically acceptable salts, acids or derivatives of any of the above. Examples of protein payloads include mammalian proteins, such as, e.g., growth hormone (GH), including human growth hormone, bovine growth hormone, and other members of the GH supergene family; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX tissue factor, and von Willebrands factor; anti-clotting factors such as Protein C; atrial natriuretic factor; lung surfactant; a plasminogen activator, such as urokinase or tissue-type plasminogen activator (t-PA); bombazine; thrombin; alpha tumor necrosis factor, beta tumor necrosis factor; enkephalinase; RANTES (regulated on activation normally T-cell expressed and secreted); human macrophage inflammatory protein (MIP-1-alpha); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-beta1, TGF-beta2, TGF-beta3, TGF-beta4, or TGF-beta5; insulin-like growth factor-I and -II (IGF-I and IGF-II); des(1-3)-IGF-I (brain IGF-D; insulin-like growth factor binding proteins; CD proteins such as CD3, CD4, CD8, CD19 and CD20; osteoinductive factors; immunotoxins; a bone morphogenetic protein (BMP); T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

The members of the GH supergene family include growth hormone, prolactin, placental lactogen, erythropoietin, thrombopoietin, interleukin-2, interleukin-3, interleukin-4, interleukin-5, interleukin-6, interleukin-7, interleukin-9, interleukin-10, interleukin-11, interleukin-12 (p35 subunit), interleukin-13, interleukin-15, oncostatin M, ciliary neurotrophic factor, leukemia inhibitory factor, alpha interferon, beta interferon, gamma interferon, omega interferon, tau interferon, granulocyte-colony stimulating factor, granulocyte-macrophage colony stimulating factor, macrophage colony stimulating factor, cardiotrophin-1 and other proteins identified and classified as members of the family.

Other payload agents that can be incorporated in the delivery vehicle include gastrointestinal therapeutic agents such as aluminum hydroxide, calcium carbonate, magnesium carbonate, sodium carbonate and the like; non-steroidal antifertility agents; parasympathomimetic agents; psychotherapeutic agents; major tranquilizers such as chloropromazine HCl, clozapine, mesoridazine, metiapine, reserpine, thioridazine and the like; minor tranquilizers such as chlordiazepoxide, diazepam, meprobamate, temazepam and the like; rhinological decongestants; sedative-hypnotics such as codeine, phenobarbital, sodium pentobarbital, sodium secobarbital and the like; other steroids such as testosterone and testosterone propionate; sulfonamides; sympathomimetic agents; vaccines; vitamins and nutrients such as the essential amino acids, essential fats and the like; antimalarials such as 4-aminoquinolines, 8-aminoquinolines, pyrimethamine and the like; anti-migraine agents such as mazindol, phentermine and the like; anti-Parkinson agents such as L-dopa; anti-spasmodics such as atropine, methscopolamine bromide and the like; antispasmodics and anticholinergic agents such as bile therapy, digestants, enzymes and the like; antitussives such as dextromethorphan, noscapine and the like; bronchodilators; cardiovascular agents such as anti-hypertensive compounds, Rauwolfia alkaloids, coronary vasodilators, nitroglycerin, organic nitrates, pentaerythritotetranitrate and the like; electrolyte replacements such as potassium chloride; ergotalkaloids such as ergotamine with and without caffeine, hydrogenated ergot alkaloids, dihydroergocristine methanesulfate, dihydroergocomine methanesulfonate, dihydroergokroyptine methanesulfate and combinations thereof; alkaloids such as atropine sulfate, Belladonna, hyoscine hydrobromide and the like; analgesics; narcotics such as codeine, dihydrocodienone, meperidine, morphine and the like; non-narcotics such as salicylates, aspirin, acetaminophen, d-propoxyphene and the like.

Antibiotic payloads include, for example, the cephalosporins, chlorarnphenical, gentamicin, kanamycin A, kanamycin B, the penicillins, ampicillin, streptomycin A, antimycin A, chloropamtheniol, metronidazole, oxytetracycline penicillin G, the tetracyclines, and the like.

Other payload agents can include vaccines or antigenic agents. For example, payload antigens derived from microorganisms such as Neisseria gonorrhea, Mycobacterium tuberculosis, Herpes virus (humonis, types 1 and 2), Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, equine herpes virus 1, equine arteritis virus, IBR-IBP virus, BVD-MB virus, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum and the like can be loaded. In other embodiments, the payload can comprise neutralizing antibodies that counteract the above microorganisms.

In other embodiments, the payload can comprise enzymes such as ribonuclease, neuramidinase, trypsin, glycogen phosphorylase, sperm lactic dehydrogenase, sperm hyaluronidase, adenossinetriphosphatase, alkaline phosphatase, alkaline phosphatase esterase, amino peptidase, trypsin chymotrypsin, amylase, muramidase, acrosomal proteinase, diesterase, glutamic acid dehydrogenase, succinic acid dehydrogenase, beta-glycophosphatase, lipase, ATP-ase alpha-peptate gamma-glutamylotranspeptidase, sterol-3-beta-ol-dehydrogenase, DPN-di-aprorase.

Peptide-payload conjugates are also encompassed by the disclosure, wherein a peptide of the disclosure is linked or fused directly to a payload molecule as set forth herein such that the peptide-payload conjugate can be directly delivery (without loading into a delivery vehicle) to target a desired tissue based upon the peptide's tropism.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CMV promoter and U6 promoter.

The term “protein”, “peptide” and “polypeptide” are used interchangeably and in their broadest sense to refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation is placed on the maximum number of amino acids which can comprise a protein's or peptide's sequence. As mentioned above, the term “amino acid” can refer to either natural and/or unnatural or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a peptide fragment that is used to link these domains together—optionally to preserve the conformation of the fused protein domains and/or prevent unfavorable interactions between the fused protein domains which can compromise their respective functions.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and can perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also can refer to both double and single stranded molecules. Unless otherwise specified or required, any embodiment of this disclosure that is a polynucleotide can encompass both the double stranded form and each of two complementary single stranded forms known or predicted to make up the double stranded form.

The term “polynucleotide sequence” can be the alphabetical representation of a polynucleotide molecule. This alphabetical representation can be input into databases in a computer having a central processing unit and used for bioinformatics applications such as functional genomics and homology searching.

Similarly, the term “polypeptide sequence”, “peptide sequence” or “protein sequence” can be the alphabetical representation of a polypeptide molecule. This alphabetical representation can be input into databases in a computer having a central processing unit and used for bioinformatics applications such as functional proteomics and homology searching.

As used herein, the term “recombinant expression system” refers to a genetic construct or constructs for the expression of certain genetic material formed by recombination.

As used herein, the term “recombinant protein” can refer to a polypeptide or peptide which is produced by recombinant DNA techniques, wherein generally, DNA encoding the polypeptide or peptide is inserted into a suitable expression vector which is in turn used to transform a host cell to produce the heterologous polypeptide or peptide.

The term “sequencing” as used herein, can comprise bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLiD sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, Enigma sequencing, or any combination thereof.

As used herein, the term “subject” is intended to mean any animal. In some embodiments, the subject may be a mammal; in further embodiments, the subject may be a bovine, equine, feline, murine, porcine, canine, human, or rat.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection (e.g., using commercially available reagents such as, for example, LIPOFECTIN® (Invitrogen Corp., San Diego, CA), LIPOFECTAMINE® (Invitrogen), FUGENE® (Roche Applied Science, Basel, Switzerland), JETPEI™ (Polyplus-transfection Inc., New York, NY), EFFECTENE® (Qiagen, Valencia, CA), DREAMFECT™ (OZ Biosciences, France) and the like), or electroporation. Suitable methods for transforming or transfecting host cells can be found in Sambrook, et al. (Molecular Cloning: A Laboratory Manual. 2nd, ed., Cold Spring harbor Laboratory, Cold Spring Harbor Laboratory Press, Cold Spring Harbor, N.Y., 1989), and other laboratory manuals. Standard recombinant DNA and molecular cloning techniques used herein are well known in the art and are described in Sambrook, J., Fritsch, E. F. and Maniatis, T., Molecular Cloning: A Laboratory Manual, 2^{nd} ed.; Cold Spring Harbor Laboratory: Cold Spring Harbor, N.Y., (1989) and by Silhavy, T. J., Bennan, M. L. and Enquist, L. W., Experiments with Gene Fusions; Cold Spring Harbor Laboratory: Cold Spring Harbor, N.Y., (1984); and by Ausubel, F. M. et. al., Current Protocols in Molecular Biology, Greene Publishing and Wiley-Interscience (1987) each of which are hereby incorporated by reference in its entirety. Additional useful methods are described in manuals including Advanced Bacterial Genetics (Davis, Roth and Botstein, Cold Spring Harbor Laboratory, 1980), Experiments with Gene Fusions (Silhavy, Berman and Enquist, Cold Spring Harbor Laboratory, 1984), Experiments in Molecular Genetics (Miller, Cold Spring Harbor Laboratory, 1972) Experimental Techniques in Bacterial Genetics (Maloy, in Jones and Bartlett, 1990), and A Short Course in Bacterial Genetics (Miller, Cold Spring Harbor Laboratory 1992) each of which are hereby incorporated by reference in its entirety.

The terms “treat”, “treating” and “treatment”, as used herein, refers to ameliorating symptoms associated with a disease or disorder (e.g., cancer, Covid-19 etc.), including preventing or delaying the onset of the disease or disorder symptoms, and/or lessening the severity or frequency of symptoms of the disease or disorder.

As used herein, the term “vector” can refer to a nucleic acid construct deigned for transfer between different hosts, including but not limited to a plasmid, a virus, a cosmid, a phage, a BAC, a YAC, etc. In some embodiments, a “viral vector” is defined as a recombinantly produced virus or viral particle that comprises a polynucleotide to be delivered into a host cell, either in vivo, ex vivo or in vitro. In some embodiments, plasmid vectors can be prepared from commercially available vectors. In other embodiments, viral vectors can be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct can refer to the polynucleotide comprising the retroviral genome or part thereof, and a gene of interest. Further details as to modern methods of vectors for use in gene transfer can be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). In one aspect, the promoter is a pol III promoter.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and ‘Vector” can be used interchangeably. However, the disclosure is intended to include such other forms of expression vectors, such as viral vectors (e.g., replication defective retroviruses, adenoviruses and adeno-associated viruses), which serve equivalent functions. Typically, the vector or plasmid contains sequences directing transcription and translation of a relevant gene or genes, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Typically, the vector or plasmid contains sequences directing transcription and translation of a gene fragment, a selectable marker, and sequences allowing autonomous replication or chromosomal integration. Suitable vectors comprise a region 5′ of the gene which harbors transcriptional initiation controls and a region 3′ of the DNA fragment which controls transcription termination. Both control regions may be derived from genes homologous to the transformed host cell, although it is to be understood that such control regions may also be derived from genes that are not native to the species chosen as a production host.

Initiation control regions or promoters, which are useful to drive expression of the relevant pathway coding regions in the desired host cell are numerous and familiar to those skilled in the art. Virtually any promoter capable of driving these genetic elements is suitable for the present invention including, but not limited to, lac, ara, tet, trp, IPL, IPR, T7, tac, and trc (useful for expression in Escherichia coli and Pseudomonas); the amy, apr, npr promoters and various phage promoters useful for expression in Bacillus subtilis, and Bacillus licheniformis; nisA (useful for expression in gram positive bacteria, Eichenbaum et al. Appl. Environ. Microbiol. 64(8):2763-2769 (1998)); and the synthetic Pll promoter (useful for expression in Lactobacillus plantarum, Rud et al., Microbiology 152:1011-1019 (2006)). Termination control regions may also be derived from various genes native to the preferred hosts.

Adeno-associated viruses (AAVs) are common gene therapy vectors, however, their effectiveness is hindered by poor target tissue transduction and off-target delivery. Hypothesizing that naturally occurring receptor-ligand interactions could be repurposed to engineer tropism. The disclosure provides a method wherein all annotated protein ligands known to bind human receptors were fragmented into tiling 20-mer peptides and displayed onto the surface loops of AAV5 and AAV9 capsids at two sites. The resulting capsid libraries, comprising >1 million AAV variants, were screened across 9 tissues in C57BL/6 mice. Tracking variant abundance, >250,000 variants were identified which packaged into capsids, and >15,000 variants which efficiently transduced at least one mouse organ. Twenty-one (21) AAV variants were validated with 74.3% of the organ tropism predictions accurately reproducing, confirming overall screen efficacy. Systematic ligand tiling enabled prediction of putative AAV-receptor interactions, were successfully validated by targeted genetic perturbations. Comprehensive peptide tiling also enabled examination of homologous peptide activity. Interestingly, functional peptides that were observed tended to be derived from specific domains on ligands. Notably, certain peptides also displayed consistent activity across mice strains, capsid insertion contexts, and capsid serotypes, including novel immune orthogonal serotypes. Further analyses of displayed peptides revealed that biophysical attributes were highly predictive of AAV variant packaging, and there was a machine learnable relationship between peptide sequence and tissue tropism. The disclosed comprehensive ligand peptide tiling and display approach can enable engineering of tropism across diverse viral, viral-like and non-viral delivery platforms, and shed light into basic receptor-ligand biology.

The disclosure provides a plurality of peptides useful for targeting a desired tissue. The disclosure also provides peptides that are immunosilent. The disclosure also provides vectors, delivery vehicles and peptide-payload conjugates comprising one or more of the peptides of the disclosure having a desired tropism.

Rational screening strategies have immense potential to expand the molecular tools available for clinical gene therapy applications. While AAV engineering efforts have been conducted for over a decade, advances in DNA synthesis have enabled us to create here a data-driven library of AAV variants leveraging existing functional biomolecules from nature. Using natural biomolecules as a defined source of inserted peptides has multiple benefits over random hexamers (and similar methods). First, natural biomolecules have been pre-filtered for biological functionality by millennia of evolutionary selection pressure. Second, a defined library allows for robust quantification of the fitness of each AAV variant, enabling facile stratification of AAV variants by infectivity across organs of interest. While this method was primarily applied to engineer AAVs, mining nature for functional biomolecules has applications in a wide range of protein engineering challenges, such as engineering orthogonal viral (including lentiviruses) and non-viral (including lipid nanoparticles) vectors or identifying biologic inhibitors of critical protein/protein interactions.

Additionally, it is believed that the rational ligand tiling approach described herein could lead to important insights into basic AAV-receptor binding and transduction more generally. Previous efforts have either utilized large-scale genetic screens or unique observations about a particular AAV variant's transduction behavior. As the AAV variants described herein display peptides derived from natural receptor-interacting ligands, it is believed that the AAV variants may maintain some of this binding capacity which partially explains their altered transduction profiles.

The disclosure focuses on adeno-associated viruses (AAVs) which have emerged as a leading vector for gene delivery in clinical applications. While multiple AAV-mediated therapies have achieved regulatory approval, efficient directing of treatment to target tissues is challenging with systemic injection. To overcome this, high viral titers are often used in treatments, which has in-turn been associated with potential for hepatotoxicity in clinical trials. Localized injections are also problematic, often requiring invasive procedures with the potential for organ damage and long recovery times. Due to these delivery challenges, some gene therapeutics have elected to pursue, where feasible, ex vivo treatment designs to overcome targeting issues, but this in-turn can lead to dependency on complex lab procedures, and high manufacturing costs.

There are thus numerous ongoing research efforts to improve in vivo therapeutic targeting, AAV variants have been engineered to specifically target tissues such as the brain and muscle. This has predominantly been established using strategies of iteratively screening random peptides inserted into the AAV capsid, or capsid shuffling, or randomly mutagenizing the capsid sequence as a whole, or direct chemical engineering. Although mutagenizing AAV capsids via random oligomers has yielded functional capsids with novel properties a stochastic mutational screening strategy limits the ability to predict future functional variants, and thus rational and programmable engineering of viral phenotypes remains an elusive goal.

Towards rational engineering of viral function, deep mutational libraries and associated screens of function have enabled systematic mapping of capsid mutation fitness, providing critical information which can be used to predict future variant activity. Additionally, defined libraries of pooled oligonucleotides have been used to insert gene fragments derived from proteins with known affinity to synapses into the AAV capsid, with the goal of improving retrograde axonal transport. While these methodologies have provided important insights for AAV engineering, much is still unknown regarding how AAV genotype impacts packaging and tissue transduction. Consequently, there is a critical need for systematic datasets mapping AAV genotype to clinically relevant properties such as organ specificity. Given the clinical danger of hepatotoxicity and other efficacy issues related to off-target transduction, leveraging screening technologies to yield highly specific AAV capsids has great value to the medical and scientific community.

With a goal to develop a rational and potentially programmable strategy for enabling tissue targeting, it was hypothesized that receptor-ligand interactions, which mediate the spectrum of naturally occurring cell-protein and cell-cell interactions, could be repurposed for engineering AAV tropism. However, as neither the interaction interfaces nor the cell type specificity profiles of receptor-ligand interactions are fully mapped to comprehensively interrogate as well as engineer these. To study and address this, all annotated protein ligands known to bind human receptors were fragmented into tiling short peptides and displayed onto surface loops of AAV capsids, an approach termed “AAV-PepTile”. Short peptides grafted into stabilizing molecular scaffolds can recapitulate local protein domain structure, and as protein-protein interface sites are typically 1200-2000 A2, with specific peptide hot loops ranging from 4-8 amino acids (AAs) contributing maximally to the binding energy of protein-protein interactions, it was hypothesized that 20 amino acid peptide insertions into the AAV capsid could drastically alter its binding ability, and therefore, its transduction profile in vivo. While insertion of still longer peptides could, in principle, more faithfully recapitulate local ligand structures, AAVs do not generally tolerate larger than 20-25 amino acid insertions without severely compromising capsid packaging efficiency. Based on this, experiments proceeded using a 20 amino acid peptide+flanking 2 amino acid linker insertions, all together a library of >1 million AAV variants was constructed by inserting corresponding oligonucleotide pool synthesized gene fragments coding for potential receptor-ligands and cell membrane permeable proteins into one of two surface loops on AAV5 and AAV9. Unlike random peptide libraries, ligand tiling enabled robust quantitation of tissue transduction rates for all variants screened. Furthermore, systematic examination of the activity of similar peptides generate predictions of putative receptor interactions driving AAV variant tropism. Quantifying transduction rates across nine organs, extremely specific variants targeting the brain and lung were identified, as well as muscle and heart targeting variants with broader organ transduction. The resulting data linking AAV variant genotype to packaging efficacy and tissue specificity expands the understanding of the AAV fitness landscape, and provides a unique resource from which further data-driven engineering efforts can be built.

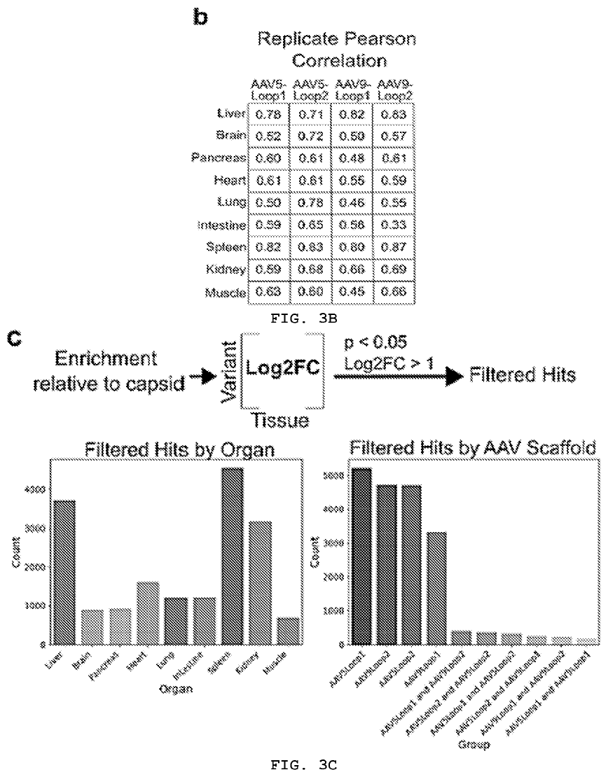

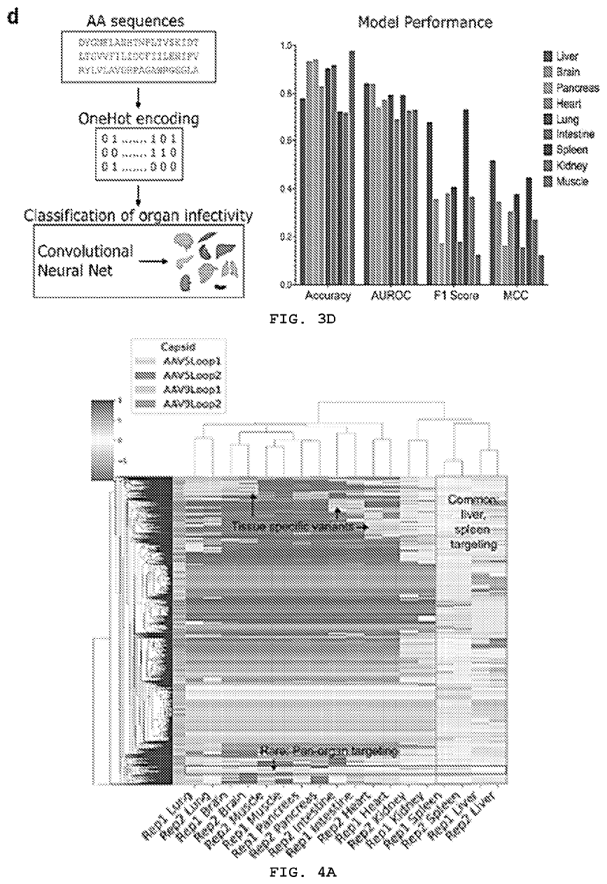

The general schematic for the AAV screens is outlined in FIG. 3a. Briefly, in the primary screen, a 20 amino acid peptide library consisting of tiled fragments from all known receptor-interacting ligands along with other interesting protein classes flanked by glycine-serine linkers were displayed into the 2 surface-exposed loops of two clinically relevant AAV serotypes, AAV5 and AAV9 with their sequences and specific amino acid site of insertion shown in Table 1.

TABLE 1
Showing both the DNA and amino acid sequences
and insertion sites of the two wild-type AAV serotypes utilized in
the screens, AAV5 and AAV9. Bolded and underlined are the flanking
residues of the of the ‘Loop 1’ site and shown in
bold/ital/underline are the flanking residues of the ‘Loop 2’ site.

| Name | DNA Sequence | AA Sequence |
|---|---|---|
| AAV5 | ATGTCTTTTGTTGATCACCCTCCAGATTGGTTGGAAGAAGTT | MSFVDHPPDWLEEVGEGLREFLGL |
| | GGTGAAGGTCTTCGCGAGTTTTTGGGCCTTGAAGCGGGCCCA | EAGPPKPKPNQQHQDQARGLVLPG |
| | CCGAAACCAAAACCCAATCAGCAGCATCAAGATCAAGCCCG | YNYLGPGNGLDRGEPVNRADEVAR |
| | TGGTCTTGTGCTGCCTGGTTATAACTATCTCGGACCCGGAAA | EHDISYNEQLEAGDNPYLKYNHAD |
| | CGGTCTCGATCGAGGAGAGCCTGTCAACAGGGCAGACGAGG | AEFQEKLADDTSFGGNLGKAVFQA |
| | TCGCGCGAGAGCACGACATCTCGTACAACGAGCAGCTTGAG | KKRVLEPFGLVEEGAKTAPTGKRID |
| | GCGGGAGACAACCCCTACCTCAAGTACAACCACGCGGACGC | DHFPKRKKARTEEDSKPSTSSDAEA |
| | CGAGTTTCAGGAGAAGCTCGCCGACGACACATCCTTCGGGG | GPSGSQQLQIPAQPASSLGADTMSA |
| | GAAACCTCGGAAAGGCAGTCTTTCAGGCCAAGAAAAGGGTT | GGGGPLGDNNQGADGVGNASGDW |
| | CTCGAACCTTTTGGCCTGGTTGAAGAGGGTGCTAAGACGGC | HCDSTWMGDRVVTKSTRTWVLPS |
| | CCCTACCGGAAAGCGGATAGACGACCACTTTCCAAAAAGAA | YNNHQYREIKSGSVDGSNANAYFG |
| | AGAAGGCTCGGACCGAAGAGGACTCCAAGCCTTCCACCTCG | YSTPWGYFDFNRFHSHWSPRDWQR |
| | TCAGACGCCGAAGCTGGACCCAGCGGATCCCAGCAGCTGCA | LINNYWGFRPRSLRVKIFNIQVKEV |
| | AATCCCAGCCCAACCAGCCTCAAGTTTGGGAGCTGATACAA | TVQDSTTTIANNLTSTVQVFTDDDY |
| | TGTCTGCGGGAGGTGGCGGCCCATTGGGCGACAATAACCAA | QLPYVVGNGTEGCLPAFPPQVFTLP |
| | GGTGCCGATGGAGTGGGCAATGCCTCGGGAGATTGGCATTG | QYGYATLNRDNTENPTERSSFFCLE |
| | CGATTCCACGTGGATGGGGGACAGAGTCGTCACCAAGTCCA | YFPSKMLRTGNNFEFTYNFEEVPFH |
| | CCCGAACCTGGGTGCTGCCCAGCTACAACAACCACCAGTAC | SSFAPSQNLFKLANPLVDQYLYRFV |
| | CGAGAGATCAAAAGCGGCTCCGTCGACGGAAGCAACGCCAA | STNNTGGVQFNKNLAGRYANTYK |
| | CGCCTACTTTGGATACAGCACCCCCTGGGGGTACTTTGACTT | NWFPGPMGRTQGWNLGSGVNRAS |
| | TAACCGCTTCCACAGCCACTGGAGCCCCCGAGACTGGCAAA | VSAFATTNRMELEGASYQVPPQPN |
| | GACTCATCAACAACTACTGGGGCTTCAGACCCCGGTCCCTCA | GMTNNLQGSNTYALENTMIFNSQP |
| | GAGTCAAAATCTTCAACATTCAAGTCAAAGAGGTCACGGTG | ANPGTTATYLEGNMLITSESETQPV |
| | CAGGACTCCACCACCACCATCGCCAACAACCTCACCTCCACC | NRVAYNVGGQMATNNQSSTTAPA |
| | GTCCAAGTGTTTACGGACGACGACTACCAGCTGCCCTACGTC | TGTYNLQEIVPGSVWMERDVYLQG |
| | GTCGGCAACGGGACCGAGGGATGCCTGCCGGCCTTCCCTCC | PIWAKIPETGAHFHPSPAMGGFGLK |
| | GCAGGTCTTTACGCTGCCGCAGTACGGTTACGCGACGCTGA | HPPPMMLIKNTPVPGNITSFSDVPVS |
| | ACCGCGACAACACAGAAAATCCCACCGAGAGGAGCAGCTTC | SFITQYSTGQVTVEMEWELKKENS |
| | TTCTGCCTAGAGTACTTTCCCAGCAAGATGCTGAGAACGGGC | KRWNPEIQYTNNYNDPQFVDFAPD |
| | AACAACTTTGAGTTTACCTACAACTTTGAGGAGGTGCCCTTC | STGEYRTTRPIGTRYLTRPL (SEQ ID |
| | CACTCCAGCTTCGCTCCCAGTCAGAACCTGTTCAAGCTGGCC | NO: 2) |
| | AACCCGCTGGTGGACCAGTACTTGTACCGCTTCGTGAGCACA | |
| | AATAACACTGGCGGAGTCCAGTTCAACAAGAACCTGGCCGG | |
| | GAGATACGCCAACACCTACAAAAACTGGTTCCCGGGGCCCA | |
| | TGGGCCGAACCCAGGGCTGGAACCTGGGCTCCGGGGTCAAC | |
| | CGCGCCAGTGTCAGCGCCTTCGCCACGACCAATAGGATGGA | |
| | GCTCGAGGGCGCGAGTTACCAGGTGCCCCCGCAGCCGAACG | |
| | GCATGACCAACAACCTCCAGGGCAGCAACACCTATGCCCTG | |
| | GAGAACACTATGATCTTCAACAGCCAGCCGGCGAACCCGGG | |
| | CACCACCGCCACGTACCTCGAGGGCAACATGCTCATCACCA | |
| | GCGAGAGCGAGACGCAGCCGGTGAACCGCGTGGCGTACAAC | |
| | GTCGGCGGGCAGATGGCCACCAACAACCAGAGCTCCACCAC | |
| | TGCCCCCGCGACCGGCACGTACAACCTCCAGGAAATCGTGC | |
| | CCGGCAGCGTGTGGATGGAGAGGGACGTGTACCTCCAAGGA | |
| | CCCATCTGGGCCAAGATCCCAGAGACGGGGGCGCACTTTCA | |
| | CCCCTCTCCGGCCATGGGCGGATTCGGACTCAAACACCCACC | |
| | GCCCATGATGCTCATCAAGAACACGCCTGTGCCCGGAAATA | |
| | TCACCAGCTTCTCGGACGTGCCCGTCAGCAGCTTCATCACCC | |
| | AGTACAGCACCGGGCAGGTCACCGTGGAGATGGAGTGGGAG | |
| | CTCAAGAAGGAAAACTCCAAGAGGTGGAACCCAGAGATCCA | |
| | GTACACAAACAACTACAACGACCCCCAGTTTGTGGACTTTGC | |
| | CCCGGACAGCACCGGGGAATACAGAACCACCAGACCTATCG | |
| | GAACCCGATACCTTACCCGACCCCTTTAA (SEQ ID NO: 1) | |
| AAV9 | ATGGCTGCCGATGGTTATCTTCCAGATTGGCTCGAGGACAAC | MAADGYLPDWLEDNLSEGIREWW |
| | CTTAGTGAAGGAATTCGCGAGTGGTGGGCTTTGAAACCTGG | ALKPGAPQPKANQQHQDNARGLVL |
| | AGCCCCTCAACCCAAGGCAAATCAACAACATCAAGACAACG | PGYKYLGPGNGLDKGEPVNAADA |
| | CTCGAGGTCTTGTGCTTCCGGGTTACAAATACCTTGGACCCG | AALEHDKAYDQQLKAGDNPYLKY |
| | GCAACGGACTCGACAAGGGGGAGCCGGTCAACGCAGCAGA | NHADAEFQERLKEDTSFGGNLGRA |
| | CGCGGCGGCCCTCGAGCACGACAAGGCCTACGACCAGCAGC | VFQAKKRLLEPLGLVEEAAKTAPG |
| | TCAAGGCCGGAGACAACCCGTACCTCAAGTACAACCACGCC | KKRPVEQSPQEPDSSAGIGKSGAQP |
| | GACGCCGAGTTCCAGGAGCGGCTCAAAGAAGATACGTCTTT | AKKRLNFGQTGDTESVPDPQPIGEP |
| | TGGGGGCAACCTCGGGCGAGCAGTCTTCCAGGCCAAAAAGA | PAAPSGVGSLTMASGGGAPVADNN |
| | GGCTTCTTGAACCTCTTGGTCTGGTTGAGGAAGCGGCTAAGA | EGADGVGSSSGNWHCDSQWLGDR |
| | CGGCTCCTGGAAAGAAGAGGCCTGTAGAGCAGTCTCCTCAG | VITTSTRTWALPTYNNHLYKQISNS |
| | GAACCGGACTCCTCCGCGGGTATTGGCAAATCGGGTGCACA | TSGGSSNDNAYFGYSTPWGYFDFN |
| | GCCCGCTAAAAAGAGACTCAATTTCGGTCAGACTGGCGACA | RFHCHFSPRDWQRLINNNWGFRPK |
| | CAGAGTCAGTCCCAGACCCTCAACCAATCGGAGAACCTCCC | RLNFKLFNIQVKEVTDNNGVKTIAN |
| | GCAGCCCCCTCAGGTGTGGGATCTCTTACAATGGCTTCAGGT | NLTSTVQVFTDSDYQLPYVLGSAHE |
| | GGTGGCGCACCAGTGGCAGACAATAACGAAGGTGCCGATGG | GCLPPFPADVFMIPQYGYLTLNDGS |
| | AGTGGGTAGTTCCTCGGGAAATTGGCATTGCGATTCCCAATG | QAVGRSSFYCLEYFPSQMLRTGNNF |
| | GCTGGGGGACAGAGTCATCACCACCAGCACCCGAACCTGGG | QFSYEFENVPFHSSYAHSQSLDRLM |
| | CCCTGCCCACCTACAACAATCACCTCTACAAGCAAATCTCCA | NPLIDQYLYYLSKTINGSGQNQQTL |
| | ACAGCACATCTGGAGGATCTTCAAATGACAACGCCTACTTC | KFSVAGPSNMAVQGRNYIPGPSYR |
| | GGCTACAGCACCCCCTGGGGGTATTTTGACTTCAACAGATTC | QQRVSTTVTQNNNSEFAWPGASSW |
| | CACTGCCACTTCTCACCACGTGACTGGCAGCGACTCATCAAC | ALNGRNSLMNPGPAMASHKEGEDR |
| | AACAACTGGGGATTCCGGCCTAAGCGACTCAACTTCAAGCT | FFPLSGSLIFGKQGTGRDNVDADKV |
| | CTTCAACATTCAGGTCAAAGAGGTTACGGACAACAATGGAG | MITNEEEIKTTNPVATESYGQVATN |
| | TCAAGACCATCGCCAATAACCTTACCAGCACGGTCCAGGTCT | HQSAQAQAQTGWVQNQGILPGMV |
| | TCACGGACTCAGACTATCAGCTCCCGTACGTGCTCGGGTCGG | WQDRDVYLQGPIWAKIPHTDGNFH |
| | CTCACGAGGGCTGCCTCCCGCCGTTCCCAGCGGACGTTTTCA | PSPLMGGFGMKHPPPQILIKNTPVP |
| | TGATTCCTCAGTACGGGTATCTGACGCTTAATGATGGAAGCC | ADPPTAFNKDKLNSFITQYSTGQVS |
| | AGGCCGTGGGTCGTTCGTCCTTTTACTGCCTGGAATATTTCC | VEIEWELQKENSKRWNPEIQYTSNY |
| | CGTCGCAAATGCTAAGAACGGGTAACAACTTCCAGTTCAGC | YKSNNVEFAVNTEGVYSEPRPIGTR |
| | TACGAGTTTGAGAACGTACCTTTCCATAGCAGCTACGCTCAC | YLTRNL (SEQ ID NO: 4) |
| | AGCCAAAGCCTGGACCGACTAATGAATCCACTCATCGACCA | |
| | ATACTTGTACTATCTCTCAAAGACTATTAACGGTTCTGGACA | |
| | GAATCAACAAACGCTAAAATTCAGTGTGGCCGGACCCAGCA | |
| | ACATGGCTGTCCAGGGAAGAAACTACATACCTGGACCCAGC | |
| | TACCGACAACAACGTGTCTCAACCACTGTGACTCAAAACAA | |
| | CAACAGCGAATTTGCTTGGCCTGGAGCTTCTTCTTGGGCTCT | |
| | CAATGGACGTAATAGCTTGATGAATCCTGGACCTGCTATGGC | |
| | CAGCCACAAAGAAGGAGAGGACCGTTTCTTTCCTTTGTCTGG | |
| | ATCTTTAATTTTTGGCAAACAAGGAACTGGAAGAGACAACG | |
| | TGGATGCGGACAAAGTCATGATAACCAACGAAGAAGAAATT | |
| | AAAACTACTAACCCGGTAGCAACGGAGTCCTATGGACAAGT | |
| | GGCCACAAACCACCAGAGTGCCCAAGCACAGGCGCAGACCG | |
| | GCTGGGTTCAAAACCAAGGAATACTTCCGGGTATGGTTTGG | |
| | CAGGACAGAGATGTGTACCTGCAAGGACCCATTTGGGCCAA | |
| | AATTCCTCACACGGACGGCAACTTTCACCCTTCTCCGCTGAT | |
| | GGGAGGGTTTGGAATGAAGCACCCGCCTCCTCAGATCCTCA | |
| | TCAAAAACACACCTGTACCTGCGGATCCTCCAACGGCCTTCA | |
| | ACAAGGACAAGCTGAACTCTTTCATCACCCAGTATTCTACTG | |
| | GCCAAGTCAGCGTGGAGATCGAGTGGGAGCTGCAGAAGGA | |
| | AAACAGCAAGCGCTGGAACCCGGAGATCCAGTACACTTCCA | |
| | ACTATTACAAGTCTAATAATGTTGAATTTGCTGTTAATACTG | |
| | AAGGTGTATATAGTGAACCCCGCCCCATTGGCACCAGATAC | |
| | CTGACTCGTAATCTGTAA (SEQ ID NO: 3) | |

These libraries were used to make AAV capsid particles which were injected systemically into mice in duplicate. After two weeks, the heart, lung, liver, small intestine, spleen, pancreas, kidney, brain, and skeletal muscle were extracted. Genomic DNA was isolated from these tissues and the variable peptide region of interest was amplified out and subjected to next generation sequencing (NGS) to quantify AAV abundance in each tissue by measuring the log₂ fold change (log 2FC) value in each organ relative to the injected pool.

A candidate list of “hit peptides” in the AAV9Loop1 and AAV9Loop2 scaffolds which were synthesized and subjected to deep mutagenesis to yield AAV libraries with millions of variants that were then profiled in a secondary screen. As done in the primary screen, AAV capsid particles were produced from these libraries and injected into mice where the tropism was quantified. Additionally, to further evaluate the species-dependent nature of the engineered viral particles, the AAV capsid libraries were pooled from the primary and secondary screens and injected into rhesus macaques as an important preclinical non-human primate (NHP) model. Similar to the experiments done in mice, various organs were isolated from the NHPs 3 weeks after injection, the variable peptide region was amplified out and the AAV abundance quantified in the liver, lung, and 4 lobes of the brain. moreover, to apply additional selected pressure, the variable peptide regions from the heart, lung, pancreas, intestine, brain, and muscle were amplified out and cloned back into the AAV9Loop1 and AAV9Loop2 scaffolds. This tertiary screen was then subjected to two weeks on in vivo selective pressure in mice before the tropism was quantified by NGS. For all screens, a “hit” was defined as an engineered AAV variant that exhibited a log 2FC relative to the capsid >1 in both replicates and a p-value <0.1 in at least one organ.

Shown in Table 2 are the peptides that were present in the starting library of the primary screen that showed prominent in vivo activity across all four scaffolds in which they were grafted. In addition to this list, there are a set of peptides that were consistently identified as hits across both the primary, secondary, tertiary, and NHP screens. These screen-independent hits for both AAV9Loop1 and AAV9Loop2 are shown in Table 3.

TABLE 2
Table for “wild-type” peptides that were present
in the primary screen library and showed in vivo activity across all
4 scaffolds in which they were grafted (i.e. AAV5Loop1, AAV5Loop2,
AAV9Loop1, AAV9Loop2) . Shown is the amino acid sequence.

| Amino Acid Sequence | Amino Acid Sequence | Amino Acid Sequence |
|---|---|---|
| SSQAGIPVRAPRSPRAPEPA | TSRMSTSQIPGRVASSGLQS | KQLNQQHSQIKEIENQLRRT |
| (SEQ ID NO: 5) | (SEQ ID NO: 6) | (SEQ ID NO: 7) |
| VLGAPRAGSGAHTAVISPQD | QGASVNSSSQTAAQVTKGVK | KALAAAGYDVEKNNSRIKLS |
| (SEQ ID NO: 8) | (SEQ ID NO: 9) | (SEQ ID NO: 10) |
| LEALSAGPGTSGSHLDRSTV | TSTMPNSSQSSHSSSGTLSS | GIKSL VSKGTL VQTKGTGAS |
| (SEQ ID NO: 11) | (SEQ ID NO: 12) | (SEQ ID NO: 13) |
| VVYTGHDTKLMQNSTKAPLK | STMPNSSQSSHSSSGTLSSL | RNNMIQTKPTGTQQSTNTAV |
| (SEQ ID NO: 14) | (SEQ ID NO: 15) | (SEQ ID NO: 16) |
| HDTKLMQNSTKAPLKRSNVE | ANGDKEIGNIISDAMKKVGR | AGLTRISKEILTQGQINSTS |
| (SEQ ID NO: 17) | (SEQ ID NO: 18) | (SEQ ID NO: 19) |
| QTAHVGVGISGNEGMQATNN | SKYVNKEIQNAVNGVKQIKT | NHFGTASSTGNLVVKDPTRV |
| (SEQ ID NO: 20) | (SEQ ID NO: 21) | (SEQ ID NO: 22) |
| TAHVGVGISGNEGMQATNNS | EQLGGTVTNPGGSGTSSRLE | YNSAGTGPSSATVNVTTRKP |
| (SEQ ID NO: 23) | (SEQ ID NO: 24) | (SEQ ID NO: 25) |
| ALAAGGYDVEKNNSRIKLGL | VDFRIHVENQTRARDDVSRK | LSL TQSDISHIGSMR VEGIV |
| (SEQ ID NO: 26) | (SEQ ID NO: 27) | (SEQ ID NO: 28) |
| SDGSLEDDDYHATTETIITM | DFRIHVENQTRARDDVSRKQ | SSRGTTGNTQVQSPSNATTA |
| (SEQ ID NO: 29) | (SEQ ID NO: 30) | (SEQ ID NO: 31) |
| HVAMEMKEDINTKKEDMMFH | AGVAHAITAACTQGNLSDCG | RGTTGNTQVQSPSNATTATG |
| (SEQ ID NO: 32) | (SEQ ID NO: 33) | (SEQ ID NO: 34) |
| AIAAGATARATTASRLPSSA | GSVGTQGRACNKTAPQASGC | GTTGNTQVQSPSNATTATGV |
| (SEQ ID NO: 35) | (SEQ ID NO: 36) | (SEQ ID NO: 37) |
| TSPATPSAGGAASSRTPNRI | AEDVRGNLRGNTEGLQKSLA | IKVSNTLESRLDLSAKQKMP |
| (SEQ ID NO: 38) | (SEQ ID NO: 39) | (SEQ ID NO: 40) |
| SPATPSAGGAASSRTPNRIS | QPAVVHLQGQGSAIQVKNDL | VSSLPTTNSTTNSATKITAN |
| (SEQ ID NO: 41) | (SEQ ID NO: 42) | (SEQ ID NO: 43) |
| QDAASSSSSSSSSATTTTPE | NNGSMRNTSQDREREVDNNR | SLPTTNSTTNSATKITANTT |
| (SEQ ID NO: 44) | (SEQ ID NO: 45) | (SEQ ID NO: 46) |
| SLKASSTMAKSENL VKSHVQ | KENGGARLAEYHAKATEHLS | AVSRVADTIMSGPSNSQQVP |
| (SEQ ID NO: 47) | (SEQ ID NO: 48) | (SEQ ID NO: 49) |
| KASSTMAKSENL VKSHVQLQ | KQINDYVEKGTQGKIVDLVK | MIVAAIQAEKNRAGSSRQSI |
| (SEQ ID NO: 50) | (SEQ ID NO: 51) | (SEQ ID NO: 52) |
| SSTMAKSENLVKSHVQLQNY | SNAIKNLPPPLGGAAGHPGS | KSEVSTDLIQANPKQGALAK |
| (SEQ ID NO: 53) | (SEQ ID NO: 54) | (SEQ ID NO: 55) |
| STMAKSENLVKSHVQLQNYS | PEFSNDIKKRKVDDKDSSHY | YGGGATNANARAKGGTGQTG |
| (SEQ ID NO: 56) | (SEQ ID NO: 57) | (SEQ ID NO: 58) |
| TMAKSENLVKSHVQLQNYSK | FSNDIKKRKVDDKDSSHYDS | GGATNANARAKGGTGQTGGL |
| (SEQ ID NO: 59) | (SEQ ID NO: 60) | (SEQ ID NO: 61) |
| SALAGQRQGTQAESNLSSKF | NVINNAILRANLQKGENPSH | ATNANARAKGGTGQTGGLTQ |
| (SEQ ID NO: 62) | (SEQ ID NO: 63) | (SEQ ID NO: 64) |
| ALAGQRQGTQAESNLSSKFQ | VASVSLLQLGQETDKLKTRN | ISSDSIGVLGYQKTVDHTKV |
| (SEQ ID NO: 65) | (SEQ ID NO: 66) | (SEQ ID NO: 67) |
| FQFSSNKEQNGVQDPQHERI | EVHNAKTKPREEQYNSTYRV | PLTPANNLPDTQSSGPAHSK |
| (SEQ ID NO: 68) | (SEQ ID NO: 69) | (SEQ ID NO: 70) |
| SSFQGSKRGLNSSFETSPKK | AARPYSATAAAATGGMANVK | TSVFLKNNKIAGVNNRTEYV |
| (SEQ ID NO: 71) | (SEQ ID NO: 72) | (SEQ ID NO: 73) |
| VEVHNAKTKPREEQFNSTYR | TNSNNHLAADSQAYDQKNIR | LKNNKIAGVNNRTEYVETTS |
| (SEQ ID NO: 74) | (SEQ ID NO: 75) | (SEQ ID NO: 76) |
| EFSVDETGQMSATAKGRVRL | KRSSQSTLERSEQQIRAASS | APANTHNNNIEGQAIVYQNK |
| (SEQ ID NO: 77) | (SEQ ID NO: 78) | (SEQ ID NO: 79) |
| AIVKANANAAAINNLASSIQ | RFVQNALNGNGDPNNMDKAV | KNTALDKNYLLNNKIPSNNV |
| (SEQ ID NO: 80) | (SEQ ID NO: 81) | (SEQ ID NO: 82) |
| NAVARVADTIGSGPSNSQAV | PLTPVNNLPDTRSSGPAHSK | |
| (SEQ ID NO: 83) | (SEQ ID NO: 84) | |

TABLE 3
Table for “wild-type” peptides that were present
as hits in both the primary, secondary, tertiary, and NHP screens.
Shown are the peptide amino acid sequences

| Amino Acid Sequence | Amino Acid Sequence | Amino Acid Sequence |
|---|---|---|
| LGIEQGVGVPGGSPGSRHRQ | YISFNPKENKITVKSKNLPE (SEQ | SATLNSVLNSNAIKNLPPPL |
| (SEQ ID NO: 85) | ID NO: 86) | (SEQ ID NO: 87) |
| GGGGPGGSGGVGGGSGGYTY | MAKSENLVKSHVQLQNYSKV | VTSGKSIDDNSTKSEKQTPR |
| (SEQ ID NO: 88) | (SEQ ID NO: 89) | (SEQ ID NO: 90) |
| SAGPGTSGSHLDRSTVSSFS | MIETYNQTSPRSAATGLPIS (SEQ | VATKGLGVHAKSSDWGLQAG |
| (SEQ ID NO: 91) | ID NO: 92) | (SEQ ID NO: 93) |
| AARDSLSLSSGSGGGSASAS | LPEAVLALYNSTRDRVAGES | LENNKTMNRAENGGRPPHHP |
| (SEQ ID NO: 94) | (SEQ ID NO: 95) | (SEQ ID NO: 96) |
| ILTQGQINSTSGLNKESFRY | HCRQEATNATRVIHDNYDQL | MWVDDQEVINKASNSNKIRL |
| (SEQ ID NO: 97) | (SEQ ID NO: 98) | (SEQ ID NO: 99) |
| RALGTSPELPTGVTGSSGTR | VINNVKDVTKLVANLPKDYM | KVQGLVPAGGSSSNSTREPP |
| (SEQ ID NO: 100) | (SEQ ID NO: 101) | (SEQ ID NO: 102) |
| LKENGGARLAEYHAKATEHL | NSVLNSNAIKNLPPPLGGAA | GIVVYTGHDTKLMQNSTKAP |
| (SEQ ID NO: 103) | (SEQ ID NO: 104) | (SEQ ID NO: 105) |
| GLRGGAGPPGPEGGKGAAGP | EPPPELENNKTMNRAENGGR | ETGWTPGLRSLENDGTKLLD |
| (SEQ ID NO: 106) | (SEQ ID NO: 107) | (SEQ ID NO: 108) |
| RTLVSSKPQAHGAPAPPSGS | ATLPQCQAANKTCPTNYMWN | WETGAVPGLRSLENDGTRLL |
| (SEQ ID NO: 109) | (SEQ ID NO: 110) | (SEQ ID NO: 111) |
| VDNEDDHGRGDLGRLKKQKD | KVVPEGIRMNKTVAVRTLDP | WRSRYPHKPEINSTTHPGAD |
| (SEQ ID NO: 112) | (SEQ ID NO: 113) | (SEQ ID NO: 114) |
| MGDMANSSIEFHPKPQQQRD | GSHGTQDRQCNKTSNGSDSC | NFSAAFSVNYDTKSGPKNMT |
| (SEQ ID NO: 115) | (SEQ ID NO: 116) | (SEQ ID NO: 117) |
| EGTICKRARGDDMDDYCNGK | LLREGNTVRSFRAAAAETLE | TSGLLETNFTASARTTGSGL |
| (SEQ ID NO: 118) | (SEQ ID NO: 119) | (SEQ ID NO: 120) |
| QDNTCEEYHTVGPHSCHIPK | LPGDPTSNATSRGAEGPLPP | QRSGASASGVYTIQVSNATK |
| (SEQ ID NO: 121) | (SEQ ID NO: 122) | (SEQ ID NO: 123) |
| GSSGTGGTATWKPGSSGPGS | TLGADLVDSTDNTKINRGIF | RTSGLLETNFTASARTTGSG |
| (SEQ ID NO: 124) | (SEQ ID NO: 125) | (SEQ ID NO: 126) |
| LQGAGNSAAGRGAIADTFRS | WNRQSSTSVIETNKTSVELS | VPEGIRMNKTVAVRTLDPER |
| (SEQ ID NO: 127) | (SEQ ID NO: 128) | (SEQ ID NO: 129) |
| MKGASRSPEDSSPDAARIRV | GSFGTRDRTCNVISHGIDGC | EHGAVSQEEVIRAYDTTKKK |
| (SEQ ID NO: 130) | (SEQ ID NO: 131) | (SEQ ID NO: 132) |
| GAPRAGSGAHTAVISPQDPT | SIGVLGYQKTVDHTKVNSKL | SNRVRVSIQTGTAFQPDAAM |
| (SEQ ID NO: 133) | (SEQ ID NO: 134) | (SEQ ID NO: 135) |
| TFFRGLESQTKLVNGQSHIS | AGVVCTNETRSTHTLDLSRE | FNDMANAAKGLEWISNKISK |
| (SEQ ID NO: 136) | (SEQ ID NO: 137) | (SEQ ID NO: 138) |
| GSAGPPGATGFPGAAGRVGP | LFGTTISNNIKYGRDDVTDE | VAEIPFNSTNKYQLSIHETE |
| (SEQ ID NO: 139) | (SEQ ID NO: 140) | (SEQ ID NO: 141) |
| MSTSQIPGRVASSGLQSVVH | EILELAGNASRDNKKTRIIP (SEQ | RTILPMDQEFTYRVKNREQA |
| (SEQ ID NO: 142) | ID NO: 143) | (SEQ ID NO: 144) |
| TPGPQGIAGQRGVVGLPGQR | GTAGRVCNLTSRGMDSCEVM | LTATLGADLVDSTDNTKINR |
| (SEQ ID NO: 145) | (SEQ ID NO: 146) | (SEQ ID NO: 147) |
| VEGTARGVVVATGDRTVMGR | YWGSVTGSQSNAVSPTPAPR | VEVGGSAKYLNNTKKYQNQS |
| (SEQ ID NO: [48) | (SEQ ID NO: 149) | (SEQ ID NO: 150) |
| PFPGLGTAAAPAQGGAHLKQ | FNSHINASNNSEPSRMNMTS | QSAKDNNIKFRAIGHSDNSV |
| (SEQ ID NO: 151) | (SEQ ID NO: 152) | (SEQ ID NO: 153) |
| ARARELSSGQKDQGQAANML | ENNKTMNRAENGGRPPHHPF | PELENNKTMNRAENGGRPPH |
| (SEQ ID NO: 154) | (SEQ ID NO: 155) | (SEQ ID NO: 156) |
| KPQKQLTAQSTPSTSGVNSQ | KHSTIFENLANKADRDQYEL | DNRTRQHIPYREDKNLTGTA |
| (SEQ ID NO: 157) | (SEQ ID NO: 158) | (SEQ ID NO: 159) |
| RLEALKENGGARLAEYHAKA | PPPELENNKTMNRAENGGRP | PRGAAPPGQKGQANATRIPA |
| (SEQ ID NO: 160) | (SEQ ID NO: 161) | (SEQ ID NO: 162) |
| GVDNEDDHGRGDLGRLKKQK | GGSSSNSTREPPASGRVALV | MLTCPEANATKVCGSDGVTY |
| (SEQ ID NO: 163) | (SEQ ID NO: 164) | (SEQ ID NO: 165) |
| TAGSAMGAASLTLSAQSRIL | LNSVLNSNAIKNLPPPLGGA | TWL VRHTENVIKTKTPEDYA |
| (SEQ ID NO: 166) | (SEQ ID NO: 167) | (SEQ ID NO: 168) |
| GRPGLPGAAGARGNDGARGS | YRWNRQSSTSVIETNKTSVE | TGHDTKLMQNSTKAPLKRSN |
| (SEQ ID NO: 169) | (SEQ ID NO: 170) | (SEQ ID NO: 171) |
| VRHIPDRGAPTRASEPASAA | CVVADGNSTRSPETNGLLCG | SQTIPQISNRTLNTLVQDNS |
| (SEQ ID NO: 172) | (SEQ ID NO: 173) | (SEQ ID NO: 174) |
| PSSAGSWNSGSSGPGSTGNR | MIQTLINKSKIQNVGGTYEL | GSSSNSTREPPASGRVALVK |
| (SEQ ID NO: 175) | (SEQ ID NO: 176) | (SEQ ID NO: 177) |
| RGSSSRQSSSSAMSSSSASS | CRHERDAGVVCTNETRSTHT | SDSIGVLGYQKTVDHTKVNS |
| (SEQ ID NO: 178) | (SEQ ID NO: 179) | (SEQ ID NO: 180) |
| DLSGITGARNLAVSQVVHKA | LLGTCDQAPKSGSHEVRCNL | L VRHTENVTKTKTPEDYADS |
| (SEQ ID NO: 181) | (SEQ ID NO: 182) | (SEQ ID NO: 183) |
| VYADSFVIRGDEVRQIAPGQ | SHGTQDRQCNKTSNGSDSCD | PELKIPLTANITKSGNTFRP |
| (SEQ ID NO: 184) | (SEQ ID NO: 185) | (SEQ ID NO: 186) |
| VRLVLTNPEGRKEGKEVTFQ | TQGRACNKTAPQASGCDLMC | GTQGRECNRTSEGADGCNLL |
| (SEQ ID NO: 187) | (SEQ ID NO: 188) | (SEQ ID NO: 189) |
| VRLVLTNPEGRKEGKEVTFQ | DKMFSQNDTRCAGPEAVKGQ | GLANGVRRYASQTELYDAPG |
| (SEQ ID NO: 187) | (SEQ ID NO: 190) | (SEQ ID NO: 191) |
| LPKPQSNLPDTKASGPAHSK | SSDSNNLKLNNVRLPRENMS | LERAASRANTVRSFHHEESL |
| (SEQ ID NO: 192) | (SEQ ID NO: 193) | (SEQ ID NO: 194) |
| TVQAARTPGSLEGGSQPWRP | TDANQLWTLKRDNTIRSNGK | LLTVTSGKSIDDNSTKSEKQ |
| (SEQ ID NO: 195) | (SEQ ID NO: 196) | (SEQ ID NO: 197) |
| GAGVAGRITGSMSLASGKSL | EKYIDQEELNKTKPIWTRNP | ENVDFRIHVENQTRARDDVS |
| (SEQ ID NO: 198) | (SEQ ID NO: 199) | (SEQ ID NO: 200) |
| TAGAAMGAASLTLSAQSRTL | ASSTMAKSENL VKSHVQLQN | PEAVLALYNSTRDRVAGESA |
| (SEQ ID NO: 201) | (SEQ ID NO: 202) | (SEQ ID NO: 203) |
| ENGGARLAEYHAKATEHLST | LGTAGRVCNLTSRGMDSCEV | PGDPTSNATSRGAEGPLPPP |
| (SEQ ID NO: 204) | (SEQ ID NO: 205) | (SEQ ID NO: 206) |
| AARLEALKENGGARLAEYHA | GTQDRQCNKTSNGSDSCDLM | EFSNDIKKRKVDDKDSSHYD |
| (SEQ ID NO: 207) | (SEQ ID NO: 208) | (SEQ ID NO: 209) |
| SLAGRAPAGAAAVSASGHGR | IAPVVSSR VSPGTRPTAMGS | LREGNTVRSFRAAAAETLER |
| (SEQ ID NO: 210) | (SEQ ID NO: 211) | (SEQ ID NO: 212) |
| VEGKVQGLVPAGGSSSNSTR | PAGGSSSNSTREPPASGRVA | AKSENLVKSHVQLQNYSKVE |
| (SEQ ID NO: 213) | (SEQ ID NO: 214) | (SEQ ID NO: 215) |
| STSTMPNSSQSSHSSSGTLS | GPEFSNDIKKRKVDDKDSSH | RMDIDVVEKTNKIKDRYWDS |
| (SEQ ID NO: 216) | (SEQ ID NO: 217) | (SEQ ID NO: 218) |
| PKPTSNVHVSVAGSHSQAAN | QQPYITNRTFMLAKEASLAD | RALTQALPAPTGQNTQVSSH |
| (SEQ ID NO: 219) | (SEQ ID NO: 220) | (SEQ ID NO: 221) |
| RDSPHSERYVSALTTPARLS | EAVLALYNSTRDRVAGESAE | PSTDAVLGNSTAVNRGDSSI |
| (SEQ ID NO: 222) | (SEQ ID NO: 223) | (SEQ ID NO: 224) |
| LAGQRQGTQAESNLSSKFQF | KLIDMEAQLLNQTSRMDAQM | TITTNEIRNKLSYSFDGAGG |
| (SEQ ID NO: 225) | (SEQ ID NO: 226) | (SEQ ID NO: 227) |
| RGGSQSVYGVDQTHSGLPLH | GQQYPGQGPPSGQPPYGGHQ | LALAPNSTARRQLASLLEGT |
| (SEQ ID NO: 228) | (SEQ ID NO: 229) | (SEQ ID NO: 230) |
| IVGDAGVRGTAQQPRLFVGM | AEILELAGNASRDNKKTRII | GDADNDMVKIENLIKVYKSR |
| (SEQ ID NO: 231) | (SEQ ID NO: 232) | (SEQ ID NO: 233) |
| KTPLTANITKSGNTFRPEVH | VTWLVRHTENVTKTKTPEDY | PGTQGRECNRTSEGADGCNL |
| (SEQ ID NO: 234) | (SEQ ID NO: 235) | (SEQ ID NO: 236) |
| KTPLTANITKSGNIFRPEVH | RVTNNVKDVTKLVANLPKDY | SMGLQLNLTYERKDNTTVTR |
| (SEQ ID NO: 234) | (SEQ ID NO: 237) | (SEQ ID NO: 238) |
| SFVIRGDEVRQIAPGQTGKI | VGTQGRLCNRTSPGADGQDT | YISNRVRVSIQTGTAFQPDA |
| (SEQ ID NO: 239) | (SEQ ID NO: 240) | (SEQ ID NO: 241) |
| GRNVQEERSAGATANLPLRS | YLTAEILELAGNASRDNKKT | SSSNSTREPPASGRVALVKV |
| (SEQ ID NO: 242) | (SEQ ID NO: 243) | (SEQ ID NO: 244) |
| IGNVGAPGAKGARGSAGPPG | IRKLTDMEAQLLNQTSRMDA | DMIQTLINKSKIQNVGGTYE |
| (SEQ ID NO: 245) | (SEQ ID NO: 246) | (SEQ ID NO: 247) |
| LLQGAGNSAAGRGAIADTER | SLGTAGRVCNLTSRGMDSCE | VLALYNSTRDRVAGESAEPE |
| (SEQ ID NO: 248) | (SEQ ID NO: 249) | (SEQ ID NO: 250) |
| EPYADQLRTQVNTQAEQLRR | HPELKTPLTANTIKSGNTFR | YLGTSLGNITRDQKILNPSA |
| (SEQ ID NO: 251) | (SEQ ID NO: 252) | (SEQ ID NO: 253) |
| LSVGRGGSPRLQPDSRALHY | NSTWDPQSNSIAKYNYPNGK | DVLYAGHNSTWDPQSNSIAK |
| (SEQ ID NO: 254) | (SEQ ID NO: 255) | (SEQ ID NO: 256) |
| AAGSTMGAASMTLTVQARQL | VKIQRVNNSSTNDNLVRKND | LTVTSGKSIDDNSTKSEKQT |
| (SEQ ID NO: 257) | (SEQ ID NO: 258) | (SEQ ID NO: 259) |
| AAMGAASLTLSAQSRTLLAG | QAAELQNDVRQAVEGAIGRV | NNRGKANSTGTL VITDPTRI |
| (SEQ ID NO: 260) | (SEQ ID NO: 261) | (SEQ ID NO: 262) |
| GATNANARAKGGTGQTGGLT | VQGLVPAGGSSSNSTREPPA | RWNRQSSTSVIETNKTSVEL |
| (SEQ ID NO: 263) | (SEQ ID NO: 264) | (SEQ ID NO: 265) |
| VLVEAAASQRSSSLASNRLK | LDLNMTRSHSGGELESLARG | KSALVLSAESSSMGGTLTVQ |
| (SEQ ID NO: 266) | (SEQ ID NO: 267) | (SEQ ID NO: 268) |
| RRAAPEASGTPSSDAVSRLE | WKAAIPSALDTNSSKSTSSF | RITMTNSVDDALINSTKIYS |
| (SEQ ID NO: 269) | (SEQ ID NO: 270) | (SEQ ID NO: 271) |
| VVSSRVSPGTRPTAMGSFSS | EENKIRQEDLTKIISSAQKV (SEQ | IPSALDTNSSKSTSSFPCPA |
| (SEQ ID NO: 272) | ID NO: 273) | (SEQ ID NO: 274) |
| TVSGKITHSPQAHVNPQEKR | LLLAGDRASSNPNATSSSSQ | EISSDSIGVLGYQKTVDHTK |
| (SEQ ID NO: 275) | (SEQ ID NO: 276) | (SEQ ID NO: 277) |
| FKGVDNEDDHGRGDLGRLKK | VLNSNAIKNLPPPLGGAAGH | QDNKIVVKNATKSNVNNAVN |
| (SEQ ID NO: 278) | (SEQ ID NO: 279) | (SEQ ID NO: 280) |
| LTGRQDTSRMSTSQIPGRVA | PFNHIKSSLLGSTSDSNLNK | LPATLPQCQAANKTCPTNYM |
| (SEQ ID NO: 281) | (SEQ ID NO: 282) | (SEQ ID NO: 283) |
| SRMSTSQIPGRVASSGLQSV | SAISNLIRKHVSEARLVEDI (SEQ | NRTSGLLETNFTASARTTGS |
| (SEQ ID NO: 284) | ID NO: 285) | (SEQ ID NO: 286) |
| GRQDTSRMSTSQIPGRVASS | QRVNNSSTNDNLVRKNDQVY | VLFGTTISNNIKYGRDDVTD |
| (SEQ ID NO: 287) | (SEQ ID NO: 288) | (SEQ ID NO: 289) |
| LGGTVTNPGGSGTSSRLEPR | TAAHPELKTPLTANITKSGN | GKSIDDNSTKSEKQTPREHS |
| (SEQ ID NO: 290) | (SEQ ID NO: 291) | (SEQ ID NO: 292) |
| ARLEALKENGGARLAEYHAK | LKASSTMAKSENLVKSHVQL | VVLANASRIHYDGGAGTNTV |
| (SEQ ID NO: 293) | (SEQ ID NO: 294) | (SEQ ID NO: 295) |
| ATAGSAMGAASLTLSAQSRT | ELKTPLTANITKSGNTFRPE | FATDRTSMPSANIIRSFKNE |
| (SEQ ID NO: 296) | (SEQ ID NO: 297) | (SEQ ID NO: 298) |
| PLPSVTKLTEDRWNEHQKMQ | YSLKASSTMAKSENLVKSHV | TSGKSIDDNSTKSEKQTPRE |
| (SEQ ID NO: 299) | (SEQ ID NO: 300) | (SEQ ID NO: 301) |
| STYMAASGLNASTYDQVGRS | IGVLGYQKTVDHTKVNSKLS | KEGGPDHNYTKEKIKIVEGI |
| (SEQ ID NO: 302) | (SEQ ID NO: 303) | (SEQ ID NO: 304) |
| SYSQMQSQAGGNNTGSTPLR | SSMSNHTKERVTMTKVTLEN | KPCAPGTFSNTTSSTDICRP |
| (SEQ ID NO: 305) | (SEQ ID NO: 306) | (SEQ ID NO: 307) |
| AATLEQPASSSHAQGTHLRL | NDWKHNITQREARALELASQ | SMSNHTKERVTMTKVTLENF |
| (SEQ ID NO: 308) | (SEQ ID NO: 309) | (SEQ ID NO: 310) |
| RQAVEGAIGRVADTIRSGPS | LLAPLNDTRVVHAAKAALAA | RSGASASGVYTIQVSNATKP |
| (SEQ ID NO: 311) | (SEQ ID NO: 312) | (SEQ ID NO: 313) |
| AMAGSAMGATSLTLSAQSRT | EILELAGNAARDNKKTRIIP | ISNRVRVSIQTGTAFQPDAA |
| (SEQ ID NO: 314) | (SEQ ID NO: 315) | (SEQ ID NO: 316) |
| LKEGTICKRARGDDMDDYCN | LVEVGGSAKYLNNIKKYQNQ | AAHPELKTPLTANITKSGNT |
| (SEQ ID NO: 317) | (SEQ ID NO: 318) | (SEQ ID NO: 319) |
| DLTGRQDTSRMSTSQIPGRV | QSAVEILLTIDDTKRAGMKE | GLVEVGGSAKYLNNTKKYQN |
| (SEQ ID NO: 320) | (SEQ ID NO: 321) | (SEQ ID NO: 322) |
| GSTYMAASGLNASTYDQVGR | VGYLDRNLEKNSTKQEILAA | TPGLRSLENDGTKLLDRIRD |
| (SEQ ID NO: 323) | (SEQ ID NO: 324) | (SEQ ID NO: 325) |
| VTVSGKITHSPQAHVNPQEK | ATLNSVLNSNAIKNLPPPLG | QAQKDKMFSQNDTRCAGPEA |
| (SEQ ID NO: 326) | (SEQ ID NO: 327) | (SEQ ID NO: 328) |
| PGVGLTPSAAQTARQHPKMH | FDNSTQMNMIRDTLGNPTVK | EYPERPASRANTVRSFHHEE |
| (SEQ ID NO: 329) | (SEQ ID NO: 330) | (SEQ ID NO: 331) |
| SVGTQGRACNKTAPQASGCD | DQEVINKASNSNKIRLEKGR | PLLAPLNDTRVVHAAKAALA |
| (SEQ ID NO: 332) | (SEQ ID NO: 333) | (SEQ ID NO: 334) |
| QNGKSYSLKASSTMAKSENL | LVQIRSNFSKPDLMASLLPQ | LQSAKDNNIKFRAIGHSDNS |
| (SEQ ID NO: 335) | (SEQ ID NO: 336) | (SEQ ID NO: 337) |
| TGGSGGFHLAQQASASGSVS | LNNAILRANLPKSKGNPAAY | TPLTANITKSGNTFRPEVHL |
| (SEQ ID NO: 338) | (SEQ ID NO: 339) | (SEQ ID NO: 340) |
| LTQGDGVAKLSINTHPSQKP | ATLGADLVDSTDNTKINRGI | TMWVDDQEVINKASNSNKIR |
| (SEQ ID NO: 341) | (SEQ ID NO: 342) | (SEQ ID NO: 343) |
| RSLAGRAPAGAAAVSASGHG | KDMIQTLINKSKIQNVGGTY | VVPEGIRMNKTVAVRTLDPE |
| (SEQ ID NO: 344) | (SEQ ID NO: 345) | (SEQ ID NO: 346) |
| ATAGSAMGAASLTVSAQSRT | SGLLETNFTASARTTGSGLL | RVNNSSTNDNLVRKNDQVYI |
| (SEQ ID NO: 347) | (SEQ ID NO: 348) | (SEQ ID NO: 349) |
| NGGARLAEYHAKATEHLSTL | YTGHDTKLMQNSTKAPLKRS | NQDNKIVVKNATKSNVNNAV |
| (SEQ ID NO: 350) | (SEQ ID NO: 351) | (SEQ ID NO: 352) |
| HQASLSKQPTSQPRGDPTGP | FRIHVENQTRARDDVSRKQL | DDVSNQTSCRLAGLKPGTVY |
| (SEQ ID NO: 353) | (SEQ ID NO: 354) | (SEQ ID NO: 355) |
| LVEAAASQRSSSLASNRLKI | SLPATLPQCQAANKTCPTNY | LVKSHVQLQNYSKVERHPVT |
| (SEQ ID NO: 356) | (SEQ ID NO: 357) | (SEQ ID NO: 358) |
| AAPMKEANIRGQGGLAYPGV | ELENNKTMNRAENGGRPPHH | GKSVSGDVELTNIIKNSSFK |
| (SEQ ID NO: 359) | (SEQ ID NO: 360) | (SEQ ID NO: 361) |
| FGPVLDKATRESAFVHAIAS | PPGQKGQANATRIPAKTPPA | VDDQEVINKASNSNKIRLEK |
| (SEQ ID NO: 362) | (SEQ ID NO: 363) | (SEQ ID NO: 364) |
| SATYDDEHHSTLVSTRHYRA | GLSTINQTRLDFHFSSDRTA | CSSMSNHTKERVTMTKVTLE |
| (SEQ ID NO: 365) | (SEQ ID NO: 366) | (SEQ ID NO: 367) |
| GAPGTPGPQGIAGQRGVVGL | RNGPEFSNDIKKRKVDDKDS | MFSQNDTRCAGPEAVKGQTL |
| (SEQ ID NO: 368) | (SEQ ID NO: 369) | (SEQ ID NO: 370) |
| SGPSVLGTGTGGSGGFHLAQ | VRHTENVTKTKTPEDYADSS | SGKSIDDNSTKSEKQTPREH |
| (SEQ ID NO: 371) | (SEQ ID NO: 372) | (SEQ ID NO: 373) |
| GVAGNIVRHIPDRGAPTRAS | VKDRTHNVTEKVTQVLSLGA | LISNLSNTIEKINTLGLDSK |
| (SEQ ID NO: 374) | (SEQ ID NO: 375) | (SEQ ID NO: 376) |
| KPTSNVHVSVAGSHSQAANF | NRQSSTSVIETNKTSVELSL (SEQ | SIPTPSNRVKTQQKSNLELL |
| (SEQ ID NO: 377) | ID NO: 378) | (SEQ ID NO: 379) |
| TSNVHVSVAGSHSQAANFAA | SVLNSNAIKNLPPPLGGAAG | LLTFSDVTSNQSSAVLGKSI |
| (SEQ ID NO: 380) | (SEQ ID NO: 381) | (SEQ ID NO: 382) |
| GPMGPRGAPGERGRPGLPGA | DKRRNGPEFSNDIKKRKVDD | YGERSLLQTNQISNFIDRKN |
| (SEQ ID NO: 383) | (SEQ ID NO: 384) | (SEQ ID NO: 385) |
| KTIVKAGPEYGQGMNPISRL | GTQGRLCNRTSPGADGCDTM | AVLALYNSTRDRVAGESAEP |
| (SEQ ID NO: 386) | (SEQ ID NO: 387) | (SEQ ID NO: 388) |
| GFENEESDYHTLPRARIMQR | RQEATNATRVIHDNYDQLEV | VPAGGSSSNSTREPPASGRV |
| (SEQ ID NO: 389) | (SEQ ID NO: 390) | (SEQ ID NO: 391) |
| RQDTSRMSTSQIPGRVASSG | IQTLINKSKIQNVGGTYELH | DTKLMQNSTKAPLKRSNVEK |
| (SEQ ID NO: 392) | (SEQ ID NO: 393) | (SEQ ID NO: 394) |
| TGRQDTSRMSTSQIPGRVAS | KIQRVNNSSTNDNLVRKNDQ | QRITMTNSVDDALINSTKIY |
| (SEQ ID NO: 395) | (SEQ ID NO: 396) | (SEQ ID NO: 397) |
| DTQRRTSMGGTQQQFVEGVR | WVDDQEVINKASNSNKIRLE | SSDSIGVLGYQKTVDHTKVN |
| (SEQ ID NO: 398) | (SEQ ID NO: 399) | (SEQ ID NO: 400) |
| REALMASGSGGHNTMPSGGN | NAIKNLPPPLGGAAGHPGSA | PLNDTRVVHAAKAALAAFNA |
| (SEQ ID NO: 401) | (SEQ ID NO: 402) | (SEQ ID NO: 403) |
| LSRVNAAVANRDSANGFINK | SHVQLQNYSKVERHPVTALE | QGLVPAGGSSSNSTREPPAS |
| (SEQ ID NO: 404) | (SEQ ID NO: 405) | (SEQ ID NO: 406) |
| GPIGNVGAPGAKGARGSAGP | LLASLQKSPEDEEKNEDQTR | SVGTQGRLCNRTSPGADGCD |
| (SEQ ID NO: 407) | (SEQ ID NO: 408) | (SEQ ID NO: 409) |
| NPRGSSSRQSSSSAMSSSSA | NGPEFSNDIKKRKVDDKDSS | KNNKIAGVNNRTEYVETTST |
| (SEQ ID NO: 410) | (SEQ ID NO: 411) | (SEQ ID NO: 412) |
| QAKFVSNQTELDREVLVGKV | LVGYLDRNLEKNSTKQEILA | WLVRHTENVTKTKTPEDYAD |
| (SEQ ID NO: 413) | (SEQ ID NO: 414) | (SEQ ID NO: 415) |
| PGAKGANGAPGIAGAPGFPG | RGDADNDMVKIENLTKVYKS | TIFDTVNGKLVKKVNLDTTH |
| (SEQ ID NO: 416) | (SEQ ID NO: 417) | (SEQ ID NO: 418) |
| GSSSRQSSSSAMSSSSASSS | SALDTNSSKSTSSFPCPAGH | SFNSHINASNNSEPSRMNMT |
| (SEQ ID NO: 419) | (SEQ ID NO: 420) | (SEQ ID NO: 421) |
| FVIRGDEVRQIAPGQTGKIA | CAPGTFSNTTSSTDICRPHQ | ISNR VGVSIRTGTAFQPDPA |
| (SEQ ID NO: 422) | (SEQ ID NO: 423) | (SEQ ID NO: 424) |
| GARGSAGPPGATGFPGAAGR | QTLINKSKIQNVGGTYELHV | ANEKHMLALAPNSTARRQLA |
| (SEQ ID NO: 425) | (SEQ ID NO: 426) | (SEQ ID NO: 427) |
| GGGATNANARAKGGTGQTGG | VVDDVSNQTSCRLAGLKPGT | ISSDSNNLKLNNVRLPRENM |
| (SEQ ID NO: 428) | (SEQ ID NO: 429) | (SEQ ID NO: 430) |
| TVL VEAAASQRSSSLASNRL | HGTQDRQCNKTSNGSDSCDL | DSIGVLGYQKTVDHTKVNSK |
| (SEQ ID NO: 431) | (SEQ ID NO: 432) | (SEQ ID NO: 433) |
| GRGSAGGNAHSPLGVPGGGL | FGTRDRTCNVTSHGIDGCDL | IQRVNNSSTNDNLVRKNDQV |
| (SEQ ID NO: 434) | (SEQ ID NO: 435) | (SEQ ID NO: 436) |
| RLVTTGVLKQTKGVGASGSF | SESIPTPSNRVKTQQKSNLE | VHAKSSDWGLQAGYIPVNPN |
| (SEQ ID NO: 437) | (SEQ ID NO: 438) | (SEQ ID NO: 439) |
| DINGFTTGRRGDLATIHGMN | GSVGTQGRLCNRTSPGADGC | LRGDADNDMVKIENLTKVYK |
| (SEQ ID NO: 440) | (SEQ ID NO: 441) | (SEQ ID NO: 442) |
| QGLSNPSTLSAQPQLSRSHS | SAGTGPSSATVNVTTRKPPP | GHDTKLMQNSTKAPLKRSNV |
| (SEQ ID NO: 443) | (SEQ ID NO: 444) | (SEQ ID NO: 445) |
| GARGPPGPAGANGAPGLRGG | QTIPQISNRTLNTLVQDNSY | LINDYVKNGTRGKITDLIKD |
| (SEQ ID NO: 446) | (SEQ ID NO: 447) | (SEQ ID NO: 448) |
| QAVPTGSFMMVNSSGRASGQ | HCVVADGNSTRSPETNGLLC | STQNKRANRTEENVSDGSPN |
| (SEQ ID NO: 449) | (SEQ ID NO: 450) | (SEQ ID NO: 451) |
| GPAGKEGGKGPRGETGPAGR | DDQEVINKASNSNKIRLEKG | VLVSRGGSQSVYGVDQTHSG |
| (SEQ ID NO: 452) | (SEQ ID NO: 453) | (SEQ ID NO: 454) |
| QMGPRGLPGERGRPGAPGPA | HINPRSSLVLAATSGNSGTT (SEQ | ERAASRANTVRSFHHEESLE |
| (SEQ ID NO: 455) | ID NO: 456) | (SEQ ID NO: 457) |
| HVPEGQTIKGRVGTVGYMAP | AAIPSALDTNSSKSTSSFPC (SEQ | SLNLSGVPSQDKTRVEPPQY |
| (SEQ ID NO: 458) | ID NO: 459) | (SEQ ID NO: 460) |
| KFKGVDNEDDHGRGDLGRLK | RAASRANTVRSFHHEESLEE | LDRNLEKNSTKQEILAALEK |
| (SEQ ID NO: 461) | (SEQ ID NO: 462) | (SEQ ID NO: 463) |
| YKAVGSLDPSADLSSQRGKV | HSDAVFTDNYTALRKQMAVK | TATLGADLVDSTDNTKINRG |
| (SEQ ID NO: 464) | (SEQ ID NO: 465) | (SEQ ID NO: 466) |
| RSADSHGHPRENRTIGATMV | CQDCPLLAPLNDTRVVHAAK | KAAIPSALDINSSKSTSSFP |
| (SEQ ID NO: 467) | (SEQ ID NO: 468) | (SEQ ID NO: 469) |
| TLRPGEVTALVGPNGSGKST | PPELENNKTMNRAENGGRPP | NVDFRIHVENQTRARDDVSR |
| (SEQ ID NO: 470) | (SEQ ID NO: 471) | (SEQ ID NO: 472) |
| RAHQNSQTHQASLSKQPTSQ | VQLQNYSKVERHPVTALEKM | VKSHVQLQNYSKVERHPVTA |
| (SEQ ID NO: 473) | (SEQ ID NO: 474) | (SEQ ID NO: 475) |
| AMGAGVAGRITGSMSLASGK | TLNSVLNSNAIKNLPPPLGG | GLVPAGGSSSNSTREPPASG |
| (SEQ ID NO: 476) | (SEQ ID NO: 477) | (SEQ ID NO: 478) |
| RPRGGGAGGSSVGTGGGGTG | GITAFNHPLNLTKQQLSEVA | DFDVIRNVIKDNATFSRKNP |
| (SEQ ID NO: 479) | (SEQ ID NO: 480) | (SEQ ID NO: 481) |
| TPKGQLPGGKAPPKAGSVPS | KSHVQLQNYSKVERHPVTAL | YPERPASRANTVRSFHHEEH |
| (SEQ ID NO: 482) | (SEQ ID NO: 483) | (SEQ ID NO: 484) |
| AGPAQTLIRPQDMKGASRSP | SGASASGVYTIQVSNATKPR | IVVYTGHDTKLMQNSTKAPL |
| (SEQ ID NO: 485) | (SEQ ID NO: 486) | (SEQ ID NO: 487) |
| AVHVPEGQTIKGRVGTVGYM | DVWGKGTTVTVSSASPTSPK | RKNGPIDISNTDNFVRKNDL |
| (SEQ ID NO: 488) | (SEQ ID NO: 489) | (SEQ ID NO: 490) |
| SKKQLSLKLSSVNAADTAVY | NRVTNNVKDVTKLVANLPKD | RRNGPEFSNDIKKRKVDDKD |
| (SEQ ID NO: 491) | (SEQ ID NO: 492) | (SEQ ID NO: 493) |
| GFPGPKGNDGAPGKNGERGG | LRGDFSSANNRDNTYNRVSE | LPGMTNQDNKIVVKNATKSN |
| (SEQ ID NO: 494) | (SEQ ID NO: 495) | (SEQ ID NO: 496) |
| KPGSSGPGSTGSWNSGSSGT | LNSNAIKNLPPPLGGAAGHP | NSQTIPQISNRTLNTLVQDN |
| (SEQ ID NO: 497) | (SEQ ID NO: 498) | (SEQ ID NO: 499) |
| GSGPSVLGTGTGGSGGFHLA | LLQSAKDNNIKFRAIGHSDN | LNLSGVPSQDKTRVEPPQYM |
| (SEQ ID NO: 500) | (SEQ ID NO: 501) | (SEQ ID NO: 502) |
| ALKENGGARLAEYHAKATEH | ERPASRANTVRSFHHEEHLE | PGMTNQDNKIVVKNATKSNV |
| (SEQ ID NO: 503) | (SEQ ID NO: 504) | (SEQ ID NO: 505) |
| GVQLGKRYNGSDPASGPSVQ | GSLGTAGRVCNLTSRGMDSC | GYLDRNLEKNSTKQEILAAL |
| (SEQ ID NO: 506) | (SEQ ID NO: 507) | (SEQ ID NO: 508) |
| GIEQGVGVPGGSPGSRHRQK | LVPAGGSSSNSTREPPASGR | ESIPTPSNRVKTQQKSNLEL |
| (SEQ ID NO: 509) | (SEQ ID NO: 510) | (SEQ ID NO: 511) |
| LKTPLTANITKSGNTFRPEV | RFDVSAMEKNASNLVKAEFR | VYLGTSLGNITRDQKILNPS |
| (SEQ ID NO: 512) | (SEQ ID NO: 513) | (SEQ ID NO: 514) |
| LKTPLTANITKSGNTFRPEV | KYIDQEELNKTKPIWTRNPD | EILELAGNAARDNKKTRIIP |
| (SEQ ID NO: 512) | (SEQ ID NO: 515) | (SEQ ID NO: 315) |
| QRIAVETAATARKSSPPASS | YISNRVGVSIRTGTAFQPDP | HVQLQNYSKVERHPVTALEK |
| (SEQ ID NO: 516) | (SEQ ID NO: 517) | (SEQ ID NO: 518) |
| AAGVAHAITAACTQGNLSDC | SFGTRDRTCNVTSHGIDGCD | TAFNHPLNLTKQQLSEVALM |
| (SEQ ID NO: 519) | (SEQ ID NO: 520) | (SEQ ID NO: 521) |
| LEYPERPASRANTVRSFHHE | QSSTSVIETNKTSVELSLPF (SEQ | MLALAPNSTARRQLASLLEG |
| (SEQ ID NO: 522) | ID NO: 523) | (SEQ ID NO: 524) |
| ETGLSTINQTRLDFHFSSDR | AGGSSSNSTREPPASGRVAL | KDNNIKFRAIGHSDNSVPPF |
| (SEQ ID NO: 525) | (SEQ ID NO: 526) | (SEQ ID NO: 527) |
| GTFSNTTSSTDICRPHQICN | RDLFFNEIKARSGQDDKTVY | |
| (SEQ ID NO: 528) | (SEQ ID NO: 529) | |

Shown in Table 4A-B are the top performing peptides across all of these described screens. These include the mutant versions of the peptides that were originally identified in the primary screen. The information includes the amino acid sequence of the peptide, the AAV scaffold in which that peptide was inserted, the organ where that peptide was a hit, the log 2FC in that organ, and the screen in which that peptide was identified. While the peptides shown here were highlighted for their ability to transduce a particular organ, many of these also display other interesting properties such as their increased ability to form functional capsids (high titers), their significant de-targeting away from the liver, and their ability to target unique combinations of organs simultaneously. It is expected that these peptides and mutants thereof to have the ability to modulate tropism of AAV capsids, such as AAV5, AAV9, and the 23 immune orthogonal capsids (shown in Table 5) and beyond as well as in tropism of other delivery vehicles and peptide-payload conjugates.

TABLE 4A
Table for mutant peptides that were identified
as the top hits in the primary, secondary, tertiary, and NHP
screens. Shown are the peptide amino acid sequences, the scaffold
where they were grafted, the log2FC in the organ they were a hit,
the organ they were a hit in, and the screen in which that log2FC
was observed. (P = pancreas; H = heart; B = Brain; L = Lung; Lv = liver;
K = kidney; M = muscle; S = spleen; I = intestine) :

| Amino | | | | Screen | Amino | | | | Screen |
|---|---|---|---|---|---|---|---|---|---|
| Acid | | | | Identified | Acid | | | | Identified |
| Sequence | Scaffold | Log2FC | Organ | In | Sequence | Scaffold | Log2FC | Organ | In |
| YSIGVLGYQK | AAV9 | 12.88569 | H | 3° | GFENEESDYHTL | AAV9 | 16.11414 | P | 3° |
| TVDHTKVNSK | Loop2 | | | | PRAGIMQK (SEQ | Loop1 | | | |
| (SEQ ID NO: | | | | | ID NO: 531) | | | | |
| 530) | | | | | | | | | |
| YSIGVLGYQK | AAV9 | 12.68698 | H | 3° | YSIGVLGYQKTE | AAV9 | 14.12717 | P | 3° |
| TEDHTKVNSK | Loop2 | | | | DHTKVNSK | Loop2 | | | |
| (SEQ ID NO: | | | | | (SEQ ID NO: 532) | | | | |
| 532) | | | | | | | | | |
| GFENEESDYH | AAV9 | 11.76037 | H | 3° | YSIGVLGYQKT | AAV9 | 13.93828 | P | 3° |
| TLPRAGIMQK | Loop1 | | | | VDHTKVNSK | Loop2 | | | |
| (SEQ ID NO: | | | | | (SEQ ID NO: 530) | | | | |
| 531) | | | | | | | | | |
| YSIGVLGNQK | AAV9 | 11.64864 | H | 3° | YSIGVLGNQKT | AAV9 | 13.13025 | P | 3° |
| TVDHTKVNSK | Loop2 | | | | VDHTKVNSK | Loop2 | | | |
| (SEQ ID NO: | | | | | (SEQ ID NO: 533) | | | | |
| 533) | | | | | | | | | |
| DAPLGTGANK | AAV9 | 11.28843 | H | 3° | DAPLGTGANKD | AAV9 | 12.72721 | P | 3° |
| DLELPWLEQQ | Loop2 | | | | LELPWLEQQ | Loop2 | | | |
| (SEQ ID NO: | | | | | (SEQ ID NO: 534) | | | | |
| 534) | | | | | | | | | |
| YSIGVLSYQKT | AAV9 | 10.70343 | H | 3° | YSIGVLGYQKT | AAV9 | 11.96429 | P | 3° |
| VDHTKVNSK | Loop2 | | | | VDHTKVNGK | Loop2 | | | |
| (SEQ ID NO: | | | | | (SEQ ID NO: 536) | | | | |
| 535) | | | | | | | | | |
| YSIGVL VYQK | AAV9 | 10.63466 | H | 3° | YSIGVLSYQKTV | AAV9 | 11.86451 | P | 3° |
| TVDHTKVNSK | Loop2 | | | | DHTKVNSK | Loop2 | | | |
| (SEQ ID NO: | | | | | (SEQ ID NO: 535) | | | | |
| 537) | | | | | | | | | |
| YSIGVLGYQK | AAV9 | 10.60146 | H | 3° | YSIGVL VYQKT | AAV9 | 11.61047 | P | 3° |
| TVDHTKVNGK | Loop2 | | | | VDHTKVNSK | Loop2 | | | |
| (SEQ ID NO: | | | | | (SEQ ID NO: 537) | | | | |
| 536) | | | | | | | | | |
| FTGEPSLLPVS | AAV9 | 10.28004 | H | 2° | YSIGVQGYQKT | AAV9 | 11.05567 | P | 3° |
| PVGHLHASL | Loop2 | | | | VDHTKVNSK | Loop2 | | | |
| (SEQ ID NO: | | | | | (SEQ ID NO: 539) | | | | |
| 538) | | | | | | | | | |
| YSIGVQGYQK | AAV9 | 9.748812 | H | 3° | LGKGTGGSGGF | AAV9 | 11.0131 | P | 3° |
| TVDHTKVNSK | Loop2 | | | | HRAQQASGS | Loop1 | | | |
| (SEQ ID NO: | | | | | (SEQ ID NO: 540) | | | | |
| 539) | | | | | | | | | |
| KKDRVTEALN | AAV9 | 9.689435 | H | 2° | HDTNLMQNSTK | AAV9 | 10.80511 | P | 3° |
| ATRAAVEEGF | Loop2 | | | | AHLKRSNVE | Loop2 | | | |
| (SEQ ID NO: | | | | | (SEQ ID NO: 542) | | | | |
| 541) | | | | | | | | | |
| HDTNLMQNST | AAV9 | 9.358717 | H | 3° | QDNTCEEYHTM | AAV9 | 10.63549P | P | 3° |
| KAHLKRSNVE | Loop2 | | | | GPHSSHIPK | Loop1 | | | |
| (SEQ ID NO: | | | | | (SEQ ID NO: 543) | | | | |
| 542) | | | | | | | | | |
| LNSNAIKNLPT | AAV9 | 9.262807 | H | 2° | FSIGVLGYQKTV | AAV9 | 10.49796 | P | 3° |
| PLGGAAGHQ | Loop2 | | | | DHTKVNSK | Loop2 | | | |
| (SEQ ID NO: | | | | | (SEQ ID NO: 545) | | | | |
| 544 | | | | | | | | | |
| DKRRNGPEFS | AAV9 | 9.215325 | H | 2° | YSIGVLGYQKS | AAV9 | 10.26756 | P | 3° |
| NYIKKRKVDD | Loop2 | | | | VDHTKVNSK | Loop2 | | | |
| (SEQ ID NO: | | | | | (SEQ ID NO: 547) | | | | |
| 546) | | | | | | | | | |
| FSIGVLGYQKT | AAV9 | 9.095547 | H | 3° | YSIGVLGYQKT | AAV9 | 10.06449 | P | 3° |
| VDHTKVNSK | Loop2 | | | | VDHTKVNST | Loop2 | | | |
| (SEQ ID NO: | | | | | (SEQ ID NO: 548) | | | | |
| 545) | | | | | | | | | |
| WGQGSTVAAS | AAV9 | 9.070575 | H | 2° | LSATQSVVSHIG | AAV9 | 9.54629 | P | 2° |
| SGSASAPTLF | Loop1 | | | | SKRVEGIV (SEQ | Loop1 | | | |
| (SEQ ID NO: | | | | | ID NO: 550) | | | | |
| 549) | | | | | | | | | |
| FGPSAALDSQ | AAV9 | 8.9294 | H | 2° | SDSICVLGYQKT | AAV9 | 9.334344 | P | 2° |
| SGLLKGQEPW | Loop2 | | | | VDHTKVNS | Loop2 | | | |
| (SEQ ID NO: | | | | | (SEQ ID NO: 552) | | | | |
| 551) | | | | | | | | | |
| YSIGVLGYQK | AAV9 | 8.773237 | H | 3° | AGGSSSNSTREP | AAV9 | 9.009486 | P | 2° |
| SVDHTKVNSK | Loop2 | | | | PASGLVAF (SEQ | Loop2 | | | |
| (SEQ ID NO: | | | | | ID NO: 553) | | | | |
| 547) | | | | | | | | | |
| YKRRNGPEFS | AAV9 | 8.732732 | H | 2° | LNSNAIKNLPTP | AAV9 | 8.997317 | P | 2° |
| NDIKKRKVDD | Loop2 | | | | LGGAAGHQ | Loop2 | | | |
| (SEQ ID NO: | | | | | (SEQ ID NO: 544) | | | | |
| 554) | | | | | | | | | |
| YSIGVLGYQK | AAV9 | 8.694392 | H | 3° | YSIGVLGNQKT | AAV9 | 8.819996 | P | 3° |
| TVDHTKVNST | Loop2 | | | | VDHTKENSK | Loop2 | | | |
| (SEQ ID NO: | | | | | (SEQ ID NO: 555) | | | | |
| 548) | | | | | | | | | |
| DINGLTTGRR | AAV9 | 8.657613 | H | 2° | NVDFSINVENQT | AAV9 | 8.789112 | P | 2° |
| GDMAAIHGM | Loop1 | | | | RARDDVSR | Loop2 | | | |
| N (SEQ ID NO: | | | | | (SEQ ID NO: 557) | | | | |
| 556) | | | | | | | | | |
| AGGSSSNSTR | AAV9 | 8.589748 | H | 2° | DSIGVLGYKKT | AAV9 | 8.735838 | P | 2° |
| EPPASGLVAF | Loop2 | | | | VDNTKVNSK | Loop2 | | | |
| (SEQ ID NO: | | | | | (SEQ ID NO: 558) | | | | |
| 553) | | | | | | | | | |
| VLNSNAIKNL | AAV9 | 8.588456 | H | 2° | ETGLSAINQTRL | AAV9 | 8.688695 | P | 2° |
| HQPLGGAAGH | Loop2 | | | | DENNSSDR (SEQ | Loop2 | | | |
| (SEQ ID NO: | | | | | ID NO: 560) | | | | |
| 559) | | | | | | | | | |
| YQEVINKASN | AAV9 | 8.463129 | H | 2° | ISSDSICALGYQ | AAV9 | 8.609108 | P | 2° |
| SNKIRLEKGR | Loop2 | | | | KTVDHTKV | Loop2 | | | |
| (SEQ ID NO: | | | | | (SEQ ID NO: 562) | | | | |
| 561) | | | | | | | | | |
| VQAGGSSSNS | AAV9 | 8.435494 | H | 2° | YSIGVLGYQKT | AAV9 | 8.596609 | P | 2° |
| TREQPASGRV | Loop2 | | | | VDHTKVNSK | Loop2 | | | |
| (SEQ ID NO: | | | | | (SEQ ID NO: 530) | | | | |
| 563) | | | | | | | | | |
| NVDFSINVEN | AAV9 | 8.420162 | H | 2° | SSDSIGVHGYQK | AAV9 | 8.58793 | P | 2° |
| QTRARDDVSR | Loop2 | | | | TVDHTKVN | Loop2 | | | |
| (SEQ ID NO: | | | | | (SEQ ID NO: 564) | | | | |
| 557) | | | | | | | | | |
| QVHPDSGISSE | AAV9 | 8.418562 | H | 2° | SSTSTMPNSSRS | AAV9 | 8.409485 | P | 2° |
| AMGIVNSFV | Loop1 | | | | SHSSSWTL (SEQ | Loop2 | | | |
| (SEQ ID NO: | | | | | ID NO: 566) | | | | |
| 565) | | | | | | | | | |
| VTAGGSSSNS | AAV9 | 8.371044 | H | 2° | TAAHPELKTQL | AAV9 | 8.392862 | P | 2° |
| TREQPASGRV | Loop2 | | | | TANITKSGN | Loop2 | | | |
| (SEQ ID NO: | | | | | (SEQ ID NO: 568) | | | | |
| 567) | | | | | | | | | |
| LVPACCSSSNS | AAV9 | 8.321773 | H | 2° | LVPACCSSSNST | AAV9 | 8.382255 | P | 2° |
| TREPPASGR | Loop2 | | | | REPPASGR (SEQ | Loop2 | | | |
| (SEQ ID NO: | | | | | ID NO: 569) | | | | |
| 569) | | | | | | | | | |
| VPAGGSSSNS | AAV9 | 8.313722 | H. | 2° | SSTSTMPNSSRS | AAV9 | 8.336041 | P | 2° |
| TREQHASGRV | Loop2 | | | | SNSSSGTL (SEQ | Loop2 | | | |
| (SEQ ID NO: | | | | | ID NO: 571) | | | | |
| 570) | | | | | | | | | |
| LQALVREMSG | AAV9 | 8.269379 | H | 2° | IARVADTISSGP | AAV9 | 8.320148P | | 2° |
| SPASGIPAEV | Loop2 | | | | VNSESFPT (SEQ | Loop1 | | | |
| (SEQ ID NO: | | | | | ID NO: 573) | | | | |
| 572) | | | | | | | | | |
| APPTELHRTR | AAV9 | 8.226368 | H | 2° | MIQTLINKSKIQ | AAV9 | 8.315111 | P | 2° |
| KEGVATYAAA | Loop1 | | | | NVGWTYEF | Loop2 | | | |
| (SEQ ID NO: | | | | | (SEQ ID NO: 575) | | | | |
| 574) | | | | | | | | | |
| VLNSNAIKNLP | AAV9 | 8.132094 | H | 2° | VQAGGSSSNST | AAV9 | 8.297221 | P | 2° |
| QQLGGAAGH | Loop2 | | | | REQPASGRV | Loop2 | | | |
| (SEQ ID NO: | | | | | (SEQ ID NO: 563) | | | | |
| 576) | | | | | | | | | |
| DKEVINKASN | AAV9 | 8.128914 | H | 2° | TATMGADLVDS | AAV9 | 8.289843 | P | 2° |
| SNKIRLEKGR | Loop2 | | | | TDNTKINRG | Loop2 | | | |
| (SEQ ID NO: | | | | | (SEQ ID NO: 578) | | | | |
| 577) | | | | | | | | | |
| RTSMGGTQQQ | AAV9 | 8.128729 | H | 2° | TATLGADLVDS | AAV9 | 8.269284 | P | 2° |
| FVKGARMEEI | Loop2 | | | | TDNTKINRC | Loop2 | | | |
| (SEQ ID NO: | | | | | (SEQ ID NO: 580) | | | | |
| 579) | | | | | | | | | |
| LGGTVANPGG | AAV9 | 8.06852 | H | 2° | SSDSICVLGYQK | AAV9 | 8.247059 | P | 2° |
| SGTSSRLEPK | Loop1 | | | | TVDHTKVN | Loop2 | | | |
| (SEQ ID NO: | | | | | (SEQ ID NO: 582) | | | | |
| 581) | | | | | | | | | |
| ACGSSSNSTRE | AAV9 | 8.035724 | H | 2° | TLGADLVDSTD | AAV9 | 8.244473 | P | 2° |
| PPASGLVAL | Loop2 | | | | NTKINLGIF (SEQ | Loop2 | | | |
| (SEQ ID NO: | | | | | ID NO: 584) | | | | |
| 583) | | | | | | | | | |
| MIQTLTNKSKI | AAV9 | 7.993694 | H | 2° | ISSDSIGVLGYK | AAV9 | 8.243375 | P | 2° |
| QNVGWTYEF | Loop2 | | | | KTVDHTKV | Loop2 | | | |
| (SEQ ID NO: | | | | | (SEQ ID NO: 585) | | | | |
| 575) | | | | | | | | | |
| SVLNSNAIKNL | AAV9 | 7.993393 | H | 2° | VTAGGSSSNSTR | AAV9 | 8.237233 | P | 2° |
| PQQLGGAAG | Loop2 | | | | EQPASGRV (SEQ | Loop2 | | | |
| (SEQ ID NO: | | | | | ID NO: 567) | | | | |
| 586) | | | | | | | | | |
| LVPAGGSSSN | AAV9 | 7.979569 | H | 2° | ENVDFRINVEN | AAV9 | 8.225842 | P | 2° |
| STREQHASGR | Loop2 | | | | QTRARDDVS | Loop2 | | | |
| (SEQ ID NO: | | | | | (SEQ ID NO: 588) | | | | |
| 587) | | | | | | | | | |
| TAAHPELKTQ | AAV9 | 7.958291 | H | 2° | AKSAVEILLTID | AAV9 | 8.199508 | P | 2° |
| LTANITKSGN | Loop2 | | | | DTKRAGMK | Loop2 | | | |
| (SEQ ID NO: | | | | | (SEQ ID NO: 589) | | | | |
| 568) | | | | | | | | | |
| DFRIHVENQTL | AAV9 | 7.887135 | H | 2° | SDSIGVLCYQKT | AAV9 | 8.1967 | P | 2° |
| ALDDVSRKQ | Loop2 | | | | VDHTKVNS | Loop2 | | | |
| (SEQ ID NO: | | | | | (SEQ ID NO: 591) | | | | |
| 590) | | | | | | | | | |
| PACGSSSNSTR | AAV9 | 7.859241 | H | 2° | ISSDSIGALGYK | AAV9 | 8.187129 | P | 2° |
| EPPASGLVA | Loop2 | | | | KTVDHTKV | Loop2 | | | |
| (SEQ ID NO: | | | | | (SEQ ID NO: 593) | | | | |
| 592) | | | | | | | | | |
| RRVGDSSGAA | AAV9 | 7.852159 | H | 2° | DSIGVLGYQKT | AAV9 | 8.183675 | P | 2° |
| AAHKVLPRGL | Loop1 | | | | VDHTNVNSN | Loop2 | | | |
| (SEQ ID NO: | | | | | (SEQ ID NO: 595) | | | | |
| 594) | | | | | | | | | |
| PAGCSSSNSTR | AAV9 | 7.847743 | H | 2° | LASIQKSPEDEE | AAV9 | 8.182704 | P | 2° |
| EPPASGLVA | Loop2 | | | | KNYDQTRF | Loop2 | | | |
| (SEQ ID NO: | | | | | (SEQ ID NO: 597) | | | | |
| 596) | | | | | | | | | |
| VPAGGTSSNS | AAV9 | 7.841973 | H | 2° | LVPAGGSSSNST | AAV9 | 8.174143 | P | 2° |
| TREPPASGLV | Loop2 | | | | REQHASGR | Loop2 | | | |
| (SEQ ID NO: | | | | | (SEQ ID NO: 587) | | | | |
| 598) | | | | | | | | | |
| LGGTVTHPGG | AAV9 | 7.817083 | H | 2° | GSATIYWHDIRT | AAV9 | 8.165904 | P | 2° |
| SGTSSRLEPK | Loop1 | | | | EMDETRKN | Loop2 | | | |
| (SEQ ID NO: | | | | | (SEQ ID NO: 600) | | | | |
| 599) | | | | | | | | | |
| RRNGPEFSNYI | AAV9 | 7.767658 | H | 2° | SDSIGVLGYQKT | AAV9 | 8.159857 | P | 2° |
| KKRKVDDKD | Loop2 | | | | VDHTNVNS | Loop2 | | | |
| (SEQ ID NO: | | | | | (SEQ ID NO: 602) | | | | |
| 601) | | | | | | | | | |
| KAAIPSALDTN | AAV9 | 7.767145 | H | 2° | DFRIHVENQTLA | AAV9 | 8.154733 | P | 2° |
| SSKSGSSFP | Loop2 | | | | LDDVSRKQ | Loop2 | | | |
| (SEQ ID NO: | | | | | (SEQ ID NO: 590) | | | | |
| 603) | | | | | | | | | |
| SVLNTNAIKN | AAV9 | 7.718368 | H | 2° | GFENEESDYHTL | AAV9 | 16.04491 | K | 3° |
| LPTPLGGAAG | Loop2 | | | | PRAGIMQK (SEQ | Loop1 | | | |
| (SEQ ID NO: | | | | | ID NO: 531) | | | | |
| 604) | | | | | | | | | |
| SRNGPEFSNDI | AAV9 | 7.717071 | H | 2° | YSIGVLGYQKTE | AAV9 | 12.57863 | K | 3° |
| KKRKVDDKD | Loop2 | | | | DHTKVNSK | Loop2 | | | |
| (SEQ ID NO: | | | | | (SEQ ID NO: 532) | | | | |
| 605) | | | | | | | | | |
| LPEAVLALHN | AAV9 | 7.713016 | H | 2° | YSIGVLGYQKT | AAV9 | 12.39794 | K | 3° |
| STRDLVAGES | Loop2 | | | | VDHTKVNSK | Loop2 | | | |
| (SEQ ID NO: | | | | | (SEQ ID NO: 530) | | | | |
| 606) | | | | | | | | | |
| GSEGTQGRAC | AAV9 | 7.712568 | H | 2° | YSIGVLGNQKT | AAV9 | 11.62058 | K | 3° |
| NKTAPQASCC | Loop2 | | | | VDHTKVNSK | Loop2 | | | |
| (SEQ ID NO: | | | | | (SEQ ID NO: 533) | | | | |
| 607) | | | | | | | | | |
| VLALNNSTSD | AAV9 | 7.688014 | H | 2° | CKFLKEGTICKR | AAV9 | 11.23581 | K | 2° |
| RVAGESAEPE | Loop2 | | | | ARCDDMDD | Loop2 | | | |
| (SEQ ID NO: | | | | | (SEQ ID NO: 609) | | | | |
| 608) | | | | | | | | | |
| VQAGGSSSNS | AAV9 | 7.679988 | H | 2° | DAPLGTGANKD | AAV9 | 11.05473 | K | 3° |
| TRELPASGRV | Loop2 | | | | LELPWLEQQ | Loop2 | | | |
| (SEQ ID NO: | | | | | (SEQ ID NO: 534) | | | | |
| 610) | | | | | | | | | |
| LNTVLNSNAI | AAV9 | 7.659771 | H | 2° | YSIGVLSYQKTV | AAV9 | 10.72641 | K | 3° |
| KNLPQPLGGA | Loop2 | | | | DHTK VNSK | Loop2 | | | |
| (SEQ ID NO: | | | | | (SEQ ID NO: 535) | | | | |
| 611) | | | | | | | | | |
| VKGAGGESRV | AAV9 | 7.633291 | H | 2° | YSIGVLGYQKT | AAV9 | 10.58271 | K | 3° |
| LPSGQGINNL | Loop2 | | | | VDHTKVNGK | Loop2 | | | |
| (SEQ ID NO: | | | | | (SEQ ID NO: 536) | | | | |
| 612) | | | | | | | | | |
| QGLVQAGGSS | AAV9 | 7.627424 | H | 2° | NRAVFKGGKDN | AAV9 | 10.37732 | K | 2° |
| SNSTRELPAS | Loop2 | | | | IPVLKRDVA | Loop2 | | | |
| (SEQ ID NO: | | | | | (SEQ ID NO: 614) | | | | |
| 613) | | | | | | | | | |
| VLNSNAIKNLP | AAV9 | 7.622054 | H | 2° | NRAVFKGCQDN | AAV9 | 10.22739 | K | 2° |
| PPLCCAAGH | Loop2 | | | | IPVLKRDVA | Loop2 | | | |
| (SEQ ID NO: | | | | | (SEQ ID NO: 616) | | | | |
| 615) | | | | | | | | | |
| GDKANSSIEFH | AAV9 | 7.610955 | H | 2° | YSIGVL VYQKT | AAV9 | 10.19363 | K | 3° |
| SKPQHQRDV | Loop 1 | | | | VDHTKVNSK | Loop2 | | | |
| (SEQ ID NO: | | | | | (SEQ ID NO: 537) | | | | |
| 617) | | | | | | | | | |
| LKTQLTANITK | AAV9 | 7.592926 | H | 2° | VMLVLTNPEGR | AAV9 | 10.19295 | K | 2° |
| SGNTFRHEV | Loop2 | | | | KEGKEVTFQ | Loop2 | | | |
| (SEQ ID NO: | | | | | (SEQ ID NO: 619) | | | | |
| 618) | | | | | | | | | |
| VPACGSSSNST | AAV9 | 7.588375 | H | 2° | ARLVLTNHEGR | AAV9 | 10.14889 | K | 2° |
| REPPASGLV | Loop2 | | | | KEGKEVTFQ | Loop2 | | | |
| (SEQ ID NO: | | | | | (SEQ ID NO: 621) | | | | |
| 620) | | | | | | | | | |
| RRNGPEFSNDI | AAV9 | 7.586276 | H | 2° | NCKFLKEGTICK | AAV9 | 10.14535 | K | 2° |
| KKRKVDYKD | Loop2 | | | | RARCDDMD | Loop2 | | | |
| (SEQ ID NO: | | | | | (SEQ ID NO: 623) | | | | |
| 622) | | | | | | | | | |
| VPAGGSSSNS | AAV9 | 7.576296 | H | 2° | QDNTCEEYHTM | AAV9 | 9.670684 | K | 3° |
| TLEPPASGLV | Loop2 | | | | GPHSSHIPK | Loop1 | | | |
| (SEQ ID NO: | | | | | (SEQ ID NO: 543) | | | | |
| 624) | | | | | | | | | |
| HVQLQNYSKV | AAV9 | 7.555717 | H | 2° | DSIGVLGYQKT | AAV9 | 9.528444 | K | 3° |
| ERHHVTALEN | Loop2 | | | | GDHTKVNSK | Loop2 | | | |
| (SEQ ID NO: | | | | | (SEQ ID NO: 626) | | | | |
| 625) | | | | | | | | | |
| GGLAYPGVRT | AAV9 | 7.552054 | H | 2° | YSIGVQGYQKT | AAV9 | 9.522257 | K | 3° |
| HGTLESVNWP | Loop2 | | | | VDHTKVNSK | Loop2 | | | |
| (SEQ ID NO: | | | | | (SEQ ID NO: 539) | | | | |
| 627) | | | | | | | | | |
| SVLNSNAIKNL | AAV9 | 7.549378 | H | 2° | VRLVLTNHEGR | AAV9 | 9.485177 | K | 2° |
| PPPLGCAAW | Loop2 | | | | KEGKEVSFQ | Loop2 | | | |
| (SEQ ID NO: | | | | | (SEQ ID NO: 629) | | | | |
| 628) | | | | | | | | | |
| LKTQLTANTT | AAV9 | 7.546106 | H | 2° | VRLVLTNPEGR | AAV9 | 9.479958 | K | 2° |
| KSGNTFRPEV | Loop2 | | | | KECKEVTFQ | Loop2 | | | |
| (SEQ ID NO: | | | | | (SEQ ID NO: 631) | | | | |
| 630) | | | | | | | | | |
| DINGLTTGRR | AAV9 | 18.96709 | L | NHP | NCKFLKEGTICK | AAV9 | 9.428763 | K | 2° |
| GDMAAIHGM | Loop1 | | | | RARGYDMD | Loop2 | | | |
| N (SEQ ID | | | | | (SEQ ID NO: 632) | | | | |
| NO: 556) | | | | | | | | | |
| GFENEESDYH | AAV9 | 13.97277 | L | 3° | HDTNLMQNSTK | AAV9 | 9.402727 | K | 3° |
| TLPRAGIMQK | Loop1 | | | | AHLKRSNVE | Loop2 | | | |
| (SEQ ID NO: | | | | | (SEQ ID NO: 542) | | | | |
| 531) | | | | | | | | | |
| SMGTQGRAC | AAV9 | 12.78786 | L | NHP | SESIPTLSNRVK | AAV9 | 9.379991 | K | 3° |
| NKTAPQASGC | Loop1 | | | | TQQKSNLE (SEQ | Loop2 | | | |
| D (SEQ ID NO: | | | | | ID NO: 634) | | | | |
| 633) | | | | | | | | | |
| QDNTCEEYHT | AAV9 | 12.39187 | L | NHP | NCKFLKECTICK | AAV9 | 9.329294 | K | 2° |
| MGPHSSHIPK | Loop1 | | | | RARGDDMD | Loop2 | | | |
| (SEQ ID NO: | | | | | (SEQ ID NO: 635) | | | | |
| 543) | | | | | | | | | |
| KLARTIRTART | AAV9 | 10.76914 | L | 20 | ARLVLTNPEGR | AAV9 | 9.299425 | K | 2° |
| QAEEREMIQ | Loop1 | | | | KDGKEVTFQ | Loop2 | | | |
| (SEQ ID NO: | | | | | (SEQ ID NO: 637) | | | | |
| 636) | | | | | | | | | |
| MVNSSGRASG | AAV9 | 10.36477 | L | 2° | SNCRHEIDAGV | AAV9 | 9.29233 | K | 2° |
| QMAHQLLPTL | Loop1 | | | | VCTNETRST | Loop2 | | | |
| (SEQ ID NO: | | | | | (SEQ ID NO: 639) | | | | |
| 638) | | | | | | | | | |
| QDNTCEEYHT | AAV9 | 10.08961 | L | 3° | CKFLKEGTICKR | AAV9 | 9.1154 | K | 2° |
| MGPHSSHIPK | Loop1 | | | | ARGDDVDV | Loop2 | | | |
| (SEQ ID NO: | | | | | (SEQ ID NO: 640) | | | | |
| 543) | | | | | | | | | |
| LNSNAIKNLPT | AAV9 | 9.993713 | L | 2° | FSIGVLGYQKTV | AAV9 | 9.097607 | K | 3° |
| PLGGAAGHQ | Loop2 | | | | DHTKVNSK | Loop2 | | | |
| (SEQ ID NO: | | | | | (SEQ ID NO: 545) | | | | |
| 544) | | | | | | | | | |
| GLESEESEYHT | AAV9 | 9.917488 | L | NHP | CRHERDAGVVC | AAV9 | 9.06325 | K | 2° |
| LPRARIMQR | Loop1 | | | | TNETRSTNT | Loop2 | | | |
| (SEQ ID NO: | | | | | (SEQ ID NO: 642) | | | | |
| 641) | | | | | | | | | |
| QDNTCEEYHT | AAV9 | 9.798377 | L | 3° | KLSSTSRLKSVG | AAV9 | 9.03007 | K | 2° |
| MGPHSNHIPK | Loop1 | | | | LNLEGNGE (SEQ | Loop1 | | | |
| (SEQ ID NO: | | | | | ID NO: 644) | | | | |
| 643) | | | | | | | | | |
| SSQAGVPVRV | AAV9 | 9.748563 | L | 2° | VRLVLTNPEGR | AAV9 | 8.979787 | K | 2° |
| PRSPRAPESA | Loop1 | | | | KEGKEVSFK | Loop2 | | | |
| (SEQ ID NO: | | | | | (SEQ ID NO: 646) | | | | |
| 645) | | | | | | | | | |
| AGGSSSNSTR | AAV9 | 9.500178 | L | 2° | VRRVLTNPEGR | AAV9 | 8.966907 | K | 2° |
| EPPASGLVAF | Loop2 | | | | KEGMEVTFQ | Loop2 | | | |
| (SEQ ID NO: | | | | | (SEQ ID NO: 647) | | | | |
| 553) | | | | | | | | | |
| LQSAKDNNIK | AAV9 | 9.472418 | L | 3° | NCKFLKEGTICK | AAV9 | 8.957443 | K | 2° |
| FRAIGHSDSG | Loop1 | | | | RARGDYMD | Loop2 | | | |
| (SEQ ID NO: | | | | | (SEQ ID NO: 649) | | | | |
| 648) | | | | | | | | | |
| QGISNSSTSSM | AAV9 | 9.337745 | L | 2° | AGGSSSNSTREP | AAV9 | 8.949626 | K | 2° |
| PNSSQSSHS | Loop1 | | | | PASGLVAF (SEQ | Loop2 | | | |
| (SEQ ID NO: | | | | | ID NO: 553) | | | | |
| 650) | | | | | | | | | |
| FLEVSEQILAK | AAV9 | 9.324263 | L | 2° | ARLVLINTEGR | AAV9 | 8.901878 | K | 2° |
| IPSGKNKQT | Loop1 | | | | KEGKEVTFQ | Loop2 | | | |
| (SEQ ID NO: | | | | | (SEQ ID NO: 652) | | | | |
| 651) | | | | | | | | | |
| FRGVAGGTAL | AAV9 | 9.322438 | L | NHP | NRAVFKGGQDN | AAV9 | 8.878608 | K | 2° |
| SRSAVHESTG | Loop2 | | | | IHVLKRDVA | Loop2 | | | |
| (SEQ ID NO: | | | | | (SEQ ID NO: 654) | | | | |
| 653) | | | | | | | | | |
| NVDFSINVEN | AAV9 | 9.203686 | L | 2° | SNCRHERYAGV | AAV9 | 8.839399 | K | 2° |
| QTRARDDVSR | Loop2 | | | | VCTNETRST | Loop2 | | | |
| (SEQ ID NO: | | | | | (SEQ ID NO: 655) | | | | |
| 557) | | | | | | | | | |
| VLNSNAIKNL | AAV9 | 9.100841 | L | 2° | YSIGVLGYQKS | AAV9 | 8.809637 | K | 3° |
| HQPLGGAAGH | Loop2 | | | | VDHTKVNSK | Loop2 | | | |
| (SEQ ID NO: | | | | | (SEQ ID NO: 547) | | | | |
| 559) | | | | | | | | | |
| GHDTKLMQNS | AAV9 | 9.066517 | L | 2° | DINGLTTGRRG | AAV9 | 21.38375 | B | NHP |
| TKAPLKSSTV | Loop1 | | | | DMAAIHGMN | Loop1 | | | |
| (SEQ ID NO: | | | | | (SEQ ID NO: 556) | | | | |
| 656) | | | | | | | | | |
| LREIRSYQRTT | AAV9 | 9.013501 | L | 2° | QDNTCEEYHTM | AAV9 | 15.55224 | B | NHP |
| EPLIRKLPF | Loop1 | | | | GPHSSHIPK | Loop1 | | | |
| (SEQ ID NO: | | | | | (SEQ ID NO: 543) | | | | |
| 657) | | | | | | | | | |
| MIQTLTNKSKI | AAV9 | 8.963172 | L | 2° | GFENEESDYHTL | AAV9 | 14.22356 | B | 3° |
| QNVGWTYEF | Loop2 | | | | PRAGIMQK (SEQ | Loop1 | | | |
| (SEQ ID NO: | | | | | ID NO: 531) | | | | |
| 575) | | | | | | | | | |
| DFRIHVENQTL | AAV9 | 8.919031 | L | 2° | GLESEESEYHTL | AAV9 | 12.48049 | B | NHP |
| ALDDVSRKQ | Loop2 | | | | PRARIMQR (SEQ | Loop1 | | | |
| (SEQ ID NO: | | | | | ID NO: 641) | | | | |
| 590) | | | | | | | | | |
| LVPACCSSSNS | AAV9 | 8.830824 | L | 20 | QDDTCEEYHSA | AAV9 | 12.37509 | B | 2° |
| TREPPASGR | Loop2 | | | | QPNSCHISK | Loop1 | | | |
| (SEQ ID NO: | | | | | (SEQ ID NO: 658) | | | | |
| 569) | | | | | | | | | |
| LLPSSTVTPRA | AAV9 | 8.786784 | L | 2° | YSIGVLGYQKTE | AAV9 | 12.3493 | B | 3° |
| PHLSHSSQP | Loop1 | | | | DHTK VNSK | Loop2 | | | |
| (SEQ ID NO: | | | | | (SEQ ID NO: 532) | | | | |
| 659) | | | | | | | | | |
| GHDTKLMQNS | AAV9 | 8.762197 | L | 2° | YSIGVLGYQKT | AAV9 | 12.17751 | B | 3° |
| TKAPLNRSYV | Loop1 | | | | VDHTKVNSK | Loop2 | | | |
| (SEQ ID NO: | | | | | (SEQ ID NO: 530) | | | | |
| 660) | | | | | | | | | |
| ELIRTIRSART | AAV9 | 8.745947 | L | 2° | WNGQDNTCEEY | AAV9 | 11.82604 | B | 2° |
| QAEERGVIQ | Loop1 | | | | HSVGPHSCH | Loop1 | | | |
| (SEQ ID NO: | | | | | (SEQ ID NO: 662) | | | | |
| 661) | | | | | | | | | |
| VPAGGSSSNS | AAV9 | 8.731578 | L | 2° | GFENEESDYHTL | AAV9 | 11.59255 | B | 2° |
| TREQHASGRV | Loop2 | | | | PRAGIMQK (SEQ | Loop1 | | | |
| (SEQ ID NO: | | | | | ID NO: 531) | | | | |
| 570) | | | | | | | | | |
| DFSIHVENQTR | AAV9 | 8.72787 | L | 2° | SSDNIGVLGYQ | AAV9 | 11.55392 | B | NHP |
| ARDDVSSKQ | Loop2 | | | | KTVDHTKVK | Loop2 | | | |
| (SEQ ID NO: | | | | | (SEQ ID NO: 664) | | | | |
| 663) | | | | | | | | | |
| PGVLPLLTVD | AAV9 | 8.712366 | L | 2° | NGGARLAEYHA | AAV9 | 11.53923 | B | 2° |
| PGSAKQRPPR | Loop1 | | | | KTTEHQSTL | Loop1 | | | |
| (SEQ ID NO: | | | | | (SEQ ID NO: 666) | | | | |
| 665) | | | | | | | | | |
| LVPAGGSSSN | AAV9 | 8.69303 | L | 2° | GLENEESDYHT | AAV9 | 11.42221 | B | 2° |
| STREQHASGR | Loop2 | | | | LPRARIMQK | Loop1 | | | |
| (SEQ ID NO: | | | | | (SEQ ID NO: 667) | | | | |
| 587) | | | | | | | | | |
| EYPERQASRA | AAV9 | 8.656612 | L | 2° | YSIGVLGNQKT | AAV9 | 11.35652 | B | 3° |
| NTVRSFNHEE | Loop2 | | | | VDHTKVNSK | Loop2 | | | |
| (SEQ ID NO: | | | | | (SEQ ID NO: 533) | | | | |
| 668) | | | | | | | | | |
| HQANLSKQPT | AAV9 | 8.654464 | L | NHP | DARHIVTQSDDP | AAV9 | 11.27287 | B | 2° |
| SQPRGDPTGP | Loop1 | | | | HFHEKKQI (SEQ | Loop1 | | | |
| (SEQ ID NO: | | | | | ID NO: 670) | | | | |
| 669) | | | | | | | | | |
| VTAGGSSSNS | AAV9 | 8.626233 | L | 2° | VARHVGSQSDD | AAV9 | 11.11897 | B | 2° |
| TREQPASGRV | Loop2 | | | | PHFHEKKQI | Loop1 | | | |
| (SEQ ID NO: | | | | | (SEQ ID NO: 671) | | | | |
| 567) | | | | | | | | | |
| IRLGLSHKAD | AAV9 | 8.606854 | L | 2° | DAPLGTGANKD | AAV9 | 11.04062 | B | 3° |
| MQTREVLMK | Loop1 | | | | LELPWLEQQ | Loop2 | | | |
| L (SEQ ID | | | | | (SEQ ID NO: 534) | | | | |
| NO: 672) | | | | | | | | | |
| VLNSNAIKNLP | AAV9 | 8.595369 | L | 2° | KWVDDQEVINK | AAV9 | 11.02815 | B | 3° |
| QQLGGAAGH | Loop2 | | | | ASNSNKIRP | Loop2 | | | |
| (SEQ ID NO: | | | | | (SEQ ID NO: 673) | | | | |
| 576) | | | | | | | | | |
| VQAGGSSSNS | AAV9 | 8.594954 | L | 2° | GFENEESEYHTL | AAV9 | 10.77684 | B | 2° |
| TREQPASGRV | Loop2 | | | | PRARIMQK (SEQ | Loop1 | | | |
| (SEQ ID NO: | | | | | ID NO: 674) | | | | |
| 563) | | | | | | | | | |
| SVLNSNAIKNL | AAV9 | 8.5523 | L | 2° | GVDNEDYHGRG | AAV9 | 10.66715 | B | 2° |
| PQQLGGAAG | Loop2 | | | | DLGRLKTQK | Loop1 | | | |
| (SEQ ID NO: | | | | | (SEQ ID NO: 675) | | | | |
| 586) | | | | | | | | | |
| TAAHPELKTQ | AAV9 | 8.493584 | L | 2° | SSDSIGVLGYQK | AAV9 | 10.45263 | B | NHP |
| LTANITKSGN | Loop2 | | | | TEDHTKVK | Loop2 | | | |
| (SEQ ID NO: | | | | | (SEQ ID NO: 676) | | | | |
| 568) | | | | | | | | | |
| GMRLPRGAPV | AAV9 | 8.489128 | L | 2° | PAFRNEEYHKS | AAV9 | 10.41258 | B | 2° |
| DIPSSDLTGR | Loop1 | | | | VLENQATFF | Loop1 | | | |
| (SEQ ID NO: | | | | | (SEQ ID NO: 678) | | | | |
| 677) | | | | | | | | | |
| ACGSSSNSTRE | AAV9 | 8.4853 | L | 2° | YLESGSINEKDY | AAV9 | 10.40359 | B | 2° |
| PPASGLVAL | Loop2 | | | | HSRLNIDE (SEQ | Loop1 | | | |
| (SEQ ID NO: | | | | | ID NO: 679) | | | | |
| 583 | | | | | | | | | |
| VDFRIHVENQ | AAV9 | 8.477405 | L | 2° | NSVLNSNAIKNL | AAV9 | 10.30484 | B | NHP |
| TLARDDVSRN | Loop2 | | | | PQPLGGTA (SEQ | Loop2 | | | |
| (SEQ ID NO: | | | | | ID NO: 681) | | | | |
| 680) | | | | | | | | | |
| LLRECNTVRS | AAV9 | 8.444189 | L | 2° | GFENEESDYHTL | AAV9 | 10.27759 | B | 2° |
| FLAAAAETLE | Loop2 | | | | PRARIVQK (SEQ | Loop1 | | | |
| (SEQ ID NO: | | | | | ID NO: 683) | | | | |
| 682) | | | | | | | | | |
| MIQTLTNKSKI | AAV9 | 8.411206 | L | 2° | YSIGVL VYQKT | AAV9 | 10.24646 | B | 3° |
| QNVGVTYEF | Loop2 | | | | VDHTKVNSK | Loop2 | | | |
| (SEQ ID NO: | | | | | (SEQ ID NO: 537) | | | | |
| 684) | | | | | | | | | |
| GHDTKLMQNS | AAV9 | 8.376499 | L | 2° | YSIGVLGYQKT | AAV9 | 10.22341 | B | 3° |
| TKAPLKRPDV | Loop1 | | | | VDHTKVNGK | Loop2 | | | |
| (SEQ ID NO: | | | | | (SEQ ID NO: 536) | | | | |
| 685) | | | | | | | | | |
| NRQSSTSVLET | AAV9 | 8.337899 | L | NHP | GDMANSSIEFHP | AAV9 | 10.20711 | B | 2° |
| NKTSVVLSS | Loop2 | | | | KPRQQREV | Loop1 | | | |
| (SEQ ID NO: | | | | | (SEQ ID NO: 687) | | | | |
| 686) | | | | | | | | | |
| VVYTGRDTKI | AAV9 | 8.333714 | L | 2° | YSIGVLSYQKTV | AAV9 | 10.19875 | B | 3° |
| MQNSTKAPLK | Loop1 | | | | DHTKVNSK | Loop2 | | | |
| (SEQ ID NO: | | | | | (SEQ ID NO: 535) | | | | |
| 688) | | | | | | | | | |
| HVHEGQTVKG | AAV9 | 8.315208 | L | 2° | HKENGGARLVE | AAV9 | 10.17384 | B | 2° |
| RVGTVGYLAP | Loop1 | | | | YHAKASEHL | Loop1 | | | |
| (SEQ ID NO: | | | | | (SEQ ID NO: 690) | | | | |
| 689) | | | | | | | | | |
| LKTQLTANITK | AAV9 | 8.303464 | L | 2° | GFENEESDYHA | AAV9 | 10.10022 | B | 2° |
| SGNTFRHEV | Loop2 | | | | LPRARIMQK | Loop1 | | | |
| (SEQ ID NO: | | | | | (SEQ ID NO: 691) | | | | |
| 618) | | | | | | | | | |
| LVPKAAGNSN | AAV9 | 8.297592 | L | 2° | SESIPTLSNRVK | AAV9 | 10.09714 | B | 3° |
| GGFVKPNPND | Loop1 | | | | TQQKSNLE (SEQ | Loop2 | | | |
| (SEQ ID NO: | | | | | ID NO: 634) | | | | |
| 692) | | | | | | | | | |
| HDTNLMQNST | AAV9 | 8.29573 | L | 2° | RSADSYGHPSE | AAV9 | 10.07147 | B | 2° |
| KAHLKRSNVE | Loop2 | | | | NRTIGTTMV | Loop1 | | | |
| (SEQ ID NO: | | | | | (SEQ ID NO: 693) | | | | |
| 542) | | | | | | | | | |
| LRLADREHSS | AAV9 | 8.276815 | L | 2° | GIESEESEYHTL | AAV9 | 9.942643 | B | NHP |
| GESSFEYTVT | Loop1 | | | | PRARIMQR (SEQ | Loop1 | | | |
| (SEQ ID NO: | | | | | ID NO: 695) | | | | |
| 694) | | | | | | | | | |
| SSTSTMPNSSR | AAV9 | 8.265266 | L | 2° | LNSNAIKNLPTP | AAV9 | 9.829831 | B | 2° |
| SSHSSSWTL | Loop2 | | | | LGGAAGHQ | Loop2 | | | |
| (SEQ ID NO: | | | | | (SEQ ID NO: 544) | | | | |
| 566) | | | | | | | | | |
| PACGSSSNSTR | AAV9 | 8.226133 | L | 2° | KFKGVDNVDYH | AAV9 | 9.75871 | B | 2° |
| EPPASGLVA | Loop2 | | | | GRGDLGRPE | Loop1 | | | |
| (SEQ ID NO: | | | | | (SEQ ID NO: 696) | | | | |
| 592) | | | | | | | | | |
| LKTQLTANTT | AAV9 | 8.191223 | L | 2° | GDSIGVLGYQK | AAV9 | 9.70596 | B | NHP |
| KSGNTFRPEV | Loop2 | | | | TVDHTKVNR | Loop2 | | | |
| (SEQ ID NO: | | | | | (SEQ ID NO: 697) | | | | |
| 630) | | | | | | | | | |
| VLNSNAIKNLP | AAV9 | 8.172553 | L | 2° | HKENGGAGLAE | AAV9 | 9.701024 | B | 2° |
| PPLCCAAGH | Loop2 | | | | YHAKATEHL | Loop1 | | | |
| (SEQ ID NO: | | | | | (SEQ ID NO: 698) | | | | |
| 615) | | | | | | | | | |
| DFRINVENKT | AAV9 | 8.16575 | L | 2° | GFENVESDYHT | AAV9 | 9.690835 | B | 2° |
| RARDDVSRKQ | Loop2 | | | | LPRARIMQK | Loop1 | | | |
| (SEQ ID NO: | | | | | (SEQ ID NO: 700) | | | | |
| 699) | | | | | | | | | |
| HDTKLMQNST | AAV9 | 8.163909 | L | 2° | QDNTCEEYHTM | AAV9 | 9.67355 | B | 3° |
| KAHLKRSNVE | Loop2 | | | | GPHSSHIPK | Loop1 | | | |
| (SEQ ID NO: | | | | | (SEQ ID NO: 543) | | | | |
| 701) | | | | | | | | | |
| ELENNKTMNR | AAV9 | 8.137323 | L | 2° | RIKLTYIHKGSA | AAV9 | 9.622513 | B | 2° |
| AENGGRPPLN | Loop2 | | | | MQDLAEVS | Loop1 | | | |
| (SEQ ID NO: | | | | | (SEQ ID NO: 703) | | | | |
| 702) | | | | | | | | | |
| NVDIRINVENQ | AAV9 | 8.121921 | L | 2° | AGGSSSNSTREP | AAV9 | 9.56453 | B | 2° |
| TRARDDVSR | Loop2 | | | | PASGLVAF (SEQ | Loop2 | | | |
| (SEQ ID NO: | | | | | ID NO: 553) | | | | |
| 704) | | | | | | | | | |
| ENNKTMNRAE | AAV9 | 8.112242 | L | 2° | GGGGPGGSGGV | AAV9 | 9.522066 | B | NHP |
| NGGRPPNHHF | Loop2 | | | | GGDSGGYTY | Loop1 | | | |
| (SEQ ID NO: | | | | | (SEQ ID NO: 706) | | | | |
| 705) | | | | | | | | | |
| PELVNNKTMN | AAV9 | 8.102343 | L | 2° | QDNTCEEYHSV | AAV9 | 9.510913 | B | 2° |
| RAENGGRPPN | Loop2 | | | | GPHSCRIPK | Loop1 | | | |
| (SEQ ID NO: | | | | | (SEQ ID NO: 708) | | | | |
| 707) | | | | | | | | | |
| LPEAVLALHN | AAV9 | 8.101657 | L | 2° | SLLESTSDSNLN | AAV9 | 9.442508 | B | 2° |
| STRDLVAGES | Loop2 | | | | KYSTIHKI (SEQ | Loop1 | | | |
| (SEQ ID NO: | | | | | ID NO: 709) | | | | |
| 606) | | | | | | | | | |
| GGSSSNSTRVP | AAV9 | 8.092907 | L | 2° | ESLVGRKESPHS | AAV9 | 9.42889 | B | 2° |
| PASGLVALV | Loop2 | | | | RSGSIVSS (SEQ | Loop1 | | | |
| (SEQ ID NO: | | | | | ID NO: 711) | | | | |
| 710) | | | | | | | | | |
| EPQPVLENNK | AAV9 | 8.088374 | L | 2° | PIGNQEKNVPLP | AAV9 | 9.412134 | B | 2° |
| TMNRAENGG | Loop2 | | | | VIAELPPK (SEQ | LoopI | | | |
| R (SEQ ID | | | | | ID NO: 713) | | | | |
| NO: 712) | | | | | | | | | |
| AESLELAGNA | AAV9 | 8.057996 | L | 2° | GFENEESDYHTL | AAV9 | 9.404316 | B | NHP |
| TRDNKKTRII | Loop1 | | | | PRAGIMQM | Loop1 | | | |
| (SEQ ID NO: | | | | | (SEQ ID NO: 715) | | | | |
| 714) | | | | | | | | | |
| ENNKTMNRAE | AAV9 | 8.057221 | L | 2° | SSDSIGVLGYQK | AAV9 | 9.301232 | B | NHP |
| NGGRPHNHPF | Loop2 | | | | TVDHTKEK | Loop2 | | | |
| (SEQ ID NO: | | | | | (SEQ ID NO: 717) | | | | |
| 716) | | | | | | | | | |
| VDFRIHVENQ | AAV9 | 8.056751 | L | 2° | YSIGVQGYQKT | AAV9 | 9.298736 | B | 3° |
| TRARDDESSK | Loop2 | | | | VDHTKVNSK | Loop2 | | | |
| (SEQ ID NO: | | | | | (SEQ ID NO: 539) | | | | |
| 718) | | | | | | | | | |
| MIQTITNKSKI | AAV9 | 8.048419 | L | 2° | YGSTTDDDDHH | AAV9 | 9.288509 | B | 2° |
| QNVGGTYES | Loop2 | | | | STL VSSRHY | Loop1 | | | |
| (SEQ ID NO: | | | | | (SEQ ID NO: 720) | | | | |
| 719) | | | | | | | | | |
| VPAGGSSSNS | AAV9 | 8.046119 | L | 2° | EDGSARLAEYH | AAV9 | 9.284193 | B | 2° |
| TLEPPASGLV | Loop2 | | | | AKATERLGT | Loop1 | | | |
| (SEQ ID NO: | | | | | (SEQ ID NO: 721) | | | | |
| 624) | | | | | | | | | |
| LKTTLTANITK | AAV9 | 8.037933 | L | 2° | RNGPEVSNDIKK | AAV9 | 9.261346 | B | 3° |
| SGNTFRHEV | Loop2 | | | | RKVDDKDS | Loop2 | | | |
| (SEQ ID NO: | | | | | (SEQ ID NO: 723) | | | | |
| 722) | | | | | | | | | |
| SALDTNSSKST | AAV9 | 8.03392 | L | 2° | GFENEESDYHTL | AAV9 | 9.205369 | B | 2° |
| SSFPCPAVH | Loop2 | | | | PRARTMQK | Loop1 | | | |
| (SEQ ID NO: | | | | | (SEQ ID NO: 725) | | | | |
| 724) | | | | | | | | | |
| DINGLTTGRR | AAV9 | 10.13873 | L | NHP | IDAQGEPHGYR | AAV9 | 9.160974 | B | 2° |
| GDMAAIHGM | Loop1 | | | | QGDPSYRSF | Loop1 | | | |
| N (SEQ ID | | | | | (SEQ ID NO: 726) | | | | |
| NO: 556) | | | | | | | | | |
| GFENEESDYH | AAV9 | 10.00615 | Lv | 3° | RHLESQSDDPHF | AAV9 | 9.117673 | B | 2° |
| TLPRAGIMQK | Loop1 | | | | HEKKQIPF (SEQ | Loop1 | | | |
| (SEQ ID NO: | | | | | ID NO: 727) | | | | |
| 531) | | | | | | | | | |
| LNSNAIKNLPT | AAV9 | 9.488507 | Lv | 2° | QDNSCEEYHTA | AAV9 | 9.094751 | B | 2° |
| PLGGAAGHQ | Loop2 | | | | GPHSGHIPK | Loop1 | | | |
| (SEQ ID NO: | | | | | (SEQ ID NO: 728) | | | | |
| 544) | | | | | | | | | |
| KENGGSRLAE | AAV9 | 8.79956 | Lv | NHP | NVDFSINVENQT | AAV9 | 9.086539 | B | 2° |
| YHAKATEHLS | Loop1 | | | | RARDDVSR | Loop2 | | | |
| (SEQ ID NO: | | | | | (SEQ ID NO: 557) | | | | |
| 729) | | | | | | | | | |
| SSQAGVPVRV | AAV9 | 8.419343 | Lv | NHP | TAAHPELKTQL | AAV9 | 9.047351 | B | 2° |
| PRSPRAPESA | Loop1 | | | | TANITKSGN | Loop2 | | | |
| (SEQ ID NO: | | | | | (SEQ ID NO: 568) | | | | |
| 645) | | | | | | | | | |
| VLNSNAIKNL | AAV9 | 8.328063 | Lv | 2° | QTFDDPHVSSFH | AAV9 | 9.022515 | B | 2° |
| HQPLGGAAGH | Loop2 | | | | GSKRGLNS (SEQ | Loop1 | | | |
| (SEQ ID NO: | | | | | ID NO: 730) | | | | |
| 559) | | | | | | | | | |
| QRGKGLSHW | AAV9 | 8.279242 | Lv | 2° | SSTSTMPNSSRS | AAV9 | 8.998521 | B | 2° |
| VPEHPALPTAS | Loop2 | | | | SHSSSWTL (SEQ | Loop2 | | | |
| (SEQ ID NO: | | | | | ID NO: 566) | | | | |
| 731) | | | | | | | | | |
| QRSGASASGV | AAV9 | 8.262213 | Lv | 2° | YGQDNTCEEYH | AAV9 | 8.996019 | B | 2° |
| YTIKASNATK | Loop2 | | | | TVGPHSSPI (SEQ | Loop1 | | | |
| (SEQ ID NO: | | | | | ID NO: 733) | | | | |
| 732) | | | | | | | | | |
| QRSWASASGV | AAV9 | 8.220422 | Lv | 2° | AKSFDWGLQAG | AAV9 | 8.992627 | B | 2° |
| YTIQASNATK | Loop2 | | | | YITVNSNLS | Loop2 | | | |
| (SEQ ID NO: | | | | | (SEQ ID NO: 735) | | | | |
| 734) | | | | | | | | | |
| QKGIQKPKSG | AAV9 | 8.190674 | Lv | 2° | GFENEESDYHTL | AAV9 | 8.978805 | B | 2° |
| TKGNYDDDW | Loop2 | | | | PRARITQK (SEQ | Loop1 | | | |
| K (SEQ ID | | | | | ID NO: 737) | | | | |
| NO: 736) | | | | | | | | | |
| DFRIHVENQTL | AAV9 | 8.158255 | Lv | 2° | HDTNLMQNSTK | AAV9 | 8.946112 | B | 3° |
| ALDDVSRKQ | Loop2 | | | | AHLKRSNVE | Loop2 | | | |
| (SEQ ID NO: | | | | | (SEQ ID NO: 542) | | | | |
| 590) | | | | | | | | | |
| VDFRIHVENQ | AAV9 | 7.790053 | Lv | 2° | VLNSNAIKNLH | AAV9 | 8.939443 | B | 2° |
| TLARDDVSRN | Loop2 | | | | QPLGGAAGH | Loop2 | | | |
| (SEQ ID NO: | | | | | (SEQ ID NO: 559) | | | | |
| 680) | | | | | | | | | |
| ICVLGYQKTV | AAV9 | 7.766946 | Lv | 2° | VKENGGARLAE | AAV9 | 8.91688B | B | 2° |
| DHTKVNSKLS | Loop2 | | | | YHTKATEHL | Loop1 | | | |
| (SEQ ID NO: | | | | | (SEQ ID NO: 739) | | | | |
| 738) | | | | | | | | | |
| AGGSSSNSTR | AAV9 | 7.746055 | Lv | 2° | LKENGGAILAE | AAV9 | 8.908043 | B | 2° |
| EPPASGLVAF | Loop2 | | | | YHTKATEHL | Loop1 | | | |
| (SEQ ID NO: | | | | | (SEQ ID NO: 740) | | | | |
| 553) | | | | | | | | | |
| VLNSNAIKNLP | AAV9 | 7.745215 | Lv | 2° | GFEDEESDYHTL | AAV9 | 8.900953 | B | 2° |
| QQLGGAAGH | Loop2 | | | | PRARITQR (SEQ | Loop1 | | | |
| (SEQ ID NO: | | | | | ID NO: 741) | | | | |
| 576) | | | | | | | | | |
| RWLQAQKDK | AAV9 | 7.694831 | Lv | 2° | LPEAVLALHNST | AAV9 | 8.872191 | B | 2° |
| MLSKNDTRSA | Loop2 | | | | RDLVAGES (SEQ | Loop2 | | | |
| G (SEQ ID | | | | | ID NO: 606) | | | | |
| NO: 742) | | | | | | | | | |
| NMTKATVNY | AAV9 | 7.644042 | Lv | 2° | FGSATLDDEHH | AAV9 | 8.868846 | B | 2° |
| RKEKTHMMS | Loop2 | | | | STLVSTSHY | Loop1 | | | |
| AV (SEQ ID | | | | | (SEQ ID NO: 744) | | | | |
| NO: 743) | | | | | | | | | |
| QRSWASASGV | AAV9 | 7.642708 | Lv | 2° | FGSATIDVEHHS | AAV9 | 8.865535 | B | 2° |
| NTIQVSNATK | Loop2 | | | | TLASTRHY (SEQ | Loop1 | | | |
| (SEQ ID NO: | | | | | ID NO: 746) | | | | |
| 745) | | | | | | | | | |
| DFSIHVENQTR | AAV9 | 7.631215 | Lv | 2° | LALYNSTRDRK | AAV9 | 8.860514 | B | 2° |
| ARDDVSSKQ | Loop2 | | | | AGVSAEPEP | Loop2 | | | |
| (SEQ ID NO: | | | | | (SEQ ID NO: 747) | | | | |
| 663) | | | | | | | | | |
| LGAPAL VQRP | AAV9 | 7.630549 | Lv | NHP | GGARLVEYHAK | AAV9 | 8.834379 | B | 2° |
| QGNRPGLAKA | Loop1 | | | | ATENLSTLS | Loop1 | | | |
| (SEQ ID NO: | | | | | (SEQ ID NO: 749) | | | | |
| 748) | | | | | | | | | |
| SIGVLGYKKT | AAV9 | 7.603106 | Lv | 2° | GSATYEDEHHS | AAV9 | 8.830555 | B | 2° |
| VDHTKVNSKP | Loop2 | | | | TLVSTRQYG | Loop1 | | | |
| (SEQ ID NO: | | | | | (SEQ ID NO: 751) | | | | |
| 750) | | | | | | | | | |
| QRSWASASGA | AAV9 | 7.588547 | Lv | 2° | GFENEESDYHTL | AAV9 | 17.18655 | M | 3° |
| YTIQVSNATK | Loop2 | | | | PRAGIMQK (SEQ | Loop1 | | | |
| (SEQ ID NO: | | | | | ID NO: 531) | | | | |
| 752) | | | | | | | | | |
| YWGSVTGSQS | AAV9 | 7.563096 | Lv | 2° | YSIGVLGYQKT | AAV9 | 14.02683 | M | 3° |
| NAVSHTQAPR | Loop2 | | | | VDHTKVNSK | Loop2 | | | |
| (SEQ ID NO: | | | | | (SEQ ID NO: 530) | | | | |
| 753) | | | | | | | | | |
| QAYKAPVVVE | AAV9 | 7.544197 | Lv | 2° | YSIGVLGYQKTE | AAV9 | 13.96413 | M | 3° |
| GKVQGLVQA | Loop2 | | | | DHTKVNSK | Loop2 | | | |
| G (SEQ ID | | | | | (SEQ ID NO: 532) | | | | |
| NO: 754) | | | | | | | | | |
| IMELAGNAAR | AAV9 | 7.541857 | Lv | NHP | YSIGVLGNQKT | AAV9 | 13.02269 | M | 3° |
| DNKTTRILPR | Loop1 | | | | VDHTKVNSK | Loop2 | | | |
| (SEQ ID NO: | | | | | (SEQ ID NO: 533) | | | | |
| 755) | | | | | | | | | |
| LYRSLAGRAP | AAV9 | 7.53098 | Lv | 29 | DAPLGTGANKD | AAV9 | 12.71293 | M | 3° |
| AWAAAVSAS | Loop2 | | | | LELPWLEQQ | Loop2 | | | |
| G (SEQ ID | | | | | (SEQ ID NO: 534) | | | | |
| NO: 756) | | | | | | | | | |
| SICVLGYQKT | AAV9 | 7.484248 | Lv | 2° | YSIGVLSYQKTV | AAV9 | 12.17668 | M | 3° |
| VDHTKANSKL | Loop2 | | | | DHTKVNSK | Loop2 | | | |
| (SEQ ID NO: | | | | | (SEQ ID NO: 535) | | | | |
| 757) | | | | | | | | | |
| SVSRALTQAL | AAV9 | 7.436854 | Lv | 2° | YSIGVLGYQKT | AAV9 | 11.92955 | M | 3° |
| PAPTCQNTQV | Loop2 | | | | VDHTKVNGK | Loop2 | | | |
| (SEQ ID NO: | | | | | (SEQ ID NO: 536) | | | | |
| 758) | | | | | | | | | |
| VLNSNAIKNLP | AAV9 | 7.41241 | Lv | 2° | YSIGVL VYQKT | AAV9 | 11.56475 | M | 3° |
| PPLCCAAGH | Loop2 | | | | VDHTKVNSK | Loop2 | | | |
| (SEQ ID NO: | | | | | (SEQ ID NO: 537) | | | | |
| 615) | | | | | | | | | |
| LKTQLTANITK | AAV9 | 7.409261 | Lv | 2° | QDNTCEEYHTM | AAV9 | 11.10573 | M | 3° |
| SGNTFRHEV | Loop2 | | | | GPHSSHIPK | Loop1 | | | |
| (SEQ ID NO: | | | | | (SEQ ID NO: 543) | | | | |
| 618) | | | | | | | | | |
| QKGIKKPKSG | AAV9 | 7.408743 | Lv | 2° | YSIGVQGYQKT | AAV9 | 10.96218 | M | 3° |
| TQGNYDDDW | Loop2 | | | | VDHTKVNSK | Loop2 | | | |
| K (SEQ ID | | | | | (SEQ ID NO: 539) | | | | |
| NO: 759) | | | | | | | | | |
| VPTGSFMMVN | AAV9 | 7.399719 | Lv | 2° | HDTNLMQNSTK | AAV9 | 10.94556 | M | 3° |
| SSGRASGKKA | Loop2 | | | | AHLKRSNVE | Loop2 | | | |
| (SEQ ID NO: | | | | | (SEQ ID NO: 542) | | | | |
| 760) | | | | | | | | | |
| GFENEESDYH | AAV9 | 16.28212 | I | 3° | FSIGVLGYQKTV | AAV9 | 10.34177 | M | 3° |
| TLPRAGIMQK | Loop1 | | | | DHTKVNSK | Loop2 | | | |
| (SEQ ID NO: | | | | | (SEQ ID NO: 545) | | | | |
| 531) | | | | | | | | | |
| YSIGVLGYQK | AAV9 | 13.38331 | I | 3° | YSIGVLGYQKS | AAV9 | 10.06095 | M | 3° |
| TVDHTKVNSK | Loop2 | | | | VDHTKVNSK | Loop2 | | | |
| (SEQ ID NO: | | | | | (SEQ ID NO: 547) | | | | |
| 530) | | | | | | | | | |
| YSIGVLGYQK | AAV9 | 13.26262 | I | 3° | RSDLTTLTESYA | AAV9 | 9.866847 | M | 3° |
| TEDHTKVNSK | Loop2 | | | | NRQGLNKN | Loop1 | | | |
| (SEQ ID NO: | | | | | (SEQ ID NO: 761) | | | | |
| 532) | | | | | | | | | |
| YSIGVLGNQK | AAV9 | 12.32805 | I | 3° | LNSNAIKNLPTP | AAV9 | 9.7937 | M | 2° |
| TVDHTKVNSK | Loop2 | | | | LGGAAGHQ | Loop2 | | | |
| (SEQ ID NO: | | | | | (SEQ ID NO: 544) | | | | |
| 533) | | | | | | | | | |
| DAPLGTGANK | AAV9 | 11.7147 | I | 3° | YSIGVLGYQKT | AAV9 | 9.791349 | M | 3° |
| DLELPWLEQQ | Loop2 | | | | VDHTKVNST | Loop2 | | | |
| (SEQ ID NO: | | | | | (SEQ ID NO: 548) | | | | |
| 534) | | | | | | | | | |
| YSIGVLSYQKT | AAV9 | 11.44192 | I | 3° | SSQAGVPVRAP | AAV9 | 9.761722 | M | 3° |
| VDHTKVNSK | Loop2 | | | | RSPRAPESA | Loop1 | | | |
| (SEQ ID NO: | | | | | (SEQ ID NO: 762) | | | | |
| 535) | | | | | | | | | |
| YSIGVLGYQK | AAV9 | 11.27777 | I | 3° | TAGSTMGAASIT | AAV9 | 9.655512 | M | 2° |
| TVDHTKVNGK | Loop2 | | | | QTVLARNL | Loop1 | | | |
| (SEQ ID NO: | | | | | (SEQ ID NO: 763) | | | | |
| 536) | | | | | | | | | |
| YSIGVL VYQK | AAV9 | 11.27179 | I | 3° | KENGGARLAEY | AAV9 | 9.605415 | M | 2° |
| TVDHTKVNSK | Loop2 | | | | HAKSTEHLS | Loop1 | | | |
| (SEQ ID NO: | | | | | (SEQ ID NO: 764) | | | | |
| 537) | | | | | | | | | |
| QDNTCEEYHT | AAV9 | 10.76647 | I | 3° | TAAHPELKTQL | AAV9 | 9.196748 | M | 2° |
| MGPHSSHIPK | Loop1 | | | | TANITKSGN | Loop2 | | | |
| (SEQ ID NO: | | | | | (SEQ ID NO: 568) | | | | |
| 543) | | | | | | | | | |
| QDNTCEEYHT | AAV9 | 10.69909 | I | 3° | WVDDQKVINKA | AAV9 | 9.097937 | M | 3° |
| MGPHSNHIPK | Loop 1 | | | | SNSNKIRLE | Loop2 | | | |
| (SEQ ID NO: | | | | | (SEQ ID NO: 765) | | | | |
| 643) | | | | | | | | | |
| HDTNLMQNST | AAV9 | 10.69498 | I | 3° | AGGSSSNSTREP | AAV9 | 9.074173 | M | 2° |
| KAHLKRSNVE | Loop2 | | | | PASGLVAF (SEQ | Loop2 | | | |
| (SEQ ID NO: | | | | | ID NO: 553) | | | | |
| 542 | | | | | | | | | |
| YSIGVQGYQK | AAV9 | 10.26071 | I | 3° | ENVDFRINVEN | AAV9 | 9.004632 | M | 2° |
| TVDHTKVNSK | Loop2 | | | | QTRARDDVS | Loop2 | | | |
| (SEQ ID NO: | | | | | (SEQ ID NO: 588) | | | | |
| 539) | | | | | | | | | |
| LLSASSVGSA | AAV9 | 10.06101 | I | 2° | GMIQTANVGVG | AAV9 | 8.954396 | M | 2° |
| HGDEGAGYTD | Loop2 | | | | ISGNEGMQA | Loop2 | | | |
| (SEQ ID NO: | | | | | (SEQ ID NO: 767) | | | | |
| 766) | | | | | | | | | |
| FSIGVLGYQKT | AAV9 | 9.780598 | I | 3° | EQLITQEPKEAN | AAV9 | 8.880195 | M | 2° |
| VDHTKVNSK | Loop2 | | | | STTSKKQS (SEQ | Loop2 | | | |
| (SEQ ID NO: | | | | | ID NO: 768) | | | | |
| 545) | | | | | | | | | |
| YSIGVLGYQK | AAV9 | 9.476502 | I | 3° | PGAVLALYNST | AAV9 | 8.795954 | M | 2° |
| SVDHTKVNSK | Loop2 | | | | SDRVAGESA | Loop2 | | | |
| (SEQ ID NO: | | | | | (SEQ ID NO: 769) | | | | |
| 547) | | | | | | | | | |
| YSIGVLGYQK | AAV9 | 9.285147 | I | 3° | NVDFSINVENQT | AAV9 | 8.79169 | M | 2° |
| TVDHTKVNST | Loop2 | | | | RARDDVSR | Loop2 | | | |
| (SEQ ID NO: | | | | | (SEQ ID NO: 557) | | | | |
| 548) | | | | | | | | | |
| ISEERFRQLE | AAV9 | 8.875858 | I | 2° | LVPACCSSSNST | AAV9 | 8.739936 | M | 2° |
| KIKTVGSTYM | Loop1 | | | | REPPASGR (SEQ | Loop2 | | | |
| (SEQ ID NO: | | | | | ID NO: 569) | | | | |
| 770) | | | | | | | | | |
| AKSAVEILLTI | AAV9 | 8.42562 | I | 2° | LVPAGGSSSNST | AAV9 | 8.722982 | M | 2° |
| DDTKRAGMK | Loop2 | | | | REQHASGR | Loop2 | | | |
| (SEQ ID NO: | | | | | (SEQ ID NO: 587) | | | | |
| 589) | | | | | | | | | |
| IQGQLQPSLKT | AAV9 | 8.372161 | I | 2° | DFRIHVENQTLA | AAV9 | 8.702759 | M | 2° |
| GSEEIRGQY | Loop1 | | | | LDDVSRKQ | Loop2 | | | |
| (SEQ ID NO: | | | | | (SEQ ID NO: 590) | | | | |
| 771) | | | | | | | | | |
| ERVPVHATVV | AAV9 | 8.282936 | I | 2° | QNAVNVVKQIK | AAV9 | 8.696219 | M | 2° |
| GDVVSPEMGE | Loop1 | | | | TLIEKTNEE | Loop2 | | | |
| (SEQ ID NO: | | | | | (SEQ ID NO: 773) | | | | |
| 772) | | | | | | | | | |
| SVAGSLSLAA | AAV9 | 8.172114 | I | 2° | VTAGGSSSNSTR | AAV9 | 8.685301 | M | 2° |
| NSAAQKTHQD | Loop1 | | | | EQPASGRV (SEQ | Loop2 | | | |
| (SEQ ID NO: | | | | | ID NO: 567) | | | | |
| 774) | | | | | | | | | |
| GLESEESEYHT | AAV9 | 8.167663 | I | 3° | VQAGGSSSNST | AAV9 | 8.685292 | M | 2° |
| LPRARIMQR | Loop1 | | | | REQPASGRV | Loop2 | | | |
| (SEQ ID NO: | | | | | (SEQ ID NO: 563) | | | | |
| 641) | | | | | | | | | |
| ARLLGSLDPS | AAV9 | 8.138603 | I | 2° | YSIGVLGNQKT | AAV9 | 8.670167 | M | 3° |
| ADLSSQRGKV | Loop1 | | | | VDHTKENSK | Loop2 | | | |
| SEQ ID NO: | | | | | (SEQ ID NO: 555) | | | | |
| 775) | | | | | | | | | |
| HYLGLARRAP | AAV9 | 8.117226 | I | 2° | VPAGGSSSNSTR | AAV9 | 8.591786 | M | 2° |
| AGAAAVSASG | Loop2 | | | | EQHASGRV | Loop2 | | | |
| SEQ ID NO: | | | | | (SEQ ID NO: 570) | | | | |
| 776) | | | | | | | | | |
| AGGSSSNSTR | AAV9 | 8.115973 | I | 2° | NVDIRINVENQT | AAV9 | 8.58057 | M | 2° |
| EPPASGLVAF | Loop2 | | | | RARDDVSR | Loop2 | | | |
| (SEQ ID NO: | | | | | (SEQ ID NO: 704) | | | | |
| 553) | | | | | | | | | |
| KQQDSLPDTK | AAV9 | 8.098824 | I | 2° | FGSASYDEEHHS | AAV9 | 8.453823 | M | 2° |
| AGGPAHSKVV | Loop1 | | | | TLVSTRHY (SEQ | Loop1 | | | |
| (SEQ ID NO: | | | | | ID NO: 778) | | | | |
| 777) | | | | | | | | | |
| KWKTTFDLKK | AAV9 | 8.013461 | I | 2° | ACGSSSNSTREP | AAV9 | 8.411724 | M | 2° |
| TRMEPFHFKT | Loop1 | | | | PASGLVAL (SEQ | Loop2 | | | |
| (SEQ ID NO: | | | | | ID NO: 583) | | | | |
| 779) | | | | | | | | | |
| SDSICVLGYQ | AAV9 | 8.003536 | I | 2° | QNAVNWVKQIK | AAV9 | 8.324026 | M | 2° |
| KTVDHTKVNS | Loop2 | | | | TLIEKTNEE | Loop2 | | | |
| (SEQ ID NO: | | | | | (SEQ ID NO: 780) | | | | |
| 552) | | | | | | | | | |
| SFMIRGDEVR | AAV9 | 7.959401 | I | 2° | LVTAGGSSSNST | AAV9 | 8.295296 | M | 2° |
| QTASGQTGKI | Loop2 | | | | REQPASGR (SEQ | Loop2 | | | |
| (SEQ ID NO: | | | | | ID NO: 782) | | | | |
| 781) | | | | | | | | | |
| GMIQTANVGV | AAV9 | 7.936933 | I | 2° | ENVDFRIHVEN | AAV9 | 8.291462 | M | 2° |
| GISGNEGMQA | Loop2 | | | | KTRARDDVS | Loop2 | | | |
| (SEQ ID NO: | | | | | (SEQ ID NO: 783) | | | | |
| 767) | | | | | | | | | |
| RGLQVTVLGE | AAV9 | 7.901321 | I | 2° | PELKTQLTANIT | AAV9 | 8.279723 | M | 2° |
| NKTGNSPVER | Loop2 | | | | KSGDTFRP (SEQ | Loop2 | | | |
| (SEQ ID NO: | | | | | ID NO: 785) | | | | |
| 784) | | | | | | | | | |
| LAMAGSAVGT | AAV9 | 7.893966 | I | 2° | LVKIRSNSSKPD | AAV9 | 8.275692 | M | 2° |
| TSLTLSAQSR | Loop2 | | | | LMASLLPQ (SEQ | Loop2 | | | |
| (SEQ ID NO: | | | | | ID NO: 787) | | | | |
| 786) | | | | | | | | | |
| TAAHPELKTQ | AAV9 | 7.789394 | I | 2° | VLNSNAIKNLH | AAV9 | 8.260826 | M | 2° |
| LTANITKSGN | Loop2 | | | | QPLGGAAGH | Loop2 | | | |
| (SEQ ID NO: | | | | | (SEQ ID NO: 559) | | | | |
| 568) | | | | | | | | | |
| TGLLPAAGEK | AAV9 | 7.788899 | 1 | 2° | TGGSSSNSTREP | AAV9 | 8.259557 | M | 2° |
| TCGPLGNLAE | Loop2 | | | | HASGRVAL | Loop2 | | | |
| (SEQ ID NO: | | | | | (SEQ ID NO: 789) | | | | |
| 788) | | | | | | | | | |
| YSIGVLGNQK | AAV9 | 7.687701 | I | 3° | DFSIHVENQTRA | AAV9 | 8.246807 | M | 2° |
| TVDHTKENSK | Loop2 | | | | RDDVSSKQ | Loop2 | | | |
| (SEQ ID NO: | | | | | (SEQ ID NO: 663) | | | | |
| 555) | | | | | | | | | |
| AKSAVEILLTI | AAV9 | 9.094878 | S | 2° | SVLNSNAIKNLP | AAV9 | 8.218652 | M | 2° |
| DDTKRAGMK | Loop2 | | | | QQLGGAAG | Loop2 | | | |
| (SEQ ID NO: | | | | | (SEQ ID NO: 586) | | | | |
| 589) | | | | | | | | | |
| YSIGVLGYQK | AAV9 | 8.994118 | S | 3° | LKTPLTTNITKS | AAV9 | 8.202921 | M | 2° |
| TEDHTKVNSK | Loop2 | | | | GNTFLPEV (SEQ | Loop2 | | | |
| (SEQ ID NO: | | | | | ID NO: 790) | | | | |
| 532) | | | | | | | | | |
| GFENEESDYH | AAV9 | 8.825712 | S | 3° | YSFGVLGYQKT | AAV9 | 8.199302 | M | 3° |
| TLPRAGIMQK | Loop1 | | | | VDHTKVNSK | Loop2 | | | |
| (SEQ ID NO: | | | | | (SEQ ID NO: 791) | | | | |
| 531) | | | | | | | | | |
| CKFLKEGTICK | AAV9 | 8.791321 | S | 2° | LLRECNTVRSFL | AAV9 | 8.170173 | M | 2° |
| RARCDDMDD | Loop2 | | | | AAAAETLE (SEQ | Loop2 | | | |
| (SEQ ID NO: | | | | | ID NO: 682) | | | | |
| 609) | | | | | | | | | |
| YSIGVLGYQK | AAV9 | 8.783985 | S | 3° | MIQTLINKSKIQ | AAV9 | 8.154686 | M | 2° |
| TVDHTKVNSK | Loop2 | | | | NVGWTYEF | Loop2 | | | |
| (SEQ ID NO: | | | | | (SEQ ID NO: 575) | | | | |
| 530) | | | | | | | | | |
| AELKPANLPV | AAV9 | 8.550499 | S | 2° | QTIPQISNRTRN | AAV9 | 8.139015 | M | 2° |
| TKPFTVGDNK | Loop2 | | | | TMVQDNSY | Loop2 | | | |
| (SEQ ID NO: | | | | | (SEQ ID NO: 793) | | | | |
| 792) | | | | | | | | | |
| PSFCTQKPRKF | AAV9 | 8.151695 | S | 2° | YWGSVTGSQSN | AAV9 | 8.124375 | M | 2° |
| YCELPDISL | Loop2 | | | | AVSHTQAPR | Loop2 | | | |
| (SEQ ID NO: | | | | | (SEQ ID NO: 753) | | | | |
| 794) | | | | | | | | | |
| YSIGVLGNQK | AAV9 | 8.113986 | S | 3° | LVQIRSNSSKPD | AAV9 | 8.111179 | M | 2° |
| TVDHTKVNSK | Loop2 | | | | LMASMLPQ | Loop2 | | | |
| (SEQ ID NO: | | | | | (SEQ ID NO: 795) | | | | |
| 533) | | | | | | | | | |
| GMIQTANVGV | AAV9 | 7.999101S | S | 2° | VNNSSTNDNLV | AAV9 | 8.098509 | M | 2° |
| GISGNEGMQA | Loop2 | | | | RKNDKVYIN | Loop2 | | | |
| (SEQ ID NO: | | | | | (SEQ ID NO: 796) | | | | |
| 767) | | | | | | | | | |
| EKMLVLDAEQ | AAV9 | 7.961541 | S | 2° | VPAGGSSSNSTL | AAV9 | 8.07234 | M | 2° |
| RVTAGEALAN | Loop2 | | | | EPPASGLV (SEQ | Loop2 | | | |
| (SEQ ID NO: | | | | | ID NO: 624) | | | | |
| 797) | | | | | | | | | |
| GSQASGIPAK | AAV9 | 7.915445 | S | 2° | PELENNKTMSR | AAV9 | 8.048644 | M | 2° |
| VSATVSSPGS | Loop2 | | | | AENGGRPPN | Loop2 | | | |
| (SEQ ID NO: | | | | | (SEQ ID NO: 799) | | | | |
| 798) | | | | | | | | | |
| WLVAKRVQD | AAV9 | 7.88424 | S | 2° | GNVDFRIHVEN | AAV9 | 8.030347 | M | 2° |
| HTTVVGDVVS | Loop2 | | | | QTRALDDVS | Loop2 | | | |
| Q (SEQ ID | | | | | (SEQ ID NO: 801) | | | | |
| NO: 800) | | | | | | | | | |
| YKVLKQVHPD | AAV9 | 7.823109 | S | 3° | QTIPKISNRTLNT | AAV9 | 7.983687 | M | 2° |
| SGVSSQAMGI | Loop1 | | | | QVQDNSY (SEQ | Loop2 | | | |
| (SEQ ID NO: | | | | | ID NO: 803) | | | | |
| 802) | | | | | | | | | |
| SPANGSLGPTL | AAV9 | 7.701629 | S | 2° | GLVQAGGSSSN | AAV9 | 7.956418 | M | 2° |
| NLSSWESRL | Loop2 | | | | STREPHASG | Loop2 | | | |
| (SEQ ID NO: | | | | | (SEQ ID NO: 805) | | | | |
| 804) | | | | | | | | | |
| QAGIPVRAPRS | AAV9 | 7.658135 | S | 2° | VPACGSSSNSTR | AAV9 | 7.928663 | M | 2° |
| PRAQEHALE | Loop2 | | | | EPPASGLV (SEQ | Loop2 | | | |
| (SEQ ID NO: | | | | | ID NO: 620) | | | | |
| 806) | | | | | | | | | |
| IGILSQGVASG | AAV9 | 7.654589 | S | 2° | TAAHPELKTTLT | AAV9 | 7.920023 | M | 2° |
| MAGSAMSSK | Loop2 | | | | ANITKSGN (SEQ | Loop2 | | | |
| (SEQ ID NO: | | | | | ID NO: 808) | | | | |
| 807) | | | | | | | | | |
| NCKFLKEGTIC | AAV9 | 7.646112 | S | 2° | LKTQLTANITKS | AAV9 | 7.902972 | M | 2° |
| KRARCDDMD | Loop2 | | | | GNTFRHEV (SEQ | Loop2 | | | |
| (SEQ ID NO: | | | | | ID NO: 618) | | | | |
| 623) | | | | | | | | | |
| LKQVHPDTCIS | AAV9 | 7.54635 | S | 2° | MSQVPLQNYSK | AAV9 | 7.901576 | M | 2° |
| SKAMGIMNS | Loop2 | | | | VERHPVTAK | Loop1 | | | |
| (SEQ ID NO: | | | | | (SEQ ID NO: 810) | | | | |
| 809) | | | | | | | | | |
| HSWTTLYNPS | AAV9 | 7.529922 | S | 2° | VPACCSSSNSTR | AAV9 | 7.898005 | M | 2° |
| LESRVTMSVD | Loop2 | | | | EPPASGRV (SEQ | Loop2 | | | |
| (SEQ ID NO: | | | | | ID NO: 812) | | | | |
| 811) | | | | | | | | | |
| QNTATILLKED | AAV9 | 7.521096 | S | 2° | AKSSDWGLQAG | AAV9 | 7.881375 | M | 2° |
| SLDATRAAI | Loop2 | | | | YIHVNPNLS | Loop2 | | | |
| (SEQ ID NO: | | | | | (SEQ ID NO: 814) | | | | |
| 813) | | | | | | | | | |
| DAPLGTGANK | AAV9 | 7.515163 | S | 3° | YSIGVLGYQKT | AAV9 | 7.866734 | M | 3° |
| DLELPWLEQQ | Loop2 | | | | VDLTKVNSK | Loop2 | | | |
| (SEQ ID NO: | | | | | (SEQ ID NO: 815) | | | | |
| 534) | | | | | | | | | |
| SDSICVLGYQ | AAV9 | 7.50278 | S | 2° | DNVDFRIHVEN | AAV9 | 7.853419 | M | 2° |
| KTVDHTKVNS | Loop2 | | | | QTRARDDVS | Loop2 | | | |
| SEQ ID NO: | | | | | (SEQ ID NO: 816) | | | | |
| 552) | | | | | | | | | |
| LLAGGLHAW | AAV9 | 7.465502 | S | 2° | | | | | |
| KVIKGEEISVV | Loop2 | | | | | | | | |
| (SEQ ID NO: | | | | | | | | | |
| 817) | | | | | | | | | |
| EGKVQGLVQA | AAV9 | 7.406399 | S | 2° | | | | | |
| GGSSSNSTRE | Loop2 | | | | | | | | |
| (SEQ ID NO: | | | | | | | | | |
| 818) | | | | | | | | | |
| QSAVEILLTID | AAV9 | 7.390435 | S | 2° | | | | | |
| DTKMAGMKE | Loop2 | | | | | | | | |
| (SEQ ID NO: | | | | | | | | | |
| 819) | | | | | | | | | |
| YSIGVLSYQKT | AAV9 | 7.340184 | S | 3° | | | | | |
| VDHTKVNSK | Loop2 | | | | | | | | |
| (SEQ ID NO: | | | | | | | | | |
| 535) | | | | | | | | | |
| ETGWTPGLRS | AAV9 | 7.318566 | S | 2° | | | | | |
| LENYGTKLLD | Loop2 | | | | | | | | |
| (SEQ ID NO: | | | | | | | | | |
| 820) | | | | | | | | | |

TABLE 4B
These sequences were identified by looking at
peptides derived from domains that exhibit consistent organ
transfectivity across many variants. Shown in the table below is the
amino acid sequence, the AAV scaffold in which the peptide was
grafted, the log2fold change value and the respective organ that it
transduces, as well as the screen in which the peptide was
identified (P = pancreas; H = heart; B = Brain; L = Lung; Lv = liver;
K = kidney; M = muscle; S = spleen; I = intestine):

| Sequence | | | | Screen | Sequence | | | | Screen |
|---|---|---|---|---|---|---|---|---|---|
| Amino | | | | Identified | Amino | | | | Identified |
| Acid | Scaffold | Log2FC | Organ | In | Acid | Scaffold | Log2FC | Organ | In |
| QQVSSPHTKAPL | AAV9 | 8.3 | B | 2° | TQNKRANRTEE | AAV9 | 5.11 | L | NHP |
| HKDSANGF | Loop1 | | | | NVSDGSPTA | Loop2 | | | |
| (SEQ ID NO: 870) | | | | | (SEQ ID NO: 891) | | | | |
| VAGHFESQSDD | AAV9 | 8.79 | B | 2° | FGPVLDNATRK | AAV9 | 4.69 | L | 2° |
| PHFRGKKQL | Loop 1 | | | | SASVHAIAS | Loop1 | | | |
| (SEQ ID NO: 871) | | | | | (SEQ ID NO: 892) | | | | |
| GVHAKSSDWGL | AAV9 | 11.14 | B | 1° | DNRTRQHIQYR | AAV9 | 7.12 | L | 2° |
| QAGYIPVNP | Loop1 | | | | EDKNLTGTA | Loop2 | | | |
| (SEQ ID NO: 872) | | | | | (SEQ ID NO: 893) | | | | |
| RWYGQDNTCEE | AAV9 | 5.34 | B | 1° | KPQVTRGDVFT | AAV9 | 5.08 | L | NHP |
| YHTVGPHSC | Loop 1 | | | | MSEDEHTVY | Loop2 | | | |
| (SEQ ID NO: 873) | | | | | (SEQ ID NO: 894) | | | | |
| FGSATYDDEHH | AAV9 | 6.55 | B | 1° | AGMDASDRQVI | AAV9 | 7.4 | L | 2° |
| STL VSTRHY | Loop1 | | | | ISSNLTRPK | Loop1 | | | |
| (SEQ ID NO: 874) | | | | | (SEQ ID NO: 895) | | | | |
| YLESRSINEKDY | AAV9 | 7.63 | B | 1° | TDRTSLPSANIT | AAV9 | 3.01 | L | 2° |
| HSRRYIDE (SEQ | Loop1 | | | | RSFKNEDL (SEQ | Loop2 | | | |
| ID NO: 875) | | | | | ID NO: 896) | | | | |
| GDMANSSIEFHP | AAV9 | 7.43 | B | 1° | NRDGTVLESNR | AAV9 | 4.22 | L | 3° |
| KPQQQRDV | Loop1 | | | | TNSLRKINS | Loop1 | | | |
| (SEQ ID NO: 876) | | | | | (SEQ ID NO: 897) | | | | |
| PQSWGRGSAGG | AAV9 | 4.7 | H | 2° | NLPGDPTSNATS | AAV9 | 3.76 | L | 1° |
| NTHSPLGVP | Loop 1 | | | | RGAEGPLP (SEQ | Loop2 | | | |
| (SEQ ID NO: 877) | | | | | ID NO: 898) | | | | |
| FKGVDNEDDHG | AAV9 | 2.9 | H | 2° | PMPDQAVPTGS | AAV9 | 4.67 | M | 2° |
| RGHLGRLKK | Loop1 | | | | FMMVNSSRR | Loop1 | | | |
| (SEQ ID NO: 878) | | | | | (SEQ ID NO: 899) | | | | |
| GKGNYAERAGA | AAV9 | 6.32 | H | 2° | QQQSYLDSGIHS | AAV9 | 3.67 | M | 1° |
| GAPAYLEAV | Loop1 | | | | GATTTAPS (SEQ | Loop1 | | | |
| (SEQ ID NO: 879) | | | | | ID NO: 900) | | | | |
| LVSKGPLVQTK | AAV9 | 6.93 | H | 2° | GTQGRLCNKTS | AAV9 | 4.67 | M | 2° |
| GTGASGSFR | Loop 1 | | | | ECMDGCELM | Loop2 | | | |
| (SEQ ID NO: 880) | | | | | (SEQ ID NO: 901) | | | | |
| GFAIGIVGAAGE | AAV9 | 6.4 | H | 2° | DHTGRHDTPRM | AAV9 | 6.91 | M | 2° |
| RGTAQQPR | Loop1 | | | | STSQTPGRA | Loop1 | | | |
| (SEQ ID NO: 881) | | | | | (SEQ ID NO: 902) | | | | |
| ATLERPASSSHA | AAV9 | 6.66 | H | 2° | VAEIPFNSTNKY | AAV9 | 5.03 | M | 2° |
| QGTHLQLR | Loop1 | | | | QLYIHETE (SEQ | Loop2 | | | |
| (SEQ ID NO: 882) | | | | | ID NO: 903) | | | | |
| LATAGSAMGAA | AAV9 | 6.33 | H | 1° | LVQIRSNSSKPD | AAV9 | 6.44 | M | 2° |
| SLTVSAQSR | Loop2 | | | | LMASLLPK | Loop2 | | | |
| (SEQ ID NO: 883) | | | | | (SEQ ID NO: 904) | | | | |
| LGAVGSTMGAG | AAV9 | 4.47 | H | 2° | VLKQGHPDTGIS | AAV9 | 6.17 | M | 2° |
| SLTLTEQAR | Loop 1 | | | | SKAMGIIY (SEQ | Loop1 | | | |
| (SEQ ID NO: 884) | | | | | ID NO: 905) | | | | |
| TAGAAMGAAG | AAV9 | 6.59 | H | 2° | ISNRVLVSNQTG | AAV9 | 7.11 | M | 2° |
| PTLSAQSRSL | Loop 1 | | | | TAFQPDAA | Loop2 | | | |
| (SEQ ID NO: 885) | | | | | (SEQ ID NO: 906) | | | | |
| IGAGVAGGITGS | AAV9 | 7.7 | H | 2° | AMGAGVVGRT | AAV9 | 2.06 | M | 3° |
| ISLANGKS (SEQ | Loop1 | | | | TGSMSLASGK | Loop1 | | | |
| ID NO: 886) | | | | | (SEQ ID NO: 907) | | | | |
| AVGGAIGRVAN | AAV9 | 7.79 | H | 2° | RLTAGTGGREV | AAV9 | 4.29 | P | 2° |
| TIRSGPSNS (SEQ | Loop1 | | | | GARGEPSGI | Loop2 | | | |
| ID NO: 887) | | | | | (SEQ ID NO: 908) | | | | |
| SQGISSSSTSTMP | AAV9 | 9.99 | H | 1° | ILQRGSGTAAV | AAV9 | 5.54 | P | 2° |
| NSSQSSH (SEQ | Loop2 | | | | DFTKKDNTA | Loop2 | | | |
| ID NO: 888) | | | | | (SEQ ID NO: 909) | | | | |
| VKDRSHNVTEN | AAV9 | 4.86 | L | 2° | WGQGKLVSVSP | AAV9 | 5.09 | P | 3° |
| VTQVLSLGA | Loop2 | | | | DSTQGPSVF | Loop1 | | | |
| (SEQ ID NO: 889) | | | | | (SEQ ID NO: 910) | | | | |
| EILELAGNATRD | AAV9 | 6.44 | L | 2° | SNLHDTKASGP | AAV9 | 2.76 | P | 2° |
| SNKTRIIP (SEQ | Loop1 | | | | AHSKEVPTL | Loop2 | | | |
| ID NO: 890) | | | | | (SEQ ID NO: 911) | | | | |

TABLE 5
table of the identified 23 immune-orthogonal AAV
serotypes with their DNA and amino acid (AA) sequences.

| Name | DNA Sequence | AA Sequence |
|---|---|---|
| BT1 | ATGTCTTTTGTTGACCACCCTCCAGATTGGTTGGA | MSFVDHPPDWLESIGDGFREFLGLEAGPP |
| | ATCGATCGGCGACGGCTTTCGTGAATTTCTCGGCC | KPKANQQKQDNARGLVLPGYKYLGPGN |
| | TTGAGGCGGGTCCCCCGAAACCCAAGGCCAATCA | GLDKGDPVNFADEVAREHDLSYQKQLE |
| | ACAGAAGCAAGATAACGCTCGAGGTCTTGTGCTTC | AGDNPYLKYNHADAEFQEKLASDTSFG |
| | CTGGGTACAAGTATCTTGGTCCTGGGAACGGCCTT | GNLGKAVFQAKKRLLEPLGLVETPDKTA |
| | GATAAGGGCGATCCTGTCAATTTTGCTGACGAGGT | PAAKKRPLEQSPQEPDSSSGFGKKGKQP |
| | TGCCCGAGAGCACGACCTCTCCTACCAGAAACAG | ARKRLNFDDEPGAGDGPPPEGPSSGAMS |
| | CTTGAGGCGGGCGATAACCCTTACCTCAAGTACAA | TETEMRAAAGGNGGDAGQGAEGVGNA |
| | CCACGCGGACGCAGAGTTTCAGGAGAAACTCGCT | SGDWHCDSTWSESHVTTTSTRTWVLPTY |
| | TCTGACACTTCTTTTGGAGGAAACCTTGGGAAGGC | NNHLYLRLGTSNASDTFNGFSTPWGYFD |
| | TGTTTTCCAGGCTAAAAAGAGGCTTCTCGAACCTC | FNRFHCHFSPRDWQRLINNHWGLRPKSM |
| | TTGGCCTGGTTGAGACGCCGGATAAAACGGCGCCT | QVRIFNIQVKEVTTSNGETTVSNNLTSTV |
| | GCGGCAAAAAAGAGGCCTCTAGAGCAGAGTCCTC | QIFADSTYELPYVMDAGQEGSLPPFPND |
| | AAGAGCCAGACTCCTCGAGCGGATTTGGCAAAAA | VFMVPQYGYCGLVTGGSSQNQTDRNAF |
| | GGGCAAACAGCCTGCCAGAAAGAGACTCAATTTT | YCLEYFPSQMLRTGNNFEMVYKFENVPF |
| | GACGACGAACCTGGAGCCGGAGACGGGCCTCCCC | HSMYAHSQSLDRLMNPLLDQYLWELQS |
| | CAGAAGGACCATCTTCCGGAGCTATGTCTACTGAG | TTSGGNLNQGTSATNFAKLTKTNFSGYR |
| | ACTGAAATGCGTGCAGCAGCTGGCGGAAATGGTG | KNWLPGPMMKQQRFSKTANQNYKIPQG |
| | GCGATGCGGGACAAGGTGCCGAGGGAGTGGGTAA | GNNSLLHYETRTTLDGRWSNFAPGTAM |
| | TGCCTCCGGTGATTGGCATTGCGATTCCACTTGGT | ATAANDATDFSQAQLIFAGPNITGNTTTD |
| | CAGAGAGCCACGTCACCACCACCTCAACCCGCAC | ANNLMFTSEDELRATNPRDTDLFGHLAT |
| | CTGGGTCCTGCCGACCTACAACAACCACCTGTACC | NQQNATTVPTADDVDGVGVYPGMVWQ |
| | TGCGGCTCGGCACGAGCAACGCCAGCGACACCTT | DRDIYYQGPIWAKIPHTDGHFHPSPLIGG |
| | CAACGGATTCTCCACCCCCTGGGGATACTTTGACT | FGLKSPPPQIFIKNTPVPANPATTFSPARIN |
| | TCAACCGCTTCCACTGCCACTTCTCGCCAAGAGAC | SFITQYSTGQVAVKIEWEIQKERSKRWNP |
| | TGGCAAAGGCTCATCAACAACCACTGGGGGCTGC | EVQFTSNYGAQDSLLWAPDNAGAYKEP |
| | GCCCCAAAAGCATGCAAGTCCGCATCTTCAACATC | RAIGSRYLTNHL (SEQ ID NO: 822) |
| | CAAGTTAAGGAGGTCACGACGTCTAACGGAGAGA | |
| | CGACCGTATCAAACAACCTCACCAGCACGGTCCA | |
| | GATCTTTGCGGACAGCACGTACGAGCTCCCGTACG | |
| | TGATGGATGCGGGTCAGGAGGGCAGCTTGCCCCCT | |
| | TTCCCCAACGACGTGTTCATGGTGCCCCAGTACGG | |
| | GTACTGCGGACTGGTAACCGGAGGCAGCTCTCAA | |
| | AACCAGACAGACAGAAACGCCTTCTACTGTCTGG | |
| | AGTACTTTCCCAGCCAGATGCTGAGAACAGGAAA | |
| | CAACTTTGAGATGGTGTACAAGTTTGAGAACGTGC | |
| | CCTTCCACTCCATGTACGCTCACAGCCAGAGCCTG | |
| | GATAGGCTGATGAACCCGCTGCTGGACCAGTACCT | |
| | GTGGGAACTCCAGTCTACCACATCTGGAGGAAATC | |
| | TCAACCAGGGCACGTCAGCCACAAACTTTGCCAA | |
| | GCTGACCAAAACAAACTTTTCTGGCTACCGCAAAA | |
| | ACTGGCTTCCGGGACCCATGATGAAGCAGCAGAG | |
| | ATTCTCCAAGACTGCCAATCAAAACTACAAGATTC | |
| | CCCAAGGGGGAAATAACAGTCTGCTCCATTATGA | |
| | GACCAGAACTACCCTCGACGGAAGATGGAGCAAT | |
| | TTTGCCCCGGGAACGGCCATGGCAACCGCGGCCA | |
| | ACGATGCCACCGACTTCTCCCAGGCCCAGCTCATC | |
| | TTTGCGGGGCCCAACATCACCGGCAATACCACGAC | |
| | AGACGCCAACAACCTGATGTTCACTTCAGAAGATG | |
| | AACTCAGGGCCACCAACCCCCGGGACACTGACCT | |
| | GTTTGGCCACCTGGCAACCAACCAGCAAAACGCC | |
| | ACCACCGTTCCTACCGCAGACGACGTGGACGGAG | |
| | TCGGCGTGTACCCGGGAATGGTGTGGCAGGACAG | |
| | AGACATTTATTACCAAGGGCCCATCTGGGCCAAGA | |
| | TTCCCCATACGGATGGACACTTTCACCCATCTCCC | |
| | CTCATTGGCGGATTTGGACTGAAAAGCCCGCCTCC | |
| | ACAAATATTCATCAAAAACACCCCTGTACCCGCCA | |
| | ATCCCGCGACGACCTTCTCTCCGGCCAGAATCAAC | |
| | AGCTTCATCACCCAGTACAGCACCGGACAGGTGG | |
| | CTGTCAAAATAGAATGGGAAATCCAGAAGGAGCG | |
| | GTCCAAGAGATGGAACCCAGAGGTCCAGTTCACG | |
| | TCCAACTACGGAGCACAGGACTCGCTTCTCTGGGC | |
| | TCCCGACAACGCCGGAGCCTACAAAGAGCCCAGG | |
| | GCCATTGGATCCCGATACCTCACCAACCACCTCTA | |
| | G (SEQ ID NO: 821) | |
| BT2 | ATGTCTTTTGTTGACCACCCTCCAGATTGGTTGGA | MSFVDHPPDWLESIGDGFREFLGLEAGPP |
| | ATCGATCGGCGACGGCTTTCGTGAATTTCTCGGCC | KPKANQQKQDNARGLVLPGYKYLGPGN |
| | TTGAGGCGGGTCCCCCGAAACCCAAGGCCAATCA | GLDKGDPVNFADEVAREHDLSYQKQLE |
| | ACAGAAGCAAGATAACGCTCGAGGTCTTGTGCTTC | AGDNPYLKYNHADAEFQEKLASDTSFG |
| | CTGGGTACAAGTATCTTGGTCCTGGGAACGGCCTT | GNLGKAVFQAKKRILEPLGLVETPDKTA |
| | GATAAGGGCGATCCTGTCAATTTTGCTGACGAGGT | PAAKKRPLEQSPQEPDSSSGVGKKGKQP |
| | TGCCCGAGAGCACGACCTCTCCTACCAGAAACAG | ARKRLNFDDEPGAGDGPPPEGPSSGAMS |
| | CTTGAGGCGGGCGATAACCCTTACCTCAAGTACAA | TETEMRAAAGGNGGDAGQGAEGVGNA |
| | CCACGCGGACGCAGAGTTTCAGGAGAAACTCGCT | SGDWHCDSTWSESHVTTTSTRTWVLPTY |
| | TCTGACACTTCTTTTGGGGGAAACCTTGGGAAGGC | NNHLYLRLGSSNASDTFNGFSTPWGYFD |
| | TGTTTTCCAGGCTAAAAAGAGGATTCTCGAACCTC | FNRFHCHFSPRDWQRLINNHWGLRPKSM |
| | TTGGCCTGGTTGAGACGCCGGATAAAACGGCGCCT | QVRIFNIQVKEVTTSNGETTVSNNLTSTV |
| | GCGGCAAAAAAGAGGCCTCTAGAGCAGAGTCCTC | QIFADSTYELPYVMDAGQEGSLPPFPND |
| | AAGAGCCAGACTCCTCGAGCGGAGTTGGCAAGAA | VFMVPQYGYCGLVTGGSSQNQTDRNAF |
| | AGGCAAACAGCCTGCCAGAAAGAGACTCAACTTT | YCLEYFPSQMLRTGNNFEMVYKFENVPF |
| | GACGACGAACCTGGAGCCGGAGACGGGCCTCCCC | HSMYAHSQSLDRLMNPLLDQYLWELQS |
| | CAGAAGGACCATCTTCCGGAGCTATGTCTACTGAG | TTSGGTLNQGNSATNFAKLTKTNFSGYR |
| | ACTGAAATGCGTGCAGCAGCTGGCGGAAATGGTG | KNWLPGPMMKQQRFSKTASQNYKIPQG |
| | GCGATGCGGGACAAGGTGCCGAGGGAGTGGGTAA | RNNSLLHYETRTTLDGRWSNFAPGTAM |
| | TGCCTCCGGTGATTGGCATTGCGATTCCACTTGGT | ATAANDATDFSQAQLIFAGPNITGNTTTD |
| | CAGAGAGCCACGTCACCACCACCTCAACCCGCAC | ANNLMFTSEDELRATNPRDTDLFGHLAT |
| | CTGGGTCCTGCCGACCTACAACAACCACCTGTACC | NQQNATTVPTVDDVDGVGVYPGMVWQ |
| | TGCGGCTCGGCTCGAGCAACGCCAGCGACACCTTC | DRDIYYQGPIWAKIPHTDGHFHPSPLIGG |
| | AACGGATTCTCCACCCCCTGGGGATACTTTGACTT | FGLKSPPPQIFIKNTPVPANPATTFSPARIN |
| | TAACCGCTTCCACTGCCACTTCTCGCCAAGAGACT | SFITQYSTGQVAVKIEWEIQKERSKRWNP |
| | GGCAAAGGCTCATCAACAACCACTGGGGACTGCG | EVQFTSNYGAQDSLLWAPDNAGAYKEP |
| | CCCCAAAAGCATGCAAGTCCGCATCTTCAACATCC | RAIGSRYLTNHL (SEQ ID NO: 824) |
| | AAGTTAAGGAGGTCACGACGTCTAACGGGGAGAC | |
| | GACCGTATCCAACAACCTCACCAGCACGGTCCAG | |
| | ATCTTTGCGGACAGCACGTACGAGCTCCCGTACGT | |
| | GATGGATGCAGGTCAGGAGGGCAGCTTGCCTCCTT | |
| | TCCCCAACGACGTGTTCATGGTGCCTCAGTACGGG | |
| | TACTGCGGACTGGTAACCGGAGGCAGCTCTCAAA | |
| | ACCAGACAGACAGAAATGCCTTCTACTGTCTGGAG | |
| | TACTTTCCCAGCCAGATGCTGAGAACCGGAAACA | |
| | ACTTTGAGATGGTGTACAAGTTTGAAAACGTGCCC | |
| | TTCCACTCCATGTACGCTCACAGCCAGAGCCTGGA | |
| | TAGGCTGATGAACCCGCTGCTGGACCAGTACCTGT | |
| | GGGAGCTCCAGTCTACCACCTCTGGAGGAACTCTC | |
| | AACCAGGGCAATTCAGCCACCAACTTTGCCAAGCT | |
| | GACCAAAACAAACTTTTCTGGCTACCGCAAAAACT | |
| | GGCTCCCGGGGCCCATGATGAAGCAGCAGAGATT | |
| | CTCCAAGACTGCCAGTCAAAACTACAAGATTCCCC | |
| | AGGGAAGAAACAACAGTCTGCTCCATTATGAGAC | |
| | CAGAACTACCCTCGACGGAAGATGGAGCAATTTT | |
| | GCCCCGGGAACGGCCATGGCAACCGCAGCCAACG | |
| | ACGCCACCGACTTCTCTCAGGCCCAGCTCATCTTT | |
| | GCGGGGCCCAACATCACCGGCAACACCACCACAG | |
| | ATGCCAATAACCTGATGTTCACTTCAGAAGATGAA | |
| | CTTAGGGCCACCAACCCCCGGGACACTGACCTGTT | |
| | TGGCCACCTGGCAACCAACCAGCAAAACGCCACC | |
| | ACCGTTCCTACCGTAGACGACGTGGACGGAGTCG | |
| | GCGTGTACCCGGGAATGGTGTGGCAGGACAGAGA | |
| | CATTTACTACCAAGGGCCCATTTGGGCCAAAATTC | |
| | CACACACGGATGGACACTTTCACCCGTCTCCTCTC | |
| | ATTGGCGGATTTGGACTGAAAAGCCCGCCTCCACA | |
| | AATATTCATCAAAAACACTCCTGTACCCGCCAATC | |
| | CCGCAACGACCTTCTCTCCGGCCAGAATCAACAGC | |
| | TTCATCACCCAGTACAGCACCGGACAGGTGGCTGT | |
| | CAAAATAGAATGGGAAATCCAGAAGGAGCGGTCC | |
| | AAGAGATGGAACCCAGAGGTCCAGTTCACGTCCA | |
| | ACTACGGAGCACAGGACTCGCTTCTCTGGGCTCCC | |
| | GACAACGCCGGAGCCTACAAAGAGCCCAGGGCCA | |
| | TTGGATCCCGATACCTCACCAACCACCTCTAG | |
| | (SEQ ID NO: 823) | |
| MG2 | ATGTCGTTCTTTGATTATCTAGGTAAAAAATACGC | MSFFDYLGKKYAQGAADWWDAKAGPP |
| | TCAAGGAGCCGCGGATTGGTGGGACGCGAAAGCA | PPKKAKGKGFVFPGHNYLGPGNDLDNG |
| | GGACCACCTCCTCCGAAAAAGGCCAAAGGGAAAG | KPVDADDAAAKKHDEAYDEQLKQGDN |
| | GCTTCGTCTTTCCCGGACACAACTACCTAGGTCCC | PYVKYNHADAQLQEDLKDVNTFEANLA |
| | GGCAACGACCTAGACAACGGTAAACCCGTTGACG | KGVFQAKKRVLEPFGLVEELAAKKRPHS |
| | CGGACGACGCTGCCGCCAAAAAGCACGACGAAGC | EIESPDSSSGIGKKGDQPARRRLDFDQED |
| | TTACGACGAGCAGCTCAAACAGGGCGACAACCCG | RQQAAQDGATGTGSLEPSSNLGSDTMST |
| | TACGTGAAATATAATCACGCGGACGCCCAGCTGC | GGGAPMAGDNQGADGVGNASGNWHCD |
| | AAGAAGACCTCAAAGACGTCAATACCTTTGAGGC | TTWMGDRVVTRSTRTWLLPSYNNHVYR |
| | CAATCTCGCCAAAGGAGTCTTCCAGGCCAAGAAA | RINAGSNSNAFFGFSTPWGYFDYNRFHC |
| | CGGGTCCTCGAACCGTTTGGTCTGGTAGAAGAACT | HFSPRDWQRLVNNNWGIRPKHLKFKLFN |
| | GGCCGCAAAGAAGCGACCTCACTCTGAGATCGAA | IQVKEYTVQEGGGETVANNLTSTVQVFT |
| | TCGCCCGATTCCAGCTCGGGAATCGGCAAGAAGG | DSDYQLPYVLGSAQEGAMPPFPADVFML |
| | GAGACCAACCCGCACGACGTCGGTTGGACTTTGAC | PQYAYCTRONGTNPTPRSAFYCLEYFPS |
| | CAGGAAGACAGACAACAAGCCGCTCAAGACGGAG | QMLRTGNNFDFSYEFENVPFHSMWAHN |
| | CCACCGGTACCGGAAGTCTCGAGCCGTCCTCAAAT | QTLDRLMNPLVDQYLWRLQNVANVSGT |
| | TTGGGATCTGATACGATGTCTACAGGCGGTGGCGC | KTNQFSQNVAGDLQLFTRNWLPGPAYR |
| | TCCAATGGCAGGAGATAACCAAGGCGCCGATGGA | TQSWSTNTAQNATQTNIPNQENVYFLEG |
| | GTGGGTAATGCCTCAGGAAATTGGCATTGCGATAC | RTNTIAPGMAMGPTTATHFQSNGVIFAK |
| | CACATGGATGGGAGACCGAGTGGTCACCCGCTCC | NESGTPATGANINQLNITRESEVNSTNPL |
| | ACTCGCACCTGGTTGCTGCCCAGCTACAACAACCA | LSRPPLAITNNEQTAAATPDRANWNLAP |
| | CGTGTACAGAAGAATCAACGCCGGATCAAACAGC | VMPGQVWMDKDIYLQGQIWAKIPHTDG |
| | AACGCCTTCTTCGGCTTCAGCACCCCGTGGGGGTA | HFHPSPLMGGFGLKHPPPQIFIKNTPVPGS |
| | CTTTGACTACAACAGATTCCACTGCCACTTTTCTCC | PAPNFTVDKVNKFINQYSTGQVTVEIEW |
| | ACGAGACTGGCAACGACTCGTCAACAACAACTGG | ELQKESSKRWNPEIQYTDDASATDGSILH |
| | GGCATTCGACCCAAGCACCTCAAGTTCAAACTCTT | FAPNDEGLYTQFRSIGTRYLTRPL (SEQ |
| | CAACATACAAGTTAAAGAATACACGGTCCAGGAG | ID NO: 826) |
| | GGCGGAGGCGAGACCGTCGCCAATAACCTCACAA | |
| | GCACCGTGCAGGTCTTTACGGACTCGGACTATCAG | |
| | CTACCGTACGTGCTAGGTTCGGCGCAAGAGGGTGC | |
| | CATGCCACCGTTCCCCGCCGACGTGTTTATGTTGC | |
| | CTCAATACGCGTACTGCACTCGGCAGAACGGAAC | |
| | CAACCCGACGCCCAGAAGCGCCTTTTACTGTCTGG | |
| | AATACTTTCCCAGTCAGATGCTGAGAACCGGCAAC | |
| | AACTTTGACTTCTCGTACGAGTTCGAGAACGTGCC | |
| | CTTCCACAGTATGTGGGCTCACAACCAGACTCTTG | |
| | ATCGTCTGATGAATCCTCTGGTGGATCAATACTTG | |
| | TGGAGACTACAAAATGTAGCTAACGTAAGCGGAA | |
| | CAAAGACCAATCAGTTCAGCCAGAACGTGGCCGG | |
| | CGACCTACAACTGTTTACTCGGAACTGGCTACCCG | |
| | GGCCCGCTTACAGAACGCAGTCTTGGTCGACCAAC | |
| | ACGGCTCAGAACGCGACTCAGACCAACATCCCGA | |
| | ATCAGGAAAACGTCTACTTTCTGGAAGGAAGAAC | |
| | TAACACCATCGCACCCGGCATGGCCATGGGGCCC | |
| | ACAACCGCTACACACTTTCAATCCAACGGAGTGAT | |
| | ATTCGCCAAGAACGAATCGGGTACTCCGGCAACT | |
| | GGTGCCAACATTAACCAGTTGAACATCACACGGG | |
| | AATCAGAAGTCAACTCGACCAATCCTCTGCTGTCA | |
| | AGACCACCCCTGGCTATTACCAACAACGAGCAGA | |
| | CCGCAGCTGCCACGCCAGACAGAGCCAACTGGAA | |
| | TCTGGCTCCCGTCATGCCCGGACAAGTCTGGATGG | |
| | ACAAAGACATCTACCTGCAAGGCCAGATCTGGGC | |
| | CAAGATCCCACACACGGACGGACACTTTCATCCGT | |
| | CTCCTCTCATGGGAGGATTCGGACTGAAACACCCT | |
| | CCACCTCAAATCTTCATCAAGAACACTCCCGTACC | |
| | CGGATCACCCGCTCCCAACTTTACCGTGGACAAGG | |
| | TCAACAAGTTCATCAACCAATACTCTACCGGTCAA | |
| | GTAACCGTGGAGATTGAATGGGAACTGCAGAAGG | |
| | AATCGTCCAAGAGATGGAATCCAGAGATCCAATA | |
| | CACGGACGACGCATCCGCCACCGACGGATCTATCC | |
| | TGCACTTTGCTCCCAACGACGAAGGATTGTACACC | |
| | CAGTTCCGATCTATCGGGACTCGGTACCTCACCCG | |
| | CCCTCTGTAA (SEQ ID NO: 825) | |
| ZC | ATGTCTTCTTTGTTTAAAGAGTACCTGCAGAGTAC | MSSLFKEYLQSTGLVGIQSGAPKPKAGQ |
| | CGGACTGGTGGGTATCCAGTCGGGAGCGCCGAAG | QKQDTGSFEWKKKEDTARGLVLPGYKY |
| | CCAAAGGCCGGCCAGCAGAAGCAGGATACAGGGT | LGPFNGLERGEPVNAADAAAQRHDRQY |
| | CCTTTGAGTGGAAAAAGAAGGAGGACACAGCCAG | DRILQQGGNPYLTYNHADREFQEELQSD |
| | AGGGCTGGTCCTTCCCGGTTACAAGTACCTCGGGC | ESFGGGNLGKAVFQAKKRILEPLGLVEEDP |
| | CCTTCAACGGTCTAGAGCGGGGCGAGCCTGTGAAT | SQPDSSSDNTAPPVKKSRLEEAQPIQSPD |
| | GCCGCGGACGCCGCGGCCCAGCGACACGACCGAC | VSSSTGGGIADVMSGDAEMAAVGGGAP |
| | AGTACGATCGTATTCTACAACAAGGGGGTAATCCA | GVDGQGAEGVGTSSGNWHCDSQWSEG |
| | TACCTCACGTACAACCACGCCGACCGAGAGTTCCA | HVRTTSTRTWVLPSYNNHLYKRLGSSAQ |
| | GGAGGAGCTCCAGTCTGACGAGTCTTTTGGGGGA | SNTYNGFSTPWGYLDFNRWHCHFSPRN |
| | AATCTTGGCAAGGCTGTGTTTCAGGCCAAGAAGCG | WQRLINNNWGIRPKRLNVKLFNIQVKEV |
| | AATCCTAGAGCCCCTCGGTCTGGTTGAAGAGGATC | TTEGGTTTVANNLTSTIQVFADNAYELP |
| | CCTCTCAGCCAGATTCGTCCTCGGACAACACGGCG | YVVDAGHEGALPPFPNDVFMIPQYGYCG |
| | CCACCCGTGAAAAAGTCCCGTCTCGAAGAAGCCC | LVSGQSQAQSDLCSFYCLEYFPSQMLRT |
| | AGCCCATTCAAAGTCCAGACGTTTCCAGCAGCACT | GNNFEMNFRFEDVPFHSMYAHSQSLDRL |
| | GGCGGAGGAATTGCCGACGTCATGTCTGGAGATG | MNPLIDQYLWSLKQTGNPTGSTTRDLKFI |
| | CGTCGACGGCCAGGGTGCCGAGGGAGTGGGTACT | KNKVPNFAHYGKNWLPGPFIRQQGWTT |
| | ACGGCTTGGAAGTAGCGCACAATCCAATACCTAC | QNINNSVVNFNDMLGKNSTFTLDTRWSS |
| | TCCTCGGGTAATTGGCATTGCGATTCCCAGTGGTC | LAPGPCMGDDGRTPSTTKFSNAQLMFGS |
| | AGAAGGACACGTCAGAACCACCAGCACCAGAACC | GTQPTEGGEDAVHITSESEVKATNPTAID |
| | TGGGTGTTGCCCAGCTACAACAACCACCTGTATAA | EYGRVADNTQNATTAPTTVGNAAMGA |
| | ACGGCTTGGAAGTAGCGCACAATCCAATACCTAC | MPGMVWQDRDIYLQGPIWGKIPHTDGH |
| | AACGGATTCTCCACCCCCTGGGGATACCTCGACTT | FHPSPLMGGFGYRKPPPQIFIKNTPVPGNP |
| | CAATAGATGGCACTGTCACTTCAGTCCTCGGAACT | ATTFSPNRINNFITQYSTGQVTVTIDWEL |
| | GGCAACGTCTCATCAACAACAACTGGGGCATCAG | QKENSKRWNPEVQFTSNFGTVDSLNWA |
| | ACCAAAAAGACTTAATGTTAAATTGTTCAACATAC | PDNAGNYKEPRVVGSRFTHIL (SEQ |
| | AAGTCAAAGAGGTCACGACGGAAGGGGGGACGAC | ID NO: 828) |
| | GACCGTCGCCAATAACCTTACCAGCACGATTCAGG | |
| | TGTTTGCGGACAACGCGTACGAACTCCCGTATGTT | |
| | GTCGACGCGGGTCACGAGGGGGCATTGCCGCCGT | |
| | TCCCAAACGACGTGTTTATGATTCCCCAATACGGA | |
| | TACTGCGGGCTGGTGTCTGGTCAAAGTCAGGCTCA | |
| | GTCGGACTTGTGTTCGTTTTATTGCCTGGAGTATTT | |
| | TCCATCCCAGATGCTGAGAACAGGAAACAATTTTG | |
| | AAATGAATTTCAGGTTTGAAGACGTTCCATTTCAC | |
| | TCCATGTACGCCCACAGCCAGTCTCTAGACAGGCT | |
| | CATGAATCCTTTAATTGATCAGTATCTGTGGAGTC | |
| | TAAAACAAACAGGAAACCCGACTGGATCAACAAC | |
| | GAGAGACTTAAAATTTATCAAGAACAAAGTTCCC | |
| | AATTTTGCACATTATGGAAAAAATTGGCTTCCTGG | |
| | ACCTTTTATTAGACAACAGGGGTGGACAACACAA | |
| | AATATTAATAATAGTGTTGTTAATTTTAATGACAT | |
| | GCTGGGAAAAAATTCGACATTTACTTTGGACACTA | |
| | GATGGAGTTCATTAGCGCCTGGTCCGTGTATGGGG | |
| | GATGACGGACGAACTCCATCCACCACCAAGTTCTC | |
| | AAATGCTCAGCTCATGTTTGGATCTGGAACACAAC | |
| | CCACCGAAGGCGGTGAAGATGCTGTACATATTAC | |
| | ATCCGAGTCGGAGGTCAAGGCAACCAACCCAACT | |
| | GCAATCGATGAATACGGACGAGTGGCCGATAATA | |
| | CGCAAAATGCAACAACCGCCCCAACCACAGTGGG | |
| | AAATGCTGCAATGGGGGCCATGCCTGGGATGGTG | |
| | TGGCAAGATAGGGATATCTATCTTCAAGGACCCAT | |
| | CTGGGGAAAAATACCCCATACAGACGGACATTTTC | |
| | ATCCGTCTCCTCTCATGGGGGGGTTTGGATACAGA | |
| | AAACCCCCTCCGCAAATTTTTATTAAAAACACCCC | |
| | CGTTCCTGGAAATCCGGCAACTACATTTTCTCCAA | |
| | ATAGAATAAACAATTTCATTACTCAATACTCAACG | |
| | GGACAAGTGACCGTTACTATTGACTGGGAGCTGCA | |
| | AAAGGAAAACTCAAAGAGATGGAACCCAGAAGTA | |
| | CAATTTACATCAAATTTTGGCACGGTCGATTCACT | |
| | AAACTGGGCACCGGACAACGCGGGAAACTACAAA | |
| | GAACCAAGGGTGGTGGGGTCTCGATTCCTTACGCA | |
| | TATACTATAA (SEQ ID NO: 827) | |
| MA1 | ATGTCTTTTTTAGAAGCGTTTGAGGACTGGTACGA | MSFLEAFEDWYEKAAATWRHLEPGAPR |
| | AAAAGCAGCGGCTACTTGGCGACATCTCGAACCT | PKANQQHQEQHHPVPAEEPKDQARGLV |
| | GGCGCCCCCCGTCCTAAAGCCAACCAGCAGCATC | LPGYKYLGPFNGLDKGDPVNQADAAAL |
| | AGGAACAGCATCATCCGGTTCCTGCTGAAGAGCCT | EHDKAYNHLLEEGQNPYLVYNEADKHF |
| | AAAGATCAAGCTCGAGGCCTTGTGCTACCTGGCTA | QETLQGDTSFGGNLGKAVFQGKKRVLEP |
| | CAAGTACCTCGGCCCCTTTAACGGCCTTGATAAAG | FGLVDEEPHELAPPPKKKTKQEYKDTLE |
| | GTGATCCGGTTAATCAAGCGGACGCTGCGGCGCTT | AIPPPSREEQTPQKGSSASKNGASSSAAA |
| | GAACACGATAAAGCGTACAACCACCTTCTCGAAG | AAPSNLGSGIMAEGGGAPMGDNQQGAD |
| | AAGGACAAAACCCGTACCTCGTGTATAACGAGGC | GVGNASGNWHCDSHWMGDKVVTRTTR |
| | TGATAAGCATTTCCAGGAAACTCTACAGGGGGAT | TWVLPTYNNHLYQAIDENTTLGSANNFY |
| | ACGAGCTTCGGCGGTAACCTCGGCAAAGCAGTCTT | GFSTPWGYFDFNRFHCHFSPRDWQRLIN |
| | CCAAGGCAAGAAACGAGTCCTAGAGCCCTTTGGA | NNWGIRPRSLRFKLFNIQVKEVTTTSGET |
| | CTCGTTGACGAGGAGCCTCACGAACTGGCTCCACC | TIANNLTSTVQVFADEEMHLPYVLGSAH |
| | TCCAAAAAAGAAGACAAAACAGGAATACAAAGAC | DGCFPPFPPDIYMLPQYGYCTLDYQGLTT |
| | ACTCTAGAGGCTATTCCTCCTCCTAGCAGAGAGGA | DRSAFYCLEYFPSQMLRTGNNFELTYNF |
| | ACAGACTCCTCAGAAGGGATCTTCAGCATCCAAA | EKLPFHSMWMHNQSLDRIMNPLIDQYLY |
| | AACGGAGCCTCATCTTCAGCAGCAGCAGCAGCCC | RFASKTGNTWTYQKGSKDDKTAQARN |
| | CCAGTAATTTGGGATCTGGTATCATGGCAGAAGGC | WLPGPSIRNQPLYSSGNQTNLSGYDIAPK |
| | GGTGGCGCACCAATGGGCGACAATCAACAGGGTG | TEVNEKQMTVFPGLSMATENERSGTIKE |
| | CCGATGGAGTGGGTAATGCCTCGGGAAATTGGCA | QPTSNLLIFANKADAATSQTTSSVGSTAT |
| | TTGCGATTCCCACTGGATGGGCGACAAAGTCGTCA | LLVTNENEVKTVNPSATEAWGSAAANK |
| | CCCGCACCACCAGAACCTGGGTCCTGCCCACCTAC | QTSATASQSVAIHAQGVVPGMVWQDRD |
| | AACAACCACCTCTACCAAGCCATCGACGAGAATA | IYFQGPIWAKIPHTDGHFHPSPLMGGFGL |
| | CTACTCTCGGATCCGCCAACAACTTCTACGGATTC | KHPPPQIFIKNTPVPSNPATTYTTAKYNSF |
| | AGCACCCCATGGGGGTACTTTGACTTTAACCGCTT | ITQYSTGQVTVEIDWELQKETSKKWNPEI |
| | CCACTGCCACTTCTCCCCACGTGACTGGCAAAGAC | QFTSNYNGTDTLGFAPDAAGDYTEPRAI |
| | TCATCAACAACAACTGGGGCATCCGACCCAGAAG | GTRYLTRPL (SEQ ID NO: 830) |
| | CCTCAGATTCAAGCTGTTCAACATCCAAGTCAAGG | |
| | AGGTCACGACGACGTCGGGCGAAACGACCATCGC | |
| | CAATAACCTTACCAGCACGGTTCAGGTTTTTGCGG | |
| | ATGAAGAAATGCACCTCCCGTACGTCTTAGGGAGT | |
| | GCCCACGACGGGTGCTTTCCTCCATTTCCACCAGA | |
| | CATTTACATGCTGCCCCAATACGGGTACTGCACTC | |
| | TGGACTATCAGGGACTCACCACAGACAGGAGCGC | |
| | CTTCTACTGTCTGGAGTACTTCCCTTCTCAGATGCT | |
| | GAGAACGGGCAACAACTTTGAGCTTACTTACAACT | |
| | TTGAGAAGCTGCCTTTCCACAGCATGTGGATGCAC | |
| | AACCAGTCCCTGGACAGGATCATGAATCCTCTCAT | |
| | CGATCAGTACCTGTATCGCTTCGCCTCCAAGACTG | |
| | GCAACACATGGACATACCAAAAAGGCAGCAAAGA | |
| | CGACAAGACCGCTCAAGCCAGAAACTGGCTTCCT | |
| | GGACCCTCCATCAGAAATCAGCCTCTGTACTCGAG | |
| | TGGTAATCAAACCAACCTCTCGGGATACGATATCG | |
| | CTCCAAAAACAGAAGTCAACGAGAAACAGATGAC | |
| | TGTGTTTCCTGGACTTTCTATGGCCACCGAAAACG | |
| | AGAGGAGCGGAACCATCAAAGAGCAGCCAACGTC | |
| | CAATCTGCTCATCTTTGCCAACAAAGCGGATGCGG | |
| | CTACCTCTCAGACCACTTCTAGCGTGGGCAGTACA | |
| | GCGACACTCCTGGTCACCAACGAAAACGAAGTCA | |
| | AGACTGTCAATCCCTCCGCCACCGAAGCGTGGGGT | |
| | TCGGCTGCGGCCAACAAACAGACATCTGCTACCGC | |
| | CTCTCAAAGCGTGGCTATTCACGCTCAAGGCGTGG | |
| | TGCCAGGCATGGTCTGGCAAGACAGAGACATCTA | |
| | CTTTCAGGGCCCCATCTGGGCCAAGATCCCGCATA | |
| | CAGATGGACACTTTCACCCATCTCCTCTCATGGGT | |
| | GGATTCGGACTCAAACATCCTCCTCCACAAATTTT | |
| | CATTAAAAACACTCCTGTCCCCTCCAATCCTGCTA | |
| | CCACGTACACCACCGCCAAGTACAACTCCTTCATC | |
| | ACCCAGTACTCCACCGGTCAAGTGACTGTGGAGAT | |
| | TGACTGGGAACTCCAGAAGGAGACCTCCAAAAAG | |
| | TGGAACCCCGAGATCCAGTTCACCTCCAATTACAA | |
| | CGGGACAGACACGCTCGGATTCGCTCCAGATGCC | |
| | GCTGGAGACTACACCGAACCACGTGCCATCGGAA | |
| | CCCGATACCTTACCCGACCCCTTTAA | |
| | (SEQ ID NO: 829) | |
| CH1 | ATGTCTTTTGTTGATCACCCTCCAGATTGGTTGGA | MSFVDHPPDWLEEVGEGLREFLGLEAGP |
| | AGAAGTTGGTGAAGGTCTTCGCGAGTTTTTGGGCC | PKPKPNQQHQDQARGLVLPGYNYLGPG |
| | TTGAAGCGGGCCCACCGAAACCAAAACCCAATCA | NGLDRGEPVNRADEVAREHDISYNEQLE |
| | GCAGCATCAAGATCAAGCCCGTGGTCTTGTGCTGC | AGDNPYLKYNHADAEFQEKLADDTSFG |
| | CTGGTTATAACTATCTCGGACCCGGAAACGGTCTC | GNLGKAVFQAKKRVLEPFGLVEEGAKT |
| | GATCGAGGAGAGCCTGTCAACAGGGCAGACGAGG | APTGKRIDDHFPKRKKARTEEDSKPSTSS |
| | TCGCGCGAGAGCACGACATCTCGTACAACGAGCA | DAEAGPSGSQQLQIPAQPASSLGADTMS |
| | GCTTGAGGCGGGAGACAACCCCTACCTCAAGTAC | AGGGGPLGDNNQGADGVGNASGDWHC |
| | AACCACGCGGACGCCGAGTTTCAGGAGAAGCTCG | DSTWMGDRVVTKSTRTWVLPSYNNHQY |
| | CCGACGACACATCCTTCGGGGGAAACCTCGGAAA | REIKSGSVDGSNANAYFGYSTPWGYFDF |
| | GGCAGTCTTTCAGGCCAAGAAAAGGGTTCTCGAA | NRFHSHWSPRDWQRLINNYWGFRPRSLR |
| | CCTTTTGGCCTGGTTGAAGAGGGTGCTAAGACGGC | VKIFNIQVKEVTVQDSTTTIANNLTSTVQ |
| | CCCTACCGGAAAGCGGATAGACGACCACTTTCCA | VFTDDDYQLPYVVGNGTEGCLPAFPPQV |
| | AAAAGAAAGAAGGCTCGGACCGAAGAGGACTCCA | FTLPQYGYATLNRDNGDNPTERSSFFCLE |
| | AGCCTTCCACCTCGTCAGACGCCGAAGCTGGACCC | YFPSKMLRTGNNFEFTYSFEEVPFHCSFA |
| | AGCGGATCCCAGCAGCTGCAAATCCCAGCACAAC | PSQNLFKLANPLVDQYLYRFVSTSATGAI |
| | CAGCCTCAAGTTTGGGAGCTGATACAATGTCTGCG | QFQKNLAGRYANTYKNWFPGPMGRTQG |
| | GGAGGTGGCGGCCCATTGGGCGACAATAACCAAG | WNTSSGSSTNRVSVNNFSVSNRMNLEGA |
| | GTGCCGATGGAGTGGGCAATGCCTCGGGAGATTG | SYQVNPQPNGMTNTLQGSNRYALENTMI |
| | GCATTGCGATTCCACGTGGATGGGGGACAGAGTC | FNAQNATPGTTSVYPEDNLLLTSESETQP |
| | GTCACCAAGTCCACCCGCACCTGGGTGCTGCCCAG | VNRVAYNTGGQMATNAQNATTAPTVGT |
| | CTACAACAACCACCAGTACCGAGAGATCAAAAGC | YNLQEVLPGSVWMERDVYLQGPIWAKIP |
| | GGCTCCGTCGACGGAAGCAACGCCAACGCCTACTT | ETGAHFHPSPAMGGFGLKHPPPMMLIKN |
| | TGGATACAGCACCCCCTGGGGGTACTTTGACTTTA | TPVPGNITSFSDVPVSSFITQYSTGQVTVE |
| | ACCGCTTCCACAGCCACTGGAGCCCCCGAGACTGG | MEWELKKENSKRWNPEIQYTNNYNDPQ |
| | CAAAGACTCATCAACAACTATTGGGGCTTCAGACC | FVDFAPDGSGEYRTTRAIGTRYLTRPL |
| | CCGGTCTCTCAGAGTCAAAATCTTCAACATCCAAG | (SEQ ID NO: 832) |
| | TCAAAGAGGTCACGGTGCAGGACTCCACCACCAC | |
| | CATCGCCAACAACCTCACCTCCACCGTCCAAGTGT | |
| | TTACGGACGACGACTACCAACTCCCGTACGTCGTC | |
| | GGCAACGGGACCGAGGGATGCCTGCCGGCCTTCC | |
| | CCCCGCAGGTCTTTACGCTGCCGCAGTACGGCTAC | |
| | GCGACGCTGAACCGAGACAACGGAGACAACCCGA | |
| | CAGAGCGGAGCAGCTTCTTTTGCCTAGAGTACTTT | |
| | CCCAGCAAGATGCTGAGGACGGGCAACAACTTTG | |
| | AGTTTACCTACAGCTTTGAAGAGGTGCCCTTCCAC | |
| | TGCAGCTTCGCCCCGAGCCAGAACCTCTTTAAGCT | |
| | GGCCAACCCGCTGGTGGACCAGTACCTGTACCGCT | |
| | TCGTGAGCACCTCGGCCACGGGCGCCATCCAGTTC | |
| | CAAAAGAACCTGGCGGGCAGATACGCCAACACCT | |
| | ACAAAAACTGGTTCCCGGGGCCCATGGGCCGAAC | |
| | CCAGGGCTGGAACACGAGCTCTGGCAGCAGCACC | |
| | AACAGAGTCAGCGTCAACAACTTTTCCGTCTCAAA | |
| | CCGGATGAACCTGGAGGGGGCCAGCTACCAAGTG | |
| | AACCCCCAGCCCAACGGGATGACAAACACGCTCC | |
| | AAGGCAGCAACCGCTACGCGCTGGAAAACACCAT | |
| | GATCTTCAACGCTCAAAACGCCACGCCGGGAACT | |
| | ACCTCGGTGTACCCAGAGGACAATCTACTGCTGAC | |
| | CAGCGAGAGCGAGACTCAGCCCGTCAACCGGGTG | |
| | GCTTACAACACGGGCGGTCAGATGGCCACCAACG | |
| | CCCAGAACGCCACCACGGCTCCCACGGTCGGGAC | |
| | CTACAACCTCCAGGAAGTGCTTCCTGGCAGCGTAT | |
| | GGATGGAGAGGGACGTGTACCTCCAAGGACCCAT | |
| | CTGGGCCAAGATCCCAGAGACGGGGGCGCACTTT | |
| | CACCCCTCTCCGGCCATGGGCGGATTCGGACTCAA | |
| | ACACCCGCCGCCCATGATGCTCATCAAAAACACGC | |
| | CGGTGCCCGGCAACATCACCAGCTTCTCGGACGTG | |
| | CCCGTCAGCAGCTTCATCACCCAGTACAGCACCGG | |
| | GCAGGTCACCGTGGAGATGGAATGGGAGCTCAAA | |
| | AAGGAAAACTCCAAGAGGTGGAACCCAGAGATCC | |
| | AGTACACCAACAACTACAACGACCCCCAGTTTGTG | |
| | GACTTTGCTCCAGACGGCTCCGGCGAATACAGAAC | |
| | CACCAGAGCCATCGGAACCCGATACCTCACCCGA | |
| | CCCCTTTAA (SEQ ID NO: 831) | |
| SD1 | ATGTCGTTTGTTGATCACCCTCCAGATTGGCTTGA | MSFVDHPPDWLEEIGEGLKEFLGLEPGPP |
| | GGAGATTGGTGAGGGTCTAAAGGAGTTTTTGGGA | KPKPNQQKQDDARGLVLPGYNYLGPGN |
| | CTCGAACCTGGCCCACCCAAACCGAAGCCCAACC | GLDRGEPVNRADEVAREHDISYNEQLQA |
| | AGCAGAAGCAAGACGACGCCCGTGGTCTTGTACT | GDNPYLKYNHADAEFQEKLADDTSFGG |
| | GCCTGGATATAATTACCTGGGACCCGGAAACGGTC | NLGKAVFQAKKRVLEPFGLVEEPVKTAA |
| | TCGACCGCGGAGAACCTGTCAACCGGGCTGACGA | KGERIDDHYPKKKKARIEETEAGTSGAQ |
| | GGTCGCGCGAGAGCACGACATCTCGTACAACGAG | QLQIPAQPASSLGADTMSAGGGSPLGDN |
| | CAGCTCCAGGCGGGAGACAACCCCTACCTCAAGT | NQGADGVGNASGDWHCDSTWMGDRVI |
| | ACAACCACGCGGACGCCGAGTTTCAGGAGAAGCT | TKSTRTWVLPSYNNHQYLEIHSGSVDGS |
| | CGCGGACGACACCTCCTTCGGGGGCAACCTCGGC | NANAYFGYSTPWGYFDFNRFHSHWSPR |
| | AAGGCAGTCTTTCAGGCCAAAAAAAGGGTTCTCG | DWQRLVNNYWGFRPRSLKVKIFNIQVKE |
| | AACCTTTTGGCCTGGTTGAGGAGCCTGTTAAGACG | VTTQDGTTTIANNLTSTVQVFTDNDYQL |
| | GCTGCTAAAGGCGAGCGGATAGACGACCACTATC | PYVIGNGTEGCLPAFPPQVFTLPQYGYAT |
| | CCAAAAAGAAGAAGGCTCGGATCGAAGAGACCGA | LNRNNTDDPTERSSFFCLEYFPSKMLRTG |
| | AGCTGGAACCAGCGGAGCCCAGCAGCTGCAGATC | NNFEFTYSFEEVPFHCSFAPSQNLFKLAN |
| | CCAGCCCAACCAGCCTCAAGTTTGGGAGCTGATAC | PLVDQYLYRFVSTDTSGNLQFQKNLKAR |
| | AATGTCTGCGGGAGGTGGCAGCCCACTGGGCGAC | YANTYKNWFPGPMCRTQGWYTSAGTY |
| | AATAACCAAGGCGCCGATGGAGTGGGCAATGCCT | NNKGVANFDTSNKMELEGASYQVNPQP |
| | CGGGAGATTGGCATTGCGATTCCACGTGGATGGG | NGMTNTLQDSNKYALENTMIFNAQNAP |
| | GGACCGAGTCATCACCAAGTCCACCCGAACCTGG | PGTTSLYQENNLLITSESETQPVNRLAYN |
| | GTGCTGCCCAGCTACAACAACCATCAGTACCTTGA | TGGQVSNNNQNSNTHPTVGVYNHQEVL |
| | GATCCACAGCGGTTCCGTCGACGGAAGCAACGCT | PGSVWMDRDVYLQGPIWAKIPETGAHF |
| | AACGCTTATTTTGGATACAGCACCCCCTGGGGGTA | HPSPAMGGFGLKHPPPMMLIKNTPVPSN |
| | CTTTGACTTCAACCGCTTCCACAGCCACTGGAGCC | VAAFSDVPVKSFITQYSTGQVTVEIEWEL |
| | CCCGAGACTGGCAGCGACTCGTCAACAACTACTG | KKENSKRWNPEIQYTNNYNNPTYFVDFAP |
| | GGGATTCAGACCCCGGTCCCTCAAGGTCAAGATCT | DTSGEYRTTRAIGTRYLTRPL (SEQ ID |
| | TTAACATCCAAGTCAAGGAAGTCACGACGCAGGA | NO: 834) |
| | CGGCACCACCACCATCGCCAACAACCTCACCTCCA | |
| | CCGTCCAAGTGTTTACGGACAACGACTACCAGCTA | |
| | CCGTACGTCATCGGCAACGGAACGGAGGGGTGCC | |
| | TGCCGGCCTTCCCTCCGCAGGTCTTTACGCTGCCG | |
| | CAGTACGGCTACGCGACACTGAACCGTAACAACA | |
| | CCGACGATCCCACCGAGCGGAGCAGTTTCTTCTGC | |
| | CTGGAATACTTTCCCAGCAAGATGCTGCGGACGGG | |
| | CAACAACTTTGAATTCACCTACAGCTTCGAGGAGG | |
| | TGCCCTTCCACTGCAGCTTCGCTCCCAGCCAGAAC | |
| | CTCTTCAAGCTGGCCAATCCGCTGGTGGACCAGTA | |
| | CCTGTACCGCTTTGTGAGCACCGACACTTCCGGTA | |
| | ACCTACAGTTCCAAAAGAACTTGAAGGCCAGATA | |
| | TGCCAACACTTACAAGAATTGGTTTCCGGGGCCCA | |
| | TGTGCCGGACCCAGGGCTGGTACACAAGCGCGGG | |
| | CACATATAACAACAAAGGCGTTGCCAACTTTGATA | |
| | CTTCAAACAAGATGGAACTGGAGGGGGCTAGTTA | |
| | CCAAGTAAACCCTCAACCAAATGGAATGACAAAC | |
| | ACGCTTCAGGATAGTAACAAATACGCGCTTGAAA | |
| | ACACCATGATCTTCAACGCACAGAACGCCCCTCCG | |
| | GGAACGACCTCTCTGTACCAGGAGAACAATCTTTT | |
| | GATAACCAGCGAGAGCGAGACGCAGCCTGTGAAC | |
| | CGATTGGCCTACAACACCGGTGGTCAGGTATCAAA | |
| | CAACAACCAGAATTCAAATACACATCCTACGGTCG | |
| | GAGTATACAATCACCAGGAAGTGTTGCCTGGTAGC | |
| | GTGTGGATGGACAGAGACGTATACCTTCAGGGCC | |
| | CCATCTGGGCCAAAATCCCGGAGACAGGGGCACA | |
| | CTTTCATCCTTCTCCGGCTATGGGCGGATTCGGAC | |
| | TCAAACACCCACCGCCCATGATGCTCATCAAGAAC | |
| | ACACCGGTACCTAGCAACGTCGCTGCCTTCTCTGA | |
| | CGTGCCCGTTAAAAGCTTCATCACCCAGTACAGCA | |
| | CCGGACAGGTCACGGTGGAGATTGAATGGGAGCT | |
| | CAAGAAAGAAAACTCCAAGAGGTGGAATCCCGAG | |
| | ATACAGTACACCAACAACTACAACAACCCTACATT | |
| | CGTGGACTTTGCTCCAGACACCTCCGGCGAGTACA | |
| | GGACTACGAGGGCTATTGGAACCCGTTACCTTACC | |
| | CGACCCCTGTAA (SEQ ID NO: 833) | |
| SD2 | ATGTCGTTTGTTGATCACCCTCCAGATTGGCTTGA | MSFVDHPPDWLEEIGEGLKEFLGLEPGPP |
| | GGAGATTGGTGAGGGTCTAAAGGAGTTTTTGGGA | KPKPNQQKQDNARGLVLPGYNYLGPGN |
| | CTCGAACCTGGCCCACCCAAACCGAAGCCCAACC | GLDRGEPVNRADEVAREHDISYNEQLQA |
| | AGCAGAAGCAAGACAACGCCCGTGGTCTTGTACT | GDNPYLKYNHADAEFQEKLADDTSFGG |
| | GCCTGGATATAATTACCTGGGACCCGGCAACGGTC | NLGKAVFQAKKRVLEPFGLVEDPVKTA |
| | TCGACCGCGGAGAACCTGTCAACCGGGCCGACGA | AKGERIDDHYPKKKKARVEETEAGTSGA |
| | GGTCGCGCGAGAGCACGACATCTCGTACAACGAG | QQLQIPAQPASSLGADTMSAGGGSPLGD |
| | CAGCTCCAGGCGGGAGACAACCCCTACCTCAAGT | NNQGADGVGNASGDWHCDSTWMGDR |
| | ACAACCACGCGGACGCCGAGTTTCAAGAGAAGCT | VITKSTRTWVLPSYNNHLYKEIHNGGVD |
| | CGCGGACGACACCTCCTTCGGGGGCAACCTCGGC | GSTANAYFGYSTPWGYFDFNRFHSHWSP |
| | AAGGCAGTCTTTCAGGCCAAGAAAAGGGTTCTCG | RDWQRLINNYWGFRPRSLKVKIFNIQVK |
| | AACCTTTTGGCCTGGTTGAGGATCCTGTTAAGACG | EVTVQDNTTTIANNLTSTVQVFTDNDYQ |
| | GCTGCTAAAGGCGAGCGGATAGACGACCACTATC | LPYVIGNGTEGCLPAFPPQVFTLPQYGYA |
| | CCAAGAAGAAGAAGGCTCGGGTTGAAGAAACCGA | TLNRNNSENPTERSSFFCLEYFPSKMLRT |
| | AGCTGGAACCAGCGGAGCCCAGCAGCTGCAGATC | GNNFEFTYSFEEVPFHCSFAPSQNLFKLA |
| | CCAGCCCAACCAGCCTCAAGTTTGGGAGCTGATAC | NPLVDQYLYRFVSTDTSGAVQFKKNLAG |
| | AATGTCTGCGGGAGGTGGCAGCCCACTGGGCGAC | RYANTYKNWFPGMPMCRTQGWYTGTGIY |
| | AATAACCAAGGCGCCGATGGAGTGGGCAATGCCT | NNKGATSFNTSNRMDLEGASYQVPPQPN |
| | CGGGAGATTGGCATTGCGATTCCACATGGATGGG | GMTNTVQDSNLYALENTMIFNAQNATP |
| | GGACAGAGTCATCACCAAGTCCACCCGAACCTGG | GTNTTYPEENLLITSESETQPVNRVAYSS |
| | GTGCTGCCCAGCTACAATAACCACCTGTACAAAGA | GGQIANNNQNTNTAPTAGTYNHQEILPG |
| | GATCCACAACAACGGGGTCGACGGCAGCACCGCC | SVWMDRDVYLQPIWAKIPETGAHFHPS |
| | AACGCTTACTTTGGATACAGTACCCCCTGGGGGTA | PAMGGFGLKYPPPMMLIKNTPVPGNITTF |
| | CTTTGACTTCAACCGCTTCCACAGCCACTGGAGCC | SDVPVQSFITQYSTGQVTVEMEWELKKE |
| | CTCGAGACTGGCAGCGACTCATCAACAACTACTGG | NSKRWNPEIQYTNNYNNPTFVDFAPDTE |
| | GGCTTCAGACCCCGGTCCCTCAAGGTCAAGATTTT | GEYRTTRAIGTRYLTRPL (SEQ ID |
| | CAACATCCAAGTCAAAGAGGTCACGGTGCAGGAC | NO: 836) |
| | AACACCACCACCATCGCCAACAACCTCACCTCCAC | |
| | CGTCCAAGTGTTTACGGACAACGACTACCAGCTGC | |
| | CGTACGTCATCGGCAACGGGACCGAGGGGTGTCT | |
| | ACCGGCCTTCCCTCCGCAGGTCTTTACGCTGCCGC | |
| | AGTATGGCTACGCGACGCTGAATCGCAACAATAG | |
| | CGAAAATCCCACCGAGCGAAGCAGCTTCTTCTGTC | |
| | TGGAGTACTTTCCCAGCAAGATGCTGCGGACGGGC | |
| | AACAACTTTGAGTTCACATACAGCTTCGAGGAGGT | |
| | ACCCTTCCACTGCAGCTTCGCGCCCAGCCAAAACC | |
| | TCTTCAAACTGGCTAACCCGTTGGTGGATCAGTAC | |
| | CTTTACCGCTTCGTGAGCACGGACACCTCCGGTGC | |
| | CGTCCAGTTCAAAAAGAACCTGGCGGGCAGATAC | |
| | GCCAACACCTACAAGAACTGGTTCCCAGGACCCAT | |
| | GTGCCGAACCCAGGGCTGGTACACAGGAACGGGT | |
| | ATATATAACAACAAAGGCGCTACCAGCTTTAACAC | |
| | CTCAAACAGAATGGACCTAGAGGGAGCCAGTTAT | |
| | CAAGTGCCTCCCCAGCCCAACGGGATGACAAACA | |
| | CGGTTCAGGACAGCAACCTTTACGCGCTGGAAAA | |
| | CACCATGATTTTTAACGCACAAAACGCCACCCCGG | |
| | GAACGAATACAACGTATCCGGAGGAGAACCTTTT | |
| | GATAACCAGTGAGAGCGAGACTCAACCCGTGAAC | |
| | AGAGTGGCTTACAGCTCCGGAGGACAAATAGCCA | |
| | ACAACAATCAGAATACCAACACGGCTCCTACTGC | |
| | AGGAACCTACAACCACCAGGAAATACTACCTGGC | |
| | AGCGTGTGGATGGACAGGGACGTGTACCTCCAGG | |
| | GTCCCATCTGGGCCAAGATCCCAGAGACAGGGGC | |
| | ACACTTTCATCCTTCTCCAGCCATGGGCGGGTTCG | |
| | GACTCAAATACCCGCCTCCCATGATGCTAATCAAG | |
| | AACACGCCAGTGCCCGGAAACATCACCACCTTCTC | |
| | GGACGTGCCCGTCCAAAGTTTTATTACCCAGTACA | |
| | GCACCGGACAAGTCACCGTGGAGATGGAGTGGGA | |
| | GCTCAAGAAGGAAAACTCTAAGAGGTGGAACCCC | |
| | GAGATACAGTACACCAACAACTACAACAACCCTA | |
| | CGTTCGTGGACTTTGCTCCAGACACAGAAGGAGA | |
| | ATACAGGACCACTAGGGCTATTGGAACCCGCTACC | |
| | TTACCCGACCTCTGTAA (SEQ ID NO: 835) | |
| MR1 | ATGTCGTTTTTTGATTACATAGGTAAAAAATACGC | MSFFDYIGKKYAQGAADWWNAEAGPPP |
| | TCAAGGAGCGGCCGATTGGTGGAACGCAGAAGCG | PKKAKGKGFVFPGHNYLGPGNDLDNGN |
| | GGACCACCTCCTCCAAAAAAAGCCAAAGGGAAAG | PVDEDDAAAKKHDEAYDEQLKQGDNPY |
| | GATTCGTCTTTCCCGGTCACAACTATCTAGGTCCG | VKYNHADAQLQEDLKDVNTFEANLAKG |
| | GGAAACGACCTAGATAACGGGAATCCCGTCGACG | IFQAKKRVLEPFGLVEELAGKKRPHTEQE |
| | AAGACGACGCTGCCGCCAAAAAGCACGACGAGGC | SPDSSAGVGKKGEQPAKRRLDFSDAEAP |
| | TTACGACGAGCAGCTCAAGCAGGGCGACAATCCG | QDGAAAAPSGVEDITMSGGGGAPMDGN |
| | TACGTGAAGTATAATCACGCGGACGCTCAGCTTCA | QQGADGVGNASGNWHCDSQWMGNRV |
| | GGAAGACCTCAAGGACGTCAATACCTTCGAGGCC | VTHSTRTWVLPSYNDHIYRQISSGAQTD |
| | AATCTCGCTAAAGGAATCTTTCAGGCCAAAAAAC | HSKQYFGYSTPWGYFDFNRFHCHFSPRD |
| | GGGTCCTAGAACCGTTTGGCCTGGTGGAAGAACTG | WQRLVNNHWGFRPKKLHVKLFNIQVKE |
| | GCCGGGAAAAAGAGGCCTCATACTGAACAAGAGT | WVTQDGTNVVSNNLTSTVQVFTDDEYQ |
| | CCCCCGATTCCAGCGCAGGAGTCGGAAAGAAAGG | LPYVLGNATEGCLPAFPPTVFTLPQYGY |
| | CGAACAGCCAGCTAAGCGTCGGTTGGATTTCTCCG | ATLNDPGDDAPTNRSSFFCLEYFPSQMLR |
| | ACGCAGAAGCGCCTCAAGACGGAGCAGCAGCAGC | TGNNFEFSYSFEDVPFHTGWAPNQHLFK |
| | GCCCTCTGGTGTGGAAGATATTACAATGTCTGGCG | TANPLVDQYLYQLVGTNNTGDVQFEKV |
| | GAGGAGGCGCTCCGATGGACGGAAATCAACAAGG | LAGNYGDAFKNWITGANRRTNSWLLGG |
| | TGCCGATGGAGTGGGCAATGCCTCGGGAAATTGG | GNRANVTALNPNYVQIEGDFYKSQPEPA |
| | CATTGCGATTCCCAATGGATGGGGAACAGAGTCGT | GMANNLSGSDAVALGNSLIFNPQQTAPG |
| | TACCCATTCCACCAGAACCTGGGTCCTGCCCAGCT | TTTRLPAQNVLVTREDETQPVNTYASNS |
| | ACAACGACCACATCTACCGACAAATCAGCAGCGG | TGYVATNQQSTTSAPQNGQYHFQGIMPG |
| | AGCCCAGACCGACCACTCCAAGCAATACTTTGGAT | AVWMDRDVYLHGPIWAKIPNTGAHFHP |
| | ACAGCACCCCCTGGGGGTACTTTGACTTTAACCGA | SPMMGGFGLKAPPPMILMKNTPVPGVTQ |
| | TTCCACTGCCACTTTTCACCGCGAGACTGGCAGCG | AFSEVVSNNFINQYSTGQVTVSIEWELQK |
| | ATTGGTCAACAACCACTGGGGATTCAGACCCAAG | ENSKRWNPEIQYTNNYNDPQFVDFAPDS |
| | AAGCTGCACGTCAAGCTCTTTAACATCCAAGTCAA | TGEYRTTRPIGTRYLTRPL (SEQ ID NO: |
| | GGAGTGGGTCACTCAAGATGGGACTAACGTCGTA | 838) |
| | TCCAACAACCTCACGAGCACCGTACAAGTGTTCAC | |
| | GGACGACGAGTACCAGCTCCCCTACGTGCTCGGCA | |
| | ACGCCACAGAAGGATGTCTCCCCGCCTTCCCTCCG | |
| | ACGGTCTTTACGCTGCCGCAGTACGGCTACGCGAC | |
| | GCTGAACGACCCGGGGGACGACGCTCCGACCAAC | |
| | AGGAGCTCGTTCTTTTGCCTGGAGTACTTTCCCAG | |
| | TCAGATGCTGAGAACCGGCAACAACTTTGAGTTCA | |
| | GCTACTCGTTCGAAGACGTGCCCTTTCACACGGGA | |
| | TGGGCTCCCAACCAGCATCTGTTTAAGACGGCGAA | |
| | TCCTCTGGTGGACCAGTACCTGTATCAACTGGTCG | |
| | GAACCAACAACACCGGAGACGTTCAGTTTGAGAA | |
| | AGTGCTGGCCGGTAACTACGGAGACGCGTTCAAG | |
| | AACTGGATCACGGGAGCCAACAGGCGTACCAACT | |
| | CTTGGCTGCTGGGCGGAGGAAACAGAGCCAACGT | |
| | CACCGCTCTGAATCCAAACTACGTCCAGATCGAAG | |
| | GAGACTTTTACAAGTCGCAGCCGGAACCCGCCGG | |
| | CATGGCCAACAACCTCAGCGGATCGGACGCGGTT | |
| | GCCCTGGGCAACAGTCTGATATTCAACCCTCAGCA | |
| | GACGGCGCCAGGAACTACCACCAGACTGCCGGCG | |
| | CAGAACGTACTGGTGACCAGAGAAGACGAGACGC | |
| | AGCCCGTCAACACGTACGCGTCCAACTCTACCGGA | |
| | TACGTGGCGACCAATCAGCAGAGTACTACGTCGG | |
| | CTCCTCAGAACGGACAGTACCACTTTCAAGGCATC | |
| | ATGCCAGGGGCCGTGTGGATGGACAGAGACGTGT | |
| | ACCTGCACGGACCCATCTGGGCCAAGATTCCGAAT | |
| | ACGGGAGCGCACTTTCATCCTTCACCCATGATGGG | |
| | CGGATTCGGATTGAAGGCTCCTCCTCCCATGATCC | |
| | TGATGAAGAATACGCCCGTTCCCGGAGTGACGCA | |
| | GGCGTTCAGCGAGGTCGTGTCCAACAACTTCATCA | |
| | ACCAGTATTCTACGGGTCAGGTGACAGTGTCCATC | |
| | GAATGGGAGCTGCAGAAGGAAAACAGCAAGCGGT | |
| | GGAATCCGGAGATTCAGTACACCAACAACTACAA | |
| | CGACCCCCAGTTTGTGGACTTTGCCCCGGACAGCA | |
| | CCGGGGAATACAGAACCACCAGACCTATCGGAAC | |
| | CCGATACCTTACCCGACCCCTTTAA (SEQ ID | |
| | NO: 837) | |
| RN1 | ATGTCTTTTTTCGATTGGATAGGTCGTAAGTACGC | MSFFDWIGRKYANGAAEFWDLEPGPPKP |
| | CAACGGCGCCGCTGAGTTCTGGGATCTCGAGCCGG | KKARVDDSAGFNFPGHKYLGPGNGLDR |
| | GTCCTCCGAAGCCCAAAAAGGCCAGAGTCGACGA | GEPVDADDAAAQKHDQEYQALLESGEN |
| | CTCGGCCGGCTTCAACTTCCCCGGGCACAAGTACT | PYLTFNHADRQLQKDLAEDQSFEGNLAR |
| | TGGGCCCCGGCAACGGTCTCGACCGAGGGGAGCC | GLFEAKKLAAQVVGVEEPELAPPVKRPH |
| | CGTGGATGCCGACGACGCGGCGGCCCAGAAACAC | SPEKTPENQKGQPRPDPRTPAKKRLEFSD |
| | GACCAGGAATATCAGGCTCTGCTTGAGAGCGGTG | QPGSSADLPASSQQSQPPAGVPGVVPGT |
| | AGAATCCTTACCTCACTTTTAATCACGCTGATCGG | MSAGGGAPVDDAQQGADGVGNASGDW |
| | CAGCTGCAGAAGGACCTCGCGGAGGACCAATCTT | HCDSKWLGNRVLTRSTRTWVLPSYNNH |
| | TCGAGGGGAACCTCGCCCGGGGTCTGTTCGAAGCC | LYKQISDASGVHSLPGSRYFGYSTPWGY |
| | AAGAAGCTCGCGGCACAGGTCGTAGGCGTCGAGG | FDFNRFHCHFSPRDWQRLVNNHWGFRP |
| | AGCCGGAGCTGGCTCCTCCAGTCAAGCGTCCACAC | KRLRVKLFNIQVKEVTTTDSTTTVSNNLT |
| | TCGCCCGAGAAAACCCCAGAGAACCAGAAGGGTC | STVQVFTDDEYQLPYVCGNATEGCLPPF |
| | AGCCGCGACCGGATCCCCGGACGCCGGCCAAGAA | PPDVFTLPQYGYATLNDSNNGNPTNRSS |
| | GAGGCTCGAGTTCTCCGATCAGCCTGGATCTTCAG | FFCLEYFPSKMLRTGNNFEFTFEFEDVPF |
| | CAGACTTACCTGCATCCTCACAACAGTCCCAGCCT | HTGFAPCQNLFKLSNPLVDQYLYRFQGT |
| | CCCGCAGGGGTTCCCGGTGTGGTTCCTGGTACGAT | DNSSSSTPGVVKFEKCVAHNYAQQYKN |
| | GTCTGCAGGAGGAGGCGCTCCAGTGGACGATGCT | WLTGAFQRNQDYNYVSGTSNYKGVVGS |
| | CAACAAGGTGCCGACGGAGTGGGCAATGCCTCGG | NONNLQRIENVQFAIAPDVPSMCNHLEG |
| | GAGATTGGCATTGCGATTCCAAATGGCTGGGCAAC | TNMIALDNSLIFRDVSTAPGDTTQYNINQ |
| | CGAGTTCTCACCCGATCCACCCGGACCTGGGTGCT | VIVTSEAETQSVNAYSGDTCGRIVNNSQ |
| | GCCCAGCTACAACAACCACCTGTACAAGCAGATCT | NSGTNAGTTGINYKGTMPSSVWMDRDV |
| | CAGACGCCTCCGGCGTGCACAGCCTCCCCGGGAG | YLQGPIWAKIPHTGAHFHPSPMMGGFGL |
| | CCGATACTTTGGCTACAGCACCCCCTGGGGGTACT | RNPPPMMLIKNTPVPGNVTTFTEVKVNQ |
| | TCGACTTCAATCGCTTCCACTGCCACTTCTCGCCCA | FITQYSTGQITVDVEWELQKENSKRWNP |
| | GAGACTGGCAGCGCCTCGTCAATAACCACTGGGG | EIQYTNNYSNNTFVDFAPNANGDYQMT |
| | CTTCCGACCCAAGAGACTGCGAGTCAAACTCTTCA | RPIGTRYPTRPP (SEQ ID NO: 840) |
| | ACATCCAGGTCAAGGAGGTCACGACTACTGATTCG | |
| | ACGACCACGGTCTCCAACAACCTCACGAGCACGG | |
| | TCCAGGTCTTCACAGACGACGAGTACCAGCTGCCG | |
| | TACGTCTGCGGCAACGCCACCGAGGGATGCCTGCC | |
| | GCCGTTCCCCCCGGACGTCTTCACGCTGCCGCAGT | |
| | ACGGCTACGCGACGCTGAACGACAGCAACAATGG | |
| | CAACCCCACCAACAGATCGAGCTTCTTCTGTCTGG | |
| | AATACTTTCCCAGCAAGATGCTGAGAACCGGGAA | |
| | CAACTTTGAATTTACCTTCGAGTTTGAAGACGTGC | |
| | CCTTCCACACGGGCTTCGCCCCCTGTCAGAACCTG | |
| | TTCAAGCTGAGCAATCCCCTGGTGGACCAGTACCT | |
| | GTACCGCTTCCAGGGTACTGACAACTCCTCCAGCA | |
| | GCACGCCCGGGGTGGTCAAGTTCGAGAAATGCGT | |
| | GGCCCACAACTACGCCCAGCAGTACAAGAACTGG | |
| | CTGACCGGTGCTTTCCAGAGGAACCAGGACTATAA | |
| | CTACGTGTCTGGTACTAGCAACTACAAGGGCGTGG | |
| | TTGGATCTAATCAGAATAACCTGCAGCGCATAGAG | |
| | AATGTGCAGTTCGCCATTGCTCCGGACGTGCCCAG | |
| | CATGTGTAACCATCTGGAGGGCACCAATATGATCG | |
| | CTCTGGACAACAGCCTGATCTTCCGGGATGTCTCT | |
| | ACCGCCCCTGGTGACACGACTCAGTATAACATCAA | |
| | CCAGGTGATCGTGACCAGCGAAGCCGAGACTCAA | |
| | TCTGTCAATGCGTACTCGGGCGACACCTGTGGCCG | |
| | CATTGTGAATAACAGCCAGAATAGCGGAACCAAC | |
| | GCTGGGACCACCGGCATCAACTACAAAGGTACCA | |
| | TGCCGTCCTCGGTCTGGATGGACCGTGACGTCTAC | |
| | CTGCAGGGCCCGATCTGGGCCAAGATTCCCCACAC | |
| | GGGGGCGCACTTTCACCCGTCTCCCATGATGGGGG | |
| | GCTTCGGCCTGAGGAACCCTCCTCCGATGATGCTG | |
| | ATCAAGAACACGCCCGTTCCTGGCAACGTGACCAC | |
| | CTTCACCGAGGTCAAGGTTAACCAGTTCATCACCC | |
| | AGTACTCGACCGGGCAGATCACCGTGGACGTGGA | |
| | ATGGGAGCTGCAGAAGGAGAACTCCAAACGATGG | |
| | AACCCCGAGATCCAGTACACAAATAACTACAGTA | |
| | ATAATACTTTTGTGGACTTTGCCCCCAATGCTAAT | |
| | GGAGATTACCAGATGACCCGCCCCATTGGAACCC | |
| | GCTACCCGACCCGCCCCCCGTAA (SEQ ID NO: 839) | |
| RN2 | ATGTCTTTTTTCGATTGGATAGGTCGTAAGTACGC | MSFFDWIGRKYANGAAEFWDLEPGPPKP |
| | CAACGGCGCCGCTGAGTTCTGGGACCTCGAGCCG | KKARVDDSAGFNFPGHKYLGPGNGLDR |
| | GGTCCTCCGAAGCCCAAAAAGGCCAGAGTCGACG | GEPVDADDAAAQKHDQEYQALLESGEN |
| | ACTCGGCCGGCTTCAACTTCCCCGGGCACAAGTAC | PYLTFNHADRQLQKDLAEDQSFEGNLAR |
| | CTGGGCCCCGGCAACGGTCTCGACCGAGGCGAGC | GLFEAKKLAAQVVGVEEPELAPPVKRPH |
| | CCGTGGATGCCGACGACGCGGCGGCCCAGAAGCA | SPEKTPEDQKGQPRPDPRTPAKKRLEFSD |
| | CGACCAGGAATATCAGGCTCTGCTTGAGAGCGGC | QPGSAADLRTSSQQSQPPAGVPGVVSGT |
| | GAGAATCCTTACCTCACTTTTAATCACGCTGATCG | MSAGGGAPVDDAQQGADGVGNASGDW |
| | GCAGCTGCAGAAGGACCTCGCGGAGGACCAATCT | HCDSKWLGNRVLTRSTRTWVLPSYNNH |
| | TTCGAGGGGAACCTCGCCCGGGGCCTGTTCGAAGC | LYKQISDASGVHGLPGSRYFGYSTPWGY |
| | CAAGAAGCTCGCGGCGCAGGTCGTAGGCGTCGAG | FDFNRFHCHFSPRDWQRLVNNHWGFRP |
| | GAGCCTGAGCTGGCTCCTCCAGTCAAGCGTCCACA | KRLRVKLFNIQVKEVTTTDSTTTVSNNLT |
| | CTCTCCCGAGAAGACCCCGGAAGACCAGAAGGGT | STVQVFTDDEYQLPYVCGNATEGCLPPF |
| | CAGCCGCGACCGGATCCCCGGACGCCGGCCAAGA | PPDVFTLPQYGYATLNDSNNGNPTNRSS |
| | AGCGGCTCGAGTTCTCCGATCAGCCTGGATCTGCA | FFCLEYFPSKMLRTGNNFEFTFEFEDVPF |
| | GCAGACTTACGTACATCCTCACAACAGTCCCAGCC | HTGFAPCQNLFKLSNPLVDQYLYRFQGT |
| | TCCCGCAGGGGTTCCCGGTGTGGTTTCTGGTACGA | DASTSSTPGIVKFEKCVAHNYAQQYKN |
| | TGTCTGCAGGAGGAGGCGCTCCAGTGGACGATGC | WLTGAFQRNQDYNYVSGTTNYKGIVGS |
| | TCAACAAGGTGCCGACGGAGTGGGCAATGCCTCG | NQNNLQRIENVQFAIAPDVPSMCNHLEG |
| | GGAGATTGGCATTGCGATTCCAAATGGCTGGGCA | TNMIALDNSLIFRDVSTAPGDTTQYNINQ |
| | ACCGAGTTCTCACCCGATCCACCCGGACCTGGGTG | VIVTSEAETQPVNSYSGDNCGRIVNNSQN |
| | CTGCCCAGCTACAACAACCACCTGTACAAGCAGAT | NGTNAGTTGINYKGTMPSSVWMDRDVY |
| | CTCCGACGCCTCCGGCGTGCACGGCCTCCCCGGGA | LQGPIWAKIPHTGAHFHPSPMMGGFGLK |
| | GTCGATACTTTGGCTACAGCACCCCCTGGGGGTAC | NPPPMMLIKNTPVPGNVTTFTEVKVNQFI |
| | TTCGACTTCAATCGCTTCCACTGCCACTTCTCGCCC | TQYSTGQITVDVEWELQKENSKRWNPEI |
| | AGAGACTGGCAGCGCCTCGTCAACAACCACTGGG | QYTNNYSNNTFVDFAPNANGDYQMTRPI |
| | GCTTCCGACCCAAGCGACTGCGAGTCAAGCTCTTC | GTRYLTRPL (SEQ ID NO: 842) |
| | AACATCCAGGTCAAGGAGGTCACGACTACTGATTC | |
| | GACGACCACGGTCTCCAACAACCTCACGAGCACG | |
| | GTCCAGGTCTTCACAGACGACGAGTACCAGCTGCC | |
| | GTACGTCTGCGGCAACGCCACCGAGGGATGCCTG | |
| | CCGCCGTTCCCTCCGGACGTCTTCACGCTGCCGCA | |
| | GTACGGCTACGCGACGCTGAACGACAGCAACAAC | |
| | GGCAACCCCACCAACAGATCGAGCTTTTTCTGTCT | |
| | GGAATACTTTCCCAGCAAGATGCTGAGGACCGGG | |
| | AACAACTTTGAATTCACCTTCGAGTTCGAGGACGT | |
| | GCCTTTCCACACGGGCTTCGCCCCCTGTCAGAACC | |
| | TGTTCAAGCTGAGTAACCCCCTGGTGGACCAGTAC | |
| | CTGTATCGCTTCCAAGGAACAGATGCCTCCACTAG | |
| | CAGTACTCCCGGAATCGTCAAGTTCGAGAAATGCG | |
| | TAGCTCATAATTACGCCCAGCAGTACAAGAACTGG | |
| | CTGACTGGAGCTTTCCAGAGGAATCAGGACTACA | |
| | ACTATGTGTCTGGTACCACTAATTATAAAGGAATC | |
| | GTGGGATCCAACCAGAATAACTTGCAGCGCATAG | |
| | AGAACGTTCAGTTTGCCATCGCTCCGGACGTGCCC | |
| | AGCATGTGTAACCATCTCGAAGGCACCAATATGAT | |
| | CGCTCTGGATAATAGCCTTATCTTCAGAGATGTGT | |
| | CTACCGCCCCCGGCGACACGACTCAGTATAACATC | |
| | AACCAGGTGATCGTGACCAGCGAGGCCGAGACTC | |
| | AACCTGTCAATTCGTACTCGGGCGACAACTGTGGC | |
| | CGCATTGTGAATAACAGCCAGAATAACGGGACCA | |
| | ACGCCGGGACTACCGGCATCAACTACAAAGGTAC | |
| | CATGCCTTCCTCGGTCTGGATGGACCGTGACGTCT | |
| | ACCTGCAGGGCCCGATCTGGGCCAAGATTCCCCAC | |
| | ACGGGGGCGCACTTTCACCCGTCTCCCATGATGGG | |
| | GGGCTTCGGCCTGAAGAACCCTCCCCCGATGATGC | |
| | TGATCAAGAACACGCCCGTTCCTGGCAACGTGACC | |
| | ACCTTCACCGAGGTCAAGGTTAACCAGTTCATCAC | |
| | CCAGTACTCGACCGGGCAGATCACCGTGGACGTG | |
| | GAATGGGAGCTGCAGAAGGAGAACTCCAAGAGAT | |
| | GGAACCCCGAGATCCAGTACACAAACAACTACAG | |
| | CAATAACACTTTTGTGGACTTTGCCCCCAATGCCA | |
| | ATGGAGATTACCAGATGACCCGCCCTATCGGAACC | |
| | CGATACCTTACCCGACCCCTTTAA (SEQ ID | |
| | NO: 841) | |
| RN3 | ATGTCTTTCTTCGATTGGATAGGTCGTAAGTACGC | MSFFDWIGRKYANGAAEFWDLEPGPPKP |
| | CAACGGCGCCGCTGAGTTCTGGGACCTCGAGCCG | KKARVDDSAGFNFPGHKYLGPGNGLDR |
| | GGTCCTCCGAAGCCCAAAAAGGCCCGAGTCGACG | GEPVDADDAAAQKHDQEYQALLESGEN |
| | ACTCGGCCGGCTTCAACTTCCCCGGGCACAAGTAC | PYLTFNHADRQLQKDLAEDQSFEGNLAR |
| | CTGGGCCCCGGCAACGGTCTCGACCGCGGGGAGC | GLFEAKKLAAQVVGVEEPELAPPVKRPH |
| | CCGTGGATGCCGACGACGCGGCGGCCCAGAAGCA | SPEKTPEDQKGQPRPDPRTPAKKRLEFSD |
| | CGACCAGGAATATCAGGCTCTGCTTGAGAGCGGC | QPGSSADLPASSQQSQPPAGVPGVVPGT |
| | GAGAATCCTTACCTCACTTTTAATCACGCTGATCG | MSAGGGAPVDDAQQGADGVGNASGDW |
| | GCAGCTGCAGAAGGACCTCGCGGAGGACCAATCT | HCDSKWLGNRVLTRSTRTWVLPSYNNH |
| | TTCGAGGGGAACCTCGCCCGGGGCCTGTTCGAAGC | LYKQISDASGVHGLPGSRYFGYSTPWGY |
| | CAAGAAGCTCGCGGCGCAGGTCGTAGGCGTCGAG | FDFNRFHCHFSPRDWQRLVNNHWGFRP |
| | GAGCCGGAGCTGGCTCCTCCAGTCAAGCGTCCACA | KRLRVKLFNIQVKEVTTTDSTTTVSNNLT |
| | CTCGCCCGAGAAGACCCCGGAGGACCAGAAGGGT | STVQVFTDDEYQLPYVCGNATEGCLPPF |
| | CAGCCGCGACCGGATCCCCGGACGCCGGCAAAGA | PPDVFTLPQYGYATLNDSNNGNPTNRSS |
| | AGCGGCTCGAGTTCTCCGATCAGCCTGGATCTTCA | FFCLEYFPSKMLRTGNNFEFTYEFEEVPF |
| | GCAGACTTACCTGCATCCTCACAACAGTCCCAGCC | HTGFAPCQNLFKLSNPLVDQYLYRFQGT |
| | TCCCGCAGGGGTTCCCGGTGTGGTTCCTGGTACGA | DASTSSTPGIVKEFKCVAHNYAQQYKN |
| | TGTCTGCAGGAGGAGGCGCTCCAGTGGACGATGC | WLTGAFQRNQDYNYVSGTTNYKGIVGS |
| | TCAACAAGGTGCCGACGGAGTGGGCAATGCCTCG | NQNNLQRIENVQFAIAPDVPSMCNHLEG |
| | GGAGATTGGCATTGCGATTCCAAATGGCTGGGCA | TNMIALDNSLIFRDVSTAPGDTTQYNINQ |
| | ACCGAGTTCTCACCCGATCCACCCGGACCTGGGTG | VIVTSEAETQPVNAYSGDNCGRIVNNSQ |
| | CTGCCCAGCTACAACAACCACCTGTACAAGCAGAT | NNGTNAGTTGINYKGTMPSSVWMDRDV |
| | CTCCGACGCCTCCGGCGTGCACGGCCTCCCCGGGA | YLQGPIWAKIPHTGAHFHPSPMMGGFGL |
| | GCCGATACTTTGGCTACAGCACCCCCTGGGGGTAC | KNPPPMMLIKNTPVPGNVITFTEVKVNQF |
| | TTCGACTTCAATCGCTTCCACTGCCACTTCTCGCCC | ITQYSTGQITVDVEWELQKENSKRWNPEI |
| | AGAGACTGGCAGCGACTCGTCAACAACCACTGGG | QYTNNYSNNTFVDFAPNANGDYQMTRPI |
| | GCTTCCGACCCAAGCGTCTCCGAGTCAAGCTCTTC | GTEYLTRPL (SEQ ID NO: 844) |
| | AACATCCAGGTCAAGGAGGTCACGACTACTGATTC | |
| | GACGACCACGGTCTCCAACAACCTCACGAGCACG | |
| | GTCCAGGTCTTCACAGACGACGAGTACCAGCTGCC | |
| | GTACGTCTGCGGCAACGCCACCGAGGGATGCCTG | |
| | CCGCCGTTCCCTCCGGACGTCTTCACGCTGCCGCA | |
| | GTACGGCTACGCGACGCTGAACGACAGCAACAAC | |
| | GGCAACCCCACCAACAGATCGAGCTTCTTCTGTCT | |
| | GGAATACTTTCCCAGCAAGATGCTGAGGACCGGG | |
| | AACAACTTTGAATTCACCTACGAGTTCGAGGAAGT | |
| | GCCCTTCCACACGGGCTTCGCCCCCTGTCAGAACC | |
| | TGTTCAAGCTGAGTAACCCCCTGGTGGACCAGTAC | |
| | CTGTATCGCTTCCAAGGAACAGATGCCTCAACTAG | |
| | CAGCACTCCCGGAATCGTCAAGTTCGAGAAATGC | |
| | GTAGCCCATAATTACGCCCAGCAGTACAAGAACT | |
| | GGCTGACTGGAGCTTTCCAGAGGAACCAGGACTA | |
| | CAACTATGTGTCTGGCACCACTAATTATAAAGGCA | |
| | TCGTGGGATCTAATCAGAATAACCTGCAGCGCATA | |
| | GAGAATGTGCAGTTCGCCATTGCTCCGGACGTGCC | |
| | CAGCATGTGTAACCATCTGGAGGGCACCAACATG | |
| | ATTGCCCTGGACAACAGCCTGATCTTCCGAGATGT | |
| | CTCTACCGCCCCCGGTGACACGACTCAGTATAACA | |
| | TCAACCAGGTGATCGTGACCAGCGAGGCCGAGAC | |
| | TCAACCCGTCAATGCGTACTCGGGCGATAACTGTG | |
| | GCCGCATTGTGAATAATAGCCAGAATAACGGGAC | |
| | CAACGCCGGGACCACCGGCATCAACTACAAAGGT | |
| | ACCATGCCTTCCTCGGTCTGGATGGACCGTGACGT | |
| | CTACCTGCAGGGCCCGATCTGGGCCAAGATTCCCC | |
| | ACACGGGGGCGCACTTTCACCCGTCTCCCATGATG | |
| | GGGGGCTTCGGCCTGAAGAACCCTCCTCCGATGAT | |
| | GCTGATCAAGAACACGCCCGTTCCTGGCAACGTGA | |
| | TCACCTTCACCGAGGTCAAGGTCAACCAGTTCATC | |
| | ACCCAGTACTCGACCGGGCAGATCACCGTGGACG | |
| | TGGAATGGGAGCTGCAGAAGGAGAACTCCAAGAG | |
| | ATGGAACCCCGAGATCCAGTACACAAACAACTAC | |
| | AGCAACAACACTTTTGTGGACTTTGCCCCCAATGC | |
| | CAATGGAGATTACCAGATGACCCGCCCTATCGGA | |
| | ACCCGATACCTTACCCGACCCTTTAA (SEQ ID | |
| | NO: 843) | |
| MM1 | ATGTCTTTCTTTGATTGGGTAGGTAAAAAGTACGC | MSFFDWVGKKYAQGAAEFWDLKSGPPA |
| | TCAAGGAGCTGCTGAATTCTGGGATCTGAAGTCCG | PKKARKDGSAGFNFPGHKYLGPGNSLDR |
| | GTCCTCCTGCACCAAAAAAGGCGCGAAAGGACGG | GDPVDADDAAAQKHDQAYQELQLEAGD |
| | ATCTGCCGGATTCAATTTCCCCGGGCATAAAATATC | NPYLKYNHADREFQEALKDDTSFEGNLA |
| | TCGGACCCGGTAACTCTCTCGATCGAGGAGATCCG | RGLFEAKKLVAEPLGLVEPELAPGKKRP |
| | GTTGACGCCGACGACGCCGCTGCTCAAAAGCACG | AASPVQSPDSSSGIGKKGAQPSRRRLDFD |
| | ATCAAGCTTATCAAGAGCAGCTTGAAGCGGGAGA | QADRETAAEAGATGTGSLPPASNLGPVT |
| | TAATCCCTATCTGAAATACAATCACGCCGATCGCG | MSAGGGAPMDDNQQGADGVGNASGN |
| | AGTTCCAAGAGGCGTTGAAAGACGATACCTCGTTT | WHCDSQWLGDRVITSTRTWSLPTYNN |
| | GAAGGAAATCTCGCGAGAGGACTCTTTGAGGCCA | HLYKKISSNEASGDKTWFGFSTPWGYFD |
| | AGAAGCTCGTGGCCGAGCCTCTAGGTCTCGTTGAA | FNRFHCHFSPRDWQRLINNHWGIRPKKM |
| | CCGGAACTGGCTCCGGGGAAGAAACGCCCAGCGG | HVKLFNIQVKEVTTQDSTTTIANNLTSTV |
| | CTTCCCCCGTTCAATCGCCAGACTCGTCTTCCGGT | QVFADSEYQLPYVAGNAHEGCLPPFPAD |
| | ATCGGCAAGAAGGGCGCGCAACCTTCCCGCCGAA | VFMLPQYGYCTLQNSASNDPVAQSAFYC |
| | GATTGGACTTTGACCAGGCCGATCGAGAAACCGCT | LEYFPSQMLRTGNNFVFSYEFEDVPFHS |
| | GCCGAGGCTGGAGCCACAGGCACCGGAAGTCTAC | MYMHNQSLDRLMNPLIDQYLYYLSATN |
| | CACCCGCCTCTAATTTGGGACCTGTTACAATGTCT | APANTNKFTKSNKGDLPSQLRNWIPGPSF |
| | GCTGGGGGAGGCGCACCGATGGACGACAATCAAC | QQAAWTLDGQSQTQTNNTWDYANKYQ |
| | AAGGTGCCGATGGAGTGGGCAATGCCTCGGGAAA | LDARKNKLAPGPVNAPREGQFQANTLIF |
| | TTGGCATTGCGATTCCCAATGGCTGGGCGACAGAG | GKDPTVSSTLAEQDKVNITRDNETRTVN |
| | TCATTACAAAGTCCACCCGAACCTGGAGCCTGCCC | PILGTAQIFDIINNDTPRANGSISKINMNIC |
| | ACCTACAACAACCACTTGTACAAGAAGATCTCCAG | PVMPGSVWQDRDIYLQGPIWAKVPRTD |
| | CAACGAAGCTTCAGGAGACAAGACTTGGTTTGGA | GKFHPSPLMGGFGLRCPPPQIFIKNTPVPG |
| | TTCAGTACCCCTTGGGGATACTTTGACTTCAACCG | NPPTTFEPTKVASFITQYSTGQVTVEMEW |
| | CTTCCACTGTCACTTCTCCCCCCGAGACTGGCAGC | ELEKENSKRWNPEIQFTNNFSSSDGNMV |
| | GTCTCATCAACAACCACTGGGGCATCCGCCCGAAG | HFAVDSTGLYSEPRPVGTRYLTRSL (SEQ |
| | AAGATGCACGTCAAGCTCTTCAATATTCAAGTTAA | ID NO: 846) |
| | GGAGGTCACGACGCAAGACTCGACGACGACCATC | |
| | GCTAATAACCTTACCAGTACGGTTCAGGTCTTTGC | |
| | GGACTCGGAATATCAGCTGCCGTACGTAGCCGGG | |
| | AATGCGCACGAAGGATGCTTGCCACCTTTTCCAGC | |
| | TGATGTCTTTATGCTGCCGCAGTACGGCTACTGCA | |
| | CTCTCCAGAACAGCGCTTCCAACGATCCAGTGGCC | |
| | CAGAGTGCCTTCTACTGTCTGGAATATTTCCCCAG | |
| | TCAGATGCTGAGAACGGGAAATAACTTTGTCTTCA | |
| | GCTACGAGTTCGAAGACGTCCCGTTCCACAGTATG | |
| | TACATGCACAACCAATCGTTGGACCGCTTGATGAA | |
| | CCCGCTGATTGATCAGTACTTGTACTATCTCTCCGC | |
| | CACCAATGCTCCCGCCAATACAAATAAATTTACCA | |
| | AGTCAAATAAAGGTGATCTACCGTCTCAGCTCAGG | |
| | AACTGGATTCCTGGACCTTCATTTCAACAAGCAGC | |
| | TTGGACTCTAGACGGACAGAGTCAAACCCAAACG | |
| | AACAACACGTGGGATTATGCCAATAAATACCAATT | |
| | GGATGCACGAAAGAACAAGTTGCTCCCGGTCCA | |
| | GTTAATGCACCTCGAGAAGGACAGTTTCAAGCAA | |
| | ATACGCTGATATTCGGCAAAGATCCAACCGTCTCT | |
| | TCTACCTTGGCTGAACAAGACAAAGTTAACATTAC | |
| | ACGAGACAATGAAACTCGGACAGTAAACCCAATT | |
| | CTGGGAACGGCTCAGATATTTGACATCATCAACAA | |
| | CGACACTCCGCGTGCGAACGGCTCAATAAGCAAA | |
| | ATCAACATGAACATCTGTCCTGTCATGCCTGGAAG | |
| | CGTCTGGCAAGATCGAGATATCTACCTACAAGGAC | |
| | CAATCTGGGCCAAGGTTCCTCGCACAGATGGAAA | |
| | GTTCCATCCCTCTCCTCTCATGGGTGGATTTGGACT | |
| | TAGATGTCCACCTCCACAGATTTTCATTAAGAATA | |
| | CTCCGGTTCCTGGCAACCCTCCAACAACATTTGAA | |
| | CCAACCAAAGTGGCCAGCTTCATCACGCAGTACTC | |
| | GACCGGACAAGTCACCGTCGAAATGGAATGGGAG | |
| | CTTGAAAAGGAAAACTCCAAACGCTGGAACCCGG | |
| | AAATACAGTTCACCAACAACTTCTCCTCTTCGGAC | |
| | GGAAACATGGTCCACTTTGCTGTGGACTCTACCGG | |
| | ACTCTACTCAGAACCCCGTCCCGTGGGCACCCGTT | |
| | ACCTTACCCGCTCTTTGTAA (SEQ ID NO: 845) | |
| MG1 | ATGTCTTTTGTCGATCACCCTCCAGATTGGTTGGA | MSFVDHPPDWLEDVLDGFNEFLGLKSGP |
| | AGACGTTCTCGACGGCTTCAACGAATTTCTGGGTC | PQPKAGQYDKPKDNTRGLVLPGYKYLG |
| | TCAAATCGGGACCACCTCAGCCAAAGGCCGGGCA | PGNGLDKGEPVNEADAAALEHDKAYDE |
| | GTACGATAAGCCCAAAGACAACACCAGAGGCCTA | QLKAGDNPYVKYNHADAEFQERLQGDT |
| | GTCCTTCCTGGCTACAAATACTTGGGTCCGGGCAA | SFGGNLARGVFQAKKRVLEPLGLVEGDE |
| | CGGACTCGATAAAGGAGAGCCCGTCAACGAGGCT | TGPIKRPHTQESPESSSGIGKKGHQPARK |
| | GACGCCGCTGCTCTCGAGCACGACAAGGCCTACG | RLNFDQAGESSSGAAGQSNGSMSADGTE |
| | ACGAGCAGCTTAAAGCGGGAGACAACCCGTACGT | RAAAGGAAVDAAQGADGVGNSSGNWH |
| | CAAATACAATCACGCAGACGCCGAATTCCAAGAG | CDSTWGDNHVTTTSTRTWVLPNYNNHL |
| | CGTCTGCAGGGAGATACGTCTTTTGGCGGGAACCT | YRRLGVSNTSNTYNGFSTPWGYFDFNRF |
| | TGCTCGTGGTGTATTCCAGGCCAAGAAGCGGGTCC | HCHFSPRDWQRLINNHWGIRPKRISVKIF |
| | TAGAGCCGCTCGGTCTGGTCGAAGGAGACGAGAC | NIQVKEVTVQDGTTTVANNLTSTIQVFA |
| | GGGTCCCATCAAGAGACCCCATACGCAAGAGAGT | DKAYDLPYVMDAGQEGSLPPFPNDVFMI |
| | CCCGAGTCCAGTTCCGGGATCGGAAAGAAGGGCC | PQYGYMGLVTGGSSQNQTDRNAFYCLE |
| | ATCAGCCTGCCCGCAAGAGGCTCAACTTTGATCAA | YFPSQMLRTGNNFEMSFDFEDVPFHSMF |
| | GCTGGAGAATCGAGCTCTGGTGCCGCAGGACAGT | AHSQSLDRLMNPLLDQYLWSLHSTTGG |
| | CTAACGGAAGCATGTCTGCAGACGGTACTGAGCG | AYGTGTKLNTFIKNQTTDFSSYRRNWLP |
| | TGCAGCAGCTGGCGGAGCTGCGGTCGATGCAGCT | GPFIKQQRWNKGNTNYSVPVSGNDSML |
| | CAAGGTGCCGATGGAGTGGGTAATTCCTCCGGTAA | HHESHTTLNGRWSKLAPGPAMATPENT |
| | TTGGCATTGCGATTCCACGTGGGGAGATAATCACG | ATSFTQGQLMFAGPGQTTTSTTNSNNIIF |
| | TCACCACCACCAGCACCAGAACCTGGGTGCTGCCC | TSEDELIATNPTDYDYWGAVTDGQQTST |
| | AACTACAACAACCACCTGTACCGAAGACTCGGCG | VAPSERDVDAMPTMPGMVWQDRDIYY |
| | TCAGCAACACCTCCAACACCTACAACGGATTCAGC | QGPIWAKIPHTDGRFHPSPLIGGFGLHKP |
| | ACCCCCTGGGGGTACTTTGACTTTAACCGATTCCA | PPQIFIKNTPVPANPATTFSATRINTELNQ |
| | CTGCCACTTCTCTCCACGAGACTGGCAGCGACTCA | YSTGQVAVQIEWELRKEKSKRWNPEIQF |
| | TCAATAACCATTGGGGAATCAGACCCAAACGCAT | TSNYGTQNSMLWAPDAAGNYEEPRAIG |
| | CAGCGTCAAAATCTTCAACATTCAAGTCAAGGAG | TRYLTHCL (SEQ ID NO: 848) |
| | GTCACGGTGCAGGACGGCACCACGACCGTCGCCA | |
| | ACAATCTCACCAGCACCATACAGGTCTTTGCGGAC | |
| | AAGGCGTACGACCTGCCGTACGTGATGGACGCCG | |
| | GACAGGAGGGCAGTCTGCCTCCGTTCCCCAACGAC | |
| | GTCTTTATGATACCGCAGTACGGCTACATGGGACT | |
| | GGTGACAGGCGGCAGCAGCCAGAACCAGACGGAC | |
| | AGAAACGCCTTCTACTGTCTAGAGTACTTTCCCTC | |
| | TCAGATGCTGAGAACGGGAAACAACTTTGAAATG | |
| | AGCTTCGACTTTGAAGACGTGCCCTTCCACTCCAT | |
| | GTTCGCTCACAGCCAGAGTTTGGACAGACTGATGA | |
| | ATCCTCTGCTGGATCAGTACCTGTGGAGTCTGCAC | |
| | TCGACCACAGGGGGAGCCTATGGCACCGGCACCA | |
| | AGCTCAATACGTTCATCAAGAACCAGACCACAGA | |
| | CTTTTCGTCTTATAGACGCAACTGGCTGCCTGGTC | |
| | CTTTCATCAAACAGCAGAGATGGAACAAGGGCAA | |
| | TACCAACTACTCGGTGCCCGTCTCTGGAAACGACT | |
| | CGATGCTCCACCACGAGTCTCACACGACTCTGAAC | |
| | GGTCGATGGAGCAAACTGGCTCCCGGACCGGCCA | |
| | TGGCAACTCCAGAAAACACGGCTACCAGCTTTACA | |
| | CAAGGCCAGCTCATGTTTGCCGGGCCCGGGCAGA | |
| | CCACTACCAGTACCACCAACTCCAACAACATCATC | |
| | TTCACCTCGGAGGACGAGCTGATTGCCACTAATCC | |
| | CACCGACTACGATTACTGGGGAGCCGTCACAGAC | |
| | GGACAGCAGACCTCTACCGTGGCACCCAGCGAAC | |
| | GAGACGTGGACGCGATGCCGACTATGCCCGGCAT | |
| | GGTCTGGCAAGACAGAGACATTTACTACCAGGGC | |
| | CCCATCTGGGCCAAGATTCCTCATACCGACGGACG | |
| | CTTCCATCCGTCTCCTTTGATTGGAGGCTTCGGTCT | |
| | GCACAAGCCTCCTCCTCAAATCTTCATCAAAAACA | |
| | CGCCGGTACCTGCCAATCCAGCAACCACCTTCTCG | |
| | GCTACTCGCATCAACACCTTCCTGAACCAGTACAG | |
| | CACCGGACAAGTCGCCGTCCAGATCGAATGGGAG | |
| | CTGCGCAAAGAAAAGTCCAAGCGATGGAACCCGG | |
| | AAATCCAGTTCACTTCCAACTACGGTACCCAGAAT | |
| | TCCATGCTCTGGGCGCCAGATGCCGCTGGAAACTA | |
| | CGAAGAGCCCAGAGCCATCGGTACCCGCTACCTC | |
| | ACCCATTGCCTGTAA (SEQ ID NO: 847) | |
| MM2 | ATGTCTTTCTTTGATTGGTTAGGTAAACAGTACGC | MSFFDWLGKQYAQGAAEFWDLKSGPPA |
| | TCAAGGAGCTGCTGAATTCTGGGATTTGAAGTCCG | PKKARKDGSAGFNFPGHKYLGPGNSLDR |
| | GTCCTCCTGCACCAAAAAAGGCGCGAAAGGACGG | GDPVDADDAAAQKHDQSYQEQLEAGD |
| | ATCAGCCGGATTCAATTTCCCTGGGCACAAATACC | NPYLKYNHADREFQEALKDDTSFEGNLA |
| | TGGGTCCTGGCAATTCATTGGATCGCGGAGATCCC | RGLFEAKKLVAEPLGLVEPELAPPSGRKR |
| | GTGGACGCTGACGACGCCGCTGCTCAAAAGCACG | PVQSSQESGYSSSQDKRPNLDVDEEDREF |
| | ACCAGTCGTACCAAGAGCAGCTTGAGGCGGGAGA | AAAAAETETGSAPPTGNLGPGTMAGGGS |
| | CAATCCCTACCTCAAGTACAACCACGCCGACCGCG | APIDDGSYGADGVGNASGDWHCDSTWL |
| | AGTTCCAGGAGGCGTTGAAGGACGACACCTCCTTT | DNCVITRTTRTWNLPTYNNHIYKRLNGT |
| | GAAGGAAATCTCGCGAGAGGACTCTTTGAGGCCA | TSGDQSYFGFSTPWGYFDFNRFHCHFSPR |
| | AGAAGCTCGTGGCAGAGCCTCTTGGTCTCGTGGAA | DWQRLINNNWGLRPKSLRFKIFNIQVKE |
| | CCAGAACTGGCGCCACCCAGTGGCCGTAAACGAC | VTTQDSTKIISNNLTSTVQVFADTEYQLP |
| | CGGTGCAATCGAGTCAAGAGTCTGGTTACTCGAGT | YVIGSAHEGCLPPFPADVFMLPQYGYCT |
| | AGCCAAGACAAGCGGCCCAACCTCGACGTAGACG | RQDGNSNNPTPRSAFYCLEYFPSKMLRT |
| | AGGAGGACCGTGAGTTCGCTGCCGCTGCAGCGGA | GNSFEFTYNFEKVPFHSMWAHNQSLDRL |
| | GACCGAAACTGGAAGCGCTCCCCCCACCGGCAAT | MNPLIDQYLYYLDVTSSTGFTYQKGVHT |
| | TTGGGACCTGGTACGATGGCTGGAGGCGGTAGCG | NLPEQERNWLPGPGIRNQAWFNSATGNN |
| | CGCCAATCGACGACGGCTCGTATGGTGCCGATGG | PLTGTWQYSNKYVLENRASKIAPGPAMG |
| | AGTGGGCAATGCCTCGGGAGATTGGCATTGCGATT | IEKTKFDGNGIIFSKEYITNVNTANPNQVN |
| | CCACATGGCTGGACAACTGTGTCATCACCCGAACC | ITRETEINSTNPLAGGSLGAHANNSQNTT |
| | ACTCGGACCTGGAATCTGCCAACCTACAACAACCA | TAPTLDHTNVMGVFPGSVWQDRDIYLQ |
| | CATCTACAAACGACTCAACGGAACGACCTCCGGA | GQIWAKIPHTDGHFHPSPLMGGFGLKNP |
| | GACCAAAGCTACTTCGGATTCAGCACCCCCTGGGG | PPQILIKNTPVPADPPTEFNANKISSFITQY |
| | ATACTTTGACTTCAACCGCTTCCACTGTCATTTCTC | STGQVTVEMEWELQKETSKRWNPEIQYS |
| | CCCTCGAGACTGGCAAAGACTCATCAACAATAACT | DDSSSTSGSILHFAPDDVGNYKEFRSIGT |
| | GGGGACTCCGACCAAAGAGCCTACGGTTCAAAAT | RYLTRPL (SEQ ID NO: 850) |
| | CTTTAACATTCAAGTTAAAGAAGTCACGACGCAAG | |
| | ACTCAACGAAGATCATCTCCAATAACCTTACCAGC | |
| | ACGGTTCAGGTATTTGCGGACACGGAGTACCAGCT | |
| | CCCGTACGTGATTGGATCGGCTCACGAAGGATGTC | |
| | TGCCTCCTTTCCCGGCTGACGTGTTCATGCTGCCGC | |
| | AGTATGGATACTGTACTCGACAAGACGGAAACAG | |
| | CAACAATCCGACCCCGAGAAGCGCCTTCTATTGTT | |
| | TGGAGTACTTTCCCAGCAAGATGCTAAGAACTGGG | |
| | AACAGTTTTGAATTTACATATAACTTTGAGAAGGT | |
| | GCCGTTCCACAGCATGTGGGCTCACAACCAGAGTC | |
| | TGGATCGATTGATGAATCCATTGATTGATCAGTAC | |
| | CTGTACTACCTTGATGTCACTTCGAGTACCGGGTT | |
| | TACCTATCAAAAAGGAGTTCACACGAACTTGCCCG | |
| | AACAAGAGCGCAACTGGCTGCCGGGACCAGGAAT | |
| | TCGAAATCAAGCTTGGTTTAATTCTGCAACTGGAA | |
| | ACAACCCACTAACTGGTACCTGGCAATATTCCAAC | |
| | AAATACGTACTTGAAAACCGTGCTTCGAAGATTGC | |
| | TCCTGGACCTGCCATGGGAATTGAATCAACAAAAT | |
| | TCGACGGGAACGGAATCATCTTTTCTAAAGAATAC | |
| | ATCACCAATGTAAATACAGCCAATCCGAATCAAGT | |
| | AAACATCACACGCGAGACCGAAATAAACTCAACG | |
| | AACCCCTTGGCTGGAGGATCTCTCGGAGCCCACGC | |
| | TAATAATTCACAAAACACAACAACGGCACCGACG | |
| | CTAGACCACACCAACGTCATGGGTGTGTTTCCGGG | |
| | TAGTGTCTGGCAAGACAGAGACATTTACCTTCAAG | |
| | GACAAATCTGGGCCAAGATTCCCCACACAGACGG | |
| | ACATTTCCACCCTTCTCCTCTCATGGGAGGATTTG | |
| | GACTGAAGAACCCGCCTCCTCAAATTCTGATCAAA | |
| | AACACACCTGTTCCGGCTGACCCACCAACTGAATT | |
| | CAATGCGAACAAAATCTCTTCTTTCATCACTCAAT | |
| | ACTCAACCGGACAAGTTACAGTGGAAATGGAATG | |
| | GGAACTTCAGAAAGAAACCTCCAAAAGATGGAAT | |
| | CCAGAAATCCAGTACAGCGACGACTCGTCTTCGAC | |
| | GTCTGGCTCCATTCTGCACTTTGCTCCGGATGATGT | |
| | TGGAAACTACAAAGAGTTCCGCTCTATCGGAACTC | |
| | GTTACCTTACCCGTCCTCTGTAA (SEQ ID NO: 849) | |
| MM3 | ATGTCTTTCTTTGATTGGATAGGTAAACAGTACGC | MSFFDWIGKQYAQGAAEFWDLKSGPPA |
| | TCAAGGAGCTGCTGAATTCTGGGATTTGAAGTCCG | PKKARKDGSAGFNFPGHKYLGPGNSLDR |
| | GTCCTCCTGCACCAAAAAAGGCGCGAAAGGACGG | GDPVDADDAAAQKHDQSYQEQLEAGD |
| | ATCCGCCGGATTCAATTTCCCTGGGCACAAGTACC | NPYLKYNHADREFQEALKGDTSFEGNLA |
| | TGGGTCCTGGCAATTCATTGGATCGCGGAGATCCC | RGLFEAKKLVAEPLGLVEPELAPPSGRKR |
| | GTGGACGCCGACGACGCTGCTGCTCAAAAGCACG | PVQSSQESGYSSSQDKRPNLDLDEEDREL |
| | ACCAGTCGTATCAAGAACAGCTTGAGGCGGGAGA | AAAAAETETGSAPPTGNLGPGTMAGGGS |
| | CAATCCCTACCTCAAGTACAATCACGCGGATCGCG | APIDDGSYGADGVGNASGDWHCDSIWM |
| | AGTTCCAGGAGGCGTTGAAAGGAGACACCTCCTTC | DDRVVTRTTRTWNLPTYNNHVYKRLNG |
| | GAAGGAAATCTCGCGAGAGGACTCTTTGAGGCCA | NTEQTTGDGSYFGFSTPWGYFDFNRFHC |
| | AGAAGCTCGTTGCAGAGCCTCTCGGCCTTGTTGAA | HFSPRDWQRLINNNWGLRPKSLRFKIFNI |
| | CCGGAACTGGCGCCACCTAGTGGCCGCAAGCGAC | QVKEVTVQDQTTIISNNLTSTVQVFADTD |
| | CGGTGCAATCGAGTCAAGAATCTGGTTACTCGAGT | YQLPYVIGSAHEGCLPPFPADVFMLPQY |
| | AGCCAAGACAAGCGGCCCAACCTCGACTTAGACG | AYCTRQDTNSSNPTPRSAFYCLEYFPSK |
| | AGGAGGATCGTGAGCTCGCTGCCGCTGCAGCGGA | MLRTGNNFEFTYNFEKVPFHSMWAHNQ |
| | GACCGAAACTGGAAGCGCTCCCCCCACCGGCAAT | SLDRLMNPLIDQYLYYLNQTTNSALSSRS |
| | TTGGGACCTGGTACGATGGCTGGAGGCGGTAGCG | KTGYEPSHRSVFVLPQSKYNSALSYNKS |
| | CGCCAATCGACGACGGCTCGTATGGTGCCGATGG | TKSNLPEQERNWLPGPGIRNQTWFSSGT |
| | AGTGGGCAATGCCTCGGGAGATTGGCATTGCGATT | GNAMVDAMWNYSNKYVLENRSSKIAP |
| | CCATATGGATGGACGACCGTGTCGTCACCCGCACC | GPAMGIESTKVDGNGLIFSKEYITNSTTA |
| | ACTCGGACCTGGAACCTGCCCACCTACAATAACCA | NADQVNVTRETEINSTNPLAGKSLGAHA |
| | CGTCTACAAACGACTCAACGGAAACACGGAACAG | NNIQNQTTLPTLANTDIMPVLPGSVWQN |
| | ACCACCGGAGATGGAAGCTACTTCGGATTCAGCA | RDIYLQGQIWAKIPHTDGHFHPSPLMGGF |
| | CCCCGTGGGGATACTTCGACTTCAACCGCTTCCAC | GLKNPPPQILIKNTPVPANPPVEFTAGKIN |
| | TGTCATTTCTCCCCCAGAGACTGGCAAAGACTCAT | SFITQYSTGQVTVEMEWELQKETSKRWN |
| | CAATAACAATTGGGGACTCAGACCAAAAAGTCTA | PEIQYSDDSSATNQSVLHFAPNETGEYKE |
| | CGGTTCAAAATCTTTAACATTCAGGTTAAAGAGGT | FRSIGSRYLTRPL (SEQ ID NO: 852) |
| | CACGGTGCAAGACCAAACGACGATCATCTCCAAT | |
| | AACCTTACCAGCACGGTTCAAGTGTTTGCGGACAC | |
| | GGACTATCAATTGCCGTACGTGATTGGTTCTGCTC | |
| | ACGAAGGTTGTCTGCCTCCTTTCCCAGCCGATGTG | |
| | TTTATGCTGCCGCAGTATGCATACTGCACTCGACA | |
| | AGACACAAACAGCAGTAATCCCACTCCCAGAAGC | |
| | GCTTTCTATTGTTTGGAATACTTTCCCAGCAAGAT | |
| | GTTGAGAACTGGTAACAACTTTGAATTTACTTACA | |
| | ATTTTGAGAAGGTGCCCTTCCACAGCATGTGGGCT | |
| | CATAACCAGTCTTTAGATCGGCTGATGAACCCTCT | |
| | CATCGATCAGTATTTGTACTACCTCAATCAAACTA | |
| | CAAATAGTGCTCTATCCAGCCGATCTAAGACTGGT | |
| | TACGAGCCTAGCCACAGAAGCGTGTTCGTGCTGCC | |
| | CCAGAGCAAGTACAATAGTGCTCTATCCTACAACA | |
| | AATCAACAAAATCAAATCTACCTGAGCAAGAACG | |
| | CAACTGGCTACCTGGACCTGGCATCAGAAATCAA | |
| | ACGTGGTTCAGCAGTGGAACTGGCAACATGGCTGT | |
| | AGACGCCATGTGGAACTACTCGAATAAGTATGTGC | |
| | TGGAGAACAGAGCTTCCAAGATTGCTCCTGGTCCA | |
| | GCTATGGGGATTGAGTCAACAAAAGTAGATGGCA | |
| | ATGGACTAATCTTCTCTAAAGAGTATATAACTAAT | |
| | TCAACCACCGCGAATGCTGATCAAGTGAACGTCAC | |
| | TAGAGAAACTGAAATCAACTCAACCAATCCCTTGG | |
| | CGGGCAAATCACTTGGAGCTCATGCGAACAATAT | |
| | ACAGAACCAGACAACACTGCCAACACTTGCTAAT | |
| | ACTGACATCATGCCGGTACTACCTGGCAGCGTTTG | |
| | GCAGAATAGAGATATCTACCTTCAAGGCCAAATCT | |
| | GGGCGAAGATTCCTCACACAGATGGACACTTTCAT | |
| | CCTTCACCGCTAATGGGAGGATTCGGTCTAAAGAA | |
| | TCCTCCTCCTCAAATACTGATCAAGAACACACCAG | |
| | TGCCAGCAAATCCTCCGGTAGAATTTACAGCTGGC | |
| | AAGATTAACTCTTTCATCACTCAGTATTCAACTGG | |
| | TCAAGTCACAGTCGAAATGGAATGGGAACTTCAG | |
| | AAAGAAACATCCAAAAGATGGAATCCAGAGATCC | |
| | AATACAGCGACGACTCATCAGCCACCAACCAATC | |
| | GGTGCTGCACTTTGCTCCAAACGAAACAGGAGAG | |
| | TACAAGGAATTCCGTTCAATTGGATCCCGTTATCT | |
| | CACTCGTCCTCTGTAA (SEQ ID NO: 851) | |
| SD3 | ATGTCGTTTGTTGATCACCCTCCAGATTGGTTAGA | MSFVDHPPDWLEEVGEGLHEFLELEAGP |
| | AGAGGTTGGTGAAGGCCTTCACGAGTTTTTGGAGC | PKPKPNQQKQDNARGLVLPGYNYLGPF |
| | TCGAAGCTGGCCCACCCAAACCGAAGCCCAATCA | NGLDKGEPVNRADAVAREHDISYNEQL |
| | GCAAAAACAAGACAACGCCCGTGGTCTTGTACTG | QAGDNPYLKYNHADAEFQEKLADDTSF |
| | CCTGGATATAATTATCTGGGACCCTTCAACGGACT | GGNLGKAIFQAKKRVLEPFGLVEEPVKT |
| | CGACAAGGGAGAGCCCGTCAACCGAGCGGACGCT | APAKKRPVEQSPAEPGPSSGIGKAGDQPA |
| | GTTGCGCGAGAGCACGATATCTCGTACAACGAGC | RKRLNFGQTGDADSEPDPQPLGEAPAAN |
| | AGCTCCAGGCGGGAGACAACCCCTACCTCAAGTA | PNLGSATMSGGGGAPMADDQQGADGV |
| | CAACCACGCGGACGCCGAGTTTCAGGAGAAGCTC | GTSSGNRHCDSTWLDKRIITKSTRTWSLP |
| | GCCGACGACACCTCCTTTGGGGGCAACCTCGGAA | TYNNHLYNKIMSTNANGDSAWFGFSTP |
| | AGGCAATCTTTCAGGCCAAGAAGAGGGTTCTCGA | WGYFDFNRFHCHFSPRDWQRLINNNWGI |
| | ACCTTTTGGCCTGGTTGAAGAGCCTGTTAAGACGG | RPKKMHVNLFNIQVKEVTTQDSTTTTTN |
| | CTCCGGCAAAGAAGAGGCCTGTAGAGCAGTCTCC | NLTSTIQVFADTEYQLPYVASNAHEGCLP |
| | TGCGGAACCGGGCCCATCCTCTGGCATCGGAAAG | PFPADVFMLPQYGYCTLQRENSNDPVAQ |
| | GCGGGCGATCAGCCTGCGAGAAAGCGACTAAACT | SSFYCLEYFPSQMLRTGNNYSISYTFEDV |
| | TTGGTCAGACTGGAGACGCCGACTCCGAACCCGA | PFHSMYMHNQSLDRLMNPLIDQYLWYL |
| | CCCCCAGCCTCTCGGAGAAGCACCAGCAGCCAAT | NTTVSTNNTNTFSKSTKGDLARQNRNWL |
| | CCCAATTTGGGTTCTGCTACAATGTCTGGAGGCGG | PGPAFQTVAFNLNGQGNFGSTNHWLYL |
| | AGGCGCGCCGATGGCAGATGATCAACAGGGTGCC | NKYTMNGKSVAIGPGPVVAPKSTTFQAD |
| | GATGGAGTGGGTACTTCCTCAGGAAATCGGCATTG | GMIFAKSADTATSAAPQNEVNITSEAETS |
| | CGATTCCACATGGCTGGACAAGCGAATCATTACCA | TVNPMMGTNPLIINSSNTPSTSAPTMSNV |
| | AGTCCACCCGAACCTGGAGCCTGCCCACCTACAAC | SVNPVMPGSVWQDRDIYLQGPIWAKIPR |
| | AACCATCTCTACAACAAGATCATGTCCACCAACGC | TDGTFHPSPLMGGFGLRRPPPQIFIKNTPV |
| | CAATGGAGACAGCGCTTGGTTTGGCTTCAGCACTC | PSNPPTTFNPAKINSFITQYSTGQVTVEME |
| | CCTGGGGATACTTTGACTTCAACCGCTTCCACTGC | WELEKENSKRWNPEIQFTNNFQVSDGN |
| | CACTTCTCGCCCCGAGACTGGCAACGACTCATCAA | MVPFCVDSRGVYSEPRPIGTRYLTRTL |
| | CAACAACTGGGGCATCCGGCCCAAGAAAATGCAC | (SEQ ID NO: 854) |
| | GTCAACCTCTTCAATATCCAAGTCAAGGAAGTCAC | |
| | GACGCAAGACTCGACGACGACCACCACCAATAAC | |
| | CTTACCAGCACGATTCAGGTGTTTGCGGACACGGA | |
| | GTACCAGCTGCCGTACGTAGCCAGCAACGCCCAC | |
| | GAGGGCTGTCTCCCGCCCTTTCCGGCGGACGTCTT | |
| | CATGCTTCCGCAGTACGGCTACTGCACGCTGCAAC | |
| | GCGAGAACTCCAACGATCCTGTGGCCCAGAGCTC | |
| | GTTCTACTGCCTGGAGTACTTCCCCAGCCAGATGC | |
| | TGAGGACCGGAAACAACTACTCCATCAGCTACAC | |
| | GTTCGAAGACGTGCCCTTCCACAGCATGTACATGC | |
| | ACAACCAGAGCCTGGACCGGCTGATGAACCCGCT | |
| | CATCGACCAGTACTTGTGGTACCTGAATACCACCG | |
| | TATCGACCAACAACACAAACACCTTCTCCAAGTCG | |
| | ACCAAAGGCGACCTGGCACGCCAGAACCGAAACT | |
| | GGCTGCCTGGACCCGCCTTCCAGACGGTGGCCTTC | |
| | AACCTGAACGGACAAGGCAACTTTGGATCGACCA | |
| | ATCACTGGCTTTACTTGAACAAGTACACAATGAAT | |
| | GGAAAAAGCGTGGCTATCGGCCCCGGCCCCGTCG | |
| | TGGCTCCCAAGAGTACAACCTTCCAGGCCGACGGC | |
| | ATGATCTTCGCCAAGAGCGCAGATACGGCGACCTC | |
| | TGCTGCCCCTCAAAATGAGGTCAACATCACCAGCG | |
| | AGGCGGAGACGTCCACCGTCAATCCCATGATGGG | |
| | CACCAATCCTCTCATTATCAATTCGAGCAATACTC | |
| | CATCTACCTCCGCTCCCACGATGTCAAATGTATCC | |
| | GTTAATCCCGTCATGCCTGGCTCCGTTTGGCAAGA | |
| | CAGAGACATCTACCTTCAAGGCCCGATCTGGGCCA | |
| | AGATTCCGCGTACCGACGGAACCTTCCACCCCTCG | |
| | CCTCTGATGGGAGGATTCGGGCTGCGTCGTCCTCC | |
| | TCCTCAAATCTTCATCAAGAACACGCCCGTTCCGT | |
| | CCAATCCTCCAACCACGTTCAATCCCGCCAAGATC | |
| | AACTCCTTCATCACCCAATACTCGACCGGACAGGT | |
| | CACCGTTGAGATGGAATGGGAGCTCGAGAAGGAA | |
| | AACAGCAAGAGGTGGAACCCGGAAATACAGTTCA | |
| | CCAACAATTTCCAGGTTTCGGACGGGAACATGGTT | |
| | CCCTTCTGCGTGGACTCACGTGGTGTCTACTCTGA | |
| | ACCCCGCCCCATCGGAACCCGCTACCTTACCCGAA | |
| | CCCTCTAA (SEQ ID NO: 853) | |
| MK1 | ATGTCGTTTGTCGATCACCCCCCAGACTGGCTCGA | MSFVDHPPDWLEEIGEGLSEFIGLEAGPP |
| | GGAGATCGGAGAGGGGCTCTCCGAGTTCATCGGA | KPKPGYQDRARGLVVPGYKYLGPFNGL |
| | CTAGAGGCGGGACCGCCGAAGCCTAAGCCGGGCT | DRGEPVNAADAAAKKHDEEYDRLLKAG |
| | ACCAGGACCGCGCGCGCGGCCTCGTCGTCCCCGGC | DNPYLAYNHADAEFQKDLSGDSSLAGN |
| | TACAAGTACCTCGGGCCCTTCAACGGACTCGACAG | AANALFQAKKRVLEPFGLVEGEPEPKKT |
| | GGGCGAGCCCGTCAACGCGGCCGACGCGGCGGCC | PSVKRPHASPDSSSGVGKKGDQPARKRL |
| | AAGAAGCACGACGAGGAGTACGACCGGCTCCTCA | DFGTEPAGQDGAGRAAQAAGDMASAEV |
| | AGGCGGGGGACAACCCGTACCTCGCCTACAACCA | AAGGGGPVGDDAQGADGVGNASGNWH |
| | CGCGGACGCCGAGTTCCAGAAGGACCTCAGCGGC | YDSVWMDGAVITKSTRTWSLPAYNNHL |
| | GACTCCAGCCTAGCCGGCAACGCCGCGAACGCGC | YRQIQSSGTGDGTYFGYSTPWGYFDFNR |
| | TCTTCCAGGCCAAGAAGAGGGTCCTCGAGCCCTTC | FHCHFSPRDWQRLINNHWGIRPKRLHFK |
| | GGACTGGTCGAGGGAGAGCCGGAGCCCAAGAAGA | LFNIQVKEVTTTDGTTTIANNLTSTIQVFA |
| | CGCCTTCCGTCAAGAGGCCGCACGCATCGCCGGAC | DTEYQLPYVLGNAHEGCLPPFPADVFML |
| | TCGTCGAGCGGCGTCGGGAAGAAGGGCGACCAGC | PQYAYLTLNANPTNQGAAALSLPQSAFY |
| | CCGCGCGGAAGAGGCTCGACTTTGGGACGGAGCC | CLEYFPSQMLRTGNNFSFSYEFEKLPFHS |
| | CGCGGGTCAAGACGGAGCGGGACGAGCGGCCCAG | MFMHSQSLDRLMNPLIDQYLWYLNATT |
| | GCAGCCGGAGATATGGCATCTGCTGAGGTGGCTG | GNNLSFNKAGAKNFPEYFRNWLPGPGQ |
| | CGGGTGGTGGCGGACCAGTGGGCGACGATGCACA | RVQQWSTIGTQNNAQTGTWASANKWIL |
| | AGGTGCCGATGGAGTGGGTAATGCCTCAGGAAAT | MGRSSKMAPGLAQPVRNAQTVTNGSQLI |
| | TGGCATTACGATTCCGTTTGGATGGACGGCGCTGT | FNNETIKGSTATASTVHSGLLVTNESETA |
| | CATCACCAAGTCCACCCGAACCTGGAGCCTGCCCG | PTNPNSATKWGVMTDNQQTTSTTPTVSD |
| | CCTACAACAACCACCTCTACCGCCAGATCCAGTCC | DLEAHVFPGMVWQDRDIYLQGPIWAKIP |
| | AGCGGCACCGGAGACGGCACGTACTTTGGTTACA | ETDGHFHPSPLMGGFGLKNPPPQILVKNT |
| | GCACGCCTTGGGGATACTTCGATTTCAATCGATTC | PVPAVPPTTFTPQKVNSFITQYSTGQVTV |
| | CACTGCCACTTTTCTCCCAGGGACTGGCAGCGGCT | EIEWELRKEKSKRWNPEIQYTSNFENSAN |
| | CATCAACAACCACTGGGGCATCCGCCCCAAGCGG | VQFSVNGDGAYEIPRPIGTRYLTHNL |
| | CTCCACTTTAAGCTCTTCAACATCCAGGTCAAGGA | (SEQ ID NO: 856) |
| | GGTCACGACGACCGACGGCACCACGACCATCGCC | |
| | AACAACCTCACCAGCACCATACAGGTCTTTGCGGA | |
| | CACGGAGTACCAGCTCCCGTACGTGCTCGGCAACG | |
| | CCCACGAGGGCTGCCTGCCGCCGTTCCCGGCGGAC | |
| | GTCTTCATGCTGCCGCAGTACGCGTACCTGACGCT | |
| | CAACGCCAACCCCACGAACCAGGGCGCGGCCGCG | |
| | CTCAGCCTGCCGCAGAGCGCCTTCTACTGCCTGGA | |
| | GTACTTTCCGAGCCAGATGCTGAGGACCGGGAAC | |
| | AACTTCTCGTTCAGCTACGAGTTCGAGAAGCTGGC | |
| | CTTCCACTCGATGTTCATGCACAGCCAGAGCCTGG | |
| | ACCGGCTCATGAACCCGCTCATCGACCAGTACCTC | |
| | TGGTACCTCAACGCCACGACCGGCAACAACCTCTC | |
| | CTTCAACAAGGCCGGCGCCAAGAACTTCCCCGAGT | |
| | ACTTCCGCAACTGGCTGCCCGGGCCCGGCCAGCGC | |
| | GTGCAGCAGTGGAGCACGATCGGAACCCAGAACA | |
| | ACGCGCAGACCGGCACGTGGGCCAGCGCCAACAA | |
| | GTGGATCCTCATGGGCAGGTCCAGCAAGATGGCC | |
| | CCCGGGCTGGCCCAGCCGGTCAGGAACGCCCAGA | |
| | CCGTCACCAACGGCTCGCAGCTCATCTTCAACAAC | |
| | GAGACCATCAAGGGCTCCACCGCGACGGCCTCCA | |
| | CCGTACACTCGGGGCTGCTCGTCACCAACGAGTCG | |
| | GAGACCGCCCCGACCAACCCCAACTCGGCGACAA | |
| | AGTGGGGCGTCATGACGGACAACCAGCAGACCAC | |
| | CTCCACTACGCCGACCGTCAGCGACGACCTCGAGG | |
| | CGCACGTCTTCCCGGGCATGGTCTGGCAGGACCGC | |
| | GACATCTACCTGCAGGGCCCGATATGGGCCAAGA | |
| | TCCCGGAGACGGACGGCCACTTCCACCCGTCCCCG | |
| | CTCATGGGCGGCTTCGGGCTCAAGAACCCGCCGCC | |
| | GCAGATCCTCGTCAAGAACACGCCCGTGCCCGCCG | |
| | TGCCGCCCACGACCTTCACACCGCAGAAGGTCAAC | |
| | TCCTTCATCACGCAGTACTCCACCGGCCAGGTCAC | |
| | CGTCGAGATCGAGTGGGAGCTGCGCAAGGAGAAG | |
| | AGCAAGCGCTGGAACCCCGAGATCCAGTACACCT | |
| | CCAACTTCGAGAACTCGGCCAACGTACAGTTCTCC | |
| | GTCAACGGCGACGGCGCGTACATCGAGCCGCGAC | |
| | CGATCGGAACCCGGTACCTCACCCACAACCTGTAA | |
| | (SEQ ID NO: 855) | |
| CT1 | ATGTCGTTTGTCGATCACCCCCCAGACTGGCTCGA | MSFVDHPPDWLEEIGEGLSEFIGLEAGPP |
| | GGAGATCGGAGAGGGGCTCTCCGAGTTTATCGGA | KPKPGYQDRARGLVVPGYKYLGPFNGL |
| | CTAGAGGCGGGACCGCCGAAGCCTAAGCCGGGCT | DRGEPVNAADAAAKKHDEEYDRLLKEG |
| | ACCAGGACCGCGCGCGCGGCCTCGTCGTCCCCGGC | DNPYLTYNHADAQFQQDLRGDASLAGN |
| | TACAAGTACCTCGGGCCCTTCAACGGACTCGACAG | AANALFQAKKRVLEPFGLVEGEPEPKKT |
| | GGGCGAGCCCGTCAACGCGGCCGACGCGGCGGCC | PSVKRPHASPDSSSGVGKKGDQPARKRL |
| | AAGAAGCACGACGAGGAGTACGACCGGCTCCTCA | DFGETPASQDGAGRAAQATGDMASAEV |
| | AGGAGGGGGACAACCCGTACCTCACCTACAACCA | AAGGGGPVGDDAQGADGVGSASGNWH |
| | CGCCGACGCCCAGTTCCAGCAGGACCTCCGGGGC | CDSVWMDGAVITKSTRTWSLPAYNNHL |
| | GACGCCAGCCTAGCCGGCAACGCCGCGAACGCGC | YRQIQSSGTGDGTYFGYSTPWGYFDFNR |
| | TCTTCCAGGCCAAGAAGAGGGTCCTCGAGCCCTTC | FHCHFSPRDWQRLINNHWGIRPKRLHFK |
| | GGACTGGTCGAGGGAGAGCCGGAGCCGAAGAAGA | LFNIQVKEVTTTDGTTTIANNLTSTIQVFA |
| | CGCCTTCCGTCAAGAGGCCGCACGCATCGCCGGAC | DTEYQLPYVLGNAHEGCLPPFPADVFML |
| | TCGTCGAGCGGCGTCGGGAAGAAGGGCGACCAGC | PQYAYLTLNANPTSQSAAALSLPQSAFY |
| | CCGCGAGGAAGAGGCTCGACTTTGGAACGGAGCC | CLEYFPSQMLRTGNNFSFSYEFEKLPFHS |
| | CGCGAGTCAAGACGGAGCCGGACGAGCGGCGCAG | MFMHSQSLDRLMNPLIDQYLWYLNATT |
| | GCAACCGGAGATATGGCATCTGCTGAAGTGGCTG | GNNLSFNKAGAKNFPEYFRNWLPGPGQ |
| | CGGGTGGTGGCGGACCAGTGGGCGACGATGCACA | RVQQWSTIGTQNNAQTGTWASANKWIL |
| | AGGTGCCGATGGAGTGGGTAGCGCCTCGGGAAAT | MGRSSKMAPGLAQPVRNAQTVTNGSQLI |
| | TGGCATTGCGATTCCGTTTGGATGGACGGCGCTGT | FNNETIKGSTATASTVHSGLLVTNESETA |
| | CATCACCAAGTCCACCCGAACCTGGAGCCTGCCCG | PTNPNSATKWGVMTDNQQTTSTTPTVSD |
| | CCTACAACAACCACCTCTACCGCCAGATCCAGTCC | DLEAHVFPGMVWQDRDIYLQGPIWAKIP |
| | AGCGGCACCGGAGACGGCACGTACTTTGGTTACA | ETDGHFHPSPLMGGFGLKNPPPQILVKNT |
| | GCACGCCTTGGGGATACTTCGATTTCAATCGATTC | PVPAVPPTTFTPQKVNSFITQYSTGQVTV |
| | CACTGCCACTTTTCTCCCAGGGACTGGCAGCGGCT | EIEWELRKEKSKRWNPEIQYTSNFENSAN |
| | CATCAACAACCACTGGGGCATCCGCCCCAAGCGG | VQFSVNGDGAYIEPRPIGTRYLTHTL |
| | CTCCACTTCAAGCTCTTCAACATCCAGGTCAAGGA | (SEQ ID NO: 858) |
| | GGTCACGACGACCGACGGCACCACGACCATCGCC | |
| | AACAACCTCACCAGCACCATACAGGTCTTTGCGGA | |
| | CACGGAGTACCAGCTCCCGTACGTGCTCGGCAACG | |
| | CCCACGAGGGCTGCCTGCCGCCGTTCCCGGCGGAC | |
| | GTCTTCATGCTGCCGCAGTACGCGTACCTGACGCT | |
| | CAACGCCAACCCGACGTCGCAGAGCGCGGCCGCG | |
| | CTCAGCCTGCCGCAGAGCGCCTTCTACTGCCTCGA | |
| | GTACTTCCCGAGCCAGATGCTGAGGACCGGGAAC | |
| | AACTTCTCGTTCAGCTACGAGTTCGAGAAGCTGCC | |
| | CTTCCACTCGATGTTCATGCACAGCCAGAGCCTGG | |
| | ACCGGCTCATGAACCCGCTCATCGACCAGTACCTC | |
| | TGGTACCTCAACGCCACGACCGGCAACAACCTCTC | |
| | CTTCAACAAGGCCGGCGCCAAGAACTTCCCCGAGT | |
| | ACTTCCGCAACTGGCTGCCCGGGCCCGGCCAGCGC | |
| | GTGCAGCAGTGGAGCACGATCGGGACCCAGAACA | |
| | ACGCGCAGACCGGCACGTGGGCCAGCGCCAACAA | |
| | GTGGATCCTCATGGGCAGGTCCAGCAAGATGGCC | |
| | CCCGGGCTGGCCCAGCCGGTCCGGAACGCCCAGA | |
| | CCGTCACCAACGGCTCGCAGCTCATCTTCAACAAC | |
| | GAGAGCCATCAAGGGCTCCACCGCGACGGCCTCCA | |
| | CCGTGCACTCGGGGCTGCTCGTCACCAACGAGTCG | |
| | GAGACCGCCCCGACCAACCCCAACTCGGCGACAA | |
| | AGTGGGGCGTCATGACGGACAATCAGCAGACCAC | |
| | CTCCACTACGCCGACCGTCAGCGACGACCTCGAGG | |
| | CGCACGTCTTCCCGGGCATGGTCTGGCAGGACCGC | |
| | GACATCTACCTGCAGGGCCCGATCTGGGCCAAGAT | |
| | CCCGGAGACGGACGGCCACTTCCACCCGTCCCCGC | |
| | TCATGGGCGGCTTCGGGCTCAAGAACCCGCCGCCG | |
| | CAGATCCTCGTCAAGAACACGCCCGTGCCCGCCGT | |
| | GCCGCCCACGACCTTCACGCCGCAGAAGGTCAACT | |
| | CCTTCATCACGCAGTACTCCACCGGCCAGGTCACC | |
| | GTCGAGATCGAGTGGGAGCTGCGCAAGGAGAAGA | |
| | GCAAGCGCTGGAACCCCGAGATCCAGTACACGTC | |
| | CAACTTCGAGAACTCGGCCAACGTTCAGTTCTCCG | |
| | TCAACGGCGACGGCGCGTACATCGAGCCCCGCCC | |
| | GATCGGAACCCGATACCTCACCCACACCCTGTAA | |
| | (SEQ ID NO: 857) | |
| CT2 | ATGTCGTTTGTCGATCACCCCCCAGACTGGCTCGA | MSFVDHPPDWLEEIGEGLSEFIGLEAGPP |
| | GGAGATCGGAGAGGGGCTCTCCGAGTTTATCGGA | KPKPGYQDRARGLVVPGYKYLGPFNGL |
| | CTAGAGGCGGGACCGCCGAAGCCTAAGCCGGGCT | DRGEPVNAADAAAKKHDEEYDRLLKAG |
| | ACCAGGACCGCGCGCGCGGCCTCGTCGTCCCCGGC | DNPYLTYNHADAEFQKDLRGDASLAGN |
| | TACAAGTACCTCGGGCCCTTCAACGGACTCGACAG | AANALFQAKKRVLEPFGLVEGEPEPKKT |
| | GGGCGAGCCCGTCAACGCGGCCGACGCGGCGGCC | PSVKRPHASPDSSSGIGKRGDQPARKRLD |
| | AAGAAGCACGACGAGGAGTACGACCGGCTCCTCA | FGTEPASQDGAGRAANAAGDMASAEVA |
| | AGGCGGGGGACAACCCGTACCTCACCTACAACCA | AGGGGPVGDDAQGADGVGQSSGNWHC |
| | CGCGGACGCCGAGTTCCAGAAGGACCTCCGGGGC | DSVWMGDRVLTKSTRTWSLPTYNNHLY |
| | GACGCCAGCCTAGCCGGCAACGCCGCGAACGCGC | KQINGSGTGDAVYFGYSTPWGYFDFNRF |
| | TCTTCCAGGCCAAGAAGAGGGTCCTCGAGCCCTTC | HCHFSPRDWQRLVNNHWGIRPRRLNFKL |
| | GGACTGGTCGAGGGAGAGCCGGAGCCGAAGAAGA | FNIQVKEVTTTDGTKTIANNLTSTVQVFA |
| | CGCCTTCCGTCAAGAGGCCGCACGCATCGCCGGAC | DTEHQLPYILGSAHEGCMPPFPADVFML |
| | TCGTCGAGCGGAATCGGGAAGAGGGGCGACCAGC | PQYGYLTLNGPGSNNNNLSTPSSAFYCLE |
| | CCGCGAGGAAGAGGCTCGACTTTGGAACGGAGCC | YFPSQMLRTGNNFVFTYEFEKVPFHSMF |
| | CGCGAGTCAAGACGGAGCGGGAAGAGCGGCCAAC | MHNQALDRLMNPLVDQYLWYLDATSG |
| | GCAGCCGGAGATATGGCATCTGCTGAGGTGGCTG | NNLTFRKAGAKNFPEYFRNWIPGPGCRN |
| | CGGGTGGTGGCGGACCAGTGGGCGACGATGCACA | QQWNKVGTKNNPQTGTWASANKWRLQ |
| | AGGTGCCGATGGAGTGGGTCAGTCCTCGGGAAAT | GRLNKYAPGQPNAPAEGFLTNAGDLAFA |
| | TGGCATTGCGATTCCGTTTGGATGGGCGACCGAGT | NAKATGATTAAGTVPADILLTSESETTTT |
| | GCTCACCAAGTCCACCCGGACCTGGAGCCTGCCCA | NMMSNNGWGAIASNNQNASVAPTVQYE |
| | CCTACAACAACCACCTGTACAAGCAGATCAACGG | DSAHVLPGMVWQDRDIYLQGPIWAKIPE |
| | CAGCGGAACCGGGGACGCGGTGTACTTTGGTTAC | TDGHFHPSPLMGGFGLKNPPPQILIKNTP |
| | AGCACGCCTTGGGGATACTTCGATTTCAATCGATT | VPADPPTQFSSQKINSFITQYSTGQMTVEI |
| | CCACTGCCACTTTTCTCCCCGAGACTGGCAGCGCC | EWELRKENSKRWNPEIQYTANFNNSAN |
| | TCGTCAACAACCACTGGGGCATCCGCCCCCGAAG | AQFSVNNNGLYIEDRTIGTRYLTHTL |
| | GCTCAACTTCAAGCTCTTCAACATCCAGGTCAAGG | (SEQ ID NO: 860) |
| | AGGTCACGACGACGGACGGCACCAAGACCATCGC | |
| | CAATAACCTCACGTCCACCGTCCAGGTGTTTGCGG | |
| | ACACGGAGCACCAGCTCCCGTACATCCTAGGCTCC | |
| | GCGCACGAGGGCTGCATGCCGCCCTTCCCCGCGGA | |
| | CGTCTTCATGCTGCCGCAGTACGGCTACCTCACGC | |
| | TCAACGGGCCCGGCAGCAACAACAACAACCTCAG | |
| | CACGCCCTCGAGCGCCTTCTACTGCCTCGAGTACT | |
| | TCCCGAGCCAGATGCTGCGCACGGGCAACAACTTC | |
| | GTCTTCACCTACGAGTTCGAGAAGGTGCCCTTCCA | |
| | CTCGATGTTCATGCACAACCAGGCGCTGGACCGCC | |
| | TCATGAACCCGCTCGTCGACCAGTACCTCTGGTAC | |
| | CTCGACGCCACCTCCGGCAACAACCTCACCTTCCG | |
| | CAAGGCCGGCGCCAAGAACTTCCCCGAGTACTTCC | |
| | GCAACTGGATCCCAGGCCCGGGCTGCCGCAACCA | |
| | GCAGTGGAACAAGGTCGGCACCAAGAACAACCCG | |
| | CAGACCGGCACGTGGGCGTCCGCCAACAAGTGGC | |
| | GGCTGCAGGGCCGCCTCAACAAGTACGCGCCCGG | |
| | ACAGCCCAACGCCCCCGCGGAGGGCTTCCTCACCA | |
| | ACGCCGGCGACCTGGCCTTCGCCAACGCCAAGGC | |
| | CACCGGCGCCACCACGGCCGCCGGAACCGTGCCC | |
| | GCCGACATCCTGCTCACCAGCGAGAGCGAGACCA | |
| | CCACCACCAACATGATGTCCAACAACGGCTGGGG | |
| | CGCGATCGCCTCCAACAACCAGAACGCGTCCGTCG | |
| | CGCCCACCGTGCAGTACGAGGACTCGGCGCACGT | |
| | GCTGCCCGGCATGGTCTGGCAGGACCGCGACATCT | |
| | ACCTGCAGGGCCCGATCTGGGCCAAGATCCCGGA | |
| | GACGGACGGCCACTTCCACCCCTCGCCGCTCATGG | |
| | GCGGCTTCGGACTCAAGAACCCGCCCCCGCAGATC | |
| | CTCATCAAGAACACGCCCGTGCCCGCCGACCCCCC | |
| | GACGCAGTTCTCCTCGCAGAAGATCAACTCCTTCA | |
| | TCACGCAGTACTCCACCGGCCAGATGACCGTCGAG | |
| | ATCGAGTGGGAGCTGCGCAAGGAGAACTCCAAGC | |
| | GCTGGAACCCCGAGATCCAGTACACGGCCAACTTC | |
| | AACAACAGCGCCAACGCGCAGTTCTCCGTCAACA | |
| | ACAACGGCCTCTACATCGAGGACCGCACCATCGG | |
| | AACCCGATACCTCACCCACACCCTGTAA (SEQ ID | |
| | NO: 859) | |
| RP1 | ATGATTCGAGTAGTCATGTCGTTTGTTGACTATCCT | MIRVVMSFVDYPPDWLKEVAGGLKEFID |
| | CCAGATTGGCTGAAAGAGGTTGCGGGTGGACTCA | LKPGAPKPKANQQKQDQRGLVLPGYKY |
| | AAGAGTTCATTGACCTCAAACCGGGTGCGCCGAA | LGPFNGLDKGQPVNKADEVAREHDIEYD |
| | GCCCAAAGCGAATCAACAAAAACAAGATCAACGC | KLLKEGDNPYVKYNHADQEFQEKLEGD |
| | GGTCTTGTCCTCCCGGGTTACAAATACCTGGGACC | ESFGGNLGRAVFQAKKRLLEPFGLSEETP |
| | CTTCAACGGCCTTGATAAAGGCCAACCGGTGAAC | PRKRPASENNQSDVPAKRRLILDPEDNG |
| | AAAGCCGACGAAGTCGCGCGGGAGCACGACATCG | AAEGEPIEQQQPANDMASAEMAAGGGG |
| | AATACGACAAACTCCTGAAAGAAGGAGACAACCC | PVGDDAQGADGVGSASGNWHCDSIWM |
| | GTACGTGAAGTACAACCACGCCGATCAAGAGTTTC | GDRVITKSTRTWSLPTYNNHLYKQINSN |
| | AAGAGAAACTCGAGGGCGACGAATCTTTTGGCGG | TTDAKETYFGYSTPWGYFDFNRFHCHFS |
| | TAACCTCGGACGAGCGGTCTTCCAAGCCAAGAAG | PRDWQRLINNHWGMRPKRLNFKLFNIQ |
| | CGGTTACTGGAACCGTTTGGTTTATCGGAAGAGAC | VKEVTTNDGTKTIANNLTSTVQVFADSE |
| | ACCTCCGAGAAAGAGACCCGCTTCCGAGAACAAC | YQLPYVLGNAHEGCFPPFPADVFMLPQY |
| | CAGTCAGACGTCCCGGCTAAGAGACGTCTGATCCT | GYLTLNSNDNKAMPQSSFYCLEYFPSQM |
| | CGATCCCGAAGACAACGGAGCCGCCGAAGGAGAA | LRTGNNFTFSYEFESVPFHSMFMHNQSL |
| | CCAATCGAACAACAACAACCAGCCAACGATATGG | DRLMNPLIDQYLWYLHSTNSLSLNFKKV |
| | CATCTGCTGAAATGGCTGCAGGTGGTGGCGGGCC | KRDHLYKYYRNWVPGPGQRRQQWSES |
| | AGTGGGCGACGATGCACAGGGTGCCGATGGAGTG | GGANKQLTDLWPYNNKYNLMGRYSSLA |
| | GGTAGCGCCTCAGGAAATTGGCATTGCGATTCCAT | PGMPQAAADGFITADGQLIFSNVLQESEE |
| | TTGGATGGGTGACCGAGTCATCACCAAATCCACTC | ASGGKPSPNLLITNESEVRPTNPAINAPW |
| | GCACCTGGAGCCTGCCCACCTACAACAACCACCTC | GDVALNQQTSTVTPSVAEDNEAHIFPGM |
| | TACAAGCAAATCAACTCTAACACCACCGATGCCA | VWQDRDIYLQGPIWAKIPDTDGSFHPSPL |
| | AAGAAACCTACTTTGGCTACAGCACTCCTTGGGGG | MGGFGLKHPPPQILIKNTPVPSDPPLEFN |
| | TATTTTGACTTCAACCGGTTCCACTGCCACTTCTCG | GKKIQSFITQYSTGQVTVEIEWELKKEHS |
| | CCGAGGGACTGGCAACGACTCATCAACAACCATT | KRWNPEIQYTSNFDNTDRVQFSVNGAGN |
| | GGGGCATGCGACCAAAGAGACTCAACTTTAAACT | YVEDRPIGTRYLTHNL (SEQ ID NO: 862) |
| | CTTTAACATCCAAGTCAAAGAGGTCACGACGAAC | |
| | GACGGTACGAAAACGATCGCGAACAATCTTACCT | |
| | CGACGGTGCAAGTCTTCGCGGACTCAGAGTACCA | |
| | ACTCCCCTACGTGCTAGGCAACGCGCACGAGGGA | |
| | TGCTTTCCACCGTTTCCGGCCGACGTCTTCATGCTA | |
| | CCGCAGTATGGATACCTGACCTTGAACTCAAACGA | |
| | CAACAAAGCCATGCCACAGAGCTCGTTCTACTGTC | |
| | TGGAATATTTTCCGAGCCAGATGCTGAGAACCGGC | |
| | AACAACTTCACCTTCAGCTATGAGTTCGAGAGCGT | |
| | TCCATTCCACAGCATGTTTATGCATAACCAATCGC | |
| | TGGACAGATTGATGAATCCATTGATTGACCAGTAC | |
| | TTATGGTACCTACACTCTACCAACAGTCTATCTCT | |
| | AAATTTTAAAAAAGTGAAGAGAGATCATCTGTAC | |
| | AAATACTACAGAAATTGGGTACCCGGACCCGGTC | |
| | AAAGAAGACAACAATGGAGCGAAAGTGGTGGAGC | |
| | AAACAAACAATTGACAGACTTGTGGCCATACAAC | |
| | AACAAATACAATCTGATGGGAAGATATTCTTCTTT | |
| | GGCACCGGGAATGCCACAAGCGGCGGCAGATGGA | |
| | TTCATCACAGCCGATGGACAATTAATATTTTCTAA | |
| | CGTGCTACAAGAATCAGAAGAAGCCAGCGGTGGA | |
| | AAACCATCTCCAAATCTATTAATTACAAACGAGTC | |
| | AGAAGTCAGACCAACAAATCCAGCTATCAACGCA | |
| | CCTTGGGGAGATGTCGCATTAAATCAACAAACGTC | |
| | AACAGTAACACCATCAGTAGCTGAAGACAACGAA | |
| | GCCCACATTTTTCCTGGCATGGTCTGGCAAGACCG | |
| | AGACATCTACCTGCAAGGACCCATCTGGGCCAAG | |
| | ATACCAGACACCGACGGCAGCTTCCACCCGTCTCC | |
| | GCTCATGGGTGGATTTGGACTCAAACACCCTCCGC | |
| | CGCAGATCCTGATCAAGAACACGCCGGTACCGTC | |
| | GGATCCTCCGCTGGAATTCAACGGCAAGAAGATC | |
| | CAGTCCTTCATCACGCAGTACTCGACCGGTCAGGT | |
| | GACGGTGGAGATCGAGTGGGAGCTGAAGAAGGAG | |
| | CACAGCAAGCGGTGGAATCCGGAGATCCAGTACA | |
| | CCTCCAACTTCGACAACACCGACCGGGTGCAGTTC | |
| | TCGGTCAACGGGGGGGGCAACTATGTCGAAGATC | |
| | GACCAATCGGGACCCGTTATCTCACCCACAATCTG | |
| | TAA (SEQ ID NO: 861) | |
| RF1 | ATGAGTCGAGTAGTCATGTCGTTTGTTGATCACCC | MSRVVMSFVDHPPDWLKRVASGLTEFID |
| | TCCAGATTGGCTGAAGCGTGTGGCGAGTGGCTTGA | LEPGPPKPKPGYQDQQRGLVVPGYKYLG |
| | CTGAGTTCATCGACCTCGAACCCGGACCCCCAAAA | PFNGLDKGEPVNKADEVAREHDLEYNK |
| | CCCAAACCCGGCTATCAGGACCAGCAGCGCGGTC | LLEQGDNPYLKYNHADKEFQEKLSDDTS |
| | TCGTGGTACCCGGATACAAATACCTCGGTCCCTTT | WGGNLGKAVFQAKKRILEPFGLAEELGG |
| | AACGGTCTCGATAAAGGAGAACCCGTTAACAAGG | EKTAPGKKRPRPPELPPSAPHSDDEELIA |
| | CTGACGAGGTTGCTCGAGAGCACGACCTCGAGTA | RPAVRARLEGONGAANAGPAAAPAADM |
| | CAACAAGCTACTTGAGCAAGGAGATAATCCTTATC | ASTEVASGGGGPMGDNNQGSDGVGSSS |
| | TCAAGTACAACCACGCCGATAAGGAGTTTCAAGA | GNWHCDSIWMDGRVITKSTRTWSLPTY |
| | GAAACTCTCTGACGACACTTCCTGGGGCGGCAACC | NNHLYKQINSNAEVKDEKYFGYSTPWG |
| | TCGGGAAGGCGGTTTTTCAAGCCAAGAAGCGTATC | YFDFNRFHCHFSPRDWQRLINNHWGMR |
| | CTCGAGCCCTTTGGCCTAGCCGAAGAGCTCGGCGG | PKRLNFKLFNIQVKEVTTNDGTTTIANNL |
| | TGAAAAGACGGCTCCTGGAAAAAAGCGTCCCAGG | TSTVQVFADTEYQLPYVLGNAHEGCLPP |
| | CCTCCGGAACTTCCACCTTCGGCTCCGCATTCGGA | FPADVFMLPQYGYLTLNDGNQVIPQSSF |
| | CGACGAAGAACTGATTGCTCGACCGGCAGTCAGA | YCLEYFPSQMLRTGNNFTFSYEFENVPFH |
| | GCTCGGCTCGAAGGTCAAAACGGAGCAGCAAACG | SMFMHNQSLDRLMNPLVDQYLWYLSAT |
| | CAGGACCAGCAGCAGCACCAGCCGCAGATATGGC | NGLNLHFRKSKKGAFASQYRNWMPGPN |
| | ATCTACTGAAGTGGCTTCAGGCGGTGGCGGCCCAA | QRRQQWSKGAGNNNALTNLWEYKNKF |
| | TGGGCGACAATAACCAAGGTTCCGATGGAGTGGG | HIMNRDSVLAPGLAQAAADGYFTADGQ |
| | TAGCTCCTCCGGAAATTGGCATTGCGATTCCATTT | LIFANATQQSDEASGGNPPSSMLITGESE |
| | GGATGGACGGCAGAGTCATCACCAAGTCTACCAG | TRPTNPGANTKWGEMADNQQSTTAKPT |
| | AACCTGGAGCCTGCCCACCTACAACAACCACCTCT | MVIDNEAHIFPGMVWQDRDIYLQGPIWA |
| | ACAAGCAAATCAACTCAAATGCCGAAGTCAAAGA | KIPETDGHFHPSPLMGGFGLKNPPPQILIK |
| | CGAAAAGTACTTTGGATATAGTACTCCCTGGGGAT | NTPVPANPPEAFTANKINSFITQYSTGQV |
| | ACTTTGATTTCAATCGATTCCACTGCCACTTTTCTC | TVEIEWELRKEHSKRWNPEVQYTSNFDS |
| | CCAGAGACTGGCAGCGACTCATCAACAACCACTG | TDSLQFTVNNDGLYIETRPIGTRFLSHNI |
| | GGGAATGCGACCCAAAAGACTCAACTTTAAGCTG | (SEQ ID NO: 864) |
| | TTCAACATCCAGGTCAAGGAGGTCACGACGAATG | |
| | ACGGAACGACAACGATCGCCAATAACCTCACGTC | |
| | GACCGTCCAGGTGTTTGCAGACACAGAGTACCAGT | |
| | TGCCGTACGTGCTCGGGAATGCTGCACGAGGGCTG | |
| | CCTCCCGCCGTTCCCGGCAGACGTGTTTATGCTGC | |
| | CGCAGTACGGATACTTGACTCTAAACGACGGCAA | |
| | CCAGGTCATTCCTCAGAGCTCTTTTTACTGCTTGGA | |
| | GTACTTTCCCAGTCAGATGCTGAGAACCGGAAACA | |
| | ACTTTACGTTCAGCTACGAGTTTGAGAACGTGCCT | |
| | TTCCACAGCATGTTTATGCATAATCAGTCTCTGGA | |
| | CCGACTGATGAATCCTTTAGTCGATCAGTACCTGT | |
| | GGTACCTCTCGGCTACCAACGGATTAAATTTACAT | |
| | TTTAGAAAATCCAAGAAGGGAGCGTTTGCCAGTC | |
| | AATACAGAAACTGGATGCCCGGTCCTAACCAGCG | |
| | ACGTCAGCAGTGGAGTAAAGGAGCTGGAAACAAC | |
| | AACGCCTTGACCAATCTCTGGGAATACAAAAACA | |
| | AGTTCCACATCATGAACAGAGACTCGGTACTGGCT | |
| | CCAGGACTCGCACAGGCTGCGGCGGATGGATATTT | |
| | CACAGCCGACGGACAATTAATTTTTGCTAACGCTA | |
| | CACAACAATCGGACGAGGCTTCCGGTGGAAATCC | |
| | ACCGAGCAGCATGTTAATTACCGGTGAATCCGAA | |
| | ACTCGACCCACTAATCCCGGAGCAAACACCAAGT | |
| | GGGGTGAAATGGCCGACAACCAACAGTCTACAA | |
| | GGCAAAGCCGACCATGGTTATCGACAACGAGGCT | |
| | CACATCTTTCCCGGCATGGTCTGGCAGGATCGGGA | |
| | CATTTACCTGCAAGGGCCCATCTGGGCCAAGATTC | |
| | CAGAGACGGACGGACACTTTCATCCTTCTCCGCTG | |
| | ATGGGCGGATTCGGTCTCAAGAATCCTCCTCCTCA | |
| | GATACTCATCAAGAATACACCCGTTCCGGCTAATC | |
| | CTCCGGAAGCGTTCACCGCCAACAAGATAAACTCC | |
| | TTCATCACGCAGTACTCGACCGGACAAGTAACGGT | |
| | GGAAATTGAATGGGAGCTCAGGAAGGAGCACAGC | |
| | AAGCGCTGGAACCCAGAGGTGCAGTACACGTCCA | |
| | ACTTTGACTCTACAGACAGTCTGCAGTTTACCGTT | |
| | AACAACGACGGACTCTACATCGAGACTCGACCAA | |
| | TCGGAACCCGTTTCCTTTCCCACAATATTTAA (SEQ | |
| | ID NO: 863) | |
| RS1 | ATGAGTCGAGTAGTCATGTCGTTTGTCGATCACCC | MSRVVMSFVDHPPDWLKEAAGGLREFL |
| | TCCAGATTGGCTGAAAGAGGCCGCGGGCGGACTA | DLEPAPKPKPNQQKQDQRGLVLPGYK |
| | CGCGAGTTTCTCGATCTCGAGCCGGGAGCGCCTAA | YLGPFNGLDKGQPVNKADEVAREHDLE |
| | ACCCAAACCTAATCAACAAAAACAGGACCAGCGA | YNKLLEAGDNPYLKYNHADQEFQEKLE |
| | GGTCTGGTTCTGCCGGGATACAAATATCTCGGACC | GDTSFGGNLGRAVFQAKKRVLEPFGLAE |
| | TTTCAACGGCCTTGATAAAGGCCAGCCCGTCAATA | EAPAKTAPGKKRPRPIEPPPSSENSVDED |
| | AGGCGGACGAGGTCGCGCGAGAGCACGATCTCGA | LVERQAQRQRLGFQDGPGNSANQVQQQ |
| | GTATAACAAGCTACTTGAGGCGGGCGACAATCCCT | PAANMASAEVAAGSGGPMGDNAQGAD |
| | ACCTCAAGTACAACCACGCAGACCAAGAGTTTCA | GVGSSSGNWHCDSLWMGDRVMRFPWM |
| | GGAGAAACTCGAGGGGGACACTTCCTTCGGAGGT | GDRVITKSTRTWSLPTYNNHLYKQINSNS |
| | AACCTCGGTCGCGCGGTATTCCAGGCCAAGAAGA | DVTDEKYFGYSTPWGYFDFNRFHCHFSP |
| | GGGTGCTAGAGCCCTTTGGATTGGCCGAAGAAGCT | RDWQRLINNHWGMRPKRLNFKLFNIQV |
| | CCGGCGAAGACGGCTCCTGGGAAGAAACGTCCCA | KEVTTNDGTTTIANNLTSTVQIFADSEYQ |
| | GGCCCATCGAACCCCCTCCTTCCTCGGAGAACTCG | LPYVLGNAHEGCLPPFPADVFMLPQYGY |
| | GTTGACGAGGACTTGGTCGAGCGCCAAGCTCAGC | LTLNDGNQVIPQSSFYCLEYFPSQMLRTG |
| | GTCAACGTCTCGGATTTCAAGACGGACCCGGAAA | NNFSFSYEFENVPFHSMFMHNQSLDRLM |
| | CAGCGCTAACCAAGTCCAGCAACACCAGCCGCA | NPLMDQYLWYLTATNGVQLHFKKSKKG |
| | AATATGGCATCTGCTGAAGTGGCTGCGGGCAGTG | AFASQFRNWLPGPNQRRQQWSKGAGNN |
| | GCGGCCCAATGGGCGACAATGCACAAGGTGCCGA | NAQTNLWSYKNKYRIMNRDSVAAPGLA |
| | TGGAGTGGGTAGCTCCTCCGGAAATTGGCATTGCG | QAAADGFITADGQLIFSNAGQQSDEAAG |
| | ATTCCCTTTGGATGGGCGATAGGGTGATGCGATTC | AKPPTTLLITDESETRPTNPGANTKWGEV |
| | CCTTGGATGGGAGACCGAGTCATCACCAAGTCCAC | ADNQQTSSTAPTMAIDNEAHIFPGMVWQ |
| | CCGAACCTGGAGCCTGCCCACCTACAACAACCATC | DRDIYLQGPIWAKIPETDGHFHPSPLMGG |
| | TCTACAAGCAAATCAATTCCAACTCGGATGTCACC | FGLRNPPPQILIKNTPVPANPPETFNANKI |
| | GACGAAAAGTACTTTGGATACAGTACCCCGTGGG | NSFITQYSTGQVTVEIEWELKKEHSKRW |
| | GATACTTTGACTTTAACCGCTTCCACTGCCACTTTT | NPEVQYTSNFASADSLQFTVNGEGVYIE |
| | CACCCCGAGACTGGCAGCGCCTCATCAACAACCAT | DRPIGTRYLSHNI (SEQ ID NO: 866) |
| | TGGGGTATGCGCCCGAAGAGGCTCAACTTTAAGCT | |
| | GTTCAACATCCAGGTTAAGGAGGTCACGACGAAC | |
| | GACGGCACCACGACCATCGCCAACAACCTCACGT | |
| | CTACCGTCCAGATTTTCGCCGACAGCGAGTACCAG | |
| | TTGCCGTACGTGCTCGGGAATGCGCACGAGGGCTG | |
| | CCTCCCGCCGTTCCCGGCAGACGTGTTCATGCTGC | |
| | CGCAGTACGGGTACCTCACGTTGAACGACGGAAA | |
| | TCAAGTGATTCCACAAAGTTCCTTCTACTGTCTGG | |
| | AGTATTTTCCCAGCCAGATGCTGAGAACCGGCAAC | |
| | AACTTTTCCTTCAGCTACGAATTTGAAAACGTGCC | |
| | TTTCCACAGCATGTTTATGCACAACCAGTCTTTGG | |
| | ATCGGTTAATGAACCCCCTCATGGACCAGTACCTG | |
| | TGGTACCTGACGGCCACCAACGGCGTTCAGTTGCA | |
| | CTTTAAAAAGTCCAAGAAGGGGGCGTTTGCCAGC | |
| | CAGTTTAGGAACTGGCTGCCGGGTCCCAACCAGCG | |
| | GCGTCAGCAGTGGAGCAAAGGGGCCGGTAACAAC | |
| | AACGCTCAGACCAACCTGTGGAGTTACAAAAACA | |
| | AGTACCGGATCATGAACCGGGACAGCGTGGCGGC | |
| | TCCTGGACTGGCTCAAGCGGCGGCCGATGGCTTCA | |
| | TCACGGCCGACGGACAGCTCATCTTCTCCAACGCC | |
| | GGACAACAGTCGGATGAAGCGGCTGGAGCTAAAC | |
| | CACCGACCACGTTGCTGATTACAGACGAGAGTGA | |
| | AACTCGTCCTACCAACCCGGGTGCCAACACCAAAT | |
| | GGGGAGAAGTGGCAGACAACCAACAGACATCTTC | |
| | CACGGCCCCCACCATGGCCATCGACAACGAGGCC | |
| | CACATCTTTCCCGGAATGGTGTGGCAGGATCGAGA | |
| | CATTTACCTGCAGGGTCCCATCTGGGCCAAGATTC | |
| | CAGAGACGGACGTTCACTTTCACCCGTCTCCTCTG | |
| | ATGGGAGGATTCGGACTGCGAAATCCTCCTCCTCA | |
| | GATTCTCATCAAAAACACACCGGTACCGGCCAATC | |
| | CACCAGAGACCTTCAACGCCAACAAGATCAACTC | |
| | ATTCATCACCCAGTACTCAACCGGACAGGTTACAG | |
| | TCGAAATAGAGTGGGAGCTGAAGAAGGAGCACAG | |
| | CAAACGCTGGAACCCGGAGGTGCAGTACACGTCC | |
| | AATTTTGCCTCGGCGGACAGCCTCCAATTCACCGT | |
| | CAACGGAGAAGGCGTGTACATCGAAGACAGACCA | |
| | ATCGGAACCCGCTACCTCTCCCACAACATTTAA | |
| | (SEQ ID NO: 865) | |

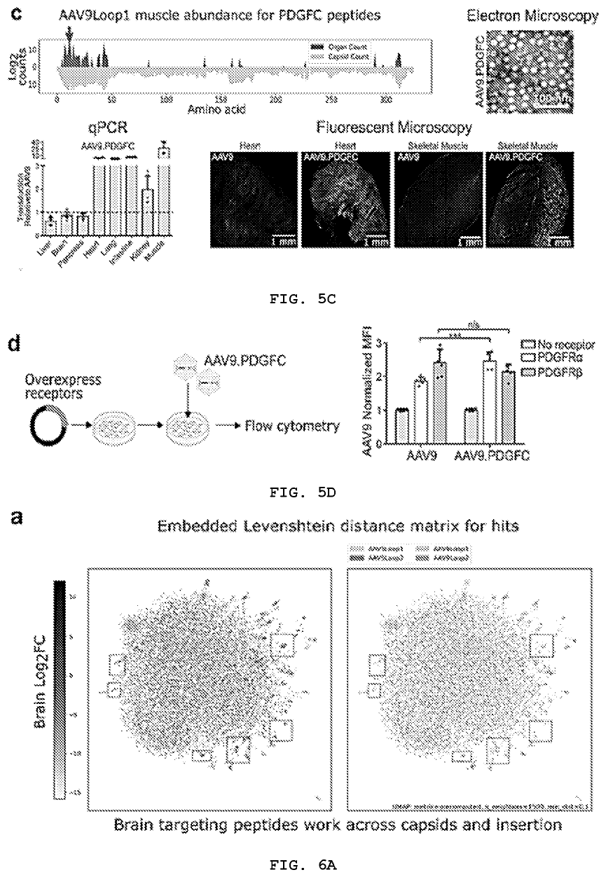

The disclosure shows that one of the discovered variants (AAV9.DKK1) exhibit deprecated transduction when the cognate receptor for the ligand from which the peptide was derived is knocked out (FIG. 5a). Furthermore, due to the systematic tiling nature of the peptide library, it was possible to generate full-length transduction maps across the entire residue space of ligands utilized in this study (FIG. 5a,c). This not only increases confidence in the screening hits, in that homologous peptides behave similarly, but could also provide insight into critical structural domains mediating virus-receptor interactions and more broadly, provide a platform for further expanding the known protein interactome especially when the dataset is integrated with the cell surfaceome.

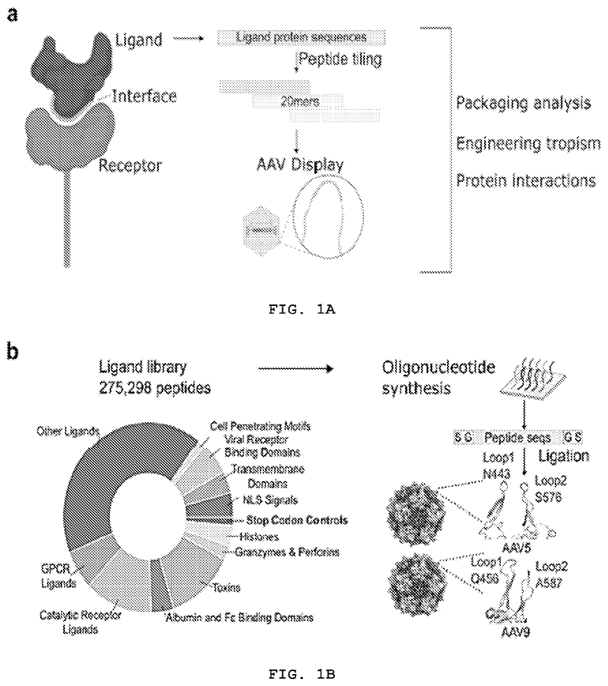

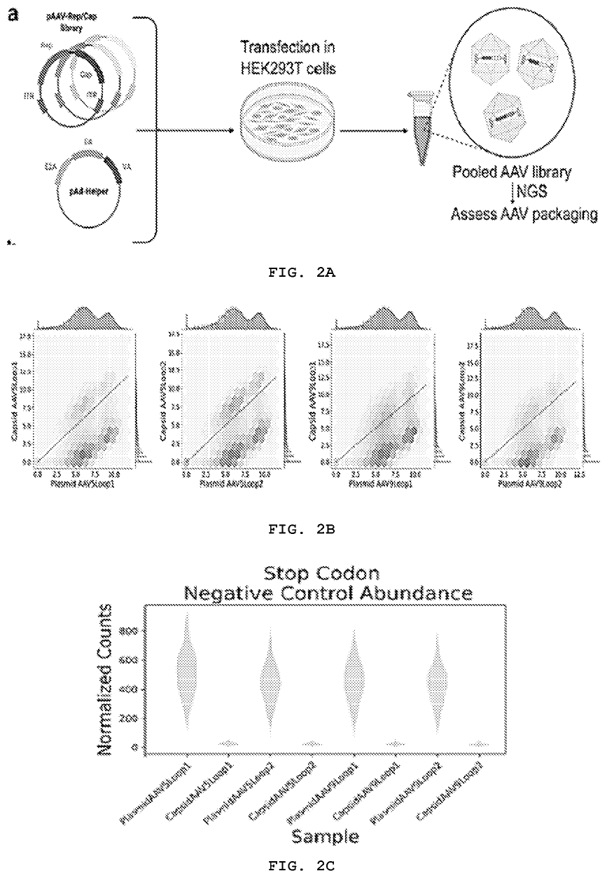

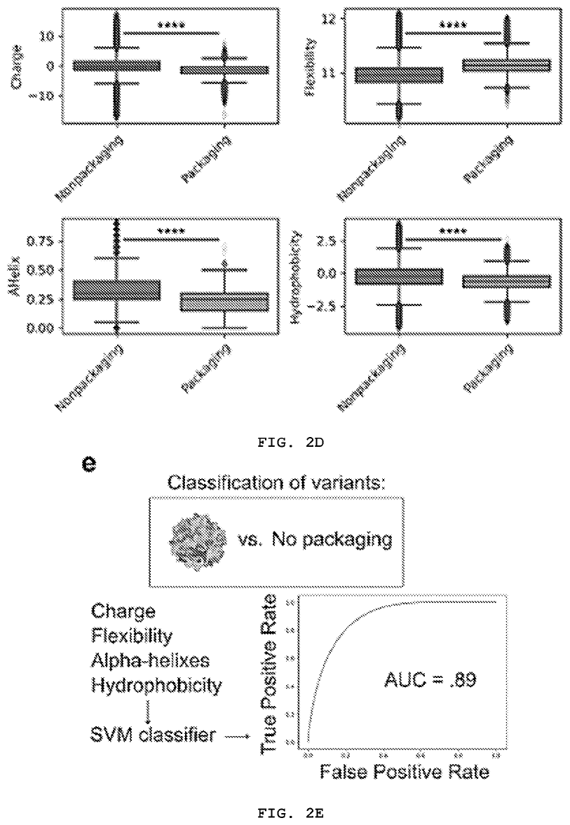

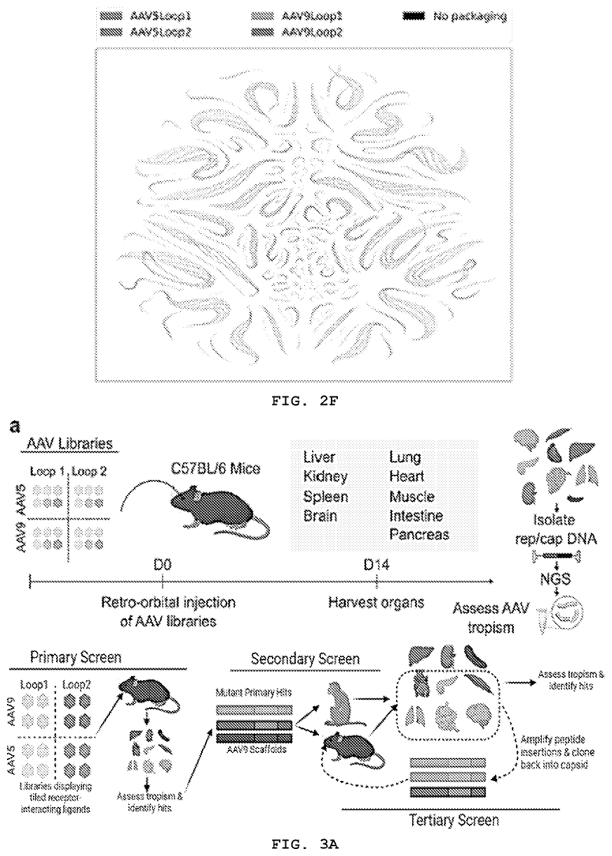

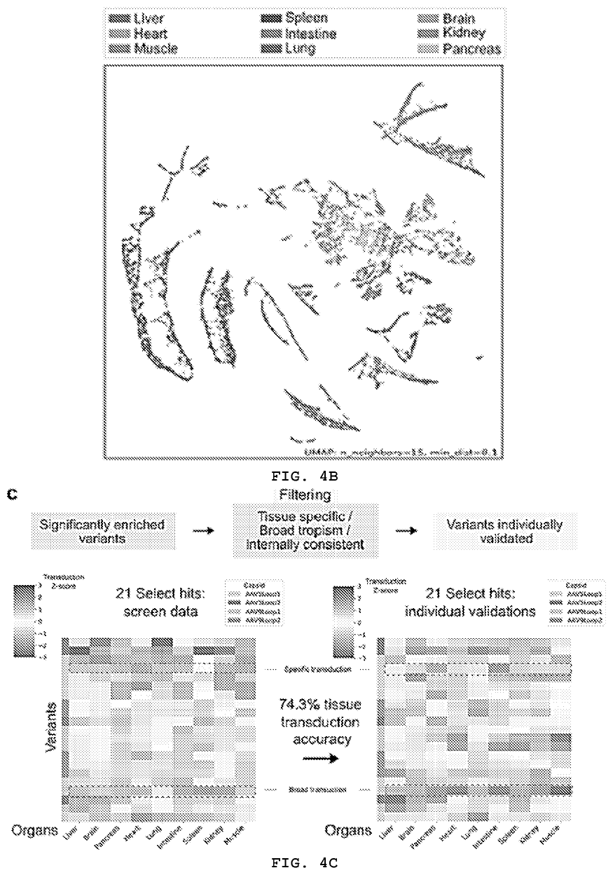

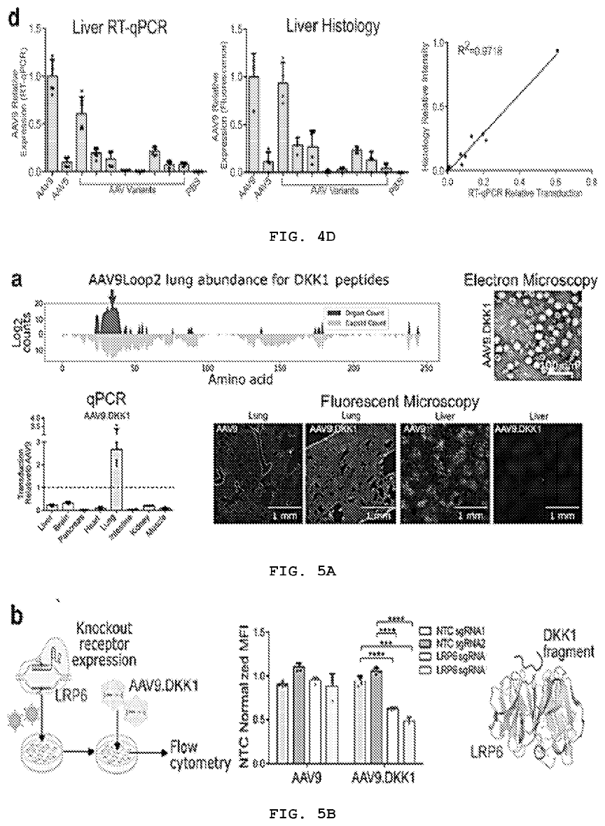

Additionally, in recent years, developing predictive models of AAV infectivity has garnered significant interest from multiple research groups. The application of machine learning to AAV engineering parallels major advances in machine learning across multiple areas of protein science such as structure prediction, enzyme activity forecasting, and antibody binding optimization. While deep learning and similar blackbox methodologies have rapidly become mature technologies, applying these methodologies to AAV engineering is still severely limited by the lack of available training data. The AAV screening data is an ideal training dataset for several reasons: (1) the experimental design features a large, defined library of variants (FIG. 1), meaning that every variant has a reliable quantification of package infectivity, (2) each variant was screened across a panel of 9 major organs to map the infectivity across diverse tissue types, (3) the library was inserted across 2 AAV serotypes and 2 surface loops, providing important functional information on the capsid context in which the peptide resides, and (4) a large cohort of variants were validated to demonstrate the screening data is trustworthy (FIG. 4) and (5) to illustrate the utility of the dataset as training data, the packaging efficiency of AAV variants could be accurately predicted from the biophysical characteristics of the inserted peptides (FIG. 2e), and also peptide amino acid sequence is directly predictive of tissue-tropism across multiple capsids and insertion sites (FIG. 3d). As such, the screening data will have great utility for the machine-learning and computational biology community.

While the variants identified via the pooled-screen have tissue transduction exceeding AAV9 in many organs (FIG. 5a,c) and several variants exhibit drastic liver de-targeting (FIG. 4d, 6c), further engineering could be performed to enhance potency and specificity. Accordingly, peptides with at least 85% identity to any one of the peptide sequences provided herein are also contemplated. This may be relevant in particular for certain peptides derived from ligands that engage receptors expressed on multiple cell types or which have promiscuous binding activity. In the validation experiments, a standard promoter (CMV) was used to drive expression of the mCherry transgene. Alternatively, tissue-specific promoters could be used to increase the specificity and magnitude of transgene expression in the organ of interest. Furthermore, the hit capsids identified here could be further engineered for increased activity. Existing hits could serve as a scaffold for further rounds of targeted mutagenesis and screening, or peptides could be inserted on both loop1 and loop2 of the AAV capsid to increase the valency of the displayed ligands. Additionally, recently developed direct chemical engineering or peptide display strategies and/or alternative peptide linkers could be utilized to enhance peptide-mediated transduction. Also, scRNAseq could be used to screen hit variants towards more specific cell-types within the organ of interest.

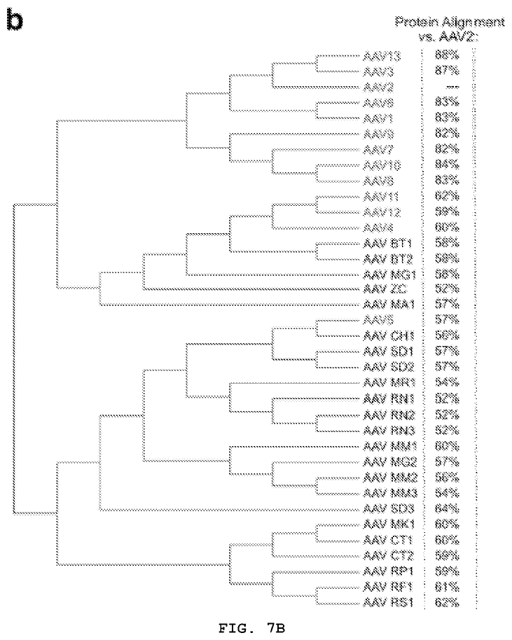

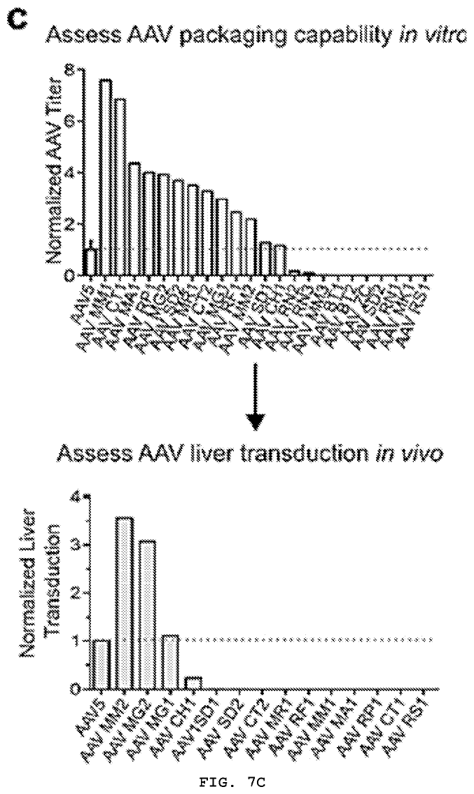

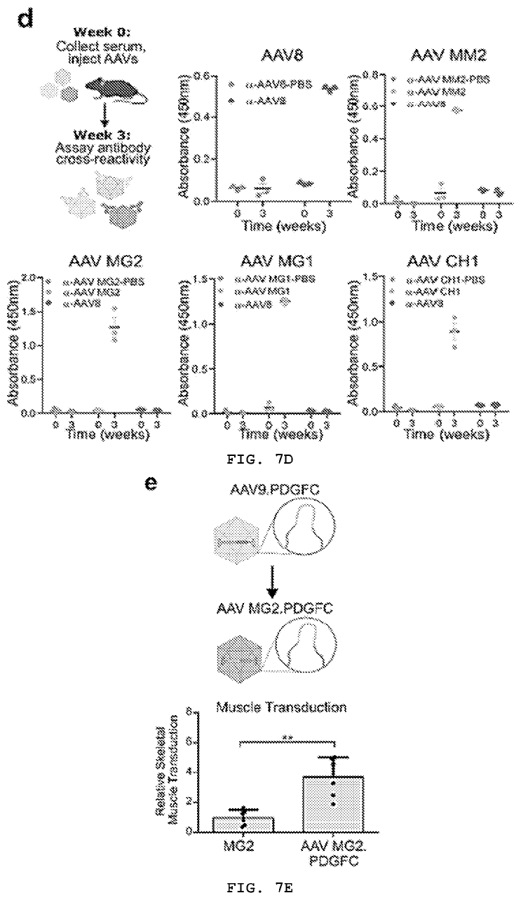

While the screening strategy addresses the gene therapy hurdle of organ targeting, the issue of pre-existing AAV immunity also can limit clinical translation. Therefore, experiments computationally identified and fully characterized a set of 4 immune-orthogonal AAV capsids (FIG. 7a-d), and showed that insertion of a screen identified re-targeting peptide can alter the in vivo tropism of one of these identified AAVs (FIG. 7e). This not only increases the number of known serotypes with interesting immunological properties to be investigated alongside the current AAV set, but also lends credence that the peptide insertions identified through the screening methodology can be grafted onto diverse biological scaffolds to achieve unique targeting across broad delivery contexts.

Taken together, the disclosure presents a massive functional screen of engineered AAV variants, spanning over one million total variants derived from two capsids and multiple sites of insertional mutagenesis. Using this screening data, validation of 21 AAV variants, identifying AAV capsids with increased organ transduction across multiple organs (heart, muscle, and lung for AAV9.PDGFC, FIG. 4c), as well as highly specific AAV capsids (AAV5.APOA1, AAV9.DKK1, FIG. 5a). Improved broad targeting AAV variants have massive potential for genetic diseases such as hemophilia A, where total factor VIII expression levels are most critical. At the same time, highly specific AAV capsids such as AAV5.APOA1 (which has less than 1% the liver infectivity of WT AAV9) would have great utility for neurodegenerative disorders, where maximizing transgene expression in the brain is essential. In addition to the novel variants identified herein, the bulk screening data itself is high value. Given the scale, reliability, and translational relevance of the screening dataset, the data set can serve as a foundation for future computational engineering of designer AAV capsids.

The following examples are intended to illustrate but not limit the disclosure. While they are typical of those that might be used, other procedures known to those skilled in the art may alternatively be used.

### Examples

### Mice

All animal care and experimental methods were performed in accordance with the University of California Institutional Animal Care and Use Committee. 6-8 week old male C57Bl/6J (JAX, #000664) and Balb/cJ (JAX, #000651) mice were purchased from the Jackson Laboratories and systemic injections were administered retro-orbitally with either AAV or PBS.

### Cell Lines and Culture Conditions

All cells were cultured in a 37 °C 5% CO2 humidified incubator. HEK293T cells were cultured in DMEM medium supplemented with 10% FBS, GlutaMAX (1×) (GIBCO), and Penicillin-Streptomycin (100 U/mL) (GIBCO).

### Visualizing AAV Capsid Structures

To obtain structural representations of AAV capsids, AAV5 and AAV9 structure files were downloaded from the Protein Data Bank. These were visualized using the PyMOL Molecular Graphics System, Version 2.0 Schrödinger, LLC with the ramp_new function utilized for coloring the surface capsid representation.

### Design of Displayed Peptide Library

Each AAV library consisted of 275,298 peptides, derived from 6,465 proteins. These protein sources were mined from a variety of protein families, including all protein ligands cataloged in the Guide to Pharmacology database, toxins, nuclear localization signals (NLS), viral receptor binding domains, albumin and Fc binding domains, transmembrane domains, histones, granzymes, and predicted cell penetrating motifs. In addition to peptides coding for functional biomolecules, 444 control peptides coding for FLAG-tags with premature stop codons were included. For all human proteins, the cDNA coding for each protein was fragmented in silico to generate DNA coding for all possible 20mer peptides. For viral proteins, cell penetrating motifs, and FLAG stop codon controls, the protein sequence was back-translated to DNA using the most abundant human codon for each amino acid.

### Oligonucleotide Array Synthesis, Amplification, and Cloning

Oligonucleotide libraries were synthesized by GenScript as three 91,766 element pools. Each oligonucleotide library was amplified using KAPA Hifi Hotstart Readymix, and the manufacturer recommended cycling conditions with an annealing temperature of 60 °C and an extension time of 30 seconds. The number of PCR cycles was optimized to avoid over-amplification of the peptide libraries. After amplifying each oligonucleotide pool and confirming amplicon size on an agarose gel, the amplified sub-libraries were pooled to yield the total 275,298 element peptide library.

These pools were cloned into loop 1 or loop 2 sites to yield pAAV5L1_Screen, pAAV5L2_Screen, pAAV9L1_Screen, and pAAV9L2_Screen. The AAV rep and cap were flanked by AAV inverted terminal repeat (ITR) sequences to facilitate packaging of cap genes into a recombinant AAV particle.

### Recombinant AAV Production

Utilizing the library plasmid pools described above (AAV5-Loop1, AAV5-Loop2, AAV9-Loop1, and AAV9-Loop2), each AAV capsid library was produced by transfecting HEK293T cells in 40 15 cm dishes with the plasmid library pool (diluted 1:100 with pUC19 filler DNA to prevent capsid cross-packaging) and an adenoviral helper plasmid (pHelper). Titers were determined via qPCR using the iTaq Universal SYBR green supermix and primers binding to the AAV ITR region. To prepare the capsid particles as templates for qPCR, 2 μL of virus was added to 50 μL of alkaline digestion buffer (25 mM NaOH, 0.2 mM EDTA) and boiled for 8 minutes. Following this, 50 μL of neutralization buffer (40 mM Tris-HCl, 0.05% Tween-20, pH 5) was added to each sample.

### In Vivo Evaluation of AAV Display Libraries

Each AAV capsid library was retro-orbitally administered to mice in duplicate at a dose of 2E12 vg/mouse for the AAV9-based libraries or 1E12 vg/mouse for the AAV5-based libraries. Two weeks after injection, the heart, lung, liver, intestine, spleen, pancreas, kidneys, brain, and gastrocnemius muscle were harvested and placed in RNAlater storage solution. Total DNA was extracted from all mouse tissues using TRIzol reagent and the TNES-6U back extraction method. The resulting precipitated DNA was centrifuged for 15 minutes at 18,000G, and the supernatant discarded. The DNA pellet was then washed three times with 70% ethanol, and finally resuspended in 300 μL of EB after allowing the pellet to air dry.

### Preparation of Plasmid and Capsid DNA for Next Generation Sequencing

To sequence the plasmid libraries (AAV5/9 and loop1/2 peptide insertions), 50 ng of plasmid was used as template for a 50 μL KAPA Hifi Hotstart Readymix PCR reaction with an annealing temperature of 60 °C and an extension time of 30 seconds. The primers were designed to amplify the peptide coding region from each sub-library. The number of cycles was optimized to avoid overamplification. The PCR reactions were purified using a QIAquick PCR Purification Kit according to the manufacturer's protocol. Following this, 50 ng of the PCR amplicon was used as template for a secondary 50 μL KAPA Hifi Hotstart Readymix PCR reaction to add illumina compatible adapters and indices (NEBNext Cat #E7600S). The PCR reaction was performed with an annealing temperature of 60 °C, an extension time of 30 seconds. To sequence the capsid libraries, a similar protocol was performed, with a modified template amount in the step-1 PCR. To prepare the capsid particles as templates for PCR, 2 μL of virus was added to 50 μL of alkaline digestion buffer (25 mM NaOH, 0.2 mM EDTA) and boiled for 8 minutes. Following this, 50 μL of neutralization buffer (40 mM Tris-HCl, 0.05% Tween-20, pH 5) was added to each sample. 1 μL of this digested capsid mix was then used as a template for a 50 μL PCR reaction. For each sample, the number of cycles was optimized to avoid overamplification, and a secondary PCR was subsequently performed to add illumina compatible adapters and indices. After generating illumina compatible libraries, the plasmid and capsid samples were sequenced on a NovaSeq 6000 with an S4 flowcell generating 100 bp paired end reads.

### Preparation of Tissue DNA for Next Generation Sequencing

To sequence the AAV cap genes from each tissue for the pooled screen, as with the plasmid/capsid libraries, a two-step PCR based library prep method was used. For each organ and replicate, a 300 μL PCR reaction was performed with 120 μL of genomic DNA used as a template. For each tissue, the number of cycles was optimized via an initial qPCR to avoid overamplification of the library. All other parameters such as primers, and melting temperatures were identical to the PCRs for the plasmid libraries. Following this initial PCR, a secondary PCR was performed as above to add illumina compatible adapters and indices. The libraries were then sequenced on a NovaSeq 6000 with an S4 flowcell generating 100 bp paired end reads.

### In Vivo Validation of AAV Variants

Either saline or the AAV-variant-mCherry, AAV9-mCherry, or AAV5-mCherry capsids were systematically administered to mice in duplicate at a dose of 5E11 vg/mouse. Three weeks after injection, the lungs were inflated with a PBS/OCT solution and the lungs, heart, liver, intestine, spleen, pancreas, kidneys, brain, and gastrocnemius muscle were harvested. Each organ was split with one portion placed in RNAlater and the other embedded in OCT blocks and flash frozen in a dry-ice/ethanol slurry. Total RNA was then isolated from all mouse tissues using TRIzol reagent and RNA Isolation kits with on-column DNase treatment (Zymo Cat #R2072). cDNA synthesis was performed with random primers from the Protoscript cDNA synthesis kit (NEB Cat #E6560S). Transgene expression was then quantified via qPCR using the iTaq Universal SYBR green supermix and primers binding to the mCherry transcript. mCherry transgene expression was normalized to that of an internal GAPDH control, using GAPDH specific primers. For histological examination, OCT frozen blocks were cryosectioned at approximately 10 μm thickness and tissue slides were then imaged on an Olympus SlideScanner S200. Exposure times between 5-1000 ms were used, with identical exposure times used for all samples of a given tissue type. mCherry expression from histological sections was then quantified using the Olympus OlyVIA software to calculate the mean pixel intensity across the entire organ section.

Quantifying AAV Variant Abundance from NGS Data

Starting with FASTQ sequencing files, the MAGeCK (94) ‘count’ function was used to generate count matrices describing AAV abundance in each sample (plasmids/capsids/tissues). Following this, the count matrices were normalized (via multiplication with a constant size-factor) for each sample to account for non-identical read depth. The sequencing counts were then transformed by taking the log base 2 of the raw counts, after addition of a pseudocount. Variants with no counts across all of the experimental samples were excluded from analysis.

### Biophysical Analysis of AAV Capsids

The biophysical characteristics of the inserted peptides was calculated using the “ProteinAnalysis” module within the Biopython Python package. A variant was considered a successful packager if it had higher abundance in the capsid particles compared to the plasmid pool. Support vector machine training and visualization was accomplished via the “svm” module within the sklearn Python package (96). UMAP projection of peptide biophysical characteristics was accomplished via the “plot” functionality within the UMAP Python package. All default parameters were used for the visualization. Boxplots and hexbin plots were generated using the matplotlib and seaborn Python packages.

### Identifying Significantly Enriched Variants in Each Tissue

To identify variants which successfully transduce each tissue, for each variant a one sample T-test was applied, comparing the abundance in the capsid particles to the abundance in the tissue. Resulting p-values were adjusted for multiple hypothesis testing via the Benjamini-Hochberg procedure. A variant was considered a significant transducer of an organ if it had an FDR adjusted p-value <0.05, and a Log₂FC>1 in both replicates. When choosing variants for validation experiments, variants were prioritized that had inserted peptides which were identified as hits in multiple capsid/loop contexts, and variants for which similar inserted peptides infecting the same organ were identified.

Visualizing Tissue Transduction from Pooled Screen

Heatmaps for visualizing AAV transduction were generated using the ‘clustermap’ function within the seaborn Python package. Rows and columns were ordered via the scipy ‘optimal_leaf_ordering’ function to minimize the euclidean distance between adjacent leaves of the dendrogram. UMAP projections visualizing AAV tissue specificity were generated by embedding the tissue level log 2 fold change into two dimensions via the “plot” functionality within the UMAP Python package. All default parameters were used for generating the embedding. The variants were colored by the organ in which they had the max log₂ fold change.

### Assessing Accuracy of Predicted AAV Variant Tropism

For each variant which was individually validated, the accuracy of both positive and negative predictions of tissue infectivity were assessed. For variants predicted to target a specific organ, a prediction was considered accurate if the individual validations showed greater than 50% of wild-type AAV9 infectivity in that organ. For variants predicted not to target a specific organ, a prediction was considered accurate if the individual validations showed less than 50% of wild-type AAV9 activity.

### Peptide Distance Projections

To calculate the Levenshtein distance between inserted peptides, the “levenshtein” function from the Python package “rapidfuzz” was used with default parameters (100). After building the pairwise distance matrix between all significantly enriched peptides, the matrix was projected into two dimensions via UMAP with metric=“precomputed”, n_neighbors=1500, and min_dist=0.1. Clusters of peptides with similar functions were then hand annotated onto the resulting plot.

### Convolutional Neural Networks

To train convolutional neural networks (CNNs) to predict the tissue specificity of AAVs, the AA sequences of the inserted peptides were converted to a one-hot encoding via the “get_dummies” function from the pandas Python package. Among the significantly enriched variants, a variant was considered a transducer of a given organ if the log₂FC relative to the capsid in both replicates was greater than 0. The data was then randomly split into training (⅔) and validation (⅓) datasets. For each variant, the one-hot encoding was reshaped to a 20×20 matrix with rows indicating residue positions, and columns indicating the presence or absence of a particular amino acid. The model architecture was instantiated via a Keras sequential model (102). In brief, a convolutional layer (Conv1D) with 32 filters, a kernel size of 3 and “relu” activation was fed into a max pooling layer (MaxPool1D) with pool size of 2. These layers were followed with another set of convolutional and max pooling layers, this time with 64 filters in the convolutional layer. These layers were followed with a dense layer with units=20. Finally a dropout layer was added with the dropout rate=0.5. A flattening layer and final dense layer (with sigmoid activation) was then used to output resulting class probabilities. A separate independent model was trained for each organ. When training the models, the classes (infective versus non-infective variants) were weighted proportionally to the inverse of the number of class examples. When calling ‘model.fit( )’ to train the CNN, a dictionary describing the class weights was passed via the ‘class_weight’ parameter. Model performance was evaluated via accuracy, area under the receiver operator characteristic curve (AUROC), F1-score, and Matthews Correlation Coefficient (MCC). Metrics were calculated via builtin Keras functions, and plotted via matplotlib. To assess how model accuracy changes as a function of the edit distance from the training data, the levenshtein distances between peptides in the testing and training datasets were calculated using the ‘rapidfuzz’ python package as above.

### Transmission Electron Microscopy (TEM)

To obtain transmission electron microscopy images of select AAV variants, 20 μL of the AAV solution was applied to formvar/carbon coated EM grids. Upon washing 3 times with water, the sample-containing grid was then negative-stained for 1 minute using a solution of 2% of Uranyl acetate in water and blot-dried. The EM grids with each AAV variant were then imaged at 68,000× magnification using the FEI Tecnai Spirit G2 BioTWIN transmission electron microscope operated at 80 keV.

### Mechanism Knockout Experiments

sgRNA sequences targeting LRP6 or non-targeting controls were identified using CRISPick and cloned into the lentiCRISPR v2 plasmid backbone. Lentivirus was then produced as described in (33). Briefly, HEK293T cells were seeded at -40% confluency the day before transfection. The day of transfection, Optimem serum reduced media was mixed with Lipofectamine 2000 (Thermo Fisher), 3 μg of pMD2.G plasmid, 12 μg of pCMV deltaR8.2 plasmid, and 9 μg of the respective lentiCRISPRv2 plasmid and added drop-wise onto the HEK293T cells following a 30 minute incubation period. 48 hours after transfection, the media was collected and replaced with fresh DMEM with 10% FBS. At 72 hours post-transfection, the supernatant containing viral particles were collected again, pooled, and concentrated to 1 mL using Amicon-15 centrifugal filters with a 100,000 NMWL cutoff (EMD Millipore). For lentiviral transduction, HEK293T cells were seeded in a 12-well plate at ~20% confluency the day before transduction. The day of transduction, lentiviral containing DMEM with 8 μg/mL polybrene was added to the cells. The media was then replaced 24 hours later and then changed into puromycin (2 μg/mL) containing DMEM 28 hours post-transfection. Post selection, once the cells reached confluency, they were passaged into a 24-well plate at ~40% confluency. 24 hours later, DMEM containing either AAV9 (4×10⁹ viral genomes) or AAV9.DKK1 (1×10⁹ viral genomes) was added to the cells. The cells were then collected 24 hours later and flow cytometry was performed to quantify mCherry transgene expression.

### Identifying Immune Orthogonal AAV Capsids

Potential AAV orthologs were curated by first identifying sequences which exhibited similarity to the AAV2 cap gene using the National Center for Biotechnology Information (NCBI) basic local alignment search tool (BLAST). Next, incomplete, heavily truncated, and highly homologous sequences to one another were filtered out of the selection criteria. Viruses within the human AAV clade and those from non-mammalian hosts were then filtered out. Finally, sequences with high similarity to previously identified human serotypes were removed, resulting in the 23 potential AAV orthologues assessed.

### Immune Orthogonal AAV Production

To clone computationally identified AAV orthologs, the capsid sequences were codon-optimized and cloned downstream of the AAV2 rep gene using Gibson Assembly. Immune orthogonal AAV capsids were produced as described for the AAV variant validation capsids and AAV production titer was measured via qPCR with primers binding to the AAV ITR region. Any ortholog with a production titer within a power of 10 of AAV5 was considered to have successfully packaged.

### Immune Orthogonal AAV In Vivo Transduction

Immune orthogonal AAV capsids with sufficient packaging titer were then injected retro-orbitally into C57BL/6 mice at a dose of 1×10¹² viral genomes/mouse. Livers were harvested 3 weeks post-injection and total RNA was isolated as described above. cDNA was then generated and transgene expression was quantified via qPCR using the iTaq Universal SYBR green supermix and primers binding to the mCherry transcript. mCherry transgene expression was then normalized to GAPDH and the relative expression was compared to AAV5.

### Assessing Immune Cross-Reactivity

The assay to assess immune antibody cross-reactivity of the identified immune orthogonal was performed as previously described. Prior to injection, serum was collected via tail snip procedure and then the mice were injected with 1×10¹² viral genomes/mouse of AAV or PBS in triplicate. 3 weeks later, serum was collected from each of the mice and the antibody cross-reactivity ELISA was performed. For this, 1×10⁹ viral genomes of AAV8, AAV MM2, AAV MG1, AAV MG2, or AAV CH1 were diluted in a 1× coating buffer and incubated overnight in each well of 96-well Nunc MaxiSorp plates. Plates were washed three times for 5 mins with 1× wash buffer (Bethyl) and blocked with 1×BSA blocking buffer (Bethyl) for 2 hours at room temperature. The wells were then washed again and serum samples were added at a 1:40 dilutions. Plates were incubated for 5 hours at 4 °C with shaking. Wells were 3× washed and 100 μL of HRP-labeled goat anti-mouse IgG1 (Bethyl; diluted 1:100,000 in 1% BSA) was added to each well. Secondary antibody was incubated for 1 hour at room temperature, wells were washed 3 times, and 100 μL of TMB substrate was added to each well. Optical density at 450 nm was measured using a microplate absorbance reader (BioRad iMark).

### Engineering Immune Orthogonal AAV Variants

To engineer the immune orthogonal ‘AAV MG2’ capsid, the AAV9 capsid amino acid sequence was aligned to the MG2 sequence using Clustal Omega (108). The PDGFC peptide coding sequence was then ligated into the ‘AAV MG2’ vector at the appropriate Loop 1 location with an identical protocol as was done for cloning peptides into AAV9. The engineered ‘AAV MG2.PDGFC’ vector capsid was then assayed in vivo identically to the above validation experiments using AAV9.

### Ethical Compliance

All experiments involving live vertebrates performed at UCSD were done in compliance with ethical regulations approved by the UCSD IACUC committee.

### Statistics

Unless otherwise noted, differences in means were calculated via an unpaired t test. When necessary, p values were adjusted for multiple hypothesis testing via the benjamini-hochberg procedure (99). For all Figures: unless otherwise noted *p<0.05, **p<0.01, ***p<0.001, ****p<0.0001.

### a Systematic Library of AAV Variants Displaying Fragmented Proteins

To create the libraries AAV5 and AAV9 were chosen as the starting serotypes. This was due to their established clinical utility as well as two key characteristics: one, AAV5 is more evolutionarily distant to other AAV serotypes in clinical use, and has previously been shown to be immune orthogonal (to other prevalent AAV serotypes thereby enabling their sequential redosing); and two, AAV9 has been used extensively for clinical trials and has been shown to cross the blood-brain barrier outperforming other AAV serotypes in most tissues. To generate the library of diverse AAV variants, a DNA oligonucleotide pool of 275,298 gene fragments was generated (FIG. 1a-b). Each gene fragment coded for a 20 amino acid peptide derived from the coding sequence of ligands with known extracellular receptors, or a gene predicted to have cell-penetrating or internalizing properties (FIG. 1a-b). Protein ligands were sourced from the Guide to Pharmacology database, an expertly curated list of pharmacological targets and their associated ligands, and cell-penetrating/internalizing functionality was inferred through text mining of UniProt entries. Examples of protein classes identified as having potential internalizing function included toxins, histones, granzymes, viral receptor binding domains, and nuclear localization signal domains (NLS). Mouse and human genomes share 80% of their protein coding genes, with 85% amino acid sequence identity between orthologs. These genomic similarities extend to the receptors for ligands in the libraries (e.g., 86% of mouse GPCR proteins have human orthologs), implying many human ligands will be similarly functional across human and mice contexts. After synthesizing the pool of single-stranded oligonucleotides coding for these gene fragments, it was amplified to double-stranded DNA via PCR, digested, and ligated into two distinct locations on the AAV5 and AAV9 cap genes. Seamless cloning was enabled by type IIS restriction enzymes cutting outside their recognition sequence. PaqCI sites engineered to yield compatible overhangs (coding for glycine-serine 2 amino acid linkers) were inserted at the ends of the peptide coding DNA library and on the AAV5/AAV9 cap plasmid DNA at sites coding for two distinct surface loops, hereon referred to as loop 1 and loop 2. Surface loop 1 (AA 443 and 456 on AAV5 and AAV9, respectively) and loop 2 (AA 576 and 587 on AAV5 and AAV9, respectively) were chosen as peptide insertion locations due to their distance from the viral particle core facilitating potential receptor engagement (FIG. 1b). While many previous AAV engineering efforts have focused on inserting peptides at surface loop 2, loop 1 sites have been shown to accommodate large insertions including even a full length fluorescent protein. Collectively, four libraries of variants were produced, spanning two AAV capsids, with two loop insertion sites each. In addition to protein coding gene fragments, 444 stop codon containing gene fragments were included as negative controls. This defined library synthesis methodology was adopted to enable quantitative inference of variant packaging and transduction efficiencies. The starting plasmid libraries were sequenced to establish initial relative variant abundances, and packaging efficiencies were quantified via comparison to this initial baseline.

### Biophysical Drivers of AAV Capsid Formation

To quantify how well different AAV cap variants package into functional capsids, recombinant AAV particles were generated with the engineered AAV5 and AAV9 cap plasmid libraries via transient triple transfection of HEK293T cells (FIG. 2a). These viral particles were treated with benzonase to degrade residual plasmid DNA, and then subjected to next generation sequencing (NGS) to quantify relative variant abundance. Packaging efficiency was quantified by ranking AAV variants by the log₂ fold change (log₂FC) of their relative capsid abundance compared to their count in the plasmid pool (FIG. 2b). Utilizing this method, over 250,000 AAV variants were identified which package efficiently into functional AAV capsids with a log₂FC>0. To validate this packaging metric quantified from screening data, 25 AAV capsids were produced including 23 identified as successful packagers (log₂FC>0) and 2 identified as non-packagers (log₂FC<0). In these individual validations, the 2 non-packagers yielded >10-fold lower titer than those identified as packagers, thus providing confidence in the AAV packaging metric. Consistent with their disruption of the AAV capsid structure there was also a depletion of non-functional stop codon control AAV variants in the capsid pool, and importantly this confirmed lack of library cross-packaging during AAV production (FIG. 2C).

To better understand what features drove successful capsid formation, the biophysical characteristics of the inserted peptides that yielded AAV variants that packaged successfully were analyzed. It was found that peptide charge, flexibility, alpha helical content, and hydrophobicity were all significantly different in packaging AAV variants versus AAV variants unable to package (FIG. 2d). The set of successfully packaged variants had a narrower charge distribution than the variants unable to package, suggesting peptides with extreme charge densities have a negative impact on capsid formation. Successfully packaging AAV variants also had inserted peptides with higher flexibility, lower alpha-helical content, and lower hydrophobicity than the variants unable to package. The observed depletion of hydrophobic peptide displaying variants is consistent with the solvent exposed nature of the AAV surface loops.

To build an integrated model predicting if AAV variants will package based on the biophysical features of the inserted peptides, a support vector machine classifier was trained using the charge, flexibility, alpha helical content, and hydrophobicity of the peptides in the dataset (FIG. 2e). While all of these biophysical features were significantly different when comparing packaging versus non-packaging AAV variants, the magnitude of this difference was relatively modest for each individual feature (FIG. 2d). However, collectively these features were sufficient to train a model which could differentiate between packaging and non-packaging AAV variants (area under the receiver operating characteristic curve=0.89). For each AAV variant, embedding the inserted peptide's charge, flexibility, alpha helix content, and hydrophobicity into two dimensions using uniform manifold approximation and projections (UMAP) enabled visualization of this class separability (FIG. 2f). The resulting embedding laid out AAV variants into distinct clusters, indicating that while each underlying biophysical feature is continuous, there are separable groups of AAV variants with similar biophysical features. AAV variants which package tended to cluster with other packaging variants in this unsupervised embedding, further supporting the predictive power of these four biophysical features. This thorough quantification and analytical framework for assessing AAV packaging, which is enabled by the diversity and length of the inserted peptide library, is of critical translational importance for identified AAV variants as AAV production costs are directly related to their packaging capability.

### High-Throughput Mapping of Engineered AAV Tissue Tropism

Having produced libraries of recombinant AAV particles packaging their own cap genes, next four viral pools were injected into C57BL/6 mice in duplicate (FIG. 3a). After two weeks, mice were sacrificed and the peptide-containing region of the AAV cap gene was amplified from DNA isolated from mouse liver, kidney, spleen, brain, lung, heart, skeletal muscle, intestine, and pancreas. The relative abundance of each AAV variant via NGS was quantified, and the log 2 enrichment calculated for each variant relative to its abundance in the capsid pool, observing good correlation between replicates (FIG. 3b). Using this data, over 15,000 variants were identified which efficiently infected at least one mouse tissue (FIG. 3c). The spleen and liver were the most frequent tissue targets of the infectious variants, consistent with the established wild-type (WT) tropism of AAV5 and AAV9 towards the liver, as well as more recent research showing AAV5 and AAV9 readily transduce the spleen. The fewest AAV variants targeting the skeletal muscle and brain were identified, in line with the high therapeutic AAV doses needed to achieve clinical efficacy for muscle targeting gene therapies, and the challenge of delivery across the blood-brain barrier. A substantial number of the AAV variants were comprised of the same peptide inserted across different AAV capsids and insertion sites, giving credence to the hypothesis that tropism reprogramming is, at least partially, peptide-specific (FIG. 3c).

Towards mapping and understanding the tissue-transduction patterns mediated by the inserted peptides, the feasibility of training predictive models linking inserted peptide sequence to tissue tropism were analyzed. Inspired by contemporary work using convolutional neural networks (CNN) to predict antibody specificity, a CNN multi-label classifier was trained to predict AAV tissue tropism using one-hot encoded inserted peptide sequences as input features (FIG. 3d). To evaluate performance, the model was trained using a random selection of 41 of the significantly enriched AAV variants, and evaluated on the b testing dataset. This CNN model architecture had good performance across all organs, with a minimum accuracy of 72% in the kidney (FIG. 3d). The highest F1 scores and Matthews Correlation Coefficients (MCC) were observed for the liver and spleen, likely due to the high number of liver and spleen targeting variants identified in the pooled screen (FIG. 3c). It was also observed that for the liver and spleen, the CNN had only modest reduction in F1 scores when evaluated on peptides very different (greater than 10 edits away) form any training examples, implying the models had learned generalizable features relevant to AAV transduction. For other organs, the models were much less accurate when predicting the tissue tropism of peptides very different from the training data, likely due to the reduced amount of positive training examples. Collectively, the ability to predict AAV tropism supports the conclusion that the inserted peptides are mediating retargeting of tissue-tropism, and that there is a learnable relationship between peptide sequence and tissue-tropism.

Engineered AAV Variants with Clinically Relevant Tissue Tropism

Like the WT scaffolds from which they were derived, transduction of multiple organs is a near ubiquitous phenotype among the infectious AAV variants identified (FIG. 4a). Significant transduction of the liver and spleen was observed for the majority of infectious variants regardless of which other organs were co-transduced. This is true even for variants with insertions in surface loops known to be involved in WT capsid receptor binding. While liver and spleen targeting were near ubiquitous, allowing for identifying variants which specifically target the liver/spleen plus one other organ, as well as variants which transduced all tissues at high levels (FIG. 4a). When variants were hierarchically clustered based on their tissue detection levels, variants derived from the same sub-library tended to cluster together, suggesting that the tissue specificity of the wild-type scaffold was at least partially a determinant of engineered variant tropism. Hierarchical clustering of the organ samples resulted in replicates clustering together, giving further confidence to the reliability of the screen results. To visualize the overall screen results, the tissue detection levels for each variant were embedded into two dimensions using UMAP, coloring the variants by the organ they most readily transduce (FIG. 4b). In this reduced dimensional space, organ specific clusters can be readily identified, with the liver and spleen targeting variants especially prominent.

To confirm the tissue tropism of the novel AAV variants that were identified via the pooled screen, 21 variants were individually produced and individually validated by quantifying their ability to package and deliver an mCherry transgene in vivo (FIG. 4c). All 21 variants were significantly enriched in at least one organ, and variants for validation which were internally consistent within the screening data were prioritized: consistent AAV capsids being defined as hits where other variants with similar inserted peptides enriching in the same organ were identified. Variants were initially characterized by in vivo quantification of tissue tropism at the mRNA level (via RT-qPCR quantification of mCherry transgene expression) (FIG. 4c). The tissue tropism of the variants largely recapitulated the screen predictions (FIG. 4c), with 74.3% of the tissue tropism predictions matching expectations. AAV variants which specifically targeted hard to infect organs such as the muscle, lung, and brain, while simultaneously de-targeting away from the liver were identified (FIG. 4c). Notably, protein level quantification of mCherry delivery to the liver, quantified via fluorescent microscopy, confirmed excellent concordance between mRNA and protein measures of tissue transduction (R²=0.97) (FIG. 4d). Altogether, across the variants individually tested, 9/21 variants were found to exceed AAV9 infectivity in at least one organ. Variants were identified which exceed AAV9 infectivity in all organs except the liver and pancreas, which had max relative transduction of 98.8% of WT AAV9 and 82.2% of WT AAV9, respectively. Additionally, 18/21 variants had less than half the liver transduction of WT AAV9, with three variants below 5% AAV9 liver transduction levels (FIG. 4d) indicating clinically important liver de-targeting. To increase confidence in the fidelity of the individually validated variants in C67BL/6 mice, 3 of the above 21 variants were individually validated in BALB/c mice. Notably, their relative tropism was consistent across the two mouse strains.

Mechanistic Insights into AAV Reprogramming

Having confirmed the efficacy of the AAV variants, experiments were performed to explore the mechanisms underlying their reprogrammed tropism, and in particular, investigate whether the hypothesis that AAV variants displaying peptides derived from distinct ligand domains could indeed drive their transduction patterns. First, experiments were performed to interrogate how peptides derived from specific ligand protein regions alter AAV tropism. This was motivated by observation that tiled peptide enrichment patterns in a screen can yield insight into functional domains of the proteins from which they were derived. Analysis of how the peptides were derived was performed. To do this, experiments were performed to interrogate tissue-specific AAV variants identified in the screen, focusing first on a lung-specific variant (termed AAV9.DKK1) that was derived via display of a peptide fragment form the DKK gene on the AAV9-Loop2 scaffold. Specifically, as shown in FIG. 5a (top left), it was observed that significantly enhanced lung transduction from peptides derived from a specific domain in the N-terminus of the protein. Upon confirming that this variant is packaged into a functional capsid via electron microscopy (FIG. 5a, top right), next experiments quantified its transduction profile in vivo. This confirmed that AAV9.DKK1 was indeed an efficient lung transducing variant with greater than 2-3 fold higher expression than AAV9, and with consistent de-targeting across all other organs compared to AAV9 when quantified at both the RNA and protein level (FIG. 5a, bottom left and right).

Next, experiments were conducted on the DKK1 displayed peptide to determine insight into the mechanism of transduction. Interestingly, the N-termini region of lung enrichment (FIG. 5a, top left) was centered on an evolutionarily conserved, linear peptide motif mediating binding to the low density lipoprotein receptor-related proteins 5 and 6 (LRP5/6). As HEK293T cells robustly express LRP6, thus lentiviral-mediated CRISPR-Cas9 was utilized to disrupt LRP6 expression in these cells to investigate the relative transducability of the AAV9.DKK1 variant in this system (FIG. 5b). Specifically, cells transduced with either non-targeting control (NTC) guides, or LRP6 targeting guides were subsequently transduced with either AAV9.DKK1 or wild-type AAV9. Using flow cytometry to quantify AAV delivery of the mCherry transgene, it was observed that a significant reduction in the infectivity of AAV9.DKK1 in the LRP6 knockout population, with no corresponding reduction in the infectivity of wild-type AAV9 (FIG. 5b). The LRP6 dependent in vitro infectivity of AAV9.DKK1, combined with the known interaction between LRP6 and the N-termini of DKK1 (see crystal structure, FIG. 5b), and its known robust expression in lung alveolar cells, to together gives strong support to the hypothesis that LRP6 mediates, at least in part, the reprogrammed tropism of AAV9.DKK1.

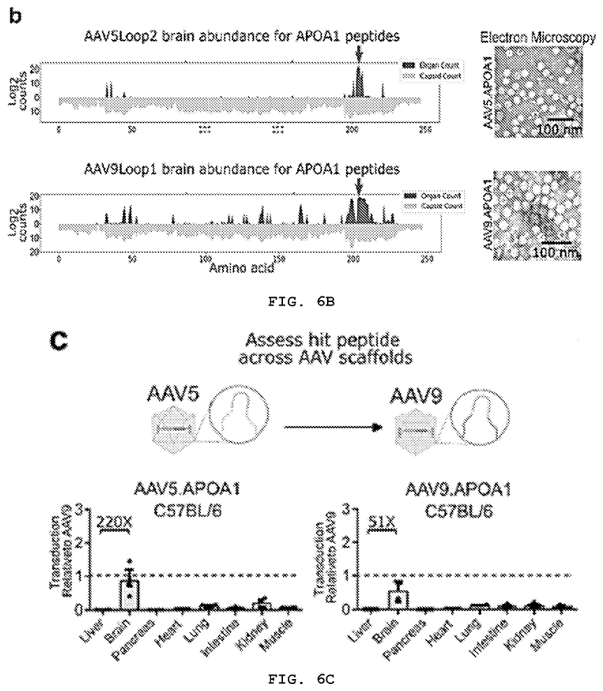

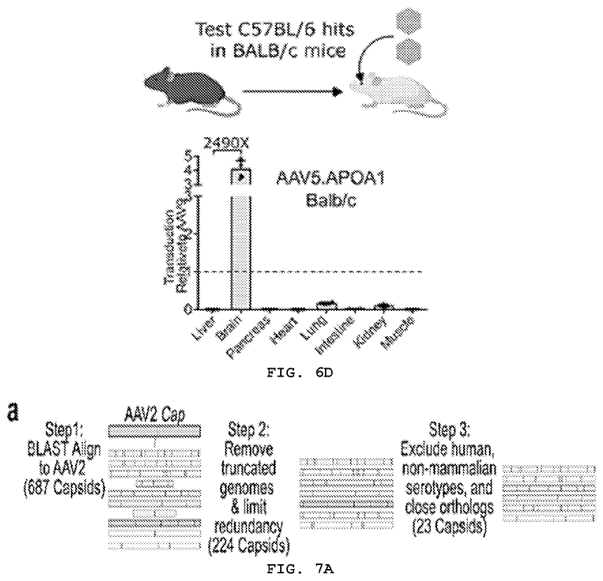

Building on these observations, and focusing on the brain as an exemplar, experiments were performed to examine more broadly the extent to which the inserted peptides were mediating re-targeting. First, a distance matrix quantifying the similarities between all peptide hits identified as significantly enriched in at least one organ was generated (FIG. 6a). By projecting this distance matrix into two dimensions, allowed for visualization of distinct clusters of homologous inserted peptides which infect the brain. It was found that families of similar inserted peptides yielded similar transduction rates (FIG. 6a). This effect was not limited to a particular capsid or insertion site, insofar as inserted peptides were often functional across all tested capsids and insertion sites (FIG. 6a). The observation that inserted peptides yielded consistent phenotypes across multiple capsids suggests that re-targeting is directly due to a peptide mediated mechanism. This result also highlights the power of peptide tiling library designs, insofar as having multiple overlapping peptides with similar sequences can function as internal controls, imparting confidence in the identification of particular AAV variant hits.

Experiments were performed to specifically interrogated two brain targeting variants (AAV5.APOA1 and AAV9.APOA1) which contained the same inserted apolipoprotein-A 1 (APOA1) derived peptide. Both variants were confirmed to package into functional capsids by electron microscopy (FIG. 6b). Across the entire APOA1 protein, functional APOA1-derived peptides were primarily from a tandem repeat region at the C-terminus of the protein (FIG. 6b). Highlighting the peptide-specific effect of altered AAV tropism, it was observed that strong detargeting away from the liver at less than 2% of AAV9 levels for both variants, with unperturbed brain transduction relative to wild-type AAV9 (FIG. 6c). This was especially remarkable for AAV5.APOA1 as the engineered variant was able to overcome the limited transduction of its parental AAV serotype across the blood-brain barrier. AAV5.APOA1's improved specificity was subsequently shown to be strain independent as it retained both its brain targeting and liver detargeting functionality in BALB/c mice (FIG. 6d).

### Engineering Re-Targeted Immune Orthogonal AAV Vectors

To generate additional variants with clinically relevant features, the AAV engineering approach was expanded to include novel capsids beyond AAV5 and AAV9. In particular, the potential of engineering tropism of highly divergent AAV orthologs was explored. This was motivated by the fact that one of the biggest challenges with AAV therapy is the associated immune responses: either, pre-existing immunity that currently excludes a significant fraction of the human population from being eligible for any AAV therapy; or induced immunity upon AAV injection which prevents subsequent AAV re-dosing. This makes it extremely difficult to create effective therapies since it only allows a single opportunity to treat a patient, thus often mandating extremely high titers for a one-time therapy which in turn can lead to dangerous levels of toxicity. Recently, the concept of immune orthogonality to address this challenge has been performed. Specifically, it has been suggested that an orthologue, given sufficient sequence divergence, will not cross-react with the immune response generated by exposure to other orthologues, thereby allowing re-dosing that avoids neutralization by existing antibodies or clearance of treated cells by activated cytotoxic T cells. While in that study experiments focused on commonly used AAV orthologs, experiments were performed to explore highly divergent natural orthologs from the full spectrum of mammalian species, including those which have not yet been thoroughly tested for use as in vivo vectors. Towards this a computational experimental pipeline was established to identify novel immune-orthogonal AAV serotypes. First, a basic local alignment search tool (BLAST) was used to identify 687 capsids with sequence homology to the AAV2 cap gene. This initial list was then filtered to exclude truncated genomes, redundant samples, human and non-mammalian serotypes, as well as close orthologs, resulting in 23 AAV capsid sequences for experimental investigation (FIG. 7a). Analyzing the evolutionary distance of these potential AAV orthologs, it was confirmed that they exhibit high dissimilarity from all wild-type AAV capsids (except AAV5 which had previously been shown to be immune-orthogonal), with many exhibiting less than 60% sequence similarity to AAV2 (FIG. 7b). To investigate if these computationally predicted AAV orthologs were functional, their ability to package into capsids was examined. Comparing titers to AAV5, 11 of the 23 AAV capsids were identified with packaging titers within 10-fold of AAV5 (FIG. 7c). Next, to evaluate in vivo transduction potential, these 11 AAV capsids carrying a mCherry transgene were administered individually to C57BL/6 mice. Given that most AAV capsids have at least a degree of liver transducing potential, the livers of these mice were harvested 3 weeks post infection. Of the 11 AAV capsids analyzed, 4 of them had detectable levels of mCherry in the liver with 2 of them generating 3-fold higher mCherry expression than AAV5 (FIG. 7c).

Having identified 4 functional AAV capsids, their immune-orthogonality was characterized by injecting C57BL/6 mice with each of these and collecting serum at day 0 and day 21 timepoints (FIG. 7d). AAV8 was selected for comparison in this study due to its propensity for transducing the liver and its similar serological profile to other wild-type serotypes. Performing an ELISA at the 3-week timepoint confirmed that antibodies developed against the 4 studied immune-orthogonal AAVs indeed exhibited no immune cross-reactivity with AAV8 (FIG. 7d). This result highlights the utility in combining computational and experimental methods to identify and characterize novel AAV serotypes which could be repurposed to enable gene therapy redosing.

Next experiments were performed to assess whether the tropism of the mined AAV immune-orthogonals could be re-engineered through insertion of one of the screen identified hit peptides. The muscle targeting AAV variants had broader tissue-tropism than the brain and lung targeting variants, insofar as they also readily infected the heart, lung, intestine, and spleen at level comparable to, or exceeding, AAV9, it was hypothesized that grafting the peptide from one of these variants onto an AAV ortholog would result in enhanced muscle targeting with minimal effects on parental AAV infectivity. To assess this AAV9.PDGFC was selected as a candidate variant, and confirmed its ability to form functional capsids (FIG. 6d) and enable efficient targeting of muscular tissue (with >10 fold expression above AAV9 as quantified by RT-qPCR) (FIG. 6d). The skeletal muscle and cardiac muscle (heart) transduction enhancement relative to AAV9 was also confirmed by protein level visualization (FIG. 6d). Grafts where then performed of the 20-mer PDGFC derived peptide onto one of the surface loops of the immune orthogonal AAV MGS (FIG. 7e), and notably observed similar enhanced muscle transduction of the engineered variant above its parental AAV (FIG. 7e). Taken together, the result highlights the fidelity of the ligand tiling display approach and further lends credence to the exciting possibility that displayed peptides identified in the initial screening approach could be utilized to re-engineer a diverse clade of AAV serotypes via peptide transfer.

It will be understood that various modifications may be made without departing from the spirit and scope of this disclosure. Accordingly, other embodiments are within the scope of the following claims.

What is claimed is:

1. A method of improving tropism of a virus or other delivery agent, the method comprising identifying ligand protein sequences derived from all known receptor-interacting ligands; systematically tile the ligand peptides into 5-50 or 10-20 or 20 amino acid peptides which are inserted into surface-exposed loops of AAV capsids; assessing the engineered capsids for their packaging capacity, in vivo tropism, and enhanced protein interactions.

2. The method of claim 1, wherein the virus is an adeno-associated virus (AAV).

3. The method of claim 2, wherein the AAV is selected from the group consisting of AAV1, AAV2, AAV5, AAV6, AAV7, AAV8, and AAV9.

4. The method of claim 3, wherein the AAV is AAV5 or AAV9.

5. The method of claim 3, wherein peptide sequences were generated via pooled oligonucleotide synthesis and inserted into 4 distinct loop regions: AAV5-Loop1 (N443), AAV5-Loop2 (S576), AAV9-Loop1 (Q456), and AAV9-Loop2 (A587) to generate over 1 million AAV variants.

6. The method of claim 3, wherein a 20'mer ligand peptide is inserted into one or both of 2 surface-exposed loops of AAV5 (SEQ ID NO: 2) or AAV9 (SEQ ID NO: 4).

7. A recombinant vector comprising capsid proteins containing one or two peptides inserted into one or both of two surface-exposed loops in the capsid protein, wherein the vector has a desired tropism or immune-orthogonality and wherein the peptides are independently selected from the group consisting of SEQ ID NOs:5 to 820 and 870-911.

8. The recombinant vector of claim 7, wherein the vector is an adeno-associated virus (AAV).

9. The recombinant vector of claim 8, wherein the AAV vector is an AAV5 serotype.

10. The recombinant vector of claim 9, wherein the AAV5 comprises a capsid protein having a sequence as set forth in SEQ ID NO:2.

11. The recombinant vector of claim 8, wherein the AAV vector is an AAV9 serotype.

12. The recombinant vector of claim 11, wherein the AAV9 comprises a capsid protein having a sequence as set forth in SEQ ID NO: 4.

13. The recombinant vector of claim 7, wherein the vector has tropism to pancreas, heart, brain, lung, liver, kidney, muscle, spleen or intestine.

14. The recombinant vector of claim 10 or 12, wherein a peptide of SEQ ID NOs:530-820, 870-910 or 911 is inserted into loop 1 and/or loop 2 of an AAV5 capsid or an AAV9 capsid.

15. The recombinant vector of claim 7, wherein the vector is immune orthogonal.

16. The recombinant vector of claim 15, wherein the vector is an adeno-associated virus (AAV).

17. The recombinant vector of claim 16, wherein the AAV comprises a capsid protein of any one of SEQ ID NOs:822, 824, 826, 828, 830, 832, 834, 836, 838, 840, 842, 844, 846, 848, 850, 852, 854, 856, 858, 860, 862, 864, or 866, or a sequence that it at least 85% to 99% identical to any of the foregoing sequences.

18. A method of making a delivery vehicle or vector with a desired tropism, the method comprising selecting a peptide sequence from any one of SEQ ID NOs: 5-820 or 870-911 and (i) cloning a nucleic acid sequence encoding the peptide into a coding sequence for a capsid protein at an exposed loop site to obtain a recombinant capsid coding sequence and producing the vector using the recombinant capsid coding sequence or (ii) inserting the peptide into an exposed surface of the delivery vehicle.

19. The method of claim 18, wherein the vector is an adeno-associated virus (AAV).

20. The method of claim 19, wherein the AAV is selected from the group consisting of AAV1, AAV2, AAV5, AAV6, AAV7, AAV8, and AAV9

21. The method of claim 20, wherein the AAV is AAV5 or AAV9.

22. The method of claim 21, wherein the AAV5 capsid coding sequence comprises SEQ ID NO:1.

23. The method of claim 21, wherein the AAV9 capsid coding sequence comprises SEQ ID NO:2.

24. An AAV vector wherein the capsid protein has been modified according to the method of claim 18.

25. An AAV vector comprising a capsid protein wherein the capsid protein expresses a peptide of any one of SEQ ID NO: 5-820 or 870-911 in surface exposed loop 1 and/or loop 2 of the capsid protein.

26. The AAV vector of claim 25, wherein the wild-type capsid protein sequence comprises SEQ ID NO:2 or 4.

27. The AAV vector of claim 25, wherein the AAV vector has a AAV5 or AAV9 serotype.

28. The AAV vector of claim 25, wherein the vector has a desired tropism.

29. The AAV vector of claim 28, wherein the AAV vector has a tropism to pancreas, heart, brain, lung, liver, kidney, muscle, spleen or intestine.

30. A viral vector having a capsid protein comprising a heterologous targeting peptide in a range of 10-30 amino acids in length inserted into a surface exposed portion of a capsid protein and wherein the targeting peptide is set forth in any one of SEQ ID NOs: 5-820 or 870-911.

31. The viral vector of claim 30, wherein the heterologous targeting peptide is about 15-25 amino acids in length.

32. The viral vector of claim 31, wherein the heterologous targeting peptide is about 20 amino acids in length.

33. The viral vector of claim 30, wherein the viral vector is an adeno-associated virus (AAV).

34. The viral vector of claim 30, wherein the viral vector is a lentiviral vector.

35. The viral vector of claim 30, wherein the capsid protein is a VP1 capsid protein.

36. The viral vector of claim 30, wherein the capsid protein is a VP2 capsid protein.

37. The viral vector of claim 30, wherein the capsid protein is a VP3 capsid protein.

38. The viral vector of claim 30, wherein the heterologous targeting peptide is inserted into an AAV capsid protein at loop 1 and/or loop 2.

39. The viral vector of claim 30, wherein the viral vector is an AAV5.

40. The viral vector of claim 30, wherein the viral vector is an AAV9.

41. The viral vector of claim 39, wherein the heterologous targeting peptide is flanked by a linker peptide at the N-terminal and C-terminal ends of the heterologous targeting peptide.

42. The viral vector of claim 40, wherein the heterologous targeting peptide is flanked by a linker peptide at the N-terminal and C-terminal ends of the heterologous targeting peptide.

43. The viral vector of claim 30, wherein the heterologous targeting peptide targets the viral vector to hepatocytes or liver tissue.

44. The viral vector of claim 30, wherein the heterologous targeting peptide targets the viral vector to neuronal cells or brain tissue.

45. The viral vector of claim 30, wherein the heterologous targeting peptide targets the viral vector to pancreatic cells or pancreas tissue.

46. The viral vector of claim 30, wherein the heterologous targeting peptide targets the viral vector to cardiac cells or heart tissue.

47. The viral vector of claim 30, wherein the heterologous targeting peptide targets the viral vector to lung tissue.

48. The viral vector of claim 30, wherein the heterologous targeting peptide targets the viral vector to intestinal tissue.

49. The viral vector of claim 30, wherein the heterologous targeting peptide targets the viral vector to spleen tissue.

50. The viral vector of claim 30, wherein the heterologous targeting peptide targets the viral vector to renal cells or kidney tissue.

51. The viral vector of claim 30, wherein the heterologous targeting peptide targets the viral vector to muscle cells or tissue.

52. An adeno-associated virus (AAV) capsid protein comprising a heterologous targeting peptide cloned into loop 1 and/or loop 2 of the capsid protein, wherein the heterologous targeting peptide is about 10-30 amino acids in length and is contained or comprises any one of the peptides of SEQ ID NO:5-820 or 870-911.

53. The AAV capsid protein of claim 52, wherein the capsid protein is a VP1 capsid protein.

54. The AAV capsid protein of claim 52, wherein the capsid protein is a VP2 capsid protein.

55. The AAV capsid protein of claim 52, wherein the capsid protein is a VP3 capsid protein.

56. The AAV capsid protein of claim 52, wherein the heterologous targeting peptide is about 15-25 amino acids in length.

57. The AAV capsid protein of claim 52, wherein the heterologous targeting peptide is about 20 amino acids in length.

58. The AAV capsid protein of claim 52, wherein the heterologous targeting peptide is flanked by a linker peptide at the N-terminal and C-terminal ends of the heterologous targeting peptide.

59. The AAV capsid protein of claim 52, wherein the heterologous targeting peptide targets hepatocytes or liver tissue.

60. The AAV capsid protein of claim 52, wherein the heterologous targeting peptide targets neuronal cells or brain tissue.

61. The AAV capsid protein of claim 52, wherein the heterologous targeting peptide targets pancreatic cells or pancreas tissue.

62. The AAV capsid protein of claim 52, wherein the heterologous targeting peptide targets cardiac cells or heart tissue.

63. The AAV capsid protein of claim 52, wherein the heterologous targeting peptide targets lung tissue.

64. The AAV capsid protein of claim 52, wherein the heterologous targeting peptide targets intestinal tissue.

65. The AAV capsid protein of claim 52, wherein the heterologous targeting peptide targets spleen tissue.

66. The AAV capsid protein of claim 52, wherein the heterologous targeting peptide targets renal cells or kidney tissue.

67. The AAV capsid protein of claim 52, wherein the heterologous targeting peptide targets muscle cells or tissue.

68. A recombinant AAV (rAAV) comprising a capsid protein of any one of claims 52-67.

69. A recombinant AAV (rAAV) comprising a capsid protein having a targeting peptide in loop 1 and/or loop 2 wherein the targeting peptide is independently selected from SEQ ID Nos: 5-820 or 870-911.

70. The recombinant AAV of claim 69, wherein the recombinant AAV further comprises a heterologous polynucleotide for gene delivery.

71. The recombinant AAV of claim 70, wherein the heterologous polynucleotide is a therapeutic gene.

72. A composition comprising the recombinant rAAV of any one of claims 69-71.

73. The composition of claim 72 further comprising a pharmaceutically acceptable carrier.

73. A method for delivering a transgene to a subject comprising: administering a recombinant AAV (rAAV) to a subject, wherein the rAAV comprises:
(i) a capsid protein of any one of claims 52-67, and
(ii) at least one transgene, and wherein the rAAV infects cells of a target tissue of the subject.

74. The method of claim 73, wherein the at least one transgene encodes a protein.

75. The method of claim 74, wherein the protein is an immunoglobulin heavy chain or light chain or fragment thereof.

76. The method of claim 73, wherein the at least one transgene encodes a small interfering nucleic acid.

77. The method of claim 76, wherein the small interfering nucleic acid is a miRNA.

78. The method of claim 76, wherein the small interfering nucleic acid is a miRNA sponge or TuD RNA that inhibits the activity of at least one miRNA in the subject or animal.

79. The method of claim 77, wherein the miRNA is expressed in a cell of the target tissue.

80. The method of claim of claim 73, wherein the target tissue is skeletal muscle, heart, liver, pancreas, brain or lung.

81. The method of claim 73, wherein the transgene expresses a transcript that comprises at least one binding site for a miRNA, wherein the miRNA inhibits activity of the transgene, in a tissue other than the target tissue, by hybridizing to the binding site.

82. The method of claim 73, wherein the at least one transgene encodes a gene product that mediates genome editing.

83. The method of claim 73, wherein the transgene comprises a tissue specific promoter or inducible promoter.

84. The method of claim 83, wherein the tissue specific promoter is a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter.

85. The method of claim 73, wherein the rAAV is administered intravenously, intravascularly, transdermally, intraocularly, intrathecally, orally, intramuscularly, subcutaneously, intranasally, or by inhalation.

86. The method of claim 73, wherein the subject is selected from a mouse, a rat, a rabbit, a dog, a cat, a sheep, a pig, and a non-human primate.

87. The method of claim 73, wherein the subject is a human.

88. An isolated nucleic acid encoding an AAV capsid protein containing an amino acid sequence selected from the group consisting of SEQ ID No: 5-820 and 870-911.

89. A delivery vehicle for delivery of a small molecule drug or biological agent having a desired tropism, wherein the delivery vehicle comprises a peptide or peptide fragment of at least 10-20 amino acids of any one of SEQ ID NOs:5-820 or 870-911.

90. The delivery vehicle of claim 89, wherein the delivery vehicle is selected from the group consisting of a liposome, a nanoparticle, a bacteria, a bacteriophage, a virus-like particle (VLP), a erythrocyte ghost, and an exosome.

91. The delivery vehicle of claim 89, wherein the biological agent comprises an siRNA, an antisense molecule, a protein or polypeptide, insulin, a vaccine, or an antibody.

92. The delivery vehicle of claim 89, wherein the small molecule drug comprises a chemotherapeutic agent, an anti-inflammatory, a steroid, and an antibiotic.

93. A biological agent having a desired tropism, the biological agent linked to a peptide or peptide fragment of at least 10-20 amino acids of any one of SEQ ID NOs:5-820 or 870-911.

94. The biological agent of claim 93, wherein the biological agent is a nucleic acid, a protein, a polypeptide, a peptide, an antibody, an antibody fragment, a non-immunoglobulin binding agent, or an enzyme.
